SEQUENCE LISTING (290 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 16 aa, synthetic construct
AKDRLSITIRPRYYGL

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 39 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 40 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 45 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 46 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 51 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 52 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 53 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 57 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 58 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 62 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 63 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 64 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 65 - protein, 5 aa, synthetic construct
RNAMF

SEQ ID NO: 66 - protein, 16 aa, synthetic construct
GIGTGGATSYADSVKG

SEQ ID NO: 67 - protein, 7 aa, synthetic construct
GRYYFDY

SEQ ID NO: 68 - protein, 12 aa, synthetic construct
RASQSVSSSYLA

SEQ ID NO: 69 - protein, 7 aa, synthetic construct
GASSRAT

SEQ ID NO: 70 - protein, 10 aa, synthetic construct
QQYDHSAGWT

SEQ ID NO: 71 - protein, 7 aa, synthetic construct
GASSRAP

SEQ ID NO: 72 - protein, 10 aa, synthetic construct
QQYGSSPPWT

SEQ ID NO: 73 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 74 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 77 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 78 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 79 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 80 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 81 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 82 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 83 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 84 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 85 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 89 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 90 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 91 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 93 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 95 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 96 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 97 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 98 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 99 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 100 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 101 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 102 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 103 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 104 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 105 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 106 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 107 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 108 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 109 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 110 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 111 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 112 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 113 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 116 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 117 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 118 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 119 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 120 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 121 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 122 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 123 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 124 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 125 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 126 - protein, 5 aa, synthetic construct
SYYMH

SEQ ID NO: 127 - protein, 17 aa, synthetic construct
IINPRGGSTSYAQKFQG

SEQ ID NO: 128 - protein, 7 aa, synthetic construct
GKYWMYD

SEQ ID NO: 129 - protein, 13 aa, synthetic construct
SGGGSSIGNSYVS

SEQ ID NO: 130 - protein, 7 aa, synthetic construct
DNNKRPS

SEQ ID NO: 131 - protein, 12 aa, synthetic construct
GTWDTSPVWEWP

SEQ ID NO: 132 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 133 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 134 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 135 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 136 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 137 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 138 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 139 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 140 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 141 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 142 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 143 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 144 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 145 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 146 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 147 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 148 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 149 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 150 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 151 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 152 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 153 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 154 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 155 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 156 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 157 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 158 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 159 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 160 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 161 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 162 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 163 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 164 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 165 - protein, 5 aa, synthetic construct
RHAMA

SEQ ID NO: 166 - protein, 17 aa, synthetic construct
AITSSGRSIYYADSVKG

SEQ ID NO: 167 - protein, 7 aa, synthetic construct
VHRAFDY

SEQ ID NO: 168 - protein, 13 aa, synthetic construct
SGSPLFPDSGSFN

SEQ ID NO: 169 - protein, 7 aa, synthetic construct
ADSHRPS

SEQ ID NO: 170 - protein, 11 aa, synthetic construct
GTWDYSLSGYV

SEQ ID NO: 171 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 172 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 173 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 174 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 175 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 176 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 177 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 178 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 179 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 180 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 181 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 182 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 183 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 184 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 185 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 186 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 187 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 188 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 189 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 190 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 191 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 192 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 193 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 194 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 195 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 196 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 197 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 198 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 199 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 200 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 201 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 202 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 203 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 204 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 205 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 206 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 207 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 208 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 209 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 210 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 211 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 212 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 213 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 214 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 215 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 216 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 217 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 218 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 219 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 220 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 221 - protein, 118 aa, synthetic construct
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 222 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 223 - protein, 121 aa, synthetic construct
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 224 - protein, 118 aa, synthetic construct
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 225 - protein, 118 aa, synthetic construct
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 226 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 227 - protein, 118 aa, synthetic construct
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 228 - protein, 118 aa, synthetic construct
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 229 - protein, 118 aa, synthetic construct
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 230 - protein, 118 aa, synthetic construct
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 231 - protein, 118 aa, synthetic construct
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 232 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 233 - protein, 118 aa, synthetic construct
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 234 - protein, 118 aa, synthetic construct
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 235 - protein, 107 aa, synthetic construct
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 236 - protein, 111 aa, synthetic construct
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 237 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 238 - protein, 111 aa, synthetic construct
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 239 - protein, 111 aa, synthetic construct
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 240 - protein, 111 aa, synthetic construct
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 241 - protein, 111 aa, synthetic construct
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 242 - protein, 107 aa, synthetic construct
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 243 - protein, 107 aa, synthetic construct
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 244 - protein, 107 aa, synthetic construct
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 245 - protein, 107 aa, synthetic construct
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 246 - protein, 107 aa, synthetic construct
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 247 - protein, 107 aa, synthetic construct
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 248 - protein, 107 aa, synthetic construct
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

SEQ ID NO: 249 - protein, 118 aa, synthetic construct
QVQLQQSGTELVKPGASVKMSCKASVNTFTGYNMHWIKQTPGQGLEWIGGLHPGNGDSSYNQKFKGRATLTDKSSNTAYMQLSSLTSEDSAVYYCALTTAGRAWFPYWGQGTLVTVSA

SEQ ID NO: 250 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEIK

SEQ ID NO: 251 - protein, 119 aa, synthetic construct
QVQLQQSGTELVKPGASVKMSCKASVNTFTGYNMHWIKQTPGQGLEWIGGLHPGNGDSSYNQKFKGRATLTADKSSNTAYMQLSSLTSEDSAVYYCALTTAGRAWFAYWGQGTLVTVSA

SEQ ID NO: 252 - protein, 106 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLHTFGGGTKLEIK

SEQ ID NO: 253 - protein, 119 aa, synthetic construct
QVQLQQSGAELVKPGASVKMSCKASVNTFAGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 254 - protein, 107 aa, synthetic construct
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 255 - protein, 119 aa, synthetic construct
QVQLQQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 256 - protein, 107 aa, synthetic construct
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLELK

SEQ ID NO: 257 - protein, 119 aa, synthetic construct
QVQLQQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFPYWGQGTLVTVSA

SEQ ID NO: 258 - protein, 107 aa, synthetic construct
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 259 - protein, 119 aa, synthetic construct
QVQLQQSGAELVKPGASVKMSCKASVNTFTGYNMHWIKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 260 - protein, 119 aa, synthetic construct
EVQLQQSGAELVKPGASVKMSCKASVNIFTGYNMHWVKQTPGQGLEWIGGLHPGNGDTSYNQKFKDKATLTADKSSNTAYMQLSSLTSEDSAVYYCALTTAGRAWFAYWGQGTLVTVSA

SEQ ID NO: 261 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 262 - protein, 119 aa, synthetic construct
EVKLVESGGGLVKPGGSLKLSCAASGFTFSRYAMSWVRQTPEKRLEWVATISSGGSYTNYADSVKGRFTISRDNVKNTLYLQMNSLRAEDTAVYYCARATARATEFAYWGQGTLVTVSS

SEQ ID NO: 263 - protein, 106 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEL

SEQ ID NO: 264 - protein, 107 aa, synthetic construct
DIQMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 265 - protein, 119 aa, synthetic construct
EVQLKESGAELVKPGASVKMSCKASVNIFTGYNMHWVKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 266 - protein, 119 aa, synthetic construct
QVQLKQSGAELVKPGASVKMSCTASVNIFTGYNMHWIKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 267 - protein, 107 aa, synthetic construct
DIQMNQSTSSLSASLGDRVTISCRASQDISNYLNWYQQKPDGTIKLLIYYTSRLYSGVPSRFSGSGSGTDYSLTISNLEEEDIATYFCQQGNTIPYTFGGGTKLEIK

SEQ ID NO: 268 - protein, 119 aa, synthetic construct
EVQLQQSGAELVKPGASVKMSCKASVNIFTGYNMHWVKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 269 - protein, 107 aa, synthetic construct
DIQMTQSTSSLSASLGDRVTISCRASQDISNYLNWYQQKPDGTIKLLIYYTSRLHSGVPSRFSGSGSGTDYSLTISNLEEEDIATYFCQQGNTLPWTFGGGTKLEIK

SEQ ID NO: 270 - protein, 119 aa, synthetic construct
EVQLQQSGAELVKPGASVKMSCKASVNIFTSYNMHWVKQTPGQGLEWIGGLHPGNGDTSYNQKFKGKATLTADKSSSTAYMQVSMLTSEDSAVYYCVLTTAGRAWFAYWGQGTLVTVSA

SEQ ID NO: 271 - protein, 119 aa, synthetic construct
QVQLQESGAELVKPGASVKMSCKASVNTFTGYNMHWVKQTPGQGLEWIGGLHPGNGDTSYNQKFKGKATLTADRSSNTAYMQLSSLTSEDSAVYYCALTTAGRAWFAYWGQGTLVTVSA

SEQ ID NO: 272 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEEEDIATYFCQQGNTLPWTFGGGTKLEIK

SEQ ID NO: 273 - protein, 119 aa, synthetic construct
QVQLKQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 274 - protein, 107 aa, synthetic construct
DIQMTQSTSSLSASLGDRVTISCRASQDISNYLNWYQQKPDGTVKLLIYYTSRLHSGVPSRFSGSGSGTDYSLTISNLEEEDIATYFCQQGNTLPWTFGGGTKLEIK

SEQ ID NO: 275 - protein, 119 aa, synthetic construct
QVQLKQSGAELVKPGASVKMSCKASVNTFTGYNMHWIKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 276 - protein, 107 aa, synthetic construct
DIQMNQSTSSLSASLGDRVTISCRASQDISNYLNWYQQKPDGTIKLLIYYTSRLYSGVPSRFSGSGSGTDYSLTISNLEEEDIATYFCQQGNTLPYTFGGGTKLEIK

SEQ ID NO: 277 - protein, 119 aa, synthetic construct
QVQLQQSGAELVKPGASVKMSCKASVNTITGYNMHWVKQTPGQGLEWIGGLHPGNGDTSYNQKFKDKATLTADKSSNTAYMQLSSLTSEDSAVYYCALTTAGRAWFAYWGQGTLVTVSA

SEQ ID NO: 278 - protein, 108 aa, synthetic construct
DIKMTQSTSSLSASLGDRVTTSCRASQDISNYLNWYQQKPDGTVKLLIYYTSRLHSGVPSRFSGSGSGTDYSLTISNLEEEDIATYFCQQGNTLPPWTFGGGTKLEIK

SEQ ID NO: 279 - protein, 119 aa, synthetic construct
EVQLVESGGGLVKPGGSLRLSCAASGFTFSRYAMSWVRQAPGKGLEWVSTISSGGSYTNYADSVKGRFTISRDNVKNTLYLQMNSLRAEDTAVYYCARATARATEFAYWGQGTLVTVSS

SEQ ID NO: 280 - protein, 119 aa, synthetic construct
EVQLLESGGGLVQPGGSLRLSCAASGFTFSRYAMSWVRQAPGKGLEWVSTISSGGSYTNYADSVKGRFTISRDNVKNTLYLQMNSLRAEDTAVYYCARATARATEFAYWGQGTLVTVSS

SEQ ID NO: 281 - protein, 119 aa, synthetic construct
QVQLVQSGAELKKPGASVKVSCKASGNTFTGYNMHWIQQSPGQGLEWMGGLHPGNGDSSYNQKFQGRVTLTADKSSNTAYMELSSLRSEDSAVYYCALTTAGRAWFPYWGQGTLVTVSS

SEQ ID NO: 282 - protein, 119 aa, synthetic construct
QVQLVQSGAEVKKPGASVKMSCKASVYTFTGYNMHWIQQSPGQGLEWMGGLHPGNGDSSYNQKFQGRATLTADKSSNTAYMELSSLRSEDSAVYYCALTTAGRAWFPYWGQGTLVTVSS

SEQ ID NO: 283 - protein, 119 aa, synthetic construct
QVQLVQSGAEVKKPGASVKVSCKASGNIFTGYNMHWVRQAPGQGLEWMGGLHPGNGDSSYNQKFQGRVTLTADKSSNTAYMELSSLRSEDSAVYYCALTTAGRAWFAYWGQGTLVTVSS

SEQ ID NO: 284 - protein, 119 aa, synthetic construct
QVQLVQSGAEVKKPGASVKMSCKASVYTFTGYNMHWVRQAPGQGLEWIGGLHPGNGDSSYNQKFQGRATLTADKSSNTAYMELSSLRSEDSAVYYCALTTAGRAWFAYWGQGTLVTVSS

SEQ ID NO: 285 - protein, 119 aa, synthetic construct
EVQLVQSGAEVKKPGATVKISCKASGNIFTSYNMHWVRQAPGQGLEWMGGLHPGNGDTSYNQKFQGRVTLTADKSSSTAYMELSSLRSEDTAVYYCVLTTAGRAWFAYWGQGTLVTVSS

SEQ ID NO: 286 - protein, 119 aa, synthetic construct
EVQLVQSGAEVKKPGATVKMSCKASVYTFTSYNMHWVRQAPGQGLEWIGGLHPGNGDTSYNQKFKGRATLTADKSSSTAYMELSSLRSEDTAVYYCVLTTAGRAWFAYWGQGTLVTVSS

SEQ ID NO: 287 - protein, 107 aa, synthetic construct
DIQMTQSPSSLSASVGDRVTITCQASQDISNYLNWYQQKPGKAPKLLIYYTSRLHSGVPSRFSGSGSGTDYTFTISSLQPEDIATYFCQQGNTLPWTFGGGTKVEIK

SEQ ID NO: 288 - protein, 107 aa, synthetic construct
DIQMTQSPSSLSASVGDRVTITCRASQDISNYLNWYQQKPGKAPKLLIYYTSRLHSGVPSRFSGSGSGTDYTLTISSLQPEDFATYFCQQGNTLPWTFGGGTKVEIK

SEQ ID NO: 289 - protein, 107 aa, synthetic construct
DIQMTQSPSSLSASVGDRVTITCQASQDISNYLNWYQQKPGKAPKLLIYYTSRLHSGVPSRFSGSGSGTDYTLTISSLQPEDFATYFCQQGNTLPLTFGGGTKVEIK

SEQ ID NO: 290 - protein, 107 aa, synthetic construct
DIQMTQSPSSLSASVGDRVTITCQASQDISNYLNWYQQKPGKAPKLLIYYTSRLHSGVPSRFSGSGSGTDYTLTISSLQPEDFATYFCQQGNTLPWTFGGGTKVEIK

(19) United States
(12) Patent Application Publication
(10) Pub. No.: US 2024/0360232 A1
(43) Pub. Date: Oct. 31, 2024

(54) METHODS FOR TREATING CHRONIC OBSTRUCTIVE PULMONARY DISEASE (COPD) BY ADMINISTERING AN IL-4R ANTAGONIST

(71) Applicants: SANOFI BIOTECHNOLOGY, Gentilly (FR); REGENERON PHARMACEUTICALS, INC., Tarrytown, NY (US)

(72) Inventors: Raolat Abdulai, Gentilly (FR); Elizabeth Laws, Gentilly (FR); Leda Mannent, Chilly-Mazarin (FR); Lacey Robinson, Gentilly (FR); Marcella Ruddy, Tarrytown, NY (US)

(21) Appl. No.: 18/612,039

(22) Filed: Mar. 21, 2024

Related U.S. Application Data
(60) Provisional application No. 63/551,557, filed on Feb. 9, 2024.
(60) Provisional application No. 63/602,616, filed on Nov. 26, 2023.
(60) Provisional application No. 63/525,197, filed on Jul. 6, 2023.
(60) Provisional application No. 63/460,981, filed on Apr. 21, 2023.
(60) Provisional application No. 63/457,188, filed on Apr. 5, 2023.
(60) Provisional application No. 63/453,786, filed on Mar. 22, 2023.

(30) Foreign Application Priority Data
Jan. 12, 2024 (EP) 24315012.5
Feb. 14, 2024 (EP) 24315047.1

(51) Int. Cl. C07K 16/28; A61K 39/00; A61K 45/06; A61P 11/00

(52) U.S. Cl. CPC C07K 16/2866; A61K 45/06; A61P 11/00; A61K 2039/505; A61K 2039/545

(57) ABSTRACT

Methods for treating or preventing chronic obstructive pulmonary disease (COPD) in a subject are provided. Methods comprising administering to a subject in need thereof a therapeutic composition comprising an interleukin-4 receptor (IL-4R) antagonist, such as an anti-IL-4R antibody or antigen-binding fragment thereof, are provided.

### RELATED APPLICATIONS

This application claims priority to U.S. Provisional Patent Application Ser. Nos. 63/453,786, filed Mar. 22, 2023, 63/457,188, filed Apr. 5, 2023, 63/460,981, filed Apr. 21, 2023, 63/525,197, filed Jul. 6, 2023, 63/602,616, filed Nov. 26, 2023, and 63/551,557, filed Feb. 9, 2024; and to European Patent Application Nos. 24315012.5, filed Jan. 12, 2024, and 24315047.1, filed Feb. 14, 2024. The entire disclosure of each of these applications is hereby incorporated by reference in its entirety for all purposes.

### SEQUENCE LISTING

The instant application contains a Sequence Listing which has been submitted electronically in XML format and is hereby incorporated by reference in its entirety. Said XML file, created on Jul. 8, 2024, is named 752189_SA9-342_ST26.xml and is 280,293 bytes in size.

### FIELD OF THE INVENTION

The disclosure relates to the treatment and/or prevention of chronic obstructive pulmonary disease (COPD) in a subject in need thereof. The disclosure relates to the administration of an interleukin-4 receptor (IL-4R) antagonist to treat or prevent COPD in a subject in need thereof.

### BACKGROUND

Chronic obstructive pulmonary disease (COPD) is a common, heterogeneous disease associated with an abnormal inflammatory immune response of the lung to noxious particles and gases. COPD results in progressive airflow obstruction that is mainly irreversible or only partially reversible, and in many cases loss of alveolar tissue and emphysema. The disease typically starts in small airways, where the chronic inflammation causes structural changes including narrowing of the small airways, and destruction of the lung parenchyma that leads to the loss of alveolar attachments to the small airways and decreases lung elastic recoil.

COPD is most commonly caused by smoking tobacco, but may also be caused by particulate and noxious gas inhalation through outdoor or indoor air pollution including occupational exposures to vapors, gas, dust (including silica) and fumes. The most common clinical symptoms include chronic dyspnea, cough, shortness of breath, and/or sputum production.

Chronic obstructive pulmonary disease is a highly prevalent, serious and progressive disease resulting in significant morbidity, mortality, and economic burden. Based on Burden of Obstructive Lung Disease (BOLD) and other large scale epidemiological studies, it is estimated that the number of COPD cases in the world was 384 million in 2010, with a global prevalence of 11.7% (95% confidence interval: 8.4%-15.0%). In the US alone there are more than 12 million diagnosed patients, and the incidence of COPD is expected to grow rapidly with an aging population. COPD is a progressive and irreversible inflammatory lung disease that is periodically punctuated by disease exacerbations that result in long term disability and mortality. In the US, COPD is the third most common cause of death.

The standard of care for moderate COPD starts with daily use of bronchodilators, mostly long-acting muscarinic antagonists (LAMA) and/or long-acting β2-agonists (LABA). As the disease progresses, in particular in patients with frequent exacerbations, bronchodilators are combined with anti-inflammatory drugs such as inhaled corticosteroids (ICS), and phosphodiesterase Type 4 (PDE-4) inhibitors (roflumilast). The major limitations of the existing agents for COPD include modest efficacy and for inhaled corticosteroids, an increased risk of respiratory infections including pneumonia, in particular at high doses of potent molecules and in patients with severe COPD. While inhaled corticosteroids have a consistent effect in reducing the risk of moderate COPD exacerbations defined by a COPD deterioration requiring the use of systemic corticosteroids and/or antibiotics, there is no consistent benefit on severe exacerbations requiring hospitalizations.

Systemic, mostly oral corticosteroids are largely reserved for the treatment of exacerbations given their unacceptable safety profile with heightened concerns for a COPD population. No approved therapeutic agent blocks the decline in FEV1 over time or modifies the progressive disease course of COPD.

Thus, significant unmet medical needs continue to exist in the growing population of patients with COPD. Accordingly, a need exists in the art for novel targeted therapies to further improve COPD symptoms, lung function, and prevent exacerbations.

### BRIEF SUMMARY OF THE INVENTION

In one aspect, the disclosure provides methods for treating a subject having chronic obstructive pulmonary disease (COPD). In some embodiments, the methods comprise administering to the subject an antibody or an antigen-binding fragment thereof that specifically binds interleukin-4 receptor (IL-4R).

In another aspect, the disclosure also provides methods for treating a subject having moderate-to-severe COPD. In some embodiments, the methods comprise administering to the subject an antibody or an antigen-binding fragment thereof that specifically binds interleukin-4 receptor (IL-4R).

In another aspect, the disclosure also provides methods for treating a subject having COPD with Type 2 inflammation. In some embodiments, the methods comprise administering to the subject an antibody or an antigen-binding fragment thereof that specifically binds interleukin-4 receptor (IL-4R).

In another aspect, the disclosure also provides methods for treating a subject having moderate-to-severe COPD with Type 2 inflammation. In some embodiments, the methods comprise administering to the subject an antibody or an antigen-binding fragment thereof that specifically binds interleukin-4 receptor (IL-4R).

In another aspect, the disclosure also provides methods for treating a subject having COPD not adequately controlled on background therapy. In some embodiments, the methods comprise administering to the subject an antibody or an antigen-binding fragment thereof that specifically binds interleukin-4 receptor (IL-4R).

In another aspect, the disclosure also provides methods for treating a subject having COPD with united airways disease (UAD). In some embodiments, the methods comprise administering to the subject an antibody or an antigen-binding fragment thereof that specifically binds interleukin-4 receptor (IL-4R).

In another aspect, the disclosure further provides methods for treating a subject having COPD, wherein the COPD is comorbid with at least one Type 2 inflammatory disease. In some embodiments, the methods comprise administering to the subject an antibody or an antigen-binding fragment thereof that specifically binds interleukin-4 receptor (IL-4R).

In another aspect, the disclosure further provides methods of improving one or more symptoms in a subject in need thereof. In some embodiments, the methods comprise administering to the subject an antibody or an antigen-binding fragment thereof that specifically binds interleukin-4 receptor (IL-4R).

In another aspect, the disclosure further provides methods of reducing or preventing moderate to severe exacerbations in a subject in need thereof. In some embodiments, the methods comprise administering to the subject an antibody or an antigen-binding fragment thereof that specifically binds interleukin-4 receptor (IL-4R).

In another aspect, the disclosure further provides methods of slowing the progression of lung function decline in a subject in need thereof. In some embodiments, the methods comprise administering to the subject an antibody or an antigen-binding fragment thereof that specifically binds interleukin-4 receptor (IL-4R).

In another aspect, the disclosure further provides methods of improving health-related quality of life in a subject in need thereof. In some embodiments, the methods comprise administering to the subject an antibody or an antigen-binding fragment thereof that specifically binds interleukin-4 receptor (IL-4R).

In another aspect, the disclosure further provides methods of delaying time to first moderate or severe exacerbation in a subject in need thereof. In some embodiments, the methods comprise administering to the subject an antibody or an antigen-binding fragment thereof that specifically binds interleukin-4 receptor (IL-4R).

In certain exemplary embodiments, the subject to be treated in the methods described herein has a baseline blood eosinophil count of ≥300 cells/μL, ≥350 cells/μL, ≥400 cells/μL, ≥450 cells/μL, or ≥500 cells/μL.

In certain exemplary embodiments, the subject to be treated in the methods described herein has a baseline blood eosinophil count of below 300 cells/μL.

In certain exemplary embodiments, the subject to be treated in the methods described herein has a baseline fractional exhaled nitric oxide (FeNO) level of ≥20 ppb, ≥25 ppb, ≥30 ppb, ≥35 ppb, or ≥40 ppb.

In certain exemplary embodiments, the subject to be treated in the methods described herein has a baseline fractional exhaled nitric oxide (FeNO) level of below 20 ppb.

In certain exemplary embodiments, the subject to be treated in the methods described herein has a baseline immunoglobulin E level (IgE) of ≥100 kU/L.

In certain exemplary embodiments, the subject to be treated in the methods described herein has a baseline immunoglobulin E level (IgE) below 100 kU/L.

In certain exemplary embodiments, the COPD of the methods described herein is oxygen-dependent COPD.

In certain exemplary embodiments, the COPD of the methods described herein is uncontrolled at baseline despite standard of care. In some embodiments, the standard of care comprises a combination of LABA+LAMA+ICS, or LABA+LAMA if ICS is contraindicated.

In certain exemplary embodiments, the subject to be treated in the methods described herein has acute or chronic bronchitis.

In certain exemplary embodiments, the subject to be treated in the methods described herein has emphysema.

In certain exemplary embodiments, the subject to be treated in the methods described herein has lung bulla.

In certain exemplary embodiments, the subject to be treated in the methods described herein has tracheobronchial COPD.

In certain exemplary embodiments, the subject to be treated in the methods described herein is a current smoker.

In certain exemplary embodiments, the subject to be treated in the methods described herein is a former smoker.

In certain exemplary embodiments, the subject to be treated in the methods described herein is a human being.

In certain exemplary embodiments, the subject to be treated to be treated in the methods described herein is an adult patient.

In certain exemplary embodiments, methods described herein further comprise a background therapy in addition to the antibody or an antigen-binding fragment thereof.

In certain exemplary embodiments, the background therapy comprises an inhaled corticosteroid (ICS), a long-acting beta-agonist (LABA), a leukotriene receptor antagonist (LTRA), a long-acting muscarinic antagonist (LAMA), a methylxanthine, an inhibitor of phosphodiesterase (e.g., roflumilast or theophylline), or a mixture thereof.

In certain exemplary embodiments, the background therapy comprises a LABA, a LAMA, and an ICS. In some embodiments, the COPD is uncontrolled at baseline despite the background therapy alone.

In certain exemplary embodiments, the background therapy comprises a high dose of ICS. In certain exemplary embodiments, the high dose ICS is beclometasone dipropionate (chlorofluorocarbon, CFC) and the dose is greater than 1000 mcg. In certain exemplary embodiments, the high dose ICS is beclometasone dipropionate (hydrofluoroalkane, HFA) and the dose is greater than 400 μg. In certain exemplary embodiments, the high dose ICS is budesonide (dry powder inhaler, DPI) and the dose is greater than 800 mcg. In certain exemplary embodiments, the high dose ICS is ciclesonide (HFA) and the dose is greater than 320 mcg. In certain exemplary embodiments, the high dose ICS is fluticasone propionate (DPI or HFA) and the dose is greater than 500 μg. In certain exemplary embodiments, the high dose ICS is mometasone furoate and the dose is greater than 440 μg. In certain exemplary embodiments, the high dose ICS is triamcinolone acetonide and the dose is greater than 2000 μg. In certain exemplary embodiments, the adult high dose of ICS for fluticasone propionate is >500 μg (DPI or HFA) or 401-800 μg (HFA) for Japanese population.

In certain exemplary embodiments, the background therapy comprises a non-high dose of ICS. In certain exemplary embodiments, the pharmaceutical composition comprising an IL-4R antagonist is administered with a non-high dose ICS, a LAMA, and a LABA. In certain exemplary embodiments, the non-high dose ICS is beclomethasone dipropionate or equivalent and the dose is below 1000 μg. In certain exemplary embodiments, the non-high dose ICS is fluticasone propionate (HFA) and the dose is below 400 μg. In certain exemplary embodiments, the non-high dose ICS is budesonide (DPI) and the dose is below 800 μg. In certain exemplary embodiments, the non-high dose ICS is ciclesonide (HFA) and the dose is below 320 μg. In certain exemplary embodiments, the high dose ICS is fluticasone propionate (DPI or HFA) and the dose is below 500 μg. In certain exemplary embodiments, the non-high dose ICS is mometasone furoate and the dose is below 440 μg. In certain exemplary embodiments, the non-high dose ICS is triamcinolone acetonide and the dose is below 2000 μg. In certain exemplary embodiments, the adult non-high dose of ICS for fluticasone propionate is no more than 500 μg (DPI or HFA) or no more than 400 μg (HFA) for a Japanese population.

In certain exemplary embodiments, the background therapy comprises a LABA and a LAMA. In certain exemplary embodiments, the COPD is uncontrolled at baseline despite the background therapy alone.

In certain exemplary embodiments, an inhaled corticosteroid (ICS) is contraindicated in the subject to be treated.

In certain exemplary embodiments, the background therapy comprises roflumilast.

In certain exemplary embodiments, the background therapy comprises theophylline.

In certain exemplary embodiments, one or more COPD-associated parameter(s) are improved in the subject, e.g., after treatment according to the methods described herein for a specified period of time (e.g., 12 weeks, 24 weeks, 36 weeks, 52 weeks, or longer). In certain exemplary embodiments, the one or more COPD-associated parameter(s) are selected from the group consisting of: (1) annualized rate of acute moderate or severe exacerbations of COPD (AECOPD), (2) annualized rate of severe AECOPD, (3) time to first moderate or severe AECOPD, (4) forced expiratory volume in 1 second (FEV1) (pre-bronchodilator or post-bronchodilator), (5) forced vital capacity (FVC), (6) forced expiratory flow (FEF) 25%-75%, (7) fractional exhaled nitric oxide (FeNO), (8) Exacerbations of Chronic Obstructive Pulmonary Disease Tool (EXACT), (9) St. George's Respiratory Questionnaire (SGRQ) score, (10) Evaluating Respiratory Symptoms in COPD (E-RS:COPD) score, (11) Body mass index, airflow Obstruction, Dyspnea, Exercise performance (BODE) index, (12) Euro Quality of Life-5 Dimension Questionnaire (EQ-5D) score, (13) Modified British Medical Research Council Questionnaire (mMRC) score, (14) Health-Related Quality of Life Questionnaire (HRQoL) score, (15) courses in days of steroids (e.g., systemic corticosteroid), (16) courses in days of an antibiotic, (17) resting respiratory rate, (18) FEV1/FVC ratio, (19) mucus plugging, (20) and any combination thereof.

In certain exemplary embodiments, methods described herein reduce the level of one or more biomarkers in the subject. In certain exemplary embodiments, the biomarker is selected from the group consisting of blood eosinophil (Eos) count, fractional exhaled nitric oxide (FeNO) level, immunoglobulin E level (IgE), eotaxin (e.g., eotaxin-3) level, pulmonary and activation-regulated chemokine (PARC) level.

In certain exemplary embodiments, the antibody or antigen-binding fragment thereof used in methods as described herein comprises three heavy chain CDR sequences comprising SEQ ID Nos: 3, 4, and 5, respectively, and three light chain CDR sequences comprising SEQ ID Nos: 6, 7, and 8, respectively. In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the antibody or antigen-binding fragment thereof is administered to the subject as an initial dose followed by one or more secondary doses.

In certain exemplary embodiments, the initial dose is about 300 mg and the one or more secondary doses are each about 300 mg.

In certain exemplary embodiments, the secondary doses are administered every other week (q2w) or using an alternative dosing regimen.

In certain exemplary embodiments, the subject is an adult. In certain exemplary embodiments, the subject is at least 40 years old.

In certain exemplary embodiments, prior to the start of treatment, the subject has a baseline Medical Research Council (MRC) Dyspnea Scale Grade score of ≥2. In some embodiments, prior to the start of treatment the subject has a baseline Modified Medical Research Council (mMRC) Dyspnea Scale Grade score of ≥2. mMRC is described in ATS (1982) Am Rev Respir Dis. No; 126(5):952-6.

In certain exemplary embodiments, the subject has a history of high exacerbation risk.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, a “subject in need thereof” is a subject who is a current smoker. In certain exemplary embodiments, the subject is a current smoker who smokes cigarettes. In certain exemplary embodiments, the subject is a current smoker who has a smoking history of smoking greater than or equal to 10 packs of cigarettes per year. In certain exemplary embodiments, the subject is a current smoker and has a smoking history of smoking fewer than 10 packs of cigarettes per year. In certain exemplary embodiments, the subject is a current smoker and has a smoking history of smoking more than 1, 5, 10, 15, 20, 25, 30, 35, 40, 45, 50 or more packs of cigarettes per year. In certain exemplary embodiments, the subject is a current smoker who has a smoking history of smoking for 6 months, 1 year, 2 years, 3 years, 5 years, 10 years or longer.

In certain exemplary embodiments, a “subject in need thereof” is a subject who is a former smoker. In certain exemplary embodiments, the subject is a former smoker who has a history of smoking cigarettes. In certain exemplary embodiments, the subject is a former smoker who has a smoking history of smoking greater than or equal to 10 packs of cigarettes per year. In certain exemplary embodiments, the subject is a former smoker who has a smoking history of smoking fewer than 10 packs per year. In certain exemplary embodiments, the subject is a former smoker who has a smoking history of smoking more than 1, 5, 10, 15, 20, 25, 30, 35, 40, 45, 50 or more packs of cigarettes per year. In certain exemplary embodiments, the subject is a former smoker who has a smoking history of smoking for 6 months, 1 year, 2 years, 3 years, 5 years, 10 years or longer.

In certain exemplary embodiments, the subject is a patient of GOLD 1 severity group (mild COPD, FEV1≥80% predicted) according to GOLD Grades. In certain exemplary embodiments, the subject is a patient of GOLD 2 severity group (moderate COPD, 50%≤FEV1<80% predicted) according to GOLD Grades. In certain exemplary embodiments, the subject is a patient of GOLD 3 severity group (severe COPD, 30%≤FEV1<50% predicted) according to GOLD Grades. In certain exemplary embodiments, the subject is a patient of GOLD 4 severity group (very severe COPD, FEV1<30% predicted) according to GOLD Grades.

In certain exemplary embodiments, the subject has (1) 0 or 1 moderate exacerbation annually not leading to hospital admission, and (2) a modified Medical Research Council (mMRC) score between 0 to 1, or a COPD assessment test (CAT) score less than 10. In certain exemplary embodiments, the subject has (1) 0 or 1 moderate exacerbation annually not leading to hospital admission, and (2) an mMRC score equal or greater than 2, or a CAT score equal or more than 10. In certain exemplary embodiments, the subject has (1) two or more moderate exacerbations annually not leading to hospital admission or at least one severe exacerbation annually leading to hospital admission, and (2) an mMRC score between 0 to 1, or a CAT score less than 10. In certain exemplary embodiments, the subject has (1) two or more moderate exacerbations annually not leading to hospital admission or at least one severe exacerbation annually leading to hospital admission, and (2) an mMRC score equal or greater than 2, or a CAT score equal or more than 10. In certain exemplary embodiments, the subject has (1) two or more moderate exacerbations annually not leading to hospital admission or at least one severe exacerbation annually leading to hospital admission, regardless the mMRC score or the CAT score. In certain exemplary embodiments, the subject has two or more severe exacerbations annually leading to hospital admission.

In another aspect, a method for treating a subject having uncontrolled chronic obstructive pulmonary disease (COPD) comprising administering to the subject an antibody or an antigen-binding fragment thereof that specifically binds interleukin-4 receptor (IL-4R) is provided.

In another aspect, the present disclosure provides a method of treating uncontrolled chronic obstructive pulmonary disease (COPD) with type 2 inflammation in a subject, comprising administering to the subject an antibody or an antigen-binding fragment thereof that specifically binds interleukin-4 receptor (IL-4R) as an add-on maintenance treatment in addition to a COPD background therapy. In certain exemplary embodiments, the uncontrolled COPD is associated with a history of exacerbations. In certain exemplary embodiments, the type 2 inflammation is guided by, identified by and/or determined by at least one biomarker (e.g., blood eosinophil level).

In another aspect, the present disclosure provides a method of decreasing moderate-to-severe exacerbations and/or improving at least one COPD-associated parameter selected from the group consisting of: (1) annualized rate of acute moderate or severe exacerbations of COPD (AECOPD); (2) annualized rate of severe AECOPD; (3) time to first moderate or severe AECOPD; (4) forced expiratory volume in 1 second (FEV1) (pre-bronchodilator or post-bronchodilator); (5) forced vital capacity (FVC); (6) forced expiratory flow (FEF) 25%-75%; (7) fractional exhaled nitric oxide (FeNO); (8) Exacerbations of Chronic Obstructive Pulmonary Disease Tool (EXACT); (9) St. George's Respiratory Questionnaire (SGRQ); (10) Evaluating Respiratory Symptoms in COPD (E-RS:COPD) or Rs-Total Score; (11) Body mass index, airflow Obstruction, Dyspnea, Exercise performance (BODE) index; (12) Euro Quality of Life-5 Dimension Questionnaire (EQ-5D); (13) Modified British Medical Research Council Questionnaire (mMRC); (14) Health-Related Quality of Life Questionnaire (HRQoL); (15) Courses in days of steroids (e.g., systemic corticosteroid); (16) Courses in days of an antibiotic; (17) resting respiratory rate, (18) FEV1/FVC ratio, (19) mucus plugging, or any combination thereof.

Also provided is a pharmaceutical composition comprising an antibody or an antigen-binding fragment thereof that specifically binds to interleukin-4 receptor (IL-4R) as an add-on maintenance treatment in addition to a COPD background therapy for use in treating uncontrolled chronic obstructive pulmonary disease (COPD) with type 2 inflammation in a subject. In certain exemplary embodiments, the uncontrolled COPD is associated with a history of exacerbations.

Also provided is a use of an antibody or an antigen-binding fragment thereof that specifically binds to interleukin-4 receptor (IL-4R) for the treatment of uncontrolled chronic obstructive pulmonary disease (COPD) with type 2 inflammation in a subject. In certain exemplary embodiments, the uncontrolled COPD is associated with a history of exacerbations.

Also provided is a combination therapy comprising an antibody or an antigen-binding fragment thereof that specifically binds to interleukin-4 receptor (IL-4R) and a COPD background therapy, for use in a method of reducing a subject's dependence on systemic steroid (e.g., corticosteroid, such as inhaled corticosteroid (ICS)) and/or LABA for the treatment of uncontrolled chronic obstructive pulmonary disease (COPD) with type 2 inflammation in the subject. In certain exemplary embodiments, the uncontrolled COPD is associated with a history of exacerbations.

Also provided is a kit comprising an antibody or an antigen-binding fragment thereof that specifically binds to interleukin-4 receptor (IL-4R) and one or more compounds for COPD background therapy. In certain exemplary embodiments, the kit is used in a treatment of uncontrolled chronic obstructive pulmonary disease (COPD) with type 2 inflammation in the subject. In certain exemplary embodiments, the uncontrolled COPD is associated with a history of exacerbations.

In certain exemplary embodiments of the methods, the pharmaceutical compositions, or the combination therapy described herein, the COPD background therapy is selected from the group consisting of (1) beta2-agonists, such as short-acting beta 2-agonists (SABA, e.g., fenoterol, levalbuterol, salbutamol, and terbutaline) or long-acting beta 2-agonists (LABA, e.g., arformoterol, formoterol, indacaterol, olodaterol, and salmeterol); (2) anticholinergics, such as short-acting anticholinergics (SAMA, e.g., ipratropium bromide, oxitropium bromide) or long-acting anticholinergics (LAMA, e.g., aclidinium bromide, glycopyrronium bromide, tiotropium, umeclidinium, glycopyrrolate, and revefenacin); (3) combinations of SABA and SAMA (e.g., fenoterol+ipratropium or salbutamol+ipratropium); (4) combinations of LABA and LAMA (formoterol+aclidinium, formoterol+glycopyrronium, indacaterol+glycopyrronium, vilanterol+umeclidinium, and olodaterol+tiotropium); (5) methylxanthines (e.g., aminophylline and theophylline); (6) combination of LABA and corticosteroid (e.g., formoterol+beclomethasone, formoterol+budesonide, formoterol+mometasone, salmeterol+fluticasone propionate, vilanterol+fluticasone furoate); (6) triple combination (e.g., LABA+LAMA+ICS, such as fluticasone+umeclidinium+vilanterol, beclomethasone+formoterol+glycopyrronium, budesodine+formoterol+glycopyrrolate); (7) phosphodiesterase inhibitors (e.g., phosphodiesterase-4 inhibitors, such as roflumilast); (8) mucolytic agents (e.g., erdosteine, catbocysterine, and N-acetylcysterine); or any combinations thereof. In certain exemplary embodiments, the background therapy is a triple therapy, such as a therapy comprising administering to the subject an inhaled corticosteroid (ICS), a long-acting beta-agonist (LABA), and a long-acting muscarinic antagonist (LAMA). In certain exemplary embodiments, the background therapy is a double therapy, such as a therapy comprising administering to the subject a long-acting beta-agonist (LABA) and a long-acting muscarinic antagonist (LAMA) if inhaled corticosteroids (ICS) are contraindicated.

In certain exemplary embodiments of the methods, the pharmaceutical compositions, or the combination therapy described herein, the subject is an adult.

In certain exemplary embodiments of the methods, the pharmaceutical compositions, or the combination therapy described herein, the subject receives triple therapy in addition to the antibody or antigen-binding fragment thereof. In certain exemplary embodiments, the triple therapy comprises treatment with an inhaled corticosteroid (ICS), a long-acting beta-agonist (LABA), and a long-acting muscarinic antagonist (LAMA).

In certain exemplary embodiments of the methods, the pharmaceutical compositions, or the combination therapy described herein, ICS is contraindicated and the subject receives double therapy in addition to the antibody or antigen-binding fragment thereof. In certain exemplary embodiments, the double therapy comprises LABA and LAMA.

In certain exemplary embodiments of the methods, the pharmaceutical compositions, or the combination therapy described herein, the antibody or an antigen-binding fragment thereof is an add-on treatment. In certain exemplary embodiments, the antibody or an antigen-binding fragment thereof is a maintenance treatment. In certain exemplary embodiments, the antibody or an antigen-binding fragment thereof is an add-on maintenance treatment.

In certain exemplary embodiments of the methods, the pharmaceutical compositions, or the combination therapy described herein, the subject has a history of COPD exacerbations.

In certain exemplary embodiments of the methods, the pharmaceutical compositions, or the combination therapy described herein, the subject has an elevated level of a biomarker of Type 2 inflammation relative to a control. In certain exemplary embodiments, the biomarker may be selected from the group consisting of pulmonary and activation-regulated chemokine (PARC), eotaxin-3, fibrinogen, IgE, sputum or blood eosinophils, sputum or blood neutrophils, fractional exhaled nitric oxide (FeNO), IL-4Rα, IL 4, IL-13, IL-33, serum periostin, calcium-activated chloride channel regulator (CLCA1), cystatin-SN (CST1), suppression of tumorigenicity 2 (ST2), thymic stromal lymphopoietin (TSLP), or any combination thereof. In certain exemplary embodiments, the biomarker is blood eosinophil count. In certain exemplary embodiments, the biomarker is FeNO.

In certain exemplary embodiments of the methods, the pharmaceutical compositions, or the combination therapy described herein, the subject has a baseline blood eosinophil count of ≥50 cells/μL, ≥100 cells/μL, ≥150 cells/μL, ≥200 cells/μL, ≥250 cells/μL, ≥300 cells/μL, ≥350 cells/μL, ≥400 cells/μL, ≥450 cells/μL, or ≥500 cells/μL. In certain exemplary embodiments, the subject has a baseline blood eosinophil count of below 300 cells/μL.

In certain exemplary embodiments of the methods, the pharmaceutical compositions, or the combination therapy described herein, the subject has a baseline FeNO level of ≥20 ppb, ≥22 ppb, ≥24 ppb, ≥26 ppb, ≥28 ppb, 30 ppb, ≥32 ppb, ≥34 ppb, ≥36 ppb, ≥38 ppb, 40 ppb, ≥42 ppb, ≥44 ppb, 46 ppb, 48 ppb, 50 ppb, or more. In certain exemplary embodiments, the subject has a baseline FeNO level of ≥20 ppb.

In certain exemplary embodiments of the methods, the pharmaceutical compositions, or the combination therapy described herein, the subject has a baseline serum IgE level ≥100 kU/L, ≥150 kU/L, ≥200 kU/L, ≥250 kU/L, ≥300 kU/L, ≥350 kU/L, ≥400 kU/L, ≥450 kU/L, ≥500 kU/L, ≥1000 kU/L, ≥1500 kU/L, ≥2000 kU/L, ≥2500 kU/L, ≥3000 kU/L, ≥3500 kU/L, ≥4000 kU/L, ≥4500 kU/L, ≥5000 kU/L, or more (e.g., as measured using the IMMUNOCAP® assay [Phadia, Inc. Portage, MI]).

In certain exemplary embodiments of the methods, the pharmaceutical compositions, or the combination therapy described herein, consistent benefits are observed in the subject irrespective of demographics, ICS dose (high/low), smoking status, GOLD severity of airflow limitation (baseline), and history of exacerbations.

In certain exemplary embodiments of the methods, the pharmaceutical compositions, or the combination therapy described herein, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments of the methods, the pharmaceutical compositions, or the combination therapy described herein, the antibody or antigen-binding fragment thereof is administered to the subject as an initial dose followed by one or more secondary doses.

In certain exemplary embodiments of the methods, the pharmaceutical compositions, or the combination therapy described herein, the initial dose is about 300 mg and the one or more secondary doses are each about 300 mg.

In certain exemplary embodiments of the methods, the pharmaceutical compositions, or the combination therapy described herein, the secondary doses are administered every other week (q2w) or using an alternative dosing regimen.

Also provided is a kit comprising an antibody or an antigen-binding fragment thereof that specifically binds to interleukin-4 receptor (IL-4R) and one or more compounds for COPD background therapy. In certain exemplary embodiments, the kit is used in a treatment of uncontrolled chronic obstructive pulmonary disease (COPD) with type 2 inflammation in the subject. In certain exemplary embodiments, the uncontrolled COPD is associated with a history of exacerbations.

### BRIEF DESCRIPTION OF THE FIGURES

The foregoing and other features and advantages of the disclosure will be more fully understood from the following detailed description of illustrative embodiments taken in conjunction with the accompanying drawings.

FIG. 1 schematically depicts the overview of the study design of the BOREAS and NOTUS studies of Example 1. These studies were multinational, randomized, double-blind, placebo-controlled, 52-week phase 3 studies to assess the efficacy, safety, and tolerability of dupilumab in patients with moderate-to-severe Type 2 inflammatory COPD, such as that driven by activation of IL-4, IL-5, and IL-13, on an established LABA, LAMA and/or ICS background therapy (triple therapy unless ICS was contraindicated). Study treatments were dupilumab 300 mg q2w or placebo q2w administered during the 52-week treatment period.

FIG. 2A-FIG. 2C depict a table of the schedule of activities for the two randomized, placebo-controlled studies to assess the efficacy, safety, and tolerability of dupilumab in patients with moderate-to-severe Type 2 inflammatory COPD (Example 1).

FIG. 3 depicts that dupilumab met all multiplicity adjusted endpoints with high statistical significance.

FIG. 4 depicts that dupilumab reduced rate of moderate and severe exacerbations in patients. Exacerbation reduction of 30% was statistically significant and clinically meaningful.

FIG. 5 depicts rapid and sustained dupilumab improvement in lung function. The data shows significant and clinically meaningful improvement in pre-BD FEV1 at Week 12 and Week 52.

FIG. 6 depicts higher efficacy observed in the subgroup of COPD patients who had a baseline FeNO level ≥20 ppb. Exacerbations and lung function improvements increase with markers of type 2 inflammation.

FIG. 7 depicts that dupilumab improved health status as measured by St. George Respiratory Questionnaire (SGRQ). Improvement in SGRQ was rapid and sustained through 52 weeks of treatment.

FIG. 8 depicts that dupilumab improved symptoms of COPD as measured by E-RS COPD:RS-Total Score. The improvement was rapid and sustained through 52 weeks.

FIG. 9 depicts higher efficacy observed in baseline FeNO ≥20 ppb subgroup. Exacerbations and lung function improvements increased with markers of type 2 inflammation.

FIG. 10 depicts consistent efficacy observed in smoker subgroups. Exacerbations and lung function improvements are similar in current smokers and former smokers.

FIG. 11 depicts that dupilumab decreased eosinophils and FeNO over time (after a transient increase).

FIG. 12 depicts dupilumab adverse event profile during the BOREAS trial.

FIG. 13A-FIG. 13E depict that baseline demographic characteristics were similar between dupilumab and placebo groups.

FIG. 14 depicts that dupilumab delayed time to first moderate-severe exacerbation in patients, with separation seen as early as week 4.

FIG. 15A-FIG. 15F depict efficacy across multiple demographic subgroups. Consistent reduction in moderate-severe exacerbations across multiple demographic subgroups.

FIG. 16A-FIG. 16B depict efficacy across multiple type 2 biomarker subgroups. Higher reduction was associated with higher levels of type 2 biomarkers.

FIG. 17 depicts reduction of exacerbations in GOLD airflow limitation subgroups.

FIG. 18 depicts that few severe exacerbations occurred during BOREAS trial. There was a trend toward reduction but no significant difference was observed in severe exacerbations between dupilumab and placebo groups.

FIG. 19 depicts FEV1 mean change from baselines during and after the 52-week treatment. Dupilumab led to rapid and sustained improvement in mean change from baseline compared to placebo.

FIG. 20A-FIG. 20F depict consistent FEV1 improvement at week 12 across subgroups.

FIG. 21A-FIG. 21B depict consistent, higher FEV1 improvement in subgroups having higher type 2 biomarkers.

FIG. 22 depicts that improvements in lung function were rapid and sustained in FVC and FEV1/FVC.

FIG. 23 depicts that dupilumab led to rapid and sustained improvement in post-bronchodilator (post-BD) FEV1.

FIG. 24A-FIG. 24B depict that dupilumab improved symptoms in all domains of E-RS:COPD at week 52 (breathlessness, cough/sputum and chest symptoms).

FIG. 25 depicts that dupilumab improved all domains of SGRQ at week 52.

FIG. 26 depicts that dupilumab reduced exposure to systemic corticosteroids (SCS) and antibiotics courses for exacerbations/respiratory conditions.

FIG. 27 depicts change in blood eosinophils over time. In the dupilumab group, blood Eos levels declined during the treatment period. A transient rise was seen in the mean Eos at week 8 and returned to baseline by week 24. No rise was observed in the median eosinophil counts.

FIG. 28 depicts type 2 biomarker distribution in patients of the BOREAS trial.

FIG. 29 depicts patient disposition in the BOREAS trial. *Note: One assigned placebo patient inadvertently received dupilumab, so the safety population is placebo (N=470) and dupilumab (N=469); Covid-19 coronavirus disease 2019.

FIG. 30A-FIG. 30B depict St. George's Respiratory Questionnaire (A) and E-RS COPD score (B) during the 52-week treatment period.

FIG. 31A-FIG. 31B depict a change from baseline in pre-bronchodilator FEF25-75% (A), and a change in baseline in pre-bronchodilator FVC percent predicted (B) over the 52-week study period.

FIG. 32 depicts the hierarchies of the BOREAS and NOTUS studies.

FIG. 33 depicts that the exacerbation reduction of COPD was 34% for the NOTUS study, which was statistically significant and clinically meaningful.

FIG. 34 depicts that Dupilumab reduced the rate of moderate and severe COPD exacerbations.

FIG. 35 depicts that Dupilumab reduced the time to first moderate and severe exacerbations with separation observed after week 4.

FIG. 36 depicts that a greater non-significant trend of reduction in severe exacerbations was observed in the NOTUS study.

FIG. 37 depicts that Dupilumab reduced the time to a first severe exacerbation of COPD. In NOTUS, dupilumab reduced the time to first severe exacerbation with nominal significance.

FIG. 38 depicts that Dupilumab improved lung function as measured by pre-BD FEV1. LS Mean Change in pre-BD FEV1 in both BOREAS and NOTUS studies at week 12 and week 52 are shown. Significant and clinically meaningful improvements in pre-BD FEV1 were observed at week 12 and week 52 in both studies.

FIG. 39 depicts that Dupilumab improved lung function as measured by pre-BD FEV1. LS mean change in pre-BD FEV1 from baseline through week 52 in both BOREAS and NOTUS studies is shown. Significant and clinically meaningful improvements in pre-BD FEV1 were observed shortly after the treatment and was maintained throughout the treatment.

FIG. 40 depicts LS Mean Δ from Baseline in post-BD FEV1. Dupilumab improved lung function as measured by post-BD FEV1. The improvement was rapid and sustained.

FIG. 41 depicts that Dupilumab improved health status as measured by St. George's Respiratory Questionnaire (SGRQ). In both studies, SGRQ improvement observed by week 12 and sustained through 52 weeks of treatment. SGRQ: Measure of impact of overall health, daily life and perceived well-being (health status). 50 items, scale 0-100, higher scores indicate more limitations. MCID: 4 units.

FIG. 42 depicts that Dupilumab improved health status as measured by SGRQ. Although significant improvement was seen in BOREAS but not NOTUS, Dupilumab response was consistent in both trials. The differential result was driven by higher placebo response in NOTUS vs. BOREAS (47% vs. 43%).

FIG. 43 depicts that Dupilumab improved symptoms of COPD as measured by E-RS COPD:RS-Total Score at week 52. E-RS: Measure severity of respiratory symptoms in stable COPD-total, breathlessness, cough and sputum, chest symptoms. Scale: 0-40, lower is better. Symptomatic improvement definition −2.0.

FIG. 44 depicts that Dupilumab improved symptoms of COPD as measured by E-RS COPD:RS-Total Score from baseline through week 52. Significant improvement in E-RS total score was observed in BOREAS and the improvement was rapid and sustained through week 52. For NOTUS, although the E-RS total score improvement was not significant at week 52, there was a clear trend to improvement in symptoms which happened rapidly after the treatment and was sustained.

FIG. 45 depicts region/countries of COPD patients in both trials (BOREAS: 939 enrolled in 24 countries; NOTUS: 935 enrolled in 29 countries).

FIG. 46 depicts that LS mean change from baseline in pre-BD FEV1 (L) for the FeNO ≥20 ppb subgroup. Lung function improvements increased with high FeNO. In NOTUS, although not significant, at week 52 there was a clear trend of improvement.

FIG. 47 depicts the annual rate of moderate-to-severe exacerbations in both trials in baseline FeNO ≥20 ppb subgroup. Dupilumab led to a 37% reduction over 52 weeks in BOREAS and a 53% reduction in NOTUS.

FIG. 48 depicts the pharmacokinetics of the BOREAS and the NOTUS studies.

FIG. 49 depicts serum pharmacokinetics over time by ADA titer category.

FIG. 50 depicts that Dupilumab decreased eosinophil levels (after a transient increase) over time. There were no cases of symptomatic eosinophilia, EGPA or eosinophilic pneumonia.

FIG. 51 depicts that Dupilumab decreased FeNO levels over time.

FIG. 52 depicts baseline demographics characteristics in NOTUS trial and compared to BOREAS. NOTUS baseline demo characteristics were similar to those in BOREAS.

FIG. 53 depicts efficacy across multiple subgroups based on demographics in the NOTUS trial. A consistent reduction in moderate-to-severe exacerbations across all demographic subgroups was observed.

FIG. 54 depicts efficacy across multiple subgroups based on disease. A consistent reduction in mod-severe exacerbations across all disease characteristic subgroups was observed.

FIG. 55 depicts efficacy across multiple subgroups based on type 2 biomarker subgroups. A higher reduction with higher levels of type 2 biomarkers was observed.

FIG. 56 depicts FEV1 levels across multiple subgroups based on demographics. Improvements in FEV1 were observed at week 12 and were consistent across multiple demographic subgroups.

FIG. 57 depicts FEV1 levels across multiple subgroups based on disease characteristics. Improvements in FEV1 were observed at week 12 was consistent across multiple disease characteristics subgroups.

FIG. 58 depicts FEV1 levels across multiple subgroups based on biomarkers. Higher improvement was seen with higher Type 2 biomarkers.

FIG. 59 depicts that lung function improvement was consistent among multiple parameters, as measured by LS Mean change from BL in post-BD FEV1/FVC over time. Improvements in lung function were rapid and sustained post-BD FEV1/FVC.

FIG. 60 depicts that lung function improvement was consistent among multiple parameters, as measured by LS Mean change from BL in pre-BD FVC over time. Improvements in lung function were seen in dupilumab group in both studies, with a smaller magnitude observed in the in the NOTUS study.

FIG. 61 depicts post-BD FEV1 slope results. No significant difference was observed in post-BD FEV1 slope in dupilumab vs. placebo.

FIG. 62 shows that Dupilumab improved breathlessness as assessed by E-RS:COPD at week 52 in the NOTUS trial.

FIG. 63 shows that Dupilumab improved all domains of SGRQ at week 52.

FIG. 64 shows the pooled Dupilumab adverse event profile. Dupilumab was well-tolerated with an acceptable safety profile in the pooled COPD safety population. No new safety concerns were identified.

FIG. 65 depicts health status and symptoms as evaluated by E-RS and SGRQ.

FIG. 66 depicts the most common SOCs (as defined as ≥5% incidence and ≥1% difference between the treatment arms).

FIG. 67 depicts the most common TEAEs (as defined as ≥5% incidence and ≥1% difference between the treatment arms).

FIG. 68 depicts the most common SOCs (as defined as ≥2% incidence and ≥1% difference between the treatment arms).

FIG. 69 depicts the most common TEAEs (as defined as ≥2% incidence and ≥1% difference between the treatment arms).

FIG. 70A-FIG. 70B depicts COPD exacerbation events during the 52-week NOTUS trial period. Error bars indicate 95% confidence intervals. Participants include those with the opportunity to reach week 52. (FIG. 70A) Cumulative moderate or severe COPD exacerbations. (FIG. 70B) Time to first moderate or severe COPD exacerbation. q2w denotes every 2 weeks.

FIG. 71 depicts change in pre-bronchodilator FEV1 over time for the NOTUS trial. Error bars indicate 95% confidence intervals. Participants include those with the opportunity to reach week 52. LS denotes least-squares, q2w every 2 weeks.

FIG. 72 is a table depicting selected demographic and clinical characteristics of patients at baseline (intention-to-treat population). *Plus-minus values are mean±SD. BD denotes bronchodilator, COPD chronic obstructive pulmonary disease, E-RS: COPD Evaluating Respiratory Symptoms in COPD, FeNO fractional exhaled nitric oxide, FEV1 forced expiratory volume in 1 s, FVC forced vital capacity, ICS inhaled corticosteroid, LABA long-acting beta-agonist, LAMA long-acting muscarinic antagonist, ppb parts per billion, Q quartile, SGRQ St. George's Respiratory Questionnaire.

FIG. 73 is a table depicting summary of endpoints included in the hierarchical testing procedure (intention-to-treat population). *End points are listed in the order in which they were hierarchically tested. †Only participants who had the opportunity to reach week 52 assessments were analyzed for the continuous and proportion-type end points at week 52. This included N=359 in placebo and N=362 in dupilumab groups (N=132 placebo and N=132 dupilumab for the subgroup with a baseline FeNO level ≥20 ppb). ‡P values from after the testing hierarchy broke were nominal.

FIG. 74 depicts adverse events (safety population). Data are presented as n (%) of patients. *The safety population consisted of all patients who received at least one full or partial dose of dupilumab or placebo; the analysis was performed according to the treatment each patient received. †Major adverse cardiovascular events (adjudicated) included cardiovascular death, non-fatal myocardial infarction, and non-fatal stroke.

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition (e.g., to prevent exacerbation of one or more symptoms of COPD).

Although any methods and materials similar or equivalent to those described herein can be used in the practice of the invention, the typical methods and materials are now described. All publications mentioned herein are incorporated herein by reference in their entirety.

### Methods for Reducing the Incidence of COPD Exacerbations

Methods for reducing the incidence of one or more COPD exacerbations (e.g., one or more chronic bronchitis exacerbations and/or pulmonary emphysema exacerbations that is not adequately controlled with existing therapy) in a subject in need thereof comprising administering a pharmaceutical composition comprising an interleukin-4 receptor (IL-4R) antagonist are provided. According to certain embodiments, the IL-4R antagonist is an antibody or antigen-binding fragment thereof that specifically binds IL-4R. Exemplary anti-IL-4R antibodies that can be used in the context of the methods featured in the disclosure are described herein.

In one aspect, a subject is identified as having “mild,” “moderate,” “severe,” or “very severe” COPD if the subject receives such a diagnosis from a physician, based on the Global Initiative for Chronic Obstructive Lung Disease (GOLD) classification system. In these aspects, a subject's COPD is classified based on airway limitation severity as tested using post-bronchodilator forced expiratory volume in 1 second (FEV1). A subject's airflow limitation from COPD is classified as “mild” using the GOLD classification system if the subject's FEV1 is greater than or equal to 80% of the predicted FEV1. A predicted value for FEV1 is based on the FEV1 value for an average person of similar age, race, height, and gender with healthy lungs. A subject's airflow limitation from COPD is classified as “moderate” on the GOLD classification system if the subject's FEV1 is greater than or equal to 50% of the predicted FEV1 but less than 80% of the predicted FEV₁. A subject's airflow limitation from COPD is classified as “severe” on the GOLD classification system if the subject's FEV1 is greater than or equal to 30% of the predicted FEV₁ but less than 50% of the predicted FEV1. A subject's airflow limitation from COPD is classified as “very severe” on the GOLD classification system if the subject's FEV1 is less than 30% of the predicted FEV1.

In some embodiments, a subject to be treated is classified as “moderate,” “severe,” or “very severe” on the Global Initiative for Chronic Obstructive Lung Disease (GOLD) classification system. In some embodiments, a subject to be treated is classified as “severe” or “very severe” on the GOLD classification system. In some embodiments, a subject to be treated is classified as “moderate” on the GOLD classification system. In some embodiments, a subject to be treated is classified as “severe” on the GOLD classification system. In some embodiments, a subject to be treated is classified as “very severe” on the GOLD classification system. In some embodiments, the subject has (1) 0 or 1 moderate exacerbation annually not leading to hospital admission, and (2) a modified Medical Research Council (mMRC) score between 0 to 1, or a COPD assessment test (CAT) score less than 10. In some embodiments, the subject has (1) 0 or 1 moderate exacerbation annually not leading to hospital admission, and (2) an mMRC score equal or greater than 2, or a CAT score equal or more than 10. In some embodiments, the subject has (1) two or more moderate exacerbations annually not leading to hospital admission or at least one severe exacerbation annually leading to hospital admission, and (2) an mMRC score between 0 to 1, or a CAT score less than 10. In some embodiments, the subject has (1) two or more moderate exacerbations annually not leading to hospital admission or at least one severe exacerbation annually leading to hospital admission, and (2) an mMRC score equal or greater than 2, or a CAT score equal or more than 10. In some embodiments, the subject has (1) two or more moderate exacerbations annually not leading to hospital admission or at least one severe exacerbation annually leading to hospital admission, regardless the mMRC score or the CAT score. mMRC is described in ATS (1982) Am Rev Respir Dis. No; 126(5):952-6. CAT score system is described in Jones et al. ERJ 2009; 34(3) 648-54. In some embodiments, the subject has two or more severe exacerbations annually leading to hospital admission.

In another aspect, methods for reducing the incidence or recurrence of COPD, or a COPD exacerbation, in a subject in need thereof are provided comprising administering a pharmaceutical composition comprising an IL-4R antagonist. A pharmaceutical composition comprising an IL-4R antagonist is provided for use to reduce the incidence or recurrence of COPD, or a COPD exacerbation in a subject in need thereof. As used herein, the expression “COPD exacerbation” means an increase in the severity and/or frequency and/or duration of one or more symptoms or indicia of COPD. A “COPD exacerbation” also includes any deterioration in the respiratory health of a subject that requires and/or is treatable by a therapeutic intervention COPD (such as, e.g., steroid treatment, antibiotic treatment, inhaled corticosteroid treatment, hospitalization, etc.).

In some embodiments, “moderate exacerbations” are defined as annualized rate of acute moderate or severe COPD exacerbation (AECOPD) that requires either systemic corticosteroids (such as intramuscular, intravenous or oral) and/or antibiotics. In some embodiments, “severe exacerbations” are defined as AECOPD requiring hospitalization, or observation for >24 hours in an emergency department/urgent care facility or resulting in death. In some embodiments, all other exacerbations are classified as “mild.” In some embodiments, for both moderate and severe events to count as separate events, they must be separated by at least 14 days.

A “reduction in the incidence or recurrence” of a COPD exacerbation means that a subject who has received the pharmaceutical compositions of the present disclosure experiences fewer COPD exacerbations (i.e., at least one fewer exacerbation) after treatment than before treatment, or experiences no COPD exacerbations for at least 4 weeks (e.g., 4, 6, 8, 12, 14, or more weeks) following initiation of treatment with a pharmaceutical composition of the present disclosure. A “reduction in the incidence or recurrence” of a COPD exacerbation alternatively means that, following administration of a pharmaceutical composition of the present disclosure, the likelihood that a subject experiences a COPD exacerbation is decreased by at least 10% (e.g., 10%, 15%, 20%, 25%, 30%, 35%, 40%, 45%, 50%, or more) as compared to a subject who has not received a pharmaceutical composition of the present disclosure.

Methods for reducing the incidence of COPD exacerbations in a subject in need thereof are provided comprising administering a pharmaceutical composition comprising an IL-4R antagonist to the subject as well as administering to the subject one or more maintenance doses of a second or a second and third controller, e.g., a long-acting beta-agonist (LABA), a long acting muscarinic antagonist (LAMA), and/or inhaled corticosteroid (ICS). In some embodiments, a pharmaceutical composition comprising an IL-4R antagonist is provided for use, in combination with one or more maintenance doses of a second or a second and third controller, e.g., a long-acting beta-agonist (LABA), a long acting muscarinic antagonist (LAMA), and/or inhaled corticosteroid (ICS), to reduce the incidence of COPD exacerbations in a subject in need thereof.

In some embodiments, a combination of a pharmaceutical composition comprising an IL-4R antagonist and one or more maintenance doses of a second or a second and third controller, e.g., a long-acting beta-agonist (LABA), a long acting muscarinic antagonist (LAMA), and/or inhaled corticosteroid (ICS), is provided for use to reduce the incidence of COPD exacerbations in a subject in need thereof.

Methods for reducing the incidence of COPD exacerbations in a subject in need thereof are provided comprising administering a pharmaceutical composition comprising an IL-4R antagonist to the subject as well as administering to the subject one or more reliever medications to eliminate or reduce one or more COPD-associated symptoms. A pharmaceutical composition comprising an IL-4R antagonist is provided for use, in combination with one or more reliever medications to eliminate or reduce one or more COPD-associated symptoms, to reduce the incidence of COPD exacerbations in a subject in need thereof. A combination comprising a pharmaceutical composition comprising an IL-4R antagonist and one or more reliever medications to eliminate or reduce one or more COPD-associated symptoms, is provided for use to reduce the incidence of COPD exacerbations in a subject in need thereof. Suitable reliever medications include, but are not limited to, quick-acting beta2-adrenergic receptor agonists such as, e.g., albuterol/salbutamol or levalbuterol/levosalbutamol (including ipratropium or ipratropium/short-acting R agonists (SABA) combinations).

In some embodiments, the IL-4R antagonist is an antibody or antigen-binding fragment thereof. In some embodiments, the antibody or antigen-binding fragment thereof comprises three heavy chain CDR sequences comprising SEQ ID Nos: 3, 4, and 5, respectively, and three light chain CDR sequences comprising SEQ ID Nos: 6, 7, and 8, respectively.

Methods for treating COPD are provided. In some embodiments, the COPD is uncontrolled COPD.

In some embodiments, a subject with COPD has chronic bronchitis and/or pulmonary emphysema that is not adequately controlled with existing therapy.

In some embodiments, a subject with uncontrolled COPD has type 2 inflammation. In some embodiments, a subject with uncontrolled COPD is on triple therapy (e.g., LABA+LAMA+ICS). In some embodiments, a subject with uncontrolled COPD is on double therapy (e.g., LABA+LAMA) because ICS is contraindicated.

In some embodiments, a subject with uncontrolled COPD is an adult. In some embodiments, a subject with uncontrolled COPD is an adult on triple therapy (e.g., LABA+LAMA+ICS). In some embodiments, a subject with uncontrolled COPD is an adult on double therapy (e.g., LABA+LAMA) because ICS is contraindicated.

In some embodiments, a subject with uncontrolled COPD is an adult that has type 2 inflammation and is on triple therapy (e.g., LABA+LAMA+ICS). In some embodiments, a subject with uncontrolled COPD is an adult that has type 2 inflammation and is on double therapy (e.g., LABA+LAMA) because ICS is contraindicated.

In some embodiments, a subject with uncontrolled COPD is treated with dupilumab as an add-on treatment. In some embodiments, a subject with uncontrolled COPD is treated with dupilumab as a maintenance treatment. In some embodiments, a subject with uncontrolled COPD is treated with dupilumab as an add-on maintenance treatment.

In some embodiments, a subject with uncontrolled COPD has type 2 inflammation and is treated with dupilumab as an add-on treatment. In some embodiments, a subject with uncontrolled COPD is on triple therapy (e.g., LABA+LAMA+ICS), and is treated with dupilumab as an add-on treatment. In some embodiments, a subject with uncontrolled COPD is on double therapy (e.g., LABA+LAMA) because ICS is contraindicated, and is treated with dupilumab as an add-on treatment.

In some embodiments, a subject with uncontrolled COPD is an adult and is treated with dupilumab as an add-on treatment. In some embodiments, a subject with uncontrolled COPD is an adult on triple therapy (e.g., LABA+LAMA+ICS), and is treated with dupilumab as an add-on treatment. In some embodiments, a subject with uncontrolled COPD is an adult on double therapy (e.g., LABA+LAMA) because ICS is contraindicated, and is treated with dupilumab as an add-on treatment.

In some embodiments, a subject with uncontrolled COPD is an adult that has type 2 inflammation and is on triple therapy (e.g., LABA+LAMA+ICS), and is treated with dupilumab as an add-on treatment. In some embodiments, a subject with uncontrolled COPD is an adult that has type 2 inflammation and is on double therapy (e.g., LABA+LAMA) because ICS is contraindicated, and is treated with dupilumab as an add-on treatment.

In some embodiments, a subject with uncontrolled COPD has type 2 inflammation and is treated with dupilumab as a maintenance treatment. In some embodiments, a subject with uncontrolled COPD is on triple therapy (e.g., LABA+LAMA+ICS), and is treated with dupilumab as a maintenance treatment. In some embodiments, a subject with uncontrolled COPD is on double therapy (e.g., LABA+LAMA) because ICS is contraindicated, and is treated with dupilumab as a maintenance treatment.

In some embodiments, a subject with uncontrolled COPD is an adult and is treated with dupilumab as a maintenance treatment. In some embodiments, a subject with uncontrolled COPD is an adult on triple therapy (e.g., LABA+LAMA+ICS), and is treated with dupilumab as a maintenance treatment. In some embodiments, a subject with uncontrolled COPD is an adult on double therapy (e.g., LABA+LAMA) because ICS is contraindicated, and is treated with dupilumab as a maintenance treatment.

In some embodiments, a subject with uncontrolled COPD is an adult that has type 2 inflammation and is on triple therapy (e.g., LABA+LAMA+ICS), and is treated with dupilumab as a maintenance treatment. In some embodiments, a subject with uncontrolled COPD is an adult that has type 2 inflammation and is on double therapy (e.g., LABA+LAMA) because ICS is contraindicated, and is treated with dupilumab as a maintenance treatment.

In some embodiments, a subject with uncontrolled COPD has type 2 inflammation and is treated with dupilumab as an add-on maintenance treatment. In some embodiments, a subject with uncontrolled COPD is on triple therapy (e.g., LABA+LAMA+ICS), and is treated with dupilumab as an add-on maintenance treatment. In some embodiments, a subject with uncontrolled COPD is on double therapy (e.g., LABA+LAMA) because ICS is contraindicated, and is treated with dupilumab as an add-on maintenance treatment.

In some embodiments, a subject with uncontrolled COPD is an adult and is treated with dupilumab as an add-on maintenance treatment. In some embodiments, a subject with uncontrolled COPD is an adult on triple therapy (e.g., LABA+LAMA+ICS), and is treated with dupilumab as an add-on maintenance treatment. In some embodiments, a subject with uncontrolled COPD is an adult on double therapy (e.g., LABA+LAMA) because ICS is contraindicated, and is treated with dupilumab as an add-on maintenance treatment.

In some embodiments, a subject with uncontrolled COPD is an adult that has type 2 inflammation and is on triple therapy (e.g., LABA+LAMA+ICS), and is treated with dupilumab as an add-on maintenance treatment. In some embodiments, a subject with uncontrolled COPD is an adult that has type 2 inflammation and is on double therapy (e.g., LABA+LAMA) because ICS is contraindicated, and is treated with dupilumab as an add-on maintenance treatment.

In some embodiments, a subject is an adult that receives dupilumab as an add-on maintenance treatment for uncontrolled COPD with type 2 inflammation on triple therapy (e.g., LABA+LAMA+ICS) or double therapy (e.g., LABA+LAMA) if ICS are contraindicated.

In some embodiments, dupilumab is indicated in adults as add-on maintenance treatment for uncontrolled chronic obstructive pulmonary disease (COPD) with type 2 inflammation on triple therapy or double therapy if inhaled corticosteroids (ICS) are contraindicated.

In some embodiments, dupilumab is indicated as an add-on maintenance treatment in adult patients with uncontrolled chronic obstructive pulmonary disease (COPD) associated with a history of exacerbations and guided by biomarkers of type 2 inflammation (e.g., blood eosinophils).

In some embodiments, a subject with uncontrolled COPD is an adult. In some embodiments, a subject with uncontrolled COPD has a history of COPD exacerbations. In some embodiments, a subject with uncontrolled COPD has an increased level of at least one biomarker of type 2 inflammation relative to a control. In some embodiments, a subject with uncontrolled COPD has an increased level of blood eosinophils relative to a control. In some embodiments, a subject with uncontrolled COPD has a history of COPD exacerbations and an increased level of at least one biomarker of type 2 inflammation relative to a control. In some embodiments, a subject with uncontrolled COPD has a history of COPD exacerbations and an increased level of blood eosinophils relative to a control.

In some embodiments, a subject with uncontrolled COPD is an adult with a history of COPD exacerbations. In some embodiments, a subject with uncontrolled COPD is an adult with an increased level of at least one biomarker of type 2 inflammation relative to a control. In some embodiments, a subject with uncontrolled COPD is an adult with an increased level of blood eosinophils relative to a control. In some embodiments, a subject with uncontrolled COPD is an adult with a history of COPD exacerbations and an increased level of at least one biomarker of type 2 inflammation relative to a control. In some embodiments, a subject with uncontrolled COPD is an adult with a history of COPD exacerbations and an increased level of blood eosinophils relative to a control.

In some embodiments, a subject with uncontrolled COPD is an adult and is treated with dupilumab as an add-on treatment. In some embodiments, a subject with uncontrolled COPD has a history of COPD exacerbations and is treated with dupilumab as an add-on treatment. In some embodiments, a subject with uncontrolled COPD has an increased level of at least one biomarker of type 2 inflammation relative to a control and is treated with dupilumab as an add-on treatment. In some embodiments, a subject with uncontrolled COPD has an increased level of blood eosinophils relative to a control and is treated with dupilumab as an add-on treatment. In some embodiments, a subject with uncontrolled COPD has a history of COPD exacerbations and an increased level of at least one biomarker of type 2 inflammation relative to a control and is treated with dupilumab as an add-on treatment. In some embodiments, a subject with uncontrolled COPD has a history of COPD exacerbations and an increased level of blood eosinophils relative to a control and is treated with dupilumab as an add-on treatment.

In some embodiments, a subject with uncontrolled COPD is an adult with a history of COPD exacerbations and is treated with dupilumab as an add-on treatment. In some embodiments, a subject with uncontrolled COPD is an adult with an increased level of at least one biomarker of type 2 inflammation relative to a control and is treated with dupilumab as an add-on treatment. In some embodiments, a subject with uncontrolled COPD is an adult with an increased level of blood eosinophils relative to a control and is treated with dupilumab as an add-on treatment. In some embodiments, a subject with uncontrolled COPD is an adult with a history of COPD exacerbations and an increased level of at least one biomarker of type 2 inflammation relative to a control and is treated with dupilumab as an add-on treatment. In some embodiments, a subject with uncontrolled COPD is an adult with a history of COPD exacerbations and an increased level of blood eosinophils relative to a control and is treated with dupilumab as an add-on treatment.

In some embodiments, a subject with uncontrolled COPD is an adult and is treated with dupilumab as a maintenance treatment. In some embodiments, a subject with uncontrolled COPD has a history of COPD exacerbations and is treated with dupilumab as a maintenance treatment. In some embodiments, a subject with uncontrolled COPD has an increased level of at least one biomarker of type 2 inflammation relative to a control and is treated with dupilumab as a maintenance treatment. In some embodiments, a subject with uncontrolled COPD has an increased level of blood eosinophils relative to a control and is treated with dupilumab as a maintenance treatment. In some embodiments, a subject with uncontrolled COPD has a history of COPD exacerbations and an increased level of at least one biomarker of type 2 inflammation relative to a control and is treated with dupilumab as a maintenance treatment. In some embodiments, a subject with uncontrolled COPD has a history of COPD exacerbations and an increased level of blood eosinophils relative to a control and is treated with dupilumab as a maintenance treatment.

In some embodiments, a subject with uncontrolled COPD is an adult with a history of COPD exacerbations and is treated with dupilumab as a maintenance treatment. In some embodiments, a subject with uncontrolled COPD is an adult with an increased level of at least one biomarker of type 2 inflammation relative to a control and is treated with dupilumab as a maintenance treatment. In some embodiments, a subject with uncontrolled COPD is an adult with an increased level of blood eosinophils relative to a control and is treated with dupilumab as a maintenance treatment. In some embodiments, a subject with uncontrolled COPD is an adult with a history of COPD exacerbations and an increased level of at least one biomarker of type 2 inflammation relative to a control and is treated with dupilumab as a maintenance treatment. In some embodiments, a subject with uncontrolled COPD is an adult with a history of COPD exacerbations and an increased level of blood eosinophils relative to a control and is treated with dupilumab as a maintenance treatment.

In some embodiments, a subject with uncontrolled COPD is an adult and is treated with dupilumab as an add-on maintenance treatment. In some embodiments, a subject with uncontrolled COPD has a history of COPD exacerbations and is treated with dupilumab as an add-on maintenance treatment. In some embodiments, a subject with uncontrolled COPD has an increased level of at least one biomarker of type 2 inflammation relative to a control and is treated with dupilumab as an add-on maintenance treatment. In some embodiments, a subject with uncontrolled COPD has an increased level of blood eosinophils relative to a control and is treated with dupilumab as an add-on maintenance treatment. In some embodiments, a subject with uncontrolled COPD has a history of COPD exacerbations and an increased level of at least one biomarker of type 2 inflammation relative to a control and is treated with dupilumab as an add-on maintenance treatment. In some embodiments, a subject with uncontrolled COPD has a history of COPD exacerbations and an increased level of blood eosinophils relative to a control and is treated with dupilumab as an add-on maintenance treatment.

In some embodiments, a subject with uncontrolled COPD is an adult with a history of COPD exacerbations and is treated with dupilumab as an add-on maintenance treatment. In some embodiments, a subject with uncontrolled COPD is an adult with an increased level of at least one biomarker of type 2 inflammation relative to a control and is treated with dupilumab as an add-on maintenance treatment. In some embodiments, a subject with uncontrolled COPD is an adult with an increased level of blood eosinophils relative to a control and is treated with dupilumab as an add-on maintenance treatment. In some embodiments, a subject with uncontrolled COPD is an adult with a history of COPD exacerbations and an increased level of at least one biomarker of type 2 inflammation relative to a control and is treated with dupilumab as an add-on maintenance treatment. In some embodiments, a subject with uncontrolled COPD is an adult with a history of COPD exacerbations and an increased level of blood eosinophils relative to a control and is treated with dupilumab as an add-on maintenance treatment.

### Methods for Improving COPD-Associated Parameters

Methods for improving one or more COPD-associated parameters (also referred to herein as “COPD modifying” or “disease modifying”) in a subject in need thereof are provided, wherein the methods comprise administering a pharmaceutical composition comprising an IL-4R antagonist to the subject. A pharmaceutical composition comprising an IL-4R antagonist is provided for use to improve one or more COPD-associated in a subject in need thereof. A reduction in the incidence of an COPD exacerbation (as described above) may correlate with an improvement in one or more COPD-associated parameters; however, such a correlation is not necessarily observed in all cases.

Examples of “COPD-associated parameters” include, but are not limited to, one or any combination of: (1) annualized rate of acute moderate or severe AECOPD; (2) annualized rate of severe AECOPD; (3) relative absolute change from baseline (e.g., week 52) in forced expiratory volume in 1 second (FEV1) pre-bronchodilator (4) relative absolute change from baseline (e.g., week 24) in forced expiratory volume in 1 second (FEV1) pre-bronchodilator; (5) relative absolute change from baseline (e.g., week 52) in forced expiratory volume in 1 second (FEV1) post-bronchodilator; (6) relative percent change from baseline (e.g., at week 24 and 52) in forced expiratory volume in 1 second (FEV1) pre-bronchodilator; (7) relative percent change from baseline (e.g., at week 24) in forced expiratory volume in 1 second (FEV1) post-bronchodilator; (8) relative rate of decline (e.g., slope) in forced expiratory volume in 1 second (FEV1) pre- and/or post-bronchodilator; (9) time to first moderate or severe AECOPD; (10) change from baseline in the Exacerbation of COPD Tool (EXACT) scores (e.g., at week 24); (11) change from baseline in the Evaluating Respiratory Symptoms in COPD (E-RS: COPD) scores (e.g., at week 24); (12) change from baseline in the St. George Respiratory Questionnaire (SGRQ) scores (e.g., at week 24); (13) change from baseline in the Euro Quality of Life 5-Dimension Questionnaire (EQ-5D) scores (e.g., at week 24); (14) rate of moderate-to-severe AECOPD; (15) change from baseline to week 16-24 in forced vital capacity (FVC); (16) change from baseline in Modified British Medical Research Council Questionnaire (mMRC) scores (e.g., at week 24); (17) change from baseline in the Health-Related Quality of Life Questionnaire (HRQOL) scores (e.g., at week 24); (18) change from baseline in Body mass index, airflow Obstruction, Dyspnea, Exercise performance (BODE) index scores (e.g., at week 24); (19) change from baseline in daily steps (e.g., at week 24); (20) days on oral corticosteroids; (21) days on antibiotics; (22) change from baseline in resting oxygen saturation (e.g., at week 24); (23) change from baseline in resting respiratory rate (e.g., at week 24); (24) maintenance of lung function (e.g., relative to no treatment or to treatment with placebo); (25) reduction in lung function decline (e.g., relative to no treatment or to treatment with placebo); (26) relative absolute change from baseline (e.g., week 52) of FEF25-75% (forced expiratory flow between 25% and 75%); (27) change from baseline in daily albuterol or levalbuterol use; (28) change from baseline in COPD assessment test (CAT) score; and (29) clinical symptoms of exacerbations of COPD.

An “improvement in a COPD-associated parameter” means an increase from baseline in FEV1, FVC, FEF25-75%, resting oxygen saturation, EQ-5D score, or time to first moderate or severe AECOPD. An improvement in a COPD-associated parameter can also mean a decrease from baseline in rate of AECOPD, daily albuterol or levalbuterol use, days on antibiotics, days on oral corticosteroids, BODE index score, CAT score, SGRQ score, EXACT score, E-RS:COPD score, or clinical symptoms of exacerbations of COPD. As used herein, the term “baseline,” with regard to a COPD-associated parameter, means the numerical value of the COPD-associated parameter for a patient prior to or at the time of administration of a pharmaceutical composition comprising an IL-4R antagonist.

To determine whether an COPD-associated parameter has “improved,” the parameter is quantified at baseline and at a time point after administration of the pharmaceutical composition described herein. For example, an COPD-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the COPD associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as an COPD-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis.”)

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Forced Expiratory Volume in 1 Second (FEV1). According to certain embodiments, administration of an IL-4R antagonist to a patient results in an increase of forced expiratory volume in 1 second (FEV1) from baseline. Methods for measuring FEV1 are known in the art. For example, a spirometer that meets the 2005 American Thoracic Society (ATS)/European Respiratory Society (ERS) recommendations can be used to measure FEV1 in a patient (See Miller, et al. “ATS/ERS TASK FORCE: Standardization of Lung Function Testing” Eur Respir J. 2005 August; 26(2):319-38). The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FEV1 (in liters.)

The disclosure includes therapeutic methods that result in an increase of FEV1 from baseline of at least 0.01 L at week 24 following initiation of treatment with a pharmaceutical composition comprising an anti-IL-4R antagonist. The disclosure includes a pharmaceutical composition comprising an anti-IL-4R antagonist for use to increase FEV1 from baseline of at least 0.01 L at week 24 following initiation of treatment with said pharmaceutical composition. For example, administration of an IL-4R antagonist causes an increase of FEV1 from baseline of about 0.01 L, 0.02 L, 0.03 L, 0.04 L, 0.05 L, 0.10 L, 0.12 L, 0.14 L, 0.16 L, 0.18 L, 0.20 L, 0.22 L, 0.24 L, 0.26 L, 0.28 L, 0.30 L, 0.32 L, 0.34 L, 0.36 L, 0.38 L, 0.40 L, 0.42 L, 0.44 L, 0.46 L, 0.48 L, 0.50 L, or more at week 24.

Forced Vital Capacity (FVC). According to certain embodiments, administration of an IL-4R antagonist to a patient results in an increase of FVC (forced vital capacity) from baseline. Methods for measuring FVC are known in the art. For example, a spirometer that meets the 2005 American Thoracic Society (ATS)/European Respiratory Society (ERS) recommendations can be used to measure FVC in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FVC (in liters).

FEF25-75%. According to certain embodiments, administration of an IL-4R antagonist to a patient results in an increase of FEF25-75% (forced expiratory flow between 25% and 75%) from baseline. Methods for measuring FEF are known in the art. For example, a spirometer that meets the 2005 American Thoracic Society (ATS)/European Respiratory Society (ERS) recommendations can be used to measure FEV1 in a patient. The FEF25-75% is the speed (in liters per second) at which a person can empty the middle half of his or her air during a maximum expiration (i.e., Forced Vital Capacity or FVC). The parameter relates to the average flow from the point at which 25 percent of the FVC has been exhaled to the point at which 75 percent of the FVC has been exhaled. The FEF25-75% of a subject provides information regarding small airway function, such that the extent of small airway disease and/or inflammation. A change in FEF25-75% is an early indicator of obstructive lung disease. In certain embodiments, an improvement and/or increase in the FEF25-75% parameter is an improvement of at least 10%, 25%, 50% or more as compared to baseline. In certain embodiments, the methods of the disclosure result in normal FEF25-75% values in a subject (e.g., values ranging from 50-60% and up to 130% of the average).

The disclosure includes therapeutic methods that result in a decrease in AECOPD from baseline of at least 5% at week 4, week 8, week 12, week 16, week 20, week 24, week 28, week 32, week 36, week 40, week 44, week 48, or week 52 following initiation of treatment with a pharmaceutical composition comprising an anti-IL-4R antagonist. The disclosure includes a pharmaceutical composition comprising an anti-IL-4R antagonist for use to decrease AECOPD from baseline of at least 5% at week 4, week 8, week 12, week 16, week 20, week 24, week 28, week 32, week 36, week 40, week 44, week 48, or week 52 following initiation of treatment with said pharmaceutical composition. For example, according to the disclosure, administration of an IL-4R antagonist to a subject in need thereof causes a decrease in AECOPD from baseline of about 5%, about 10%, about 15%, about 20%, about 25%, about 30%, about 35%, about 40%, about 45%, about 50%, about 55%, about 60%, about 65%, about 70%, or more at week 4, week 8, week 12, week 16, week 20, week 24, week 28, week 32, week 36, week 40, week 44, week 48, or week 52.

The disclosure includes therapeutic methods that result in a reduction in the probability of first AECOPD at a specific time point of at least 5% at week 24 following initiation of treatment with a pharmaceutical composition comprising an anti-IL-4R antagonist versus baseline. The disclosure includes a pharmaceutical composition comprising an anti-IL-4R antagonist for use to reduce the probability of first AECOPD at a specific time point of at least 5% at week 24 following initiation of treatment with said pharmaceutical composition. For example, according to the disclosure, administration of an IL-4R antagonist to a subject in need thereof causes a reduction in the probability of first AECOPD at a specific time point of about 5%, about 10%, about 15%, about 20%, about 25%, about 30%, about 35%, about 40%, about 45%, about 50%, about 55%, about 60%, about 65%, about 70%, or more at week 24 versus baseline.

Albuterol/Levalbuterol Use. According to certain embodiments, administration of an IL-4R antagonist to a patient results in a decrease from baseline of daily albuterol or levalbuterol use. The number of albuterol/levalbuterol inhalations can be recorded daily by the patients in a diary, PEF meter, or other recording device. During treatment with the pharmaceutical composition described herein, use of albuterol/levalbuterol typically may be on an as-needed basis for symptoms, not on a regular basis or prophylactically. The baseline number of albuterol/levalbuterol inhalations/day may be calculated based on the mean for the 7 days prior to administration of the first dose of pharmaceutical composition comprising the IL-4R antagonist.

The disclosure includes therapeutic methods that result in a decrease in albuterol/levalbuterol use from baseline of at least 0.25 puffs per day at week 12 following initiation of treatment with a pharmaceutical composition comprising an anti-IL-4R antagonist. For example, administration of an IL-4R antagonist to a subject in need thereof causes a decrease in albuterol/levalbuterol use from baseline of about 0.25 puffs per day, 0.50 puffs per day, 0.75 puffs per day, 1.00 puff per day, 1.25 puffs per day, 1.5 puffs per day, 1.75 puffs per day, 2.00 puffs per day, 2.25 puffs per day, 2.5 puffs per day, 2.75 puffs per day, 3.00 puffs per day, or more at week 12.

Daily Steps. According to certain embodiments, administration of an IL-4R antagonist to a patient results in a change from baseline in daily steps, e.g., results in an increase in daily steps over a defined period of time relative to daily steps over a defined period of time prior to administration of the IL-4R antagonist.

Corticosteroid/Antibiotic Use. According to certain embodiments, administration of an IL-4R antagonist to a patient results in a reduction of days on oral corticosteroids and/or a reduction of oral corticosteroid dose that is required. According to certain embodiments, administration of an IL-4R antagonist to a patient results in a reduction of days on antibiotics over a defined period of time relative to number of days the patient was on antibiotics over a defined period of time prior to administration of the IL-4R antagonist.

Oxygen Saturation. In some embodiments, administration of an IL-4R antagonist to a patient results in a change from baseline in resting oxygen saturation, e.g., results in increased resting oxygen saturation than is obtained prior to administration of the IL-4R antagonist.

Respiratory Rate. In some embodiments, administration of an IL-4R antagonist to a patient results in a change from baseline in resting respiratory rate, e.g., a decrease or an increase in respiratory rate. In certain exemplary embodiments, administration of an IL-4R antagonist to a patient results in a decrease from baseline in resting respiratory rate relative to resting respiratory rate prior to administration of the IL-4R antagonist.

Body Mass Index, Airflow Obstruction, Dyspnea, Exercise Performance (BODE) Index. According to certain embodiments, administration of an IL-4R antagonist to a patient results in an improvement from baseline of BODE index score, wherein an improvement from baseline is a decrease in BODE index score. In some embodiments, administration of an IL-4R antagonist to a patient results in a decrease from baseline of BODE index score of greater than 1 point. The BODE Index is a composite measure composed of a Performance Outcome Measure, a Patient-Reported Outcome Measure and a Biomarker. The BODE Index is a multidimensional grading system to assess the respiratory and systemic expressions of COPD. (Celli et al. “The Body-mass Index, Airflow obstruction, Dyspnea, and Exercise Capacity Index in Chronic Obstructive Pulmonary Disease” N Engl J Med. 2004 Mar. 4; 350(10):1005-12.) It comprises 4 domains: 1) degree of pulmonary impairment (FEV1); 2) patient's perception of symptoms (mMRC); and 2 independent domains: the 6 Minute Walking Distance (6MWD) and the Body-Mass Index (BMI). Each domain can be scored independently; the global score ranges from 0 to 10, with a higher score indicating a higher risk of death.

Therapeutic methods are provided that result in a decrease in BODE score from baseline. For example, administration of an IL-4R antagonist to a subject in need thereof causes a decrease in BODE score from baseline of about 1, 2, 3, 4, 5, 6, 7, 8, 9, or 10 points.

COPD Assessment Test (CAT) Score. According to certain embodiments, administration of an IL-4R antagonist to a patient results in a decrease from baseline of CAT score. An anti-IL-4R antagonist is provided for use in a patient to decrease CAT score from baseline. The CAT is a questionnaire that is designed for patients with COPD to measure the effects of the disease on their quality of lives. The CAT is an 8-item self-administered questionnaire which has been developed for use in routine clinical practice to measure the health status of patients with COPD. The CAT score ranges from 0 to 40, a higher score indicating a higher impact on health status. The test is about cough, phlegm, chest tightness, dyspnea, activity limitation, confidence, sleep and energy. Patients score questions from 1-5 according to their own feelings about the disease (1=I am very happy; 5=I am very sad).

Therapeutic methods are provided that result in a decrease in CAT score from baseline. For example, administration of an IL-4R antagonist to a subject in need thereof causes a decrease in CAT score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 points.

St. George's Respiratory Questionnaire (SGRQ). According to certain embodiments, administration of an IL-4R antagonist to a patient results in a decrease from baseline of SGRQ score. An anti-IL-4R antagonist is provided for use in a patient to decrease SGRQ score from baseline. The St. George's Respiratory Questionnaire (SGRQ) is a 50-item questionnaire designed to measure and quantify health status in adult patients with chronic airflow limitation. (See Jones et al. “The St George's Respiratory Questionnaire” Respir Med. 1991 September; 85 Suppl B:25-31; discussion 33-7.) A global score ranges from 0 to 100. Scores by dimension are calculated for three domains: symptoms, activity, and impacts (Psycho-social) as well as a total score. Lower score indicates better quality of life (QoL).

The first part (“symptoms”) evaluates symptomatology, including frequency and severity of cough, sputum production, wheeze, breathlessness and the duration and frequency of attacks of breathlessness or wheeze. The second part has two components: “activity” and “impacts”. The “activity” section addresses disturbances to patients' daily physical activities. The “impacts” section covers a range of effects that chest troubles may have on patients' daily life and psycho-social functions (e.g., daily life activities and functioning, employment, physical functioning, emotional impact, stigmatization, and patients' perceptions when treated). The recall period of the questionnaire is over the past 4 weeks.

Psychometric testing has demonstrated its repeatability, reliability and validity. Sensitivity has been demonstrated in clinical trials. A minimum change in score of 4 units was established as clinically relevant after patient and clinician testing. The SGRQ has been used in a range of disease groups including asthma, COPD and bronchiectasis.

Therapeutic methods are provided that result in a decrease in SGRQ score from baseline. For example, administration of an IL-4R antagonist to a subject in need thereof causes a decrease in SGRQ score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

Exacerbations of Chronic Obstructive Pulmonary Disease Tool (EXACT). According to certain embodiments, administration of an IL-4R antagonist to a patient results in a decrease from baseline of EXACT score. An IL-4R antagonist is provided for use in a patient to decrease EXACT score from baseline. The EXACT tool quantifies and measures exacerbations of COPD and assesses the symptomatic manifestations of these COPD exacerbations. The instrument is a daily diary composed of a total of 14 items representing the following domains: breathlessness (5 items), cough and sputum (2 items), chest symptoms (3 items), difficulty bringing up sputum (1 item), tired or weak (1 item), sleep disturbance (1 item), and scared or worried (1 item). Development and validation history of the tool is consistent with guidelines proposed by the FDA, EMA and well-known measurement principles. The EXACT total score assesses COPD exacerbations. The higher the score, the more severe are the symptoms.

Evaluating Respiratory Symptoms in COPD (E-RS: COPD). According to certain embodiments, administration of an IL-4R antagonist to a patient results in a decrease from baseline of E-RS:COPD score. An IL-4R antagonist is provided for use in a patient to decrease E-RS:COPD score from baseline. The E-RS: COPD scale is a part of the EXACT tool. It is a derivative instrument used to measure the effect of treatment on the severity of respiratory symptoms in stable COPD. The E-RS utilizes the 11 respiratory symptom items contained in the 14-item EXACT. The RS-Total score represents respiratory symptom severity, overall. Three subscales can be used that assess: 1) breathlessness (RS-Breathlessness), 2) cough and sputum (RS-Cough and Sputum), and 3) chest-related symptoms (RS-Chest Symptoms). The higher the score the more severe are the symptoms.

Euroqol-5 dimensions (EQ-5D): According to certain embodiments, administration of an IL-4R antagonist to a patient results in an increase from baseline of EQ-5D. An IL-4R antagonist is provided for use in a patient to increase EQ-5D score from baseline. The Euroqol-5 dimensions (EQ-5D) is a standardized PRO measure of health status developed by the EuroQol Group in order to provide a simple, generic measure of health for clinical and economic appraisal. The adult version of the questionnaire is adapted to patients aged 16 and older. The EQ-5D consists of 2 parts: the descriptive system and the EQ visual analogue scale (EQ VAS). The EQ-5D 5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “inability to do the activity.” (See Herdman M, et al. Development and preliminary testing of the new five-level version of EQ-5D (EQ-5D-5L). Qual. Life Res. 2011; 20(10):1727-36.) The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent's health state. The EQ VAS records the respondent's self-rated health on a vertical, VAS where the endpoints are labeled “best imaginable health state (100)” and “worst imaginable health state (0).” This information can be used as a quantitative measure of health outcome as judged by the individual respondents.

Therapeutic methods are provided that result in an increase in EQ VAS score from baseline. For example, administration of an IL-4R antagonist to a subject in need thereof causes an increase in EQ VAS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

Medical Research Council (MRC) dyspnoea scale. According to certain embodiments, administration of an IL-4R antagonist to a patient results in a patient reporting better health status in a Medical Research Council (MRC) dyspnoea scale. An IL-4R antagonist is provided for use in a patient to have this patient reporting better health status in a Medical Research Council (MRC) dyspnoea scale. (See Bestall, et al. “Usefulness of the Medical Research Council (MRC) Dyspnea Scale as a Measure of Disability in Patients with Chronic Obstructive Pulmonary Disease” Thorax 1999; 54:581-586.)

Modified British Medical Research Council Questionnaire (mMRC). According to certain embodiments, administration of an IL-4R antagonist to a patient results in a patient reporting better health status in a Modified British Medical Research Council Questionnaire (mMRC). An IL-4R antagonist is provided for use in a patient to have this patient reporting better health status in a Modified British Medical Research Council Questionnaire (mMRC). The Modified British Medical Research Council Questionnaire (mMRC) is a questionnaire that assesses breathlessness. (Fletcher et al. Standardized questionnaire on respiratory symptoms: a statement prepared and approved by the MRC Committee on the Aetiology of Chronic Bronchitis (MRC breathlessness score). BMJ 1960; 2: 1662.)

Health-Related Quality of Life (HRQOL) Questionnaire. According to certain embodiments, administration of an IL-4R antagonist to a patient results in a patient reporting better health status in a Health-Related Quality of Life (HRQOL) Questionnaire. Centers for Disease Control and Prevention. Measuring Healthy Days. Atlanta, Georgia: CDC, November 2000, Available at the website: cdc.gov/hrqol/pdfs/mhd.pdf). An IL-4R antagonist is provided for use in a patient to have this patient reporting better health status in a HRQOL Questionnaire.

Clinical Symptoms of Exacerbations of COPD. According to certain embodiments, administration of an IL-4R antagonist to a patient results in a decrease from baseline of clinical symptoms of exacerbations of COPD. An IL-4R antagonist is provided for use in a patient to decrease clinical symptoms of exacerbations of COPD from baseline. Clinical symptoms of exacerbations of COPD can include but are not limited to dyspnea, increase in wheezing, increase in cough, increase in sputum volume and/or increase in sputum purulence.

Biomarkers. In certain embodiments, the subject experiences an improvement in lung function as measured by a biomarker. In certain exemplary embodiments, a subject experiences an increase in a biomarker level after administration of an IL-4R antagonist (relative to the biomarker level before administration of the IL-4R antagonist). In certain exemplary embodiments, a subject experiences a decrease in a biomarker level after administration of IL-4R antagonist (relative to the biomarker level before administration of the anti-IL-4R antagonist). In certain exemplary embodiments, a subject experiences a normalization of one or more biomarkers after administration of IL-4R antagonist (relative to the expression level of the biomarker before administration of the anti-IL-4R antagonist). For example, the biomarker may be selected from the group consisting of pulmonary and activation-regulated chemokine (PARC), eotaxin-3, fibrinogen, IgE, sputum or blood eosinophils, sputum or blood neutrophils, fractional exhaled nitric oxide (FeNO), IL-4Rα, IL 4, IL-13, IL-33, serum periostin, calcium-activated chloride channel regulator (CLCA1), cystatin-SN (CST1), suppression of tumorigenicity 2 (ST2), thymic stromal lymphopoietin (TSLP), and the like. In certain embodiments, whole blood mRNA samples are obtained for sequencing or whole transcriptome analysis. In certain embodiments, serum and/or plasma samples are obtained and optionally archived for research regarding exploratory biomarkers of disease or drug effect. In certain embodiments, samples are used for research to develop methods, assays, prognostics and/or companion diagnostics related to IL-4R, disease process(es), pathways associated with disease state and/or mechanism of action of the study intervention. In certain embodiments, an improvement in lung function is indicated by a reduction or increase (as appropriate) of a biomarker at week 4, week 12 or week 24 following treatment.

In some embodiments, methods to treat or alleviate one or more conditions or complications associated with COPD or comorbid with COPD, such as a Type 2 inflammatory condition, e.g., one or more of asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), allergic conjunctivitis, atopic conjunctivitis, atopic dermatitis, vasculitis, cystic fibrosis (CF), chronic obstructive pulmonary disease (COPD), eosinophilic esophagitis (EoE), chronic rhinosinusitis with nasal polyps (CRSwNP), aspirin hypersensitivity, non-steroidal anti-inflammatory drug (NSAID) hypersensitivity (e.g., NSAIDs exacerbated respiratory disease, or NSAID-ERD), perennial allergic rhinitis (PAR), atopic dermatitis (AD), food allergy, hives or urticaria, chronic eosinophilic pneumonia (CEP) and exercise induced bronchospasm, are provided.

In some embodiments, methods to treat or alleviate one or more conditions or complications associated with COPD or comorbid with COPD, such as pulmonary artery disease, coronary heart disease, heart failure, endothelial dysfunction, coagulopathy, systemic venous thromboembolism, hypertension, metabolic syndrome, diabetes mellitus, dyslipidemia, anemia, arthritis, osteoporosis, muscle weakness (e.g., musculoskeletal dysfunction), gastroesophageal reflux, gastrointestinal disturbances, sleep disturbance, obstructive sleep apnea syndrome (OSAS), malnutrition, anemia, obesity, psychological disturbances (e.g., anxiety, depression, cognitive impairment), malignancies (e.g., lung cancer), lung fibrosis, pulmonary embolism, pneumonia, and the like, are provided.

In some embodiments, the IL-4R antagonist is an antibody or antigen-binding fragment thereof. In some embodiments, the antibody or antigen-binding fragment thereof comprises three heavy chain CDR sequences comprising SEQ ID Nos: 3, 4, and 5, respectively, and three light chain CDR sequences comprising SEQ ID Nos: 6, 7, and 8, respectively.

### Methods for Treating COPD

In some embodiments, methods for treating COPD, including, e.g. moderate-to-severe COPD and/or COPD with Type 2 inflammation, in a subject in need thereof are provided, wherein the methods comprise administering a pharmaceutical composition comprising an IL-4R antagonist. In certain embodiments, the methods are useful for treating moderate-to-severe COPD in a subject. In certain embodiments, the methods are useful for treating COPD with Type 2 inflammation in a subject. In certain embodiments, the methods are useful for treating moderate-to-severe COPD with Type 2 inflammation in a subject. In certain embodiments, the methods are useful for reducing one or more AECOPD events. A pharmaceutical composition comprising an IL-4R antagonist is provided to treat COPD, including, e.g., moderate-to-severe COPD and/or COPD with Type 2 inflammation, in a subject in need thereof. A pharmaceutical composition comprising an IL-4R antagonist to reduce one or more AECOPD events in a patient is provided.

In one aspect, methods for treating COPD are provided comprising: (a) selecting a patient that exhibits Type 2 inflammation; and (b) administering to the patient a pharmaceutical composition comprising an IL-4R antagonist. In some embodiments, Type 2 inflammatory COPD is driven by the activation of IL-4, IL-5, and/or IL-13. In some embodiments, Type 2 inflammatory COPD is driven by an increased level of eosinophils. In some embodiments, Type 2 inflammatory COPD is driven by an increased level of neutrophils. In some embodiments, the patient with Type 2 inflammation exhibits a blood eosinophil level of equal to or greater than 300 cells per microliter.

In one aspect, methods for treating COPD are provided comprising: (a) selecting a patient that exhibits a blood eosinophil level of equal to or greater than 300 cells per microliter; and (b) administering to the patient a pharmaceutical composition comprising an IL-4R antagonist. In one aspect of the composition for use, the patient exhibits a blood eosinophil level of equal to or greater than 300 cells per microliter.

In one aspect, methods for treating COPD are provided comprising: (a) selecting a patient that exhibits a blood eosinophil level of equal to or greater than 250 cells per microliter; and (b) administering to the patient a pharmaceutical composition comprising an IL-4R antagonist. In one aspect of the composition for use, the patient exhibits a blood eosinophil level of equal to or greater than 250 cells per microliter.

In one aspect, methods for treating COPD are provided comprising: (a) selecting a patient that exhibits a blood eosinophil level of equal to or greater than 300 cells per microliter; and (b) administering to the patient a pharmaceutical composition comprising an IL-4R antagonist. In one aspect of the composition for use, the patient exhibits a blood eosinophil level of equal to or greater than 300 cells per microliter.

In one aspect, methods for treating COPD are provided comprising: (a) selecting a patient that exhibits a blood eosinophil level of equal to or greater than 500 cells per microliter; and (b) administering to the patient a pharmaceutical composition comprising an IL-4R antagonist. In one aspect of the composition for use, the patient exhibits a blood eosinophil level of equal to or greater than 500 cells per microliter.

In one aspect, methods for treating COPD are provided comprising: (a) selecting a patient that exhibits a blood eosinophil level of less than 300 cells per microliter; and (b) administering to the patient a pharmaceutical composition comprising an IL-4R antagonist. In one aspect of the composition for use, the patient exhibits a blood eosinophil level of less than 300 cells per microliter.

In another aspect, methods for treating COPD are provided comprising: (a) selecting a patient that exhibits a blood eosinophil level of 150-299 cells per microliter; and (b) administering to the patient a pharmaceutical composition comprising an IL-4R antagonist. In one aspect of the composition for use, the patient exhibits a blood eosinophil level of 150-299 cells per microliter.

In another aspect, methods for treating COPD are provided comprising: (a) selecting a patient that exhibits a blood eosinophil level of less than 150 cells per microliter; and (b) administering to the patient a pharmaceutical composition comprising an IL-4R antagonist. In one aspect of the composition for use, the patient exhibits a blood eosinophil level of less than 150 cells per microliter.

In a related aspect, methods for treating COPD comprising an add-on therapy to background therapy are provided. In a related aspect, an IL-4R antagonist is provided for use to treat COPD in a patient, wherein IL-4R antagonist is used as an add-on therapy to background therapy. In certain embodiments, an IL-4R antagonist is administered as an add-on therapy to a COPD patient who is on background therapy for a certain period of time (e.g., 1 week, 2 weeks, 3 weeks, 1 month, 2 months, 5 months, 12 months, 18 months, 24 months, or longer) (also called the “stable phase”).

COPD background therapies include, but are not limited to (1) beta2-agonists, such as short-acting beta 2-agonists (SABA, e.g., fenoterol, levalbuterol, salbutamol, and terbutaline) or long-acting beta 2-agonists (LABA, e.g., arformoterol, formoterol, indacaterol, olodaterol, and salmeterol); (2) anticholinergics, such as short-acting anticholinergics (SAMA, e.g., ipratropium bromide, oxitropium bromide) or long-acting anticholinergics (LAMA, e.g., aclidinium bromide, glycopyrronium bromide, tiotropium, umeclidinium, glycopyrrolate, and revefenacin); (3) combinations of SABA and SAMA (e.g., fenoterol+ipratropium or salbutamol+ipratropium); (4) combinations of LABA and LAMA (formoterol+aclidinium, formoterol+glycopyrronium, indacaterol+glycopyrronium, vilanterol+umeclidinium, and olodaterol+tiotropium); (5) methylxanthines (e.g., aminophylline and theophylline); (6) combination of LABA and corticosteroid (e.g., formoterol+beclomethasone, formoterol+budesonide, formoterol+mometasone, salmeterol+fluticasone propionate, vilanterol+fluticasone furoate); (6) triple combination (e.g., LABA+LAMA+ICS, such as fluticasone+umeclidinium+vilanterol, beclomethasone+formoterol+glycopyrronium, budesodine+formoterol+glycopyrrolate); (7) phosphodiesterase inhibitors (e.g., phosphodiesterase-4 inhibitors, such as roflumilast); (8) mucolytic agents (e.g., erdosteine, catbocysterine, and N-acetylcysterine); or any combinations thereof.

In some embodiments, the subject receives at least one non-pharmacological therapy, including but not limited to smoking cessation, pulmonary rehabilitation, long-term oxygen therapy, noninvasive positive pressure ventilation, and lung volume reduction surgery.

In an exemplary embodiment, the background therapy comprises a LABA, a LAMA, and an ICS. In other exemplary embodiments, the background therapy comprises a LABA and a LAMA, and an ICS is contraindicated.

In some embodiments, the disclosure includes a method for reducing a COPD patient's dependence on one or more of LABA, LAMA, and ICS for the treatment of one or more COPD exacerbations comprising: (a) selecting a patient who has moderate-to-severe COPD that is not well-controlled with a background therapy comprising a LABA, a LAMA, and an ICS; and administering to the patient a pharmaceutical composition comprising an IL-4R antagonist. A pharmaceutical composition comprising an IL-4R antagonist is provided for use to reduce a COPD patient's dependence on one or more of ICS, LAMA, and LABA for the treatment of one or more COPD exacerbations, in a patient who has moderate-to-severe COPD that is not well-controlled with a background COPD therapy comprising a LABA, a LAMA, and an ICS, or a combination thereof.

In some embodiments, the disclosure includes a method for reducing a COPD patient's dependence on one or more of LABA, LAMA, and ICS for the treatment of one or more COPD exacerbations comprising: (a) selecting a patient who has COPD with Type 2 inflammation that is not well-controlled with a background therapy comprising a LABA, a LAMA, and an ICS; and administering to the patient a pharmaceutical composition comprising an IL-4R antagonist. A pharmaceutical composition comprising an IL-4R antagonist is provided for use to reduce a COPD patient's dependence on one or more of ICS, LAMA, and LABA for the treatment of one or more COPD exacerbations, in a patient who has COPD with Type 2 inflammation that is not well-controlled with a background COPD therapy comprising a LABA, a LAMA, and an ICS, or a combination thereof.

In some embodiments, the disclosure includes a method for reducing a COPD patient's dependence on one or more of LABA, LAMA, and ICS for the treatment of one or more COPD exacerbations comprising: (a) selecting a patient who has moderate-to-severe COPD with Type 2 inflammation that is not well-controlled with a background therapy comprising a LABA, a LAMA, and an ICS; and administering to the patient a pharmaceutical composition comprising an IL-4R antagonist. A pharmaceutical composition comprising an IL-4R antagonist is provided for use to reduce a COPD patient's dependence on one or more of ICS, LAMA, and LABA for the treatment of one or more COPD exacerbations, in a patient who has moderate-to-severe COPD with Type 2 inflammation that is not well-controlled with a background COPD therapy comprising a LABA, a LAMA, and an ICS, or a combination thereof.

In some embodiments, the disclosure includes a method for reducing a COPD patient's dependence on one or both of LABA and LAMA for the treatment of one or more COPD exacerbations comprising: (a) selecting a patient who has moderate-to-severe COPD that is not well-controlled with a background therapy comprising a LABA and a LAMA; and administering to the patient a pharmaceutical composition comprising an IL-4R antagonist. A pharmaceutical composition comprising an IL-4R antagonist is provided for use to reduce a COPD patient's dependence on one or both of LAMA and LABA for the treatment of one or more COPD exacerbations, in a patient who has moderate-to-severe COPD that is not well-controlled with a background COPD therapy comprising a LABA and a LAMA, or a combination thereof.

In some embodiments, the disclosure includes a method for reducing a COPD patient's dependence on one or both of LABA and LAMA for the treatment of one or more COPD exacerbations comprising: (a) selecting a patient who has COPD with Type 2 inflammation that is not well-controlled with a background therapy comprising a LABA and a LAMA; and administering to the patient a pharmaceutical composition comprising an IL-4R antagonist. A pharmaceutical composition comprising an IL-4R antagonist is provided for use to reduce a COPD patient's dependence on one or both of LAMA and LABA for the treatment of one or more COPD exacerbations, in a patient who has COPD with Type 2 inflammation that is not well-controlled with a background COPD therapy comprising a LABA and a LAMA, or a combination thereof.

In some embodiments, the disclosure includes a method for reducing a COPD patient's dependence on one or both of LABA and LAMA for the treatment of one or more COPD exacerbations comprising: (a) selecting a patient who has moderate-to-severe COPD with Type 2 inflammation that is not well-controlled with a background therapy comprising a LABA and a LAMA; and administering to the patient a pharmaceutical composition comprising an IL-4R antagonist. A pharmaceutical composition comprising an IL-4R antagonist is provided for use to reduce a COPD patient's dependence on one or both of LAMA and LABA for the treatment of one or more COPD exacerbations, in a patient who has moderate-to-severe COPD with Type 2 inflammation that is not well-controlled with a background COPD therapy comprising a LABA and a LAMA, or a combination thereof.

In some embodiments, the IL-4R antagonist is an antibody or antigen-binding fragment thereof. In some embodiments, the antibody or antigen-binding fragment thereof comprises three heavy chain CDR sequences comprising SEQ ID Nos: 3, 4, and 5, respectively, and three light chain CDR sequences comprising SEQ ID Nos: 6, 7, and 8, respectively.

In some embodiments, methods of the present disclosure lead to one or more of the following benefits in the treated subject:
(1) statistically significant and clinically meaningful reduction in annualized rate of moderate to severe exacerbations, such as at least 5%, at least 10%, at least 15%, at least 20%, at least 25%, at least 30% or more relative risk reduction (RRR);
(2) rapid and sustained improvement in lung function, such as improvement at Week 2, 3, 5, 6, 7, 8, 9, 10, 11, or 12 after the initial dose, which lasts for at least 26 week, at least 52 weeks, at least 78 weeks, at least 104 weeks, at least 130 weeks, at least 156 weeks, or more. In some embodiments, the subject has an improvement of FEV1 of at least +10 ml, at least +20 ml, at least +30 ml, at least +40 ml, at least +50 ml, at least +60 ml, at least +70 ml, at least 80 ml, at least 90 ml, at least 100 m, or more;
(3) consistent and higher benefit in subjects having an FeNO level ≥20 ppb. Such benefits may include reduced exacerbations and improved FEV1.
(4) rapid and sustained improvement in Health-Related Quality of Life Questionnaire score. In some embodiments, the improvement is measured by SGRQ score. (In some embodiments, the SGRQ score in a treated subject is at least 1, at least 2, at least 3, at least 4, at least 5, at least 6, at least 7, at least 8, at least 9 or more below that of a placebo group. In some embodiment, improvement of SGRQ score in a treated subject is at least 1, at least 2, at least 3, at least 4, at least 5, or more compared to untreated baseline);
(5) rapid and sustained improvement in total symptoms (breathlessness, cough/sputum, chest). (In some embodiments, the improvement is measured by ERS-COPD score. In some embodiments, the ERS-COPD score in a treated subject is at least 0.2, at least 0.4, at least 0.6, at least 0.8, at least 1.0, at least 1.1, at least 1.2 below that of a placebo group); and
(6) benefits observed across multiple subgroups of demographic, clinical and biomarkers subgroups.

### Interleukin-4 Receptor Antagonists

The methods featured herein comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo. Non-limiting examples of categories of IL-4R antagonists include small molecule IL-4R antagonists, anti-IL-4R aptamers, peptide-based IL-4R antagonists (e.g., “peptibody” molecules), and antibodies or antigen-binding fragments of antibodies that specifically bind human IL-4R. According to certain embodiments, the IL-4R antagonist comprises an anti-IL-4R antibody that can be used in the context of the methods described elsewhere herein. For example, in one embodiment, the IL-4R antagonist is an antibody or antigen-binding fragment thereof that specifically binds to an IL-4R, and comprises the heavy chain and light chain (complementarity determining region) CDR sequences from the heavy chain variable region (HCVR) and light chain variable region (LCVR) of SEQ ID Nos:1 and 2, respectively.

The term “human IL4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies described herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. Antibodies having one or more mutations in the hinge, C_{H}2, or C_{H}3 region, which may be desirable, for example, in production, to improve the yield of the desired antibody form, are provided.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4R includes antibodies that bind IL-4R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-4R may, however, have cross-reactivity to other antigens, such as IL-4R molecules from other (non-human) species.

The anti-IL-4R antibodies useful for the methods may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the disclosure.

Methods involving the use of anti-IL-4R antibodies comprising variants of any of the HCVR, LCVR, and/or CDR amino acid sequences disclosed herein having one or more conservative substitutions. For example, the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein, are provided.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, or at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, or at least 98% or 99% sequence identity. In exemplary embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. (See, e.g., Pearson (1994) Methods Mol. Biol. 24: 307-331, herein incorporated by reference). Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. (See, e.g., GCG Version 6.1.) Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another exemplary algorithm when comparing a sequence of the disclosure to a database containing a large number of sequences from different organisms is the computer program BLAST, especially BLASTP or TBLASTN, using default parameters. (See, e.g., Altschul et al. (1990) J. Mol. Biol. 215:403-410 and Altschul et al. (1997) Nucleic Acids Res. 25:3389-402, each of which is herein incorporated by reference.)

### Preparation of Human Antibodies

Methods for generating human antibodies in transgenic mice are known in the art. Any such known methods can be used to make human antibodies that specifically bind to human IL-4R.

Using VELOCIMMUNE® technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody described herein, for example wild-type or modified IgG1 or IgG4. While the constant region selected may vary according to specific use, high affinity antigen-binding and target specificity characteristics reside in the variable region.

In general, the antibodies that can be used in the methods described herein possess high affinities, as described above, when measured by binding to antigen either immobilized on solid phase or in solution phase. The mouse constant regions are replaced with desired human constant regions to generate the fully-human antibodies described herein. While the constant region selected may vary according to specific use, high affinity antigen-binding and target specificity characteristics reside in the variable region.

In one embodiment, human antibody or antigen-binding fragment thereof that specifically binds IL-4R that can be used in the context of the methods described herein comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 3/4/5/6/7/8.

In certain embodiments, the antibody or antigen-binding fragment thereof comprises HCVR/LCVR amino acid sequence pair of SEQ ID NOs: 1 and 2.

In certain embodiments, the antibody is dupilumab, which comprises the HCVR/LCVR amino acid sequence pair of SEQ ID NOs: 1 and 2.

In certain embodiments, the antibody sequence is dupilumab, which comprises the heavy chain/light chain amino acid sequence pair of SEQ ID NOs: 9 and 10.

Dupilumab HCVR amino acid sequence:
(SEQ ID NO: 1)
EVQLVESGGGLEQPGGSLRLSCAGSGFTFRDYAMTWVRQAPGKGLEWVS
SISGSGGNTYYADSVKGRFTISRDNSKNTLYLQMNSLRAEDTAVYYCAK
DRLSITIRPRYYGLDVWGQGTTVTVS.
Dupilumab LCVR amino acid sequence:
(SEQ ID NO: 2)
DIVMTQSPLSLPVTPGEPASISCRSSQSLLYSIGYNYLDWYLQKSGQSP
QLLIYLGSNRASGVPDRFSGSGSGTDFTLKISRVEAEDVGFYYCMQALQ
TPYTFGQGTKLEIK.
Dupilumab HCDR1 amino acid sequence:
(SEQ ID NO: 3)
GFTFRDYA.
Dupilumab HCDR2 amino acid sequence:
(SEQ ID NO: 4)
ISGSGGNT.
Dupilumab HCDR3 amino acid sequence:
(SEQ ID NO: 5)
AKDRLSITIRPRYYGL.
Dupilumab LCDR1 amino acid sequence:
(SEQ ID NO: 6)
QSLLYSIGYNY.
Dupilumab LCDR2 amino acid sequence:
(SEQ ID NO: 7)
LGS.
Dupilumab LCDR3 amino acid sequence:
(SEQ ID NO: 8)
MQALQTPYT.
Dupilumab HC amino acid sequence:
(SEQ ID NO: 9)
EVQLVESGGGLEQPGGSLRLSCAGSGFTFRDYAMTWVRQAPGKGLEWVS
SISGSGGNTYYADSVKGRFTISRDNSKNTLYLQMNSLRAEDTAVYYCAK
DRLSITIRPRYYGLDVWGQGTTVTVSSASTKGPSVFPLAPCSRSTSEST
AALGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHY
TQKSLSLSLG (amino acids 1-124 = HCVR; amino acids
125-451 = HC constant).
Dupilumab LC amino acid sequence:
(SEQ ID NO: 10)
DIVMTQSPLSLPVTPGEPASISCRSSQSLLYSIGYNYLDWYLQKSGQSP
QLLIYLGSNRASGVPDRFSGSGSGTDFTLKISRVEAEDVGFYYCMQALQ
TPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC (amino acids 1-112 =
LCVR; amino acids 112-219 = LC constant).

In certain embodiments, an antibody or antigen-binding fragment thereof of the disclosure comprises light chain variable region (LCVR) and heavy chain variable region (HCVR) sequence pairs (LCVR/HCVR) selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In certain embodiments, an antibody or antigen-binding fragment thereof of the disclosure comprises a LCVR/HCVR sequence pair of SCB-VL-44/SCB-VH-92.

In certain embodiments, an antibody or antigen-binding fragment thereof of the disclosure comprises a LCVR/HCVR sequence pair of SCB-VL-54/SCB-VH-92.

In certain embodiments, an antibody or antigen-binding fragment thereof of the disclosure comprises a LCVR/HCVR sequence pair of SCB-VL-55/SCB-VH-92.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-55-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-44-LCDR3.

The antibodies recited below in Table 1 are described in more detail in U.S. Pat. No. 10,774,141, incorporated herein by reference in its entirety for all purposes.

| Sequence ID | Sequence |
|---|---|
| SCB-VL-39 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYGSSPP |
| | WTFGQGTKVEIK (SEQ ID NO: 11) |
| SCB-VL-40 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYGSSPP |
| | WTFGQGTKVEIK (SEQ ID NO: 12) |
| SCB-VL-41 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYGSSPP |
| | WTFGQGTKVEIK (SEQ ID NO: 13) |
| SCB-VL-42 | EIVLTQSPGTLSLSPGERATLSCRASQSVSNSYLAWYQQKPGQAPRLL |
| | IYGASSRATGIPDRFSGSGSGTDFTLTISRLEPEDFAVYYCQQYGSSPP |
| | WTFGQGTKVEIK (SEQ ID NO: 14) |
| SCB-VL-43 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYGSSPP |
| | WTFGQGTKVEIK (SEQ ID NO: 15) |
| SCB-VL-44 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYGSSPP |
| | WTFGQGTKVEIK (SEQ ID NO: 16) |
| SCB-VL-45 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYDHSPP |
| | WTFGQGTKVEIK (SEQ ID NO: 17) |
| SCB-VL-46 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSAG |
| | WTFGQGTKVEIK (SEQ ID NO: 18) |
| SCB-VL-47 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYDHSAG |
| | WTFGQGTKVEIK (SEQ ID NO: 19) |
| SCB-VL-48 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYDHSPP |
| | WTFGQGTKVEIK (SEQ ID NO: 20) |
| SCB-VL-49 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSPP |
| | WTFGQGTKVEIK (SEQ ID NO: 21) |
| SCB-VL-50 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYDHSPP |
| | WTFGQGTKVEIK (SEQ ID NO: 22) |
| SCB-VL-51 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYDHSA |
| | GWTFGQGTKVEIK (SEQ ID NO: 23) |
| SCB-VL-52 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYDHSAG |
| | WTFGQGTKVEIK (SEQ ID NO: 24) |
| SCB-VL-53 | EIVLTQSPGTLSLSPGERATLSCRASQSVSNSYLAWYQQKPGQAPRLL |
| | IYGASSRATGIPDRFSGSGSGTDFTLTISRLEPEDFAVYYCQQYDHSA |
| | GWTFGQGTKVEIK (SEQ ID NO: 25) |
| SCB-VL-54 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYDHSAG |
| | WTFGQGTKVEIK (SEQ ID NO: 26) |
| SCB-VL-55 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSA |
| | GWTFGQGTKVEIK (SEQ ID NO: 27) |
| SCB-VL-56 | EIVLTQSPGTLSLSPGERATLSCRASQSVSNSYLAWYQQKPGQAPRLL |
| | IFGASSRATGIPDFSGSGSGTDFTLTISRLEPEDFAVYYCQQYDHSAG |
| | WTFGQGTKVEIK (SEQ ID NO: 28) |
| SCB-VL-57 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSPP |
| | WTFGQGTKVEIK (SEQ ID NO: 29) |
| SCB-VL-58 | EIVLTQSPGTLSLSPGERATLSCRASQSVSNSYLAWYQQKPGQAPRLL |
| | IYGASSRAPGIPDRFSGSGSGTDFTLTISRLEPEDFAVYYCQQYDHSA |
| | GWTFGQGTKVEIK (SEQ ID NO: 30) |
| SCB-VH-59 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 31) |
| SCB-VH-60 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 32) |
| SCB-VH-61 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 33) |
| SCB-VH-62 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFDYWGQGTLVTVSS (SEQ ID NO: 34) |
| SCB-VH-63 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFDYWGQGTLVTVSS (SEQ ID NO: 35) |
| SCB-VH-64 | EVQLVESGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 36) |
| SCB-VH-65 | EVQLVESGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 37) |
| SCB-VH-66 | EVQLVQSGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 37) |
| SCB-VH-67 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS (SEQ ID NO: 38) |
| SCB-VH-68 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 39) |
| SCB-VH-69 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 40) |
| SCB-VH-70 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 41) |
| SCB-VH-71 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 42) |
| SCB-VH-72 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS (SEQ ID NO: 43) |
| SCB-VH-73 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS (SEQ ID NO: 44) |
| SCB-VH-74 | EVQLVQSGGGLVHPGRSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 45) |
| SCB-VH-75 | EVQLVQSGGGLVHPGGSLRLTCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 46) |
| SCB-VH-76 | EVQLVQSGGGLVHPGGSLRLSCAGSGFTFSRNAMHWVRQAPGKGL |
| | EWVSGIGTGGATNYADSVKGRFTISRDNAKNSLYLQMNSLRAEDMA |
| | VYYCARGRYYFDYWGQGTLVTVSS (SEQ ID NO: 47) |
| SCB-VH-77 | EVQLVQSGGGLVHPGGSLRLSCAGSGFTFSRNAMFWVRQAPGEGLE |
| | WVSGIGTGGATNYADSVKGRFTISRDNAKNSLYLQMNSLRAEDMA |
| | VYYCARGRYYFDYWGQGTLVTVSS (SEQ ID NO: 48) |
| SCB-VH-78 | EVQLVQSGGGLVHPGGSLRLSCAGSGFTFSRNAMFWVRQAPGKGLE |
| | WVSGIGTGGATNYADSVKGRFTISRDEAKNSLYLQMNSLRAEDMAV |
| | YYCARGRYYFDYWGQGTLVTVSS (SEQ ID NO: 49) |
| SCB-VH-79 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAGDMA |
| | VYYCARGRYYFDYWGQGTLVTVSS (SEQ ID NO: 50) |
| SCB-VH-80 | EVQLVQSGGGLVHPGGSLRLSCAGSGFTFDDYAMFWVRQAPGKGL |
| | EWVSGIGTGGATNYADSVKGRFTISRDNAKNSLYLQMNSLRAEDMA |
| | VYYCARGRYYFDYWGQGTLVTVSS (SEQ ID NO: 51) |
| SCB-VH-81 | EVQLVQSGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 52) |
| SCB-VH-82 | EVQLVESGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 53) |
| SCB-VH-83 | EVQLVESGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 54) |
| SCB-VH-84 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS (SEQ ID NO: 55) |
| SCB-VH-85 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS (SEQ ID NO: 56) |
| SCB-VH-86 | EVQLVQSGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 57) |
| SCB-VH-87 | EVQLVQSGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 58) |
| SCB-VH-88 | EVQLVESGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 59) |
| SCB-VH-89 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 60) |
| SCB-VH-90 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 61) |
| SCB-VH-91 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS (SEQ ID NO: 62) |
| SCB-VH-92 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 63) |
| SCB-VH-93 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 64) |
| SCB-92- | RNAMF (SEQ ID NO: 65) |
| HCDR1 | |
| SCB-92- | GIGTGGATSYADSVKG (SEQ ID NO: 66) |
| HCDR3 | |
| SCB-92- | GRYYFDY (SEQ ID NO: 67) |
| HCDR3 | |
| SCB-55- | RASQSVSSSYLA (SEQ ID NO :68) |
| LCDR1 | |
| SCB-55- | GASSRAT (SEQ ID NO: 69) |
| LCDR2 | |
| SCB-55- | QQYDHSAGWT (SEQ ID NO: 70) |
| LCDR3 | |
| SCB-54- | GASSRAP (SEQ ID NO: 71) |
| LCDR2 | |
| SCB-44- | QQYGSSPPWT (SEQ ID NO: 72) |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of MEDI-1-VL/MEDI-1-VH through MEDI-42-VL/MEDI-42-VH.

In certain embodiments, an antibody or antigen-binding fragment thereof of the disclosure comprises a LCVR/HCVR sequence pair of MEDI-37GL-VL/MEDI-37GL-VH.

In certain embodiments, an antibody or antigen-binding fragment thereof of the disclosure comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 of MEDI-37GL-LCDR1, and LCDR2 of MEDI-37GL-LCDR2, and an LCDR3 of MEDI-37GL-LCDR3.

The antibodies recited below in Table 2 are described in more detail in U.S. Pat. No. 8,877,189, incorporated herein by reference in its entirety for all purposes.

TABLE 2

| Sequence ID | Sequence |
|---|---|
| MEDI-1-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLDYWGKGTLVTVSS (SEQ ID NO: 74) |
| MEDI-1-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SLSANYVFGTGTKLTVL (SEQ ID NO: 75) |
| MEDI-2-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 76) |
| MEDI-2-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SQPPNPLFGTGTKLTVL (SEQ ID NO: 77) |
| MEDI-3-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKLLKNPWGKGTLVTVSS (SEQ ID NO: 78) |
| MEDI-3-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWFG |
| | TPASNYVFGTGTKLTVL (SEQ ID NO: 79) |
| MEDI-4-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 76) |
| MEDI-4-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL (SEQ ID NO: 80) |
| MEDI-5-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS (SEQ ID NO: 81) |
| MEDI-5-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL (SEQ ID NO: 80) |
| MEDI-6-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-6-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 83) |
| MEDI-7-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS (SEQ ID NO: 84) |
| MEDI-7-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL (SEQ ID NO: 80) |
| MEDI-8-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS (SEQ ID NO: 84) |
| MEDI-8-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 83) |
| MEDI-9-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 76) |
| MEDI-9-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL (SEQ ID NO: 85) |
| MEDI-10-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS (SEQ ID NO: 81) |
| MEDI-10-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVLTPIFGTGTKLTVL (SEQ ID NO: 86) |
| MEDI-11-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS (SEQ ID NO: 87) |
| MEDI-11-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPSMIPLFGTGTKLTVL (SEQ ID NO: 73) |
| MEDI-12-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS (SEQ ID NO: 87) |
| MEDI-12-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL (SEQ ID NO: 85) |
| MEDI-13-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS (SEQ ID NO: 81) |
| MEDI-13-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTLQPLFGTGTKLTVL (SEQ ID NO: 88) |
| MEDI-14-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 76) |
| MEDI-14-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPPTKPLFGTGTKLTVL (SEQ ID NO: 89) |
| MEDI-15-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 76) |
| MEDI-15-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STHRHPLFGTGTKLTVL (SEQ ID NO: 90) |
| MEDI-16-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 76) |
| MEDI-16-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 83) |
| MEDI-17-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS (SEQ ID NO: 91) |
| MEDI-17-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVDRPIFGTGTKLTVL (SEQ ID NO: 92) |
| MEDI-18-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS (SEQ ID NO: 91) |
| MEDI-18-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTPMPVFGTGTKLTVL (SEQ ID NO: 93) |
| MEDI-19-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS (SEQ ID NO: 91) |
| MEDI-19-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 83) |
| MEDI-20-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-20-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVWEWPFGTGTKLTVL (SEQ ID NO: 94) |
| MEDI-21-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS (SEQ ID NO: 95) |
| MEDI-21-VL | QSVLTQPPSVSAAPGQKVTISCSGGSSNIGNSYVSWYQQLPGTAPKL |
| | LIYDNNKRPSGIPDRFSGSKSGTSATLAITGLQTGDEAVYFCGTWDT |
| | STVWEWPFGTGTKLTVL (SEQ ID NO: 96) |
| MEDI-22-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-22-VL | QPVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 97) |
| MEDI-23-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 98) |
| MEDI-23-VL | QSVLTQPPSVSAAPGQKVTISCSGGSSNIGNNYVSWYQQLPGTAPKL |
| | LIYDNNKRPPGIPDRFSGSKSGTSATLAITGLQTGDEADYYCGTWDT |
| | STVWEWPFGTGTKLTVL (SEQ ID NO: 99) |
| MEDI-24-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS (SEQ ID NO: 100) |
| MEDI-24-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 101) |
| MEDI-25-VH | QVQLVQSGAEVKKPGASVKVSCKASGYAFTSYYMHWARQAPGQG |
| | LEWMGIINPRGGSASYAQKFQGRVSMTRDTSTSTVYMELSSLRSED |
| | TAVYYCARGKYWMYDWGKGTLVTVSS (SEQ ID NO: 102) |
| MEDI-25-VL | QSVLTQPPSVSAAPGQKVTISCSGGSSNIGNSYVSWYQQLPGTAPKL |
| | LIYDNNKRPSGIPDRFSGSKSGTTATLAITGLQTGDEADYYCGTWVT |
| | STVWEWPFGTGTKLTVL (SEQ ID NO: 103) |
| MEDI-26-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-26-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 101) |
| MEDI-27-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTQVTVSS (SEQ ID NO: 104) |
| MEDI-27-VL | QSVLTQPPLVSAAPGQKVTISCSGGSSNIGNSYVSWYQRLPGTAPKL |
| | LIYDNNKRPSGIPDRFSGSKSGTSATLAITGLQTGDEADYYCGTWDT |
| | STVWEWPFGTGTKLTVL (SEQ ID NO: 105) |
| MEDI-28-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGNGTLVTVSS (SEQ ID NO: 106) |
| MEDI-28-VL | LPVLTQPPSVSAAPGQKVTISCSGGSSSIGNSYVSWYQQLPGAAPKL |
| | LIYDNNKRPSGIPDRFSGFRSGTSATLAITGLQTGDEADYYCGTWDT |
| | SPVWEWPFGTGTKLTVL (SEQ ID NO: 107) |
| MEDI-29-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTRVTVSS (SEQ ID NO: 108) |
| MEDI-29-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL (SEQ ID NO: 109) |
| MEDI-30-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-30-VL | QSVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STVWEWPFGTGTKLTVL (SEQ ID NO: 110) |
| MEDI-31-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-31-VL | QSVLTQPPSVSAAPGQKVTISCSGGSSSIGNSYVSWYQQLPGTAPKL |
| | LIYDNNKRPSGIPDRFSGSKSGTSATLAITGLQTGDEADYYCGTWAT |
| | SPVWEWPFGTGTKLTVL (SEQ ID NO: 111) |
| MEDI-32-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-32-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STAWEWPFGTGTKLTVL (SEQ ID NO: 112) |
| MEDI-33-VH | QVQLVQSGAEEKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 113) |
| MEDI-33-VL | QSALTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 114) |
| MEDI-34-VH | QVQLVQSGAEVKKPGASVKVSCKASGYAFTSYYMHWARQAPGQG |
| | LEWMGIINPSGGSTSYAQKFQGRVSMTRDTSTSTVYMELSSLRSEDT |
| | AVYYCARGKYWMYDWGKGTLVTVSS (SEQ ID NO: 115) |
| MEDI-34-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 101) |
| MEDI-35-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-35-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL (SEQ ID NO: 109) |
| MEDI-36-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS (SEQ ID NO: 95) |
| MEDI-36-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL (SEQ ID NO: 116) |
| MEDI-37-VH | QVQLVQSGAEVKKPGASVKVSCKASGYAFTSYYMHWARQAPGQG |
| | LEWMGIINPRGGSTSYAQKFQGRVAMTRDTSTSTVYMELSSLRPED |
| | TAVYYCARGKYWMYDWGKGTLVTVSS (SEQ ID NO: 117) |
| MEDI-37-VL | QSVLTQPPSVSAAPGQKVTISCSGGGSSIGNSYVSWYQQLPGTAPKL |
| | LIYDNNKRPSGVPDRFSGSKSGTSATLAITGLQTGDEADYYCGTWD |
| | TSPVWEWPFGTGTKLTVL (SEQ ID NO: 118) |
| MEDI-38-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS (SEQ ID NO: 95) |
| MEDI-38-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 101) |
| MEDI-39-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS (SEQ ID NO: 100) |
| MEDI-39-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STAWEWPFGTGTKLTVL (SEQ ID NO: 119) |
| MEDI-40-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 82) |
| MEDI-40-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL (SEQ ID NO: 116) |
| MEDI-41-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTLVTVSG (SEQ ID NO: 120) |
| MEDI-41-VL | QSVLTQPPSVSAAPGQKVTISCSGGSTNIGNSYVSWYQRLPGTAPKL |
| | LIYDNNKRPPGIPDRFSGSKSGTSATLAITGLQTGDEADYYCGTWDT |
| | STVWEWPFGTGTKLTVL (SEQ ID NO: 121) |
| MEDI-42-VH | QVQLVQSGAEVKKPGASVKVSCKASGYAFTSYYMHWARQAPGQG |
| | LEWVGIINPSGGSTSYAQKFQGRVTMTRDTSTSTVYMELSSLRSGDT |
| | AVYYCARGKYWMYDWGKGTLVTVSS (SEQ ID NO: 122) |
| MEDI-42-VL | QAVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STGWEWPFGTGTKLTVL (SEQ ID NO: 123) |
| MEDI-37GL- | QVQLVQSGAEVKKPGASVKVSCKASGYAFTSYYMHWVRQAPGQG |
| VH | LEWMGIINPRGGSTSYAQKFQGRVTMTRDTSTSTVYMELSSLRSED |
| | TAVYYCARGKYWMYDWGKGTLVTVSS (SEQ ID NO: 124) |
| MEDI-37GL- | QSVLTQPPSVSAAPGQKVTISCSGGGSSIGNSYVSWYQQLPGTAPKL |
| VL | LIYDNNKRPSGIPDRFSGSKSGTSATLGITGLQTGDEADYYCGTWDT |
| | SPVWEWPFGTGTKLTVL (SEQ ID NO: 125) |
| MEDI-37GL- | SYYMH (SEQ ID NO: 126) |
| HCDR1 | |
| MEDI-37GL- | IINPRGGSTSYAQKFQG (SEQ ID NO: 127) |
| HCDR2 | |
| MEDI-37GL- | GKYWMYD (SEQ ID NO: 128) |
| HCDR3 | |
| MEDI-37GL- | SGGGSSIGNSYVS (SEQ ID NO: 129) |
| LCDR1 | |
| MEDI-37GL- | DNNKRPS (SEQ ID NO: 130) |
| LCDR2 | |
| MEDI-37GL- | GTWDTSPVWEWP (SEQ ID NO: 131) |
| LCDR3 | |

In certain embodiments, an antibody or antigen-binding fragment thereof of the disclosure comprises a LCVR/HCVR sequence pair of AJOU-90-VL/AJOU-83-VH.

In certain embodiments, an antibody or antigen-binding fragment thereof of the disclosure comprises an HCVR comprising an HCDR1 sequence of AJOU-84-HCDR1, an CHDR2 sequence of AJOU-85-HCDR2, and an HCDR3 sequence of AJOU-32-HCDR3, and an LCVR comprising an LCDR1 of AJOU-96-LCDR1, and LCDR2 of AJOU-60-LCDR2, and an LCDR3 of AJOU-68-LCDR3.

The antibodies recited below in Table 3 are described in more detail in WO2020/096381 and Kim et al. (Scientific Reports. 9: 7772. 2019), incorporated herein by reference in their entireties for all purposes.

TABLE 3

| Sequence ID | Sequence |
|---|---|
| AJOU-1-VH | EVQLLESGGGLVQPGGSLRLSCAVSGFTFSNYAMSWVRQAPGKGL |
| | EWVSAISSGGGNIYYADSVKGRFTISRDNSKNTLYLQMNSLRAEDT |
| | AVYYCAKLRRYFDYWGQGTLVTVSS (SEQ ID NO: 132) |
| AJOU-2-VH | EVQLLESGGGLVQPGGSLRLSCAASGFTFSDYAMSWVRQAPGKGL |
| | EWVSAISSGGSSIYYADSVKGRFTISRDNSKNTLHLQMNSLRAEDT |
| | AVYYCARGPQRSATAVFDYWGQGTLVTVSS (SEQ ID NO: 133) |
| AJOU-3-VH | EVQLLESGGGLVQPGGSLRLSCAASGFTFSNYAMSWVRQAPGKGL |
| | EWVSWISPNSGNIYYADSVKGRFTISRDNSKNTLYLQMNSLRAEDT |
| | AVYYCARRPLSAAWSHSSYYNAMDVWGQGTLVTVSS (SEQ ID |
| | NO: 134) |
| AJOU-4-VH | EVQLLESGGGLVQPGGSLRLSCAASGFTFSGYAMSWVRQAPGKGL |
| | EWVSLISHSGSNTYYADSVKGRFTISRDNSKNTLYLQMNSLRAEDT |
| | AVYYCARPHRAFDYWGQGTLVTVSS (SEQ ID NO: 135) |
| AJOU-5-VH | EVQLLESGGGLVQPGGSLRLSCAASGFTFSNYAMSWVRQAPGKGL |
| | EWVSGISHGSGSIYYADSVKGRFTISRDNSKNTLYLQMNSLRAEDT |
| | AVYYCARPHRAFDYWGQGTLVTVSS (SEQ ID NO: 136) |
| AJOU-6-VH | EVQLLESGGGLVQPGGSLRLSCAASGFTFSNYAMSWVRQAPGKGL |
| | EWVSGISHGNGSIYYADSVKGRFTISRDNSKNTLYLQMNSLRAEDT |
| | AVYYCAKTGRHFDYWGQGTLVTVSS (SEQ ID NO: 137) |
| AJOU-7-VH | EVQLLESGGGLVQPGGSLRLSCAASGFTFSNYAMSWVRQAPGKGL |
| | EWVSSISPSGSSIYYADSVKGRFTISRDNSKNTLYLQMNSLRAEDTA |
| | VYYCARSYRAFDYWGQGTLVTVSS (SEQ ID NO: 138) |
| AJOU-8-VH | EVQLLESGGGLVQPGGSLRLSCAASGFTFSNYAMSWVRQAPGKGL |
| | EWVSAISPSGGSIYYADSVKGRFTISRDNSKNTLYLQMNSLRAEDT |
| | AVYYCARAKRAFDYWGQGTLVTVSS (SEQ ID NO: 139) |
| AJOU-9-VH | EVQLLESGGGLVQPGGSLRLSCAASGFTFSNYAMSWVRQAPGKGL |
| | EWVSAISPGSGSTYYADSVKGRFTISRDNSKNTLYLQMNSLRAEDT |
| | AVYYCAKFRRHFDYWGQGTLVTVSS (SEQ ID NO: 140) |
| AJOU-10-VH | EVQLLESGGGLVQPGGSLRLSCAASGFTFSNYAMSWVRQAPGKGL |
| | EWVSAISSGGGNIYYADSVKGRFTISRDNSKNTLYLQMNSLRAEDT |
| | AVYYCARVHRAFDYWGQGTLVTVSS (SEQ ID NO: 141) |
| AJOU-69-VH | EVQLLESGGGLVQPGGSLRLSCAASGFTFSNYAMSWVRQAPGKGL |
| | EWVSAITSSGRSIYYADSVKGRFTISRDNSKNTLYLQMNSLRAEDT |
| | AVYYCARVHRAFDYWGQGTLVTVSS (SEQ ID NO: 142) |
| AJOU-70-VH | EVQLLESGGGLVQPGGSLRLSCAASGFTFSNYAMSWVRQAPGKGL |
| | EWVSAITSSGANIYYADSVKGRFTISRDNSKNTLYLQMNSLRAEDT |
| | AVYYCARVHRAFDYWGQGTLVTVSS (SEQ ID NO: 143) |
| AJOU-71-VH | EVQLLESGGGLVQPGGSLRLSCAASGFTFSNYAMSWVRQAPGKGL |
| | EWVSAITSSGGNIYYADSVKGRFTISRDNSKNTLYLQMNSLRAEDT |
| | AVYYCARVHRAFDYWGQGTLVTVSS (SEQ ID NO: 144) |
| AJOU-72-VH | EVQLLESGGGLVQPGGSLRLSCAASGFTFSNYAMSWVRQAPGKGL |
| | EWVSAITAGGGSIYYADSVKGRFTISRDNSKNTLYLQMNSLRAEDT |
| | AVYYCARVHRAFDYWGQGTLVTVSS (SEQ ID NO: 145) |
| AJOU-83-VH | EVQLLESGGGLVQPGGSLRLSCAASGFTFSRHAMAWVRQAPGKGL |
| | EWVSAITSSGRSIYYADSVKGRFTISRDNSKNTLYLQMNSLRAEDT |
| | AVYYCARVHRAFDYWGQGTLVTVSS (SEQ ID NO: 146) |
| AJOU-33-VL | QSVLTQPPSASGTPGQRVTISCSGSSSNIGNNYVNWYQQLPGTAPK |
| | LLIYDNSHRPSGVPDRFSGSKSGTSASLAISGLRSEDEADYYCGTW |
| | DASLSAYVFGGGTKLTVL (SEQ ID NO: 147) |
| AJOU-34-VL | QSVLTQPPSASGTPGQRVTISCSGSSSNIGNNNVSWYQQLPGTAPKL |
| | LIYANSKRPSGVPDRFSGSKSGTSASLAISGLRSEDEADYYCGSWD |
| | DSLSAYVFGGGTKLTVL (SEQ ID NO: 148) |
| AJOU-35-VL | QSVLTQPPSAPGTPGQRVTISCTGSSSNIGSNSVNWYQQLPGTAPKL |
| | LIYDDSHRPSGVPDRFSGSKSGTSASLAISGLRSEDEADYYCDAWD |
| | SSLSAYVFGGGTKLTVL (SEQ ID NO: 149) |
| AJOU-36-VL | QSVLTQPPSASGTPGQRVTLSCTGSSSNIGSNYVSWYQQLPGTAPK |
| | LLIYADSQRPSGVPDRFSGSKSGTSASLAISGLRSEDEADYYCGTW |
| | DDSLSGYVFGGGTKLTVL (SEQ ID NO: 150) |
| AJOU-37-VL | QSVLTQPPSASGTPGQRVTISCSSSSSNIGSNYVSWYQQLPGTAPKL |
| | LIYSDSHRPSGVPDRFSGSKSGTSASLAISGLRSEDEADYYCGSWDY |
| | SLSAYVFGGGTKLTVL (SEQ ID NO: 151) |
| AJOU-38-VL | QSVLTQPPSASGTPGQRVTISCTGSSSNIGNNTVSWYQQLPGTAPKL |
| | LIYDNSHRPSGVPDRFSGSKSGTSASLAISGLQSEDEADYYCGSWD |
| | YSLSAYVFGGGTKLTVL (SEQ ID NO: 152) |
| AJOU-39-VL | QSVLTQPPSASGTPGQRVTISCTGSSSNIGNNDVNWYQQLPGTAPK |
| | LLIYYDSQRPSGVPDRFSGSKSGTSASLAISGLRSEDEADYYCATW |
| | DASLSAYVFGGGTKLTVL (SEQ ID NO: 153) |
| AJOU-40-VL | QSVLTQPPSASGTPGQRVTISCSGSSSNIGSNAVNWYQQLPGTAPKL |
| | LIYYDNQRPSGVPDRFSGSKSGTSASLAISGLRSEDEADYYCGTWD |
| | DSLNGYVFGGGTKLTVL (SEQ ID NO: 154) |
| AJOU-41-VL | QSVLTQPPSASGTPGQRVTISCSGSSSNIGNNAVTWYQQLPGTAPK |
| | LLIYDDSHRPSGVPDRFSGSKSGTSASLAISGLRSEDEADYYCGSW |
| | DYSLSAYVFGGGTKLTVL (SEQ ID NO: 155) |
| AJOU-42-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL (SEQ ID NO: 156) |
| AJOU-77-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL (SEQ ID NO: 156) |
| AJOU-78-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLRGYVLGGGTKLTVL (SEQ ID NO: 157) |
| AJOU-79-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGYWD |
| | YSLSGYVLGGGTKLTVL (SEQ ID NO: 158) |
| AJOU-80-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL (SEQ ID NO: 156) |
| AJOU-86-VL | QSVLTQPPSASGTPGQRVTISCSGSSANSRTDGFNWYQQLPGTAPK |
| | LLIYADSHRPSGVPDRFSGSKSGTSASLAISGLRSEDEADYYCGTW |
| | DYSLSGYVLGGGTKLTVLG (SEQ ID NO: 159) |
| AJOU-87-VL | QSVLTQPPSASGTPGQRVTISCSGSAQFGSRDNFNWYQQLPGTAPK |
| | LLIYADSHRPSGVPDRFSGSKSGTSASLAISGLRSEDEADYYCGTW |
| | DYSLSGYVLGGGTKLTVLG (SEQ ID NO: 160) |
| AJOU-88-VL | QSVLTQPPSASGTPGQRVTISCSGSTKQMHNYQFNWYQQLPGTAP |
| | KLLIYADSHRPSGVPDRFSGSKSGTSASLAISGLRSEDEADYYCGT |
| | WDYSLSGYVLGGGTKLTVLG (SEQ ID NO: 161) |
| AJOU-89-VL | QSVLTQPPSASGTPGQRVTISCSGSLLRGENLQFNWYQQLPGTAPK |
| | LLIYADSHRPSGVPDRFSGSKSGTSASLAISGLRSEDEADYYCGTW |
| | DYSLSGYVLGGGTKLTVLG (SEQ ID NO: 162) |
| AJOU-90-VL | QSVLTQPPSASGTPGQRVTISCSGSPLFPDSGSFNWYQQLPGTAPKL |
| | LIYADSHRPSGVPDRFSGSKSGTSASLAISGLRSEDEADYYCGTWD |
| | YSLSGYVLGGGTKLTVLG (SEQ ID NO: 163) |
| AJOU-91-VL | QSVLTQPPSASGTPGQRVTISCSGSAALDLSPSFNWYQQLPGTAPKL |
| | LIYADSHRPSGVPDRFSGSKSGTSASLAISGLRSEDEADYYCGTWD |
| | YSLSGYVLGGGTKLTVLG (SEQ ID NO: 164) |
| AJOU-84- | RHAMA (SEQ ID NO: 165) |
| HCDR1 | |
| AJOU-85- | AITSSGRSIYYADSVKG (SEQ ID NO: 166) |
| HCDR2 | |
| AJOU-32- | VHRAFDY (SEQ ID NO: 167) |
| HCDR3 | |
| AJOU-96- | SGSPLFPDSGSFN (SEQ ID NO: 168) |
| LCDR1 | |
| AJOU-60- | ADSHRPS (SEQ ID NO: 169) |
| LCDR2 | |
| AJOU-68- | GTWDYSLSGYV (SEQ ID NO: 170) |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of 11/3, 27/19, 43/35, 59/51, 75/67, 91/83, 107/99, 123/115, 155/147, and 171/163.

The antibodies recited below in Table 4 are described in more detail in U.S. Pat. Nos. 7,605,237 and 7,608,693, incorporated herein by reference in their entireties for all purposes.

TABLE 4

| Sequence ID | Sequence |
|---|---|
| REGN-VH-3 | QVQLVQSGAEVKKPGASVKVSCKASGYTFTNYGISWVRQAPGQG |
| | LEWMGWISVYNGKTNYAQKLQGRVTMTTDTSTTTAYMEMRSLR |
| | SDDTAVYYCARGSGYDLDYWGQGTLVSVSS (SEQ ID NO: 171) |
| REGN-VH-19 | EVQLVESGGGLVQPGGSLRLSCAASGFTFSSFWMTWVRQAPGKG |
| | LEWVANIKQDGSEKYYVDSVKGRFTISRDNAKNSLYLQMNSLRA |
| | EDTAVYYCARDPGRTMVRGGIRYYYGMDVWGQGTTVTVSS |
| | (SEQ ID NO: 172) |
| REGN-VH-35 | EVKLAESGGGLVQPGGSLRLSCAASGFTFSSHWMNWVRQAPGKG |
| | LEWVANIKQDGSDKYYVDSVKGRFTISRDNAKNSLYLQLNSLIAE |
| | DTAVYYCARDRGVRPPRGAFDIWGQGTMVTVSS (SEQ ID NO: 173) |
| REGN-VH-51 | QVQLVQSGAEVKKPGASVKVSCKASGYTENSYGISWVRQAPGQG |
| | LEWMGWIRTYNGNTNYAQKLQGRVTMTTDTSTSTAYMELRSLR |
| | SDDTAVYYCARDEARIVVAGTTPYYYGMDVWGQGTTVTVSS |
| | (SEQ ID NO: 174) |
| REGN-VH-67 | QVQLVESGGGLVQPGGSLRLSCAVSGFTISDHYMSWIRQAPGKGL |
| | EWISYISSSGSKIYYADSVKGRFTISRDNAKNSLFLQMNSLRAEDT |
| | AVYYCARTRQLVGDYWGQGTLVTVSS (SEQ ID NO: 175) |
| REGN-VH-83 | EVQLVESGGGLVQPGRSLRLSCAASGFTFDNYAMHWVRQAPGK |
| | GLEWVSGIRWNSGSIGYADSVKGRFTISRDNAKNSLYLQMNSLRA |
| | EDTALYYCAKEGGYSGYRPGPFFDYWGQGTLVTVSS (SEQ ID |
| | NO: 176) |
| REGN-VH-99 | QVQLVQSGAEVKKPGASVKVSCKASGYTFTNYGISWVRQAPGQG |
| | LEWMGWISVYNGHTNYAQKLQGRVTMTTDTSTSTAYMELRSLR |
| | SDDTAVYYCARGSGYDFDSWGQGTLVTVSS (SEQ ID NO: 177) |
| REGN-VH-115 | QVQLVQSGAEVKKPGASVKVSCKASRYTFTSYDINWVRQATGQG |
| | LEWMGWMNPNSGNTGYAQKFQGRVTMTRNTSTSTAYMELSSLR |
| | SEDTAVYYCARVRRFFDYWGQGTLVTVSS (SEQ ID NO: 178) |
| REGN-VH-147 | QVQLVQSGPEVKKPGASVKVSCKASGYTFTNYGISWVRQAPGQG |
| | LEWMGWISVYNGNINYAQKLQGRVTMTTDTSTSTAYMDLRSLRS |
| | DDTAVYYCARGSGYDFDYWGQGTLVTVSS (SEQ ID NO: 179) |
| REGN-VH-163 | QVQLVQSGAEVKKPGASVKVSCKDSAYTFNRYGISWVRQAPGQG |
| | LEWMGWISAYTGNTVYAQKLQGRVTMTTDNSTSTAYMELRSLR |
| | SDDTAVYYCARDKSIFGVVRGFDYWGQGTLVTVSS (SEQ ID |
| | NO: 180) |
| REGN-VL-11 | AIQMTQSPSSLSASVGDRVTITCRASQGIRNALGWYQQKPGKAPK |
| | LLIYAASSLQSGVPSRFSGSGSGTDFTLTFSSLQPEDFATYYCLQDF |
| | NYPYTFGQGTKLEIK (SEQ ID NO: 181) |
| REGN-VL-27 | DIQMTQSPSSVSASVGDRVTISCRASQGVSSWLAWYQQKPGNAP |
| | KLLISAASSIQSGVPSRFSGSGSGTDFTLTISSLQPEDFATYYCQQA |
| | NSFPLTFGGGTKVEIK (SEQ ID NO: 182) |
| REGN-VL-43 | DIQMTQSPSSVSASVGDRVTITCRASQGISSWLAWYQQKPGKAPK |
| | LLIYAASSFQSGVPSRFSGSGSGTDFTLTISSLQPEDFATYFCQQAN |
| | SFPLTFGGGTTVEIK (SEQ ID NO: 183) |
| REGN-VL-59 | DIQMTQSPSSVSASVGDRVTITCRASQDISIWLAWYQQSPGKAPKL |
| | LINVASRLQSGVPSRFSGSGSGTDFTLTINSLQPEDFVTYYCQQAN |
| | SFPITFGQGTRLATK (SEQ ID NO: 184) |
| REGN-VL-75 | DIQLTQSPSFLSASVGDRVTITCWASQGISSYLAWYQQKPGKAPKL |
| | LIFAASTLQSGVPSRFSGSGSGTEFTLTISSLQPEDFATYYCQQLNS |
| | YPLTFGGGTKVEIR (SEQ ID NO: 185) |
| REGN-VL-91 | EIVMTQSPATLSVSPGERATLSCRASQSVNYNLAWYQHKPGQAPR |
| | LLIYGASTRATGIPARFSGSGSGTEFTLTISSLQSEDFAVYYCQQYN |
| | NWPLTFGGGTKVEIK (SEQ ID NO: 186) |
| REGN-VL-107 | AIQMTQSSSSLSASVGDRVTITCRASQAIRNALGWYQQKPGKAPK |
| | VLIYAASSLQSGIPSRFSGSGSGTDFTLTISSLQPEDFATYYCLQDY |
| | DYPYTFGQGTKLEIK (SEQ ID NO: 187) |
| REGN-VL-123 | DIQLTQSPSFLSASVGDRVTITCWASQGIISYLAWYQQKPGKAPKL |
| | LIYAASTLHSGVPSRFSGSGSGTEFTLTISSLQPEDFATYYCHQLKS |
| | YPITFGQGTRLEIK (SEQ ID NO: 188) |
| REGN-VL-155 | AIQMTQSPSSLSASVGDRVTITCRASQDIRNALGWYQQKPGKAPK |
| | LLIYAASSLQSGVPSRFSGSASGTDFTLTISSLQPEDFAAYYCLQDY |
| | NYPYTFGQGTKLEIK (SEQ ID NO: 189) |
| REGN-VL-171 | EIVMTQSPVTLSLSPGERATLPCRASQSVSSSLAWYQQKAGQSPRL |
| | LIYGASTRATGIPARFSGSGSGTEFTLTISNLQSEDFAVYYCQQYN |
| | NWPLTFGGGTKVEIK (SEQ ID NO: 190) |

The antibodies recited below in Table 5 are described in more detail in WO2022/052974, incorporated herein by reference in its entirety for all purposes.

TABLE 5

| | |
|---|---|
| EVQLLESGGGLVQPGGSLRLSCAASGFTLSSYAMHWVR | STSA-C27-VH |
| QAPGKGLEYVSGISSNGGSTYYANSVKGRFTISRDNPKN | |
| TLFLQMSSLRAEDTAVYYCVRVKVGYRGGMDVWGQG | |
| TTVTVSS (SEQ ID NO: 191) | |
| EVQLLESGGGLVQPGGSLRLSCAASGFTLSSYAMHWVR | STSA-C27-6-33-VH |
| QAPGKGLEYVSGISPSGSSTYYANSVKGRFTISRDNPKNT | |
| LFLQMSSLRAEDTAVYYCVRSKVRYRGGMDVWGQGTT | |
| VTVSS (SEQ ID NO: 192) | |
| EVQLLESGGGLVQPGGSLRLSCAASGFTLSSYAMHWVR | STSA-C27-7-33-VH |
| QAPGKGLEYVSGISPSGVSTYYANSVKGRFTISRDNPKN | |
| TLFLQMSSLRAEDTAVYYCVRVKVKYRGGMDVWGQG | |
| TTVTVSS (SEQ ID NO: 193) | |
| EVQLLESGGGLVQPGGSLRLSCAASGFTLSSYAMHWVR | STSA-C27-24-56- |
| QAPGKGLEYVSGISPTSGSTYYANSVKGRFTISRDNPKNT | VH |
| LFLQMSSLRAEDTAVYYCVRVKVRYRGGMDVWGQGTT | |
| VTVSS (SEQ ID NO: 194) | |
| EVQLLESGGGLVQPGGSLRLSCAASGFTLSSYAMHWVR | STSA-C27-47-56- |
| QAPGKGLEYVSGISPTGTSTYYANSVKGRFTISRDNPKNT | VH |
| LFLQMSSLRAEDTAVYYCVRVKGAYRGGMDVWGQGT | |
| TVTVSS (SEQ ID NO: 195) | |
| EVQLLESGGGLVQPGGSLRLSCAASGFTLSSYAMHWVR | STSA-C27-33-33- |
| QAPGKGLEYVSGISSSGSSTYYANSVKGRFTISRDNPKNT | VH |
| LFLQMSSLRAEDTAVYYCVRVKVAYRGGMDVWGQGT | |
| TVTVSS (SEQ ID NO: 196) | |
| EVQLLESGGGLVQPGGSLRLSCAASGFTLSSYAMHWVR | STSA-C27-56-56- |
| QAPGKGLEYVSGISPSSTSTYYANSVKGRFTISRDNPKNT | VH |
| LFLQMSSLRAEDTAVYYCVRVKVLYRGGMDVWGQGTT | |
| VTVSS (SEQ ID NO: 197) | |
| EVQLLESGGGLVQPGGSLRLSCAASGFTLSSYAMHWVR | STSA-C27-78-78- |
| QAPGKGLEYVSGISPSSASTYYANSVKGRFTISRDNPKNT | VH |
| LFLQMSSLRAEDTAVYYCVRVKSKYRGGMDVWGQGTT | |
| VTVSS (SEQ ID NO: 198) | |
| EVQLLESGGGLVQPGGSLRLSCAASGFTLSSYAMHWVR | STSA-C27-82-58- |
| QAPGKGLEYVSGISGNSASTYYANSVKGRFTISRDNPKN | VH |
| TLFLQMSSLRAEDTAVYYCVRVKLKYRGGMDVWGQGT | |
| TVTVSS (SEQ ID NO: 199) | |
| EVQLLESGGGLVQPGGSLRLSCAASGFTLSSYAMHWVR | STSA-C27-54-54- |
| QAPGKGLEYVSGISHSGTSTYYANSVKGRFTISRDNPKN | VH |
| TLFLQMSSLRAEDTAVYYCVRVRVLYRGGMDVWGQGT | |
| TVTVSS (SEQ ID NO: 200) | |
| EVQLLESGGGLVQPGGSLRLSCAASGFTLSSYAMHWVR | STSA-C27-36-36- |
| QAPGKGLEYVSGISPSGVSTYYANSVKGRFTISRDNPKN | VH |
| TLFLQMSSLRAEDTAVYYCVRVKVKYRGGMDVWGQG | |
| TTVTVSS (SEQ ID NO: 193) | |
| EVQLLESGGGLVQPGGSLRLSCAASGFTLSSYAMHWVR | STSA-C27-53-53- |
| QAPGKGLEYVSGISSNGGSTYYANSVKGRFTISRDNPKN | VH |
| TLFLQMSSLRAEDTAVYYCVRVFVRYRGGMDVWGQGT | |
| TVTVSS (SEQ ID NO: 201) | |
| EVQLLESGGGLVQPGGSLRLSCAASGFTLSSYAMHWVR | STSA-C27-67-67- |
| QAPGKGLEYVSGISPTSASTYYANSVKGRFTISRDNPKNT | VH |
| LFLQMSSLRAEDTAVYYCVRVKGRYRGGMDVWGQGTT | |
| VTVSS (SEQ ID NO: 202) | |
| EVQLLESGGGLVQPGGSLRLSCAASGFTLSSYAMHWVR | STSA-C27-55-55- |
| QAPGKGLEYVSGISPTGGSTYYANSVKGRFTISRDNPKN | VH |
| TLFLQMSSLRAEDTAVYYCVRVKGRYRGGMDVWGQGT | |
| TVTVSS (SEQ ID NO: 203) | |
| EVQLLESGGGLVQPGGSLRLSCAASGFTLSSYAMHWVR | STSA-C27-59-59- |
| QAPGKGLEYVSGISHSGNSTYYANSVKGRFTISRDNPKN | VH |
| TLFLQMSSLRAEDTAVYYCVRVKRRYRGGMDVWGQGT | |
| TVTVSS (SEQ ID NO: 204) | |
| EVQLLESGGGLVQPGGSLRLSCAASGFTLSSYAMHWVR | STSA-C27-58-58- |
| QAPGKGLEYVSGISPSSNSTYYANSVKGRFTISRDNPKNT | VH |
| LFLQMSSLRAEDTAVYYCVRVKVRYRGGMDVWGQGTT | |
| VTVSS (SEQ ID NO: 205) | |
| EVQLLESGGGLVQPGGSLRLSCAASGFTLSSYAMHWVR | STSA-C27-52-52- |
| QAPGKGLEYVSGISSSGSSTYYANSVKGRFTISRDNPKNT | VH |
| LFLQMSSLRAEDTAVYYCVRVKPAYRGGMDVWGQGTT | |
| VTVSS (SEQ ID NO: 206) | |
| EVQLLESGGGLVQPGGSLRLSCAASGFTLSSYAMHWVR | STSA-C27-Y2-Y2- |
| QAPGKGLEYVSGISYSSASTYYANSVKGRFTISRDNPKN | VH |
| TLFLQMSSLRAEDTAVYYCVRVKVRYRGGMDVWGQGT | |
| TVTVSS (SEQ ID NO: 207) | |
| ETTLTQSPDTLPLSPGDRASLSCRASQSVSSAYLAWYQQ | STSA-C27-VL |
| KPGQAPRLLIYGTSRRATGVPGRFSGSGSGTDFTLTISRL | |
| EPEDFAVYYCQLYGSSSVTFGQGTKLEIK (SEQ ID | |
| NO: 208) | |
| EIVLTQSPGTLSLSPGERATLSCRASQGISSAYLAWYQQK | STSA-C27-6-33-VL |
| PGQAPRLLIYGTSRRATGIPDRFSGSGSGTDFTLTISRLEP | |
| EDFAVYYCQLYGATSVTFGQGTKLEIK (SEQ ID NO: 209) | |
| EIVLTQSPGTLSLSPGERATLSCRASQGISSAYLAWYQQK | STSA-C27-7-33-VL |
| PGQAPRLLIYGTSRRATGIPDRFSGSGSGTDFTLTISRLEP | |
| EDFAVYYCQLYGATSVTFGQGTKLEIK (SEQ ID NO: 209) | |
| EIVLTQSPGTLSLSPGERATLSCRASQSVSSAYLAWYQQ | STSA-C27-24-56- |
| KPGQAPRLLIYGTSRRATGIPDRFSGSGSGTDFTLTISRLE | VL |
| PEDFAVYYCQLYGASSVTFGQGTKLEIK (SEQ ID NO: 210) | |
| EIVLTQSPGTLSLSPGERATLSCRASQSVSSAYLAWYQQ | STSA-C27-47-56- |
| KPGQAPRLLIYGTSRRATGIPDRFSGSGSGTDFTLTISRLE | VL |
| PEDFAVYYCQLYGASSVTFGQGTKLEIK (SEQ ID NO: 210) | |
| EIVLTQSPGTLSLSPGERATLSCRASQGISSAYLAWYQQK | STSA-C27-33-33- |
| PGQAPRLLIYGTSRRATGIPDRFSGSGSGTDFTLTISRLEP | VL |
| EDFAVYYCQLYGATSVTFGQGTKLEIK (SEQ ID NO: 209) | |
| EIVLTQSPGTLSLSPGERATLSCRASQSVSSAYLAWYQQ | STSA-C27-56-56- |
| KPGQAPRLLIYGTSRRATGIPDRFSGSGSGTDFTLTISRLE | VL |
| PEDFAVYYCQLYGASSVTFGQGTKLEIK (SEQ ID NO: 210) | |
| EIVLTQSPGTLSLSPGERATLSCRASQSISTAYLAWYQQK | STSA-C27-78-78- |
| PGQAPRLLIYGTSRRATGIPDRFSGSGSGTDFTLTISRLEP | VL |
| EDFAVYYCQLYGASSVTFGQGTKLEIK (SEQ ID NO: 211) | |
| EIVLTQSPGTLSLSPGERATLSCRASQDISSAYLAWYQQK | STSA-C27-82-58- |
| PGQAPRLLIYGTSRRATGIPDRFSGSGSGTDFTLTISRLEP | VL |
| EDFAVYYCQLYGATSVTFGQGTKLEIK (SEQ ID NO: 212) | |
| EIVLTQSPGTLSLSPGERATLSCRASQDVSSAYLAWYQQ | STSA-C27-54-54- |
| KPGQAPRLLIYGTSRRATGIPDRFSGSGSGTDFTLTISRLE | VL |
| PEDFAVYYCQLYGATSVTFGQGTKLEIK (SEQ ID NO: 213) | |
| EIVLTQSPGTLSLSPGERATLSCRASQNISTAYLAWYQQK | STSA-C27-36-36- |
| PGQAPRLLIYGTSRRATGIPDRFSGSGSGTDFTLTISRLEP | VL |
| EDFAVYYCQLYGATSVTFGQGTKLEIK (SEQ ID NO: 214) | |
| EIVLTQSPGTLSLSPGERATLSCRASQDASNAYLAWYQQ | STSA-C27-53-53- |
| KPGQAPRLLIYGTSRRATGIPDRFSGSGSGTDFTLTISRLE | VL |
| PEDFAVYYCQLYGSSSVTFGQGTKLEIK (SEQ ID NO: 215) | |
| EIVLTQSPGTLSLSPGERATLSCRASQGVSSAYLAWYQQ | STSA-C27-67-67- |
| KPGQAPRLLIYGTSRRATGIPDRFSGSGSGTDFTLTISRLE | VL |
| PEDFAVYYCQLYGRSSVTFGQGTKLEIK (SEQ ID NO: 216) | |
| EIVLTQSPGTLSLSPGERATLSCRASQNISTAYLAWYQQK | STSA-C27-55-55- |
| PGQAPRLLIYGTSRRATGIPDRFSGSGSGTDFTLTISRLEP | VL |
| EDFAVYYCQLYGTSSVTFGQGTKLEIK (SEQ ID NO: 217) | |
| EIVLTQSPGTLSLSPGERATLSCRASQSVSTAYLAWYQQ | STSA-C27-59-59- |
| KPGQAPRLLIYGTSRRATGIPDRFSGSGSGTDFTLTISRLE | VL |
| PEDFAVYYCQLYGATSVTFGQGTKLEIK (SEQ ID NO: 218) | |
| EIVLTQSPGTLSLSPGERATLSCRASQDISSAYLAWYQQK | STSA-C27-58-58- |
| PGQAPRLLIYGTSRRATGIPDRFSGSGSGTDFTLTISRLEP | VL |
| EDFAVYYCQLYGATSVTFGQGTKLEIK (SEQ ID NO: 212) | |
| EIVLTQSPGTLSLSPGERATLSCRASQGVSTAYLAWYQQ | STSA-C27-52-52- |
| KPGQAPRLLIYGTSRRATGIPDRFSGSGSGTDFTLTISRLE | VL |
| PEDFAVYYCQLYGATSVTFGQGTKLEIK (SEQ ID NO: 219) | |
| EIVLPQSPGTLSLSPGERATLSCRASQGVSSAYLAWYQQ | STSA-C27-Y2-Y2- |
| KPGQAPRLLIYGTSRRATGIPDRFSGSGSGTDFTLTISRLE | VL |
| PEDFAVYYCQLYGSTSVTFGQGTKLEIK (SEQ ID | |
| NO: 220) | |

In certain embodiments, an antibody or antigen-binding fragment thereof of the disclosure comprises heavy chain variable region (HCVR) and light chain variable region (LCVR) sequence pairs (HCVR/LCVR) selected from the group consisting of: Y0188-1/Y0188-1; Y0188-2/Y0188-2; Y0188-3/Y0188-3; Y0188-4/Y0188-4; Y0188-6/Y0188-6; Y0188-8/Y0188-8; Y0188-9/Y0188-9; Y0188-10/Y0188-10; Y0188-14/Y0188-14; HV3-15-14/Y01-14; HV3-15-14/164-14; HV3-15-14/KV4-14; HV3-15-14/KV1-27-14; HV3-15-14/KV1-9-14; HV3-15-14/KV1-NL1-14; HV3-15-14/KV1D-43-14; HV3-48-14/Y01-14; HV3-48-14/164-14; HV3-48-14/KV4-14; HV3-48-14/KV1-27-14; HV3-48-14/KV1-9-14; HV3-48-14/KV1-NL1-14; HV3-48-14/KV1D-43-14; HV3-73*2-14/Y01-14; HV3-73*2-14/164-14; HV3-73*2-14/KV4-14; HV3-73*2-14/KV1-27-14; HV3-73*2-14/KV1-9-14; HV3-73*2-14/KV1-NL1-14; HV3-73*2-14/KV1D-43-14; HV3-72-14/Y01-14; HV3-72-14/164-14; HV3-72-14/KV4-14; HV3-72-14/KV1-27-14; HV3-72-14/KV1-9-14; HV3-72-14/KV1-NL1-14; HV3-72-14/KV1D-43-14; Y01-14/Y01-14; Y01-14/164-14; Y01-14/KV4-14; Y01-14/KV1-27-14; Y01-14/KV1-9-14; Y01-14/KV1-NL1-14; Y01-14/KV1D-43-14; 162-14/Y01-14; 162-14/164-14; 162-14/KV4-14; 162-14/KV1-27-14; 162-14/KV1-9-14; 162-14/KV1-NL1-14; 162-14/KV1D-43-1L; VH73-14/Y01-14; VH73-14/164-14; VH73-14/KV4-14; VH73-14/KV1-27-14; VH73-14/KV1-9-14; VH73-14/KV1-NL1-14; and VH73-14/KV1D-43-14.

The antibodies recited below in Table 6 are described in more detail in WO2021/213329, incorporated herein by reference in its entirety for all purposes.

TABLE 6

| | |
|---|---|
| Y0188-1 | EVQLVESGGGLVQPKGSLKLSCAASGFTFNTYGMHWVRQAPG |
| VH | KGLEWVAHIRSKSSNYATYYADSVKDRFTISRDDSQSMLYLQM |
| | NNLKTEDTAMYYCVRWFRAMDYWGQGTSVTVSS (SEQ ID |
| | NO: 221) |
| Y0188-2 | EVQLIESGGGLVQPKGSLKLSCAASGFTFNMYAMDWVRQAPGK |
| VH | GLEWVARIRSKGSNFETNYADSVKDRFTISRDDSQSMVYLQMIN |
| | LKTEDTAMYYCVRHRGGAWFAYWGQGTLVSVSA (SEQ ID |
| | NO: 222) |
| Y0188-3 | QVQLVETGGGLVRPGNSLKLSCVTSGFTFSNYRMHWLRQPPGK |
| VH | RLEWIAVITVKSNNYGANYAESVKGRFAISRDDSKSSVYLEMNR |
| | LREEDTATYFCSRERAYGNPFDYWGQGTTLTVSS (SEQ ID |
| | NO: 223) |
| Y0188-4 | EVQLVESGGGLVQPKGSLKLSCAASGFTFNMYAMNWVRQAPG |
| VH | QGLEWVARIRSKSNNYATYYADSVKDRFIISRDDSESMVYLQMS |
| | NLRAADTAMYYCVRHLRAMDYWGQGTSVTVSS (SEQ ID |
| | NO: 224) |
| Y0188-6 | EVQLVESGGGLVQPKGSLKLSCAASGFSFNMYAMNWVRQAPG |
| VH | KGLEWVARIRTKSNHYSTYYADSVKDRFTISRDDSASMFYLQM |
| | NNLKTEDTAMYFCVRHLRAMDYWGQGTSVTVSS (SEQ ID |
| | NO: 225) |
| Y0188-8 | EVQLIESGGGLVQPKGSLKLSCAASGFTFNMYAMDWVRQAPGK |
| VH | GLEWVARIRSKGSNFETNYADSVKDRFTISRDDSQSMVYLQMN |
| | NLKTEDTAMYYCVRHRGGAWFAYWGQGTLVTVSA (SEQ ID |
| | NO: 226) |
| Y0188-9 | EVQLVESGGGLVRPKGSLKLSCAASGFSFNTYAMNWVRQAPGK |
| VH | GLEWIVWIRSKSHNYATYYADSVKDRFTISRDDSESMLYLQMN |
| | NLKTEDTAMYYCVRHLRAMDYWGQGTSVTVSS (SEQ ID |
| | NO: 227) |
| Y0188-10 | EVRLVESGGGLVQPKGSLKLSCEASGFSFNMYAMNWVRQAPG |
| VH | KGLEWITHIRSKSNNYATYYADSVKDRFIISRDDSESMVYLQMN |
| | NLKTEDTAMYYCVRLLRALDYWGQGTSVTVSS (SEQ ID |
| | NO: 228) |
| Y0188-14 | EVQLVESGGGLVQPKGSLKLSCAASGFTFNMYGMHWVRQAPG |
| VH | KGLEWVAHIRSKSSNYATYYADSVKDRLTISRDDSQSMLYLQM |
| | NNLKTEDTAMYYCVRWFRAMDYWGQGTSVTVSS (SEQ ID |
| | NO: 229) |
| HV3-15-14 | EVQLVESGGGLVKPGGSLRLSCAASGFTFSMYGMHWVRQAPG |
| VH | KGLEWVGHIRSKSSNYATYYADSVKDRFTISRDDSKNTLYLQM |
| | NSLKTEDTAVYYCTTWFRAMDYWGQGTLVTVSS (SEQ ID |
| | NO: 230) |
| HV3-48-14 | EVQLVESGGGLVQPGGSLRLSCAASGFTFSMYGMHWVRQAPG |
| VH | KGLEWVSHIRSKSSNYATYYADSVKDRFTISRDNAKNSLYLQM |
| | NSLRAEDTAVYYCARWFRAMDYWGQGTLVTVSS (SEQ ID |
| | NO: 231) |
| HV3-73*2- | EVQLVESGGGLVQPGGSLKLSCAASGFTFSMYGMHWVRQASG |
| 14 | KGLEWVGHIRSKSSNYATYYADSVKDRFTISRDDSKNTAYLQM |
| VH | NSLKTEDTAVYYCTRWFRAMDYWGQGTLVTVSS (SEQ ID |
| | NO: 232) |
| HV3-72-14 | EVQLVESGGGLVQPGGSLRLSCAASGFTFSMYGMHWVRQAPG |
| VH | KGLEWVGHIRSKSSNYATYYADSVKDRFTISRDDSKNSLYLQM |
| | NSLKTEDTAVYYCARWFRAMDYWGQGTLVTVSS (SEQ ID |
| | NO: 233) |
| Y01-14 | EVQLVESGGGLVQPGGSLRLSCAASGFTFSMYGMHWVRQAPG |
| VH | KGLEWVSHIRSKSSNYATYYADSVKDRFTISRDNAKNSLYLQM |
| | NSLRAEDTAVYYCARWFRAMDYWGQGTLVTVSS (SEQ ID |
| | NO: 231) |
| 162-14 | EVQLVESGGGLEQPGGSLRLSCAGSGFTFRMYGMHWVRQAPG |
| VH | KGLEWVSHIRSKSSNYATYYADSVKDRFTISRDNSKNTLYLQM |
| | NSLRAEDTAVYYCAKWFRAMDYWGQGTTVTVSS (SEQ ID |
| | NO: 234) |
| VH73-14 | EVQLVESGGGLVQPGGSLKLSCAASGFTFSMYGMHWVRQASG |
| VH | KGLEWVGHIRSKSSNYATYYADSVKDRFTISRDDSKNTAYLQM |
| | NSLKTEDTAVYYCTRWFRAMDYWGQGTTVTVSS (SEQ ID |
| | NO: 234) |
| Y0188-1 | DIVMTQSHKFMSTSVGDRVSITCKASQDVSTAVAWYQEKPGQS |
| VL | PKLLIYWASTRHTGVPDRFTGSGSGTDYTLTISSVQAEDLALYY |
| | CQQHYSTPLTFGAGTKLELK (SEQ ID NO: 235) |
| Y0188-2 | DIVVTQSPASLAVSLGQRATISCRASKSVSTSGYSYMHWYQQKP |
| VL | GQPPKLLIYLASNLESGVPARFSGSGSGTDFTLNIHPVEEEDVAIY |
| | YCQHSRELPLTFGAGTKLELK (SEQ ID NO: 236) |
| Y0188-3 | DIQMTQSPSSLSASLGERVSLTCRASQEISGYLSWLQQKPDGTIK |
| VL | RLIYAASTLDSGVPKRFSGSRSGSDYSLTISSLESEDFADYYCLQY |
| | GSYPYTFGGGTKLEIK (SEQ ID NO: 237) |
| Y0188-4 | DIVLTQSPASLTVSLGQRATISCRASKSVSTSGYSYMHWYQQKP |
| VL | GQPPKLLIYLASNLESGVPARFSGSGSGTDFTLNIHPVEEEDAAT |
| | YYCQHSRELPITFGSGTKLEIK (SEQ ID NO: 238) |
| Y0188-6 | DIVLTQSPASLVVSLGQRATISCRASQSVSTSGYSYMHWYQQKP |
| VL | GQPPKLLIYLASNVQSGVPARFSGSGSGTDFTLNIHPVEEEDVAT |
| | YYCHHNRDLPFTFGSGTKLEIK (SEQ ID NO: 239) |
| Y0188-8 | DIVVTQSPASLAVSLGQRATISCRASKSVSTSGYSYMHWYQQKP |
| VL | GQPPKLLIYLASNLESGVPARFSGSGSGTDFTLNIHPVEEEDVAIY |
| | YCQHSRELPLTFGAGTKLELK (SEQ ID NO: 236) |
| Y0188-9 | DIVLTQSPASLAVSLGQRATISCRASKSVSASGYSYMHWYQQKP |
| VL | GQPPKLLIYLASNLQSGVPARFSGSGSGTDFTLNIHPVEEEDAAT |
| | YYCQHSRELPPTFGGGTKLEIK (SEQ ID NO: 240) |
| Y0188-10 | DIVLTQSPASLAVFLGQRATISCRASKSVSTSGYSYMHWYQQKA |
| VL | GQPPKLLIYLASNLESGVPARFSGSGSGTDFTLNIHPVEEEDAAT |
| | YYCHHSRELPITFGSGTKLEMK (SEQ ID NO: 241) |
| Y0188-14 | DIVMTQSHKFMSTSVGDRVSITCKASQDVSTAVAWYQEKPGQS |
| VL | PKLLIYWASTRHTGVPDRFTGSGSGTDYTLTISSVQAEDLALYY |
| | CQQHYSTPLTFGAGTKLELK (SEQ ID NO: 235) |
| Y01-14 | EIVLTQSPGTLSLSPGERATLSCKASQDVSTAVAWYQQKPGQAP |
| VL | RLLIYWASTRHTGIPDRFSGSGSGTDFTLTISRLEPEDFAVYYCQ |
| | QHYSTPLTFGQGTKVEIK (SEQ ID NO: 242) |
| 164-14 | DIVMTQSPLSLPVTPGEPASISCKASQDVSTAVAWYLQKSGQSP |
| VL | QLLIYWASTRHTGVPDRFSGSGSGTDFTLKISRVEAEDVGFYYC |
| | QQHYSTPLTFGQGTKLEIK (SEQ ID NO: 243) |
| KV4-14 | DIVMTQSPDSLAVSLGERATINCKASQDVSTAVAWYQQKPGQP |
| VL | PKLLIYWASTRHTGVPDRFSGSGSGTDFTLTISSLQAEDVAVYYC |
| | QQHYSTPLTFGGGTKVEIK (SEQ ID NO: 244) |
| KV1-27-14 | DIQMTQSPSSLSASVGDRVTITCKASQDVSTAVAWYQQKPGKVP |
| VL | KLLIYWASTRHTGVPSRFSGSGSGTDFTLTISSLQPEDVATYYCQ |
| | QHYSTPLTFGGGTKVEIK (SEQ ID NO: 245) |
| KV1-9-14 | DIQLTQSPSFLSASVGDRVTITCKASQDVSTAVAWYQQKPGKAP |
| VL | KLLIYWASTRHTGVPSRFSGSGSGTEFTLTISSLQPEDFATYYCQ |
| | QHYSTPLTFGGGTKVEIK (SEQ ID NO: 246) |
| KV1-NL1- | DIQMTQSPSSLSASVGDRVTITCKASQDVSTAVAWYQQKPGKAP |
| 14 | KLLLYWASTRHTGVPSRFSGSGSGTDYTLTISSLQPEDFATYYCQ |
| VL | QHYSTPLTFGGGTKVEIK (SEQ ID NO: 247) |
| KV1D-43- | AIRMTQSPFSLSASVGDRVTITCKASQDVSTAVAWYQQKPAKAP |
| 14 | KLFIYWASTRHTGVPSRFSGSGSGTDYTLTISSLQPEDFATYYCQ |
| VL | QHYSTPLTFGGGTKVEIK (SEQ ID NO: 248) |

In certain embodiments, an antibody or antigen-binding fragment thereof of the disclosure comprises heavy chain variable region (HCVR) and light chain variable region (LCVR) sequence pairs (HCVR/LCVR) selected from the group of clones consisting of: 1A6 VH, 1D8 VH, 1H9 VH, 2H1 VH, 2F8 VH, 9B34 VH, 9E7 VH, 24G10 VH, 25D6 VH, 25G9 VH, 31B9 VH, 34A2 VH, 34H11 VH, 35D5 VH, 35A7-1 VH, 35A7-2 VH, 36F4 VH, 1A6 VL, 1D8 VL, 1H9 VL, 2H1 VL, 2F8 VL, 9B4 VL, 9E7 VL, 24G10 VL, 25D6 VL, 25G9 VL, 31B9 VL, 34A2 VL, 34H11 VL, 35D5 VL, 35A7-1 VL, 35A7-2 VL, 36F4 VL, VH1021, VH1022, VH1023, VH1024, VH1025, VH1026, VH1027, VH1028, VL1011, VL1012, VL1013, and VL1014.

The antibodies recited below in Table 7 are described in more detail in U.S. Pat. No. 11,725,057 B2, incorporated herein by reference in its entirety for all purposes.

TABLE 7

| | |
|---|---|
| 1A6 | QVQLQQSGTELVKPGASVKMSCKASVNTFTGYNMHWIKQTPGQGLEWIG |
| VH | GLHPGNGDSSYNQKFKGRATLTDKSSNTAYMQLSSLTSEDSAVYYCAL |
| | TTAGRAWFPYWGQGTLVTVSA (SEQ ID NO: 249) |
| 1A6 | DIQMTQTTSSLSASLGDRVTISCRASQDISNYLNWYQQKPDGTVKLLIYYT |
| VL | SRLHSGVPSRFSGSGSGTDYSLTISNLEQEDIATYFCQQGNTLPPT |
| | FGGGTKLEIK (SEQ ID NO: 250) |
| 1D8 | QVQLQQSGTELVKPGASVKMSCKASVNTFTGYNMHWIKQTPGQGLEWIG |
| VH | GLHPGNGDSSYNQKFKGRATLTADKSSNTAYMQLSSLTSEDSAVYYCAL |
| | TTAGRAWFAYWGQGTLVTVSA (SEQ ID NO: 251) |
| 1D8 | DIQMTQTTSSLSASLGDRVTISCRASQDISNYLNWYQQKPDGTVKLLIYYT |
| VL | SRLHSGVPSRFSGSGSGTDYSLTISNLEQEDIATYFCQQGNTLHTFGGGTKL |
| | EIK (SEQ ID NO: 252) |
| 1H9 | QVQLQQSGAELVKPGASVKMSCKASVNTFAGYNMHWVKQTPGQGLEWI |
| VH | GGLHPGNGDTSYNQKFKGKATLTADKSSNTAYMQLSSLTSEDSAVYYCA |
| | LTTAGRAWFAYWGQGTLVTVSA (SEQ ID NO: 253) |
| 1H9 | DIQMIQSTSSLSASLGDRVTISCRASQDISNYLNWYQQKPDGTVKLLIYYTS |
| VL | RLHSGVPSRFSGSGSGTDYSLTISNLEQEDIATYFCQQGNTLPWTFGGGTK |
| | LEIK (SEQ ID NO: 254) |
| 2H1 | QVQLQQSGAELVKPGASVKMSCKASVNTFTGYNMHWVKQTPGQGLEWI |
| VH | GGLHPGNGDTSYNQKFKGKATLTADKSSNTAYMQLSSLTSEDSAVYYCA |
| | LTTAGRAWFAYWGQGTLVTVSA (SEQ ID NO: 255) |
| 2H1 | DIQMIQSTSSLSASLGDRVTISCRASQDISNYLNWYQQKPDGTVKLLIYYTS |
| VL | RLHSGVPSRFSGSGSGTDYSLTISNLEQEDIATYFCQQGNTLPLTFGAGTKL |
| | ELK (SEQ ID NO: 256) |
| 2F8 | QVQLQQSGAELVKPGASVKMSCKASVNTFTGYNMHWVKQTPGQGLEWI |
| VH | GGLHPGNGDTSYNQKFKGKATLTADKSSNTAYMQLSSLTSEDSAVYYCA |
| | LTTAGRAWFPYWGQGTLVTVSA (SEQ ID NO: 257) |
| 2F8 | DIQMIQSTSSLSASLGDRVTISCRASQDISNYLNWYQQKPDGTVKLLIYYTS |
| VL | RLHSGVPSRFSGSGSGTDYSLTISNLEQEDIATYFCQQGNTLPWTFGGGTK |
| | LELK (SEQ ID NO: 258) |
| 9B4 | QVQLQQSGAELVKPGASVKMSCKASVNTFTGYNMHWIKQTPGQGLEWI |
| VH | GGLHPGNGDTSYNQKFKGKATLTADKSSNTAYMQLSSLTSEDSAVYYCA |
| | LTTAGRAWFAYWGQGTLVTVSA (SEQ ID NO: 259) |
| 9B4 | DIQMIQSTSSLSASLGDRVTISCRASQDISNYLNWYQQKPDGTVKLLIYYTS |
| VL | RLHSGVPSRFSGSGSGTDYSLTISNLEQEDIATYFCQQGNTLPWTFGGGTK |
| | LELK (SEQ ID NO: 258) |
| 9E7 | EVQLQQSGAELVKPGASVKMSCKASVNIFTGYNMHWVKQTPGQGLEWI |
| | GGLHPGNGDTSYNQKFKDKATLTADKSSNTAYMQLSSLTSEDSAVYYCA |
| VH | LTTAGRAWFAYWGQGTLVTVSA (SEQ ID NO: 260) |
| 9E7 | DIQMTQTTSSLSASLGDRVTISCRASQDISNYLNWYQQKPDGTVKLLIYYT |
| VL | SRLHSGVPSRFSGSGSGTDYSLTISNLEQEDIATYFCQQGNTLPWTFGGGT |
| | KLELK (SEQ ID NO: 261) |
| 24G10 | EVKLVESGGGLVKPGGSLKLSCAASGFTFSRYAMSWVRQTPEKRLEWVA |
| VH | TISSGGSYTNYADSVKGRFTISRDNVKNTLYLQMNSLRAEDTAVYYCARA |
| | TARATEFAYWGQGTLVTVSS (SEQ ID NO: 262) |
| 24G10 | DIQMTQTTSSLSASLGDRVTISCRASQDISNYLNWYQQKPDGTVKLLIY |
| VL | YTSRLHSGVPSRFSGSGSGTDYSLTISNLEQEDIATYFCQQGNTLPPTFGGG |
| | TKLEL (SEQ ID NO: 263) |
| 25D6 | EVQLQQSGAELVKPGASVKMSCKASVNIFTGYNMHWVKQTPGQGLEWI |
| VH | GGLHPGNGDTSYNQKFKDKATLTADKSSNTAYMQLSSLTSEDSAVYYCA |
| | LTTAGRAWFAYWGQGTLVTVSA (SEQ ID NO: 260) |
| 25D6 | DIQMTQSTSSLSASLGDRVTISCRASQDISNYLNWYQQKPDGTVKLLIYYT |
| VL | SRLHSGVPSRESGSGSGTDYSLTISNLEQEDIATYFCQQGNTLPWTFGGGT |
| | KLEIK (SEQ ID NO: 264) |
| 25G9 | EVQLKESGAELVKPGASVKMSCKASVNIFTGYNMHWVKQTPGQGLEWIG |
| VH | GLHPGNGDTSYNQKFKDKATLTADRSSNTAYMQLSSLTSEDSAVYYCAL |
| | TTAGRAWFAYWGQGTLVTVSA (SEQ ID NO: 265) |
| 25G9 | DIQMTQSTSSLSASLGDRVTISCRASQDISNYLNWYQQKPDGTVKLLIYYT |
| VL | SRLHSGVPSRFSGSGSGTDYSLTISNLEQEDIATYFCQQGNTLPWTFGGGT |
| | KLEIK (SEQ ID NO: 264) |
| 35A7-1 | QVQLKQSGAELVKPGASVKMSCTASVNIFTGYNMHWIKQTPGQGLEWIG |
| VH | GLHPGNGDTSYNQKFKDKATLTADRSSNTAYMQLSSLTSEDSAVYYCAL |
| | TTAGRAWFAYWGQGTLVTVSA (SEQ ID NO: 266) |
| 35A7-1 | DIQMNQSTSSLSASLGDRVTISCRASQDISNYLNWYQQKPDGTIKLLIYYTS |
| VL | RLYSGVPSRFSGSGSGTDYSLTISNLEEEDIATYFCQQGNTIPYTFGGGTKL |
| | EIK (SEQ ID NO: 267) |
| 31B9 | EVQLQQSGAELVKPGASVKMSCKASVNIFTGYNMHWVKQTPGQGLEWI |
| VH | GGLHPGNGDTSYNQKFKDKATLTADRSSNTAYMQLSSLTSEDSAVYYCA |
| | LTTAGRAWFAYWGQGTLVTVSA (SEQ ID NO: 268) |
| 31B9 | DIQMTQSTSSLSASLGDRVTISCRASQDISNYLNWYQQKPDGTIKLLIYYTS |
| VL | RLHSGVPSRFSGSGSGTDYSLTISNLEEEDIATYFCQQGNTLPWTFGGGTK |
| | LEIK (SEQ ID NO: 269) |
| 34A2 | EVQLQQSGAELVKPGASVKMSCKASVNIFTSYNMHWVKQTPGQGLEWIG |
| VH | GLHPGNGDTSYNQKFKGKATLTADKSSSTAYMQVSMLTSEDSAVYYCVL |
| | TTAGRAWFAYWGQGTLVTVSA (SEQ ID NO: 270) |
| 34A2 | DIQMTQSTSSLSASLGDRVTISCRASQDISNYLNWYQQKPDGTIKLLIYYTS |
| VL | RLHSGVPSRFSGSGSGTDYSLTISNLEEEDIATYFCQQGNTLPWTFGGGTK |
| | LEIK (SEQ ID NO: 269) |
| 34H11 | QVQLQESGAELVKPGASVKMSCKASVNTFTGYNMHWVKQTPGQGLEWI |
| VH | GGLHPGNGDTSYNQKFKGKATLTADRSSNTAYMQLSSLTSEDSAVYYCA |
| | LTTAGRAWFAYWGQGTLVTVSA (SEQ ID NO: 271) |
| 34H11 | DIQMTQTTSSLSASLGDRVTISCRASQDISNYLNWYQQKPDGTVKLLIYYT |
| VL | SRLHSGVPSRFSGSGSGTDYSLTISNLEEEDIATYFCQQGNTLPWTFGGGT |
| | KLEIK (SEQ ID NO: 272) |
| 35D5 | QVQLKQSGAELVKPGASVKMSCKASVNTFTGYNMHWVKQTPGQGLEWI |
| VH | GGLHPGNGDTSYNQKFKGKATLTADKSSNTAYMQLSSLTSEDSAVYYCA |
| | LTTAGRAWFAYWGQGTLVTVSA (SEQ ID NO: 273) |
| 35D5 | DIQMTQSTSSLSASLGDRVTISCRASQDISNYLNWYQQKPDGTVKLLIYYT |
| VL | SRLHSGVPSRESGSGSGTDYSLTISNLEEEDIATYFCQQGNTLPWTFGGGT |
| | KLEIK (SEQ ID NO: 274) |
| 35A7-2 | QVQLKQSGAELVKPGASVKMSCKASVNTFTGYNMHWIKQTPGQGLEWI |
| VH | GGLHPGNGDTSYNQKFKGKATLTADKSSNTAYMQLSSLTSEDSAVYYCA |
| | LTTAGRAWFAYWGQGTLVTVSA (SEQ ID NO: 275) |
| 35A7-2 | DIQMNQSTSSLSASLGDRVTISCRASQDISNYLNWYQQKPDGTIKLLIYYTS |
| VL | RLYSGVPSRFSGSGSGTDYSLTISNLEEEDIATYFCQQGNTLPYTFGGGTKL |
| | EIK (SEQ ID NO: 276) |
| 36F4 | QVQLQQSGAELVKPGASVKMSCKASVNTITGYNMHWVKQTPGQGLEWI |
| VH | GGLHPGNGDTSYNQKFKDKATLTADKSSNTAYMQLSSLTSEDSAVYYCA |
| | LTTAGRAWFAYWGQGTLVTVSA (SEQ ID NO: 277) |
| 36F4 | DIKMTQSTSSLSASLGDRVTTSCRASQDISNYLNWYQQKPDGTVKLLIYYT |
| VL | SRLHSGVPSRFSGSGSGTDYSLTISNLEEEDIATYFCQQGNTLPPWTFGGGT |
| | KLEIK (SEQ ID NO: 278) |
| VH1021 | EVQLVESGGGLVKPGGSLRLSCAASGFTFSRYAMSWVRQAPGKGLEWVS |
| | TISSGGSYTNYADSVKGRFTISRDNVKNTLYLQMNSLRAEDTAVYYCARA |
| | TARATEFAYWGQGTLVTVSS (SEQ ID NO: 279) |
| VH1022 | EVQLLESGGGLVQPGGSLRLSCAASGFTFSRYAMSWVRQAPGKGLEWVS |
| | TISSGGSYTNYADSVKGRFTISRDNVKNTLYLQMNSLRAEDTAVYYCARA |
| | TARATEFAYWGQGTLVTVSS (SEQ ID NO: 280) |
| VH1023 | QVQLVQSGAELKKPGASVKVSCKASGNTFTGYNMHWIQQSPGQGLEWM |
| | GGLHPGNGDSSYNQKFQGRVTLTADKSSNTAYMELSSLRSEDSAVYYCA |
| | LTTAGRAWFPYWGQGTLVTVSS (SEQ ID NO: 281) |
| VH1024 | QVQLVQSGAEVKKPGASVKMSCKASVYTFTGYNMHWIQQSPGQGLEWM |
| | GGLHPGNGDSSYNQKFQGRATLTADKSSNTAYMELSSLRSEDSAVYYCA |
| | LTTAGRAWFPYWGQGTLVTVSS (SEQ ID NO: 282) |
| VH1025 | QVQLVQSGAEVKKPGASVKVSCKASGNIFTGYNMHWVRQAPGQGLEW |
| | MGGLHPGNGDSSYNQKFQGRVTLTADKSSNTAYMELSSLRSEDSAVYYC |
| | ALTTAGRAWFAYWGQGTLVTVSS (SEQ ID NO: 283) |
| VH1026 | QVQLVQSGAEVKKPGASVKMSCKASVYTFTGYNMHWVRQAPGQGLEWI |
| | GGLHPGNGDSSYNQKFQGRATLTADKSSNTAYMELSSLRSEDSAVYYCA |
| | LTTAGRAWFAYWGQGTLVTVSS (SEQ ID NO: 284) |
| VH1027 | EVQLVQSGAEVKKPGATVKISCKASGNIFTSYNMHWVRQAPGQGLEWM |
| | GGLHPGNGDTSYNQKFQGRVTLTADKSSSTAYMELSSLRSEDTAVYYCV |
| | LTTAGRAWFAYWGQGTLVTVSS (SEQ ID NO: 285) |
| VH1028 | EVQLVQSGAEVKKPGATVKMSCKASVYTFTSYNMHWVRQAPGQGLEWI |
| | GGLHPGNGDTSYNQKFKGRATLTADKSSSTAYMELSSLRSEDTAVYYCV |
| | LTTAGRAWFAYWGQGTLVTVSS (SEQ ID NO: 286) |
| VL1011 | DIQMTQSPSSLSASVGDRVTITCQASQDISNYLNWYQQKPGKAPKLLIYYT |
| | SRLHSGVPSRFSGSGSGTDYTFTISSLQPEDIATYFCQQGNTLPWTFGGGTK |
| | VEIK (SEQ ID NO: 287) |
| VL1012 | DIQMTQSPSSLSASVGDRVTITCRASQDISNYLNWYQQKPGKAPKLLIYYT |
| | SRLHSGVPSRFSGSGSGTDYTLTISSLQPEDFATYFCQQGNTLPWTFGGGT |
| | KVEIK (SEQ ID NO: 288) |
| VL1013 | DIQMTQSPSSLSASVGDRVTITCQASQDISNYLNWYQQKPGKAPKLLIYYT |
| | SRLHSGVPSRFSGSGSGTDYTLTISSLQPEDFATYFCQQGNTLPLTFGGGTK |
| | VEIK (SEQ ID NO: 289) |
| VL1014 | DIQMTQSPSSLSASVGDRVTITCQASQDISNYLNWYQQKPGKAPKLLIYYT |
| | SRLHSGVPSRFSGSGSGTDYTLTISSLQPEDFATYFCQQGNTLPWTFGGGT |
| | KVEIK (SEQ ID NO: 290) |

### Pharmaceutical Compositions

Methods that comprise administering an IL-4R antagonist to a patient, wherein the IL-4R antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, Ypsomed YpsoDose, Bespak Lapas, and the like.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An example sleeve-triggered auto-injector with manual needle insertion is described in International Publication WO2015/004052. Example audible end-of-dose feedback mechanisms are described in International Publications WO2016/193346 and WO2016/193348. An example needle-safety mechanism after using an auto-injector is described in International Publication WO2016/193352. An example needle sheath remover mechanism for a syringe auto-injector is described in International Publication WO2016/193353. An example support mechanism for supporting an axial position of a syringe is described in International Publication WO2016/193355.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an IL-4R antagonist to a subject in need thereof, in an aerosolized formulation. For example, aerosolized antibodies to IL-4R may be administered to treat COPD in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used as described herein are disclosed, e.g., in U.S. Pat. No. 8,945,559.

### Dosage

The amount of IL-4R antagonist (e.g., an anti-IL-4R antibody or antigen-binding fragment thereof) administered to a subject according to the methods featured in the invention or for use according to the invention is, generally, a therapeutically effective amount. As used herein, the phrase “therapeutically effective amount” means an amount of IL-4R antagonist that results in one or more of: (a) a reduction in the incidence of COPD exacerbations; (b) an improvement in one or more COPD-associated parameters (as defined elsewhere herein); and/or (c) a detectable improvement in one or more symptoms or indicia of an upper airway inflammatory condition. A “therapeutically effective amount” also includes an amount of IL-4R antagonist that inhibits, prevents, lessens, or delays the progression of COPD in a subject.

In the case of an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, about 300 mg of an anti-IL-4R antibody is administered.

The amount of IL-4R antagonist contained within the individual doses may be expressed in terms of milligrams of antibody per kilogram of subject body weight (i.e., mg/kg). For example, the IL-4R antagonist may be administered to a patient at a dose of about 0.0001 to about 10 mg/kg of subject body weight. For example, the IL-4R antagonist can be administered at a dose of 1 mg/kg, 2 mg/kg, 3 mg/kg, 4 mg/kg, 5 mg/kg or 6 mg/kg.

In certain embodiments, the initial dose is about the same as the loading dose. In certain embodiments, the initial dose is about 1.1×, about 1.2×, about 1.3×, about 1.4×, about 1.5×, about 1.6×, about 1.7×, about 1.8×, about 1.9×, about 2.0×, about 2.5×, about 3.0×, or more of the loading dose.

In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “secondary doses” or “maintenance doses”). In one embodiment, the maintenance dose may be lower than the loading or initial dose. For example, one or more loading doses of 600 mg of IL-4R antagonist may be administered followed by maintenance doses of about 75 mg to about 300 mg. In certain embodiments, the methods comprise an initial dose or loading dose of about 400 mg or about 600 mg of an IL-4R antagonist. In certain embodiments, the methods comprise one or more secondary doses or maintenance doses of about 200 mg or about 300 mg of the IL-4R antagonist.

In one embodiment, the maintenance dose is the same dose as the loading or initial dose. For example, both the loading dose and the maintenance doses of the IL-4R antagonist may be administered in doses of about 75 mg to about 300 mg. In certain embodiments, the methods comprise an initial dose and maintenance doses of about 300 mg of an IL-4R antagonist.

In certain exemplary embodiments, a subject is a pediatric subject having a body weight of more than 30 kg, and the IL-4R antagonist is administered at a dose of about 150 mg, about 200 mg, about 250 mg, about 300 mg, about 350 mg, about 400 mg, about 450 mg, about 500 mg, about 550 mg, or about 600 mg. In some embodiments, a subject is a pediatric subject having a body weight of more than 30 kg, and the IL-4R antagonist is administered at an initial dose or loading dose of about 400 mg and one or more secondary doses or maintenance doses of about 200 mg, and the secondary doses are administered every other week (q2w). In some embodiments, a subject is a pediatric subject having a body weight of more than 30 kg, and the IL-4R antagonist is administered at an initial dose and maintenance doses of about 200 mg, and the maintenance doses are administered every other week (q2w).

In certain exemplary embodiments, a subject is a pediatric subject having a body weight of 30 kg or less and a body weight of at least 15 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is a pediatric subject having a body weight of 30 kg or less and a body weight of at least 15 kg, and the IL-4R antagonist is administered at an initial dose of about 600 mg and one or more secondary doses or maintenance doses of about 300 mg, and the secondary doses are administered every four weeks (q4w). In some embodiments, a subject is a pediatric subject having a body weight of 30 kg or less and a body weight of at least 15 kg, and the IL-4R antagonist is administered at an initial dose and maintenance doses of about 300 mg, and the maintenance doses are administered every four weeks (q4w).

In certain exemplary embodiments, a subject is an adolescent subject having a body weight of less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is an adolescent subject having a body weight of less than 60 kg, and the IL-4R antagonist is administered at an initial dose of about 400 mg and one or more secondary doses or maintenance doses of about 200 mg, and the secondary doses are administered every other week (q2w). In other embodiments, a subject is an adolescent subject having a body weight of less than 60 kg, and the IL-4R antagonist is administered at an initial dose and maintenance doses of about 200 mg, and the maintenance doses are administered every other week (q2w). In certain embodiments, a subject is an adolescent subject having a body weight that is greater than or equal to 30 kg and less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is an adolescent subject having a body weight that is greater than or equal to 30 kg and less than 60 kg, and the IL-4R antagonist is administered at an initial dose of about 400 mg and one or more secondary doses or maintenance doses of about 200 mg, and the secondary doses are administered every other week (q2w). In other embodiments, a subject is an adolescent subject having a body weight that is greater than or equal to 30 kg and less than 60 kg, and the IL-4R antagonist is administered at an initial dose and maintenance doses of about 200 mg, and the maintenance doses are administered every other week (q2w).

In certain exemplary embodiments, a subject is an adolescent subject having a body weight of at least 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adolescent subject having a body weight of at least 60 kg, and the IL-4R antagonist is administered at an initial dose of about 600 mg and one or more secondary doses or maintenance doses of about 300 mg, and the secondary doses are administered every other week (q2w). In other embodiments, a subject is an adolescent subject having a body weight of at least 60 kg, and the IL-4R antagonist is administered at an initial dose and maintenance doses of about 300 mg, and the maintenance doses are administered every other week (q2w).

In certain exemplary embodiments, a subject is an adult, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adult, and the IL-4R antagonist is administered at an initial dose of about 600 mg and one or more secondary doses or maintenance doses of about 300 mg, and the secondary doses are administered every other week (q2w). In other exemplary embodiments, a subject is an adult, and the IL-4R antagonist is administered at an initial dose of about 400 mg and one or more secondary doses or maintenance doses of about 200 mg, and the secondary doses are administered every other week (q2w). In certain embodiments, a subject is an adult, the initial dose comprises about 300 mg of the IL-4R antagonist, and the one or more subsequent doses comprise about 300 mg of the IL-4R antagonist administered every other week (q2w). In certain exemplary embodiments, the subject has an age between 40 years and 85 years.

In certain exemplary embodiments, an IL-4R antagonist is administered at a concentration of 150 mg/mL using a prefilled device. In some embodiments, a 150 mg/mL IL-4R antagonist solution in a pre-filled device is used to deliver about 300 mg IL-4R antagonist in a 2 mL injection. In certain exemplary embodiments, an IL-4R antagonist is administered at a concentration of 175 mg/mL using a prefilled device. In some embodiments, a 175 mg/mL IL-4R antagonist solution in a pre-filled device is used to deliver about 200 mg IL-4R antagonist in a 1.14 mL injection.

### Combination Therapies

Certain embodiments of the methods described herein comprise administering to the subject one or more additional therapeutic agents in combination with the IL-4R antagonist. As used herein, the expression “in combination with” means that the additional therapeutic agents are administered before, after, or concurrent with the pharmaceutical composition comprising the IL-4R antagonist. In some embodiments, the term “in combination with” includes sequential or concomitant administration of an IL-4R antagonist and a second therapeutic agent. Methods to treat COPD or an associated condition or complication or to reduce at least one COPD exacerbation comprising administration of an IL-4R antagonist in combination with a second therapeutic agent for additive or synergistic activity, are provided.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

The additional therapeutic agent may be, e.g., another IL-4R antagonist, an IL-33 antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), a TNF antagonist, an IL-8 antagonist, an IL-9 antagonist, an IL-17 antagonist, an IL-5 antagonist, an IgE antagonist, a CD48 antagonist, a leukotriene inhibitor, an anti-fungal agent, an NSAID, a long-acting muscarinic antagonist (LAMA), a long-acting beta₂ agonist (LABA), an inhaled corticosteroid (ICS), a systemic corticosteroid (e.g., oral or intravenous), methylxanthine, nedocromil sodium, cromolyn sodium, or combinations thereof. In exemplary embodiments, an additional therapeutic agent administered in combination with the IL-4R antagonist is a background therapy.

In certain embodiments, the pharmaceutical composition comprising an IL-4R antagonist is administered with a combination comprising a LABA, a LAMA, and an ICS. In other embodiments, an ICS is contraindicated and the pharmaceutical composition comprising an IL-4R antagonist is administered with a combination comprising a LABA and a LAMA.

In certain embodiments, the pharmaceutical composition comprising an IL-4R antagonist is administered with a background therapy comprising a LABA, a LAMA, and an ICS. In other embodiments, an ICS is contraindicated and the pharmaceutical composition comprising an IL-4R antagonist is administered with a background therapy comprising a LABA and a LAMA.

In certain embodiments, the pharmaceutical composition comprising an IL-4R antagonist is administered with a high dose ICS. In some embodiments, the pharmaceutical composition comprising an IL-4R antagonist is administered with a high dose ICS, a LAMA, and a LABA. In some embodiments, the high dose ICS is beclometasone dipropionate (CFC) and the dose is greater than 1000 mcg. In some embodiments, the high dose ICS is beclometasone dipropionate (HFA) and the dose is greater than 400 μg. In some embodiments, the high dose ICS is budesonide (DPI) and the dose is greater than 800 mcg. In some embodiments, the high dose ICS is ciclesonide (HFA) and the dose is greater than 320 mcg. In some embodiments, the high dose ICS is fluticasone propionate (DPI or HFA) and the dose is greater than 500 μg. In some embodiments, the high dose ICS is mometasone furoate and the dose is greater than 440 μg. In some embodiments, the high dose ICS is triamcinolone acetonide and the dose is greater than 2000 μg.

In certain embodiments, the pharmaceutical composition comprising an IL-4R antagonist is administered with a non-high dose ICS. In some embodiments, the pharmaceutical composition comprising an IL-4R antagonist is administered with a non-high dose ICS, a LAMA, and a LABA. In some embodiments, the non-high dose ICS is beclometasone dipropionate (CFC) and the dose is equal or less than 1000 mcg. In some embodiments, the non-high dose ICS is beclometasone dipropionate (HFA) and the dose is equal or below 400 μg. In some embodiments, the non-high dose ICS is budesonide (DPI) and the dose is equal or below 800 mcg. In some embodiments, the non-high dose ICS is ciclesonide (HFA) and the dose is equal or below 320 mcg. In some embodiments, the non-high dose ICS is fluticasone propionate (DPI or HFA) and the dose is equal or below 500 μg. In some embodiments, the non-high dose ICS is mometasone furoate and the dose is equal or below 440 μg. In some embodiments, the non-high dose ICS is triamcinolone acetonide and the dose is equal or below 2000 μg.

Suitable LABAs include, but are not limited to, salmeterol (e.g., SEREVENT®), formoterol (e.g., FORADIL®, PERFOROMIST®), indacaterol (e.g., ARCAPTA®), arformoterol (e.g., BROVANA®), olodaterol (e.g., STIVERDI®), and the like.

Suitable ICSs include, but are not limited to, fluticasone (e.g., fluticasone propionate, e.g., FLOVENT®), budesonide, mometasone (e.g., mometasone furoate, e.g., ASMANEX®), flunisolide (e.g., AEROBID®), dexamethasone acetate/phenobarbital/theophylline (e.g., AZMACORT®), beclomethasone dipropionate HFA (QVAR®), beclomethasone dipropionate (CFC), ciclesonide (HFA), triamcinolone acetonide and the like.

Suitable LAMAs include, but are not limited to, tiotropium bromide (e.g., SPIRIVA®), aclidinium bromide (e.g., EKLIRA®, TUDORZA®), glycopyrronium bromide (e.g., SEEBRI®), umeclidinium (e.g., INCRUSE®) and the like.

Suitable LAMA and LABA combinations include, but are not limited to, umeclidinium and vilanterol (e.g., Anoro), olodaterol and tiotropium (e.g., Stiolto), indacaterol and glycopyrrolate (e.g., Utibron), and glycopyrrolate and formoterol (e.g., Bevespi).

Suitable oral corticosteroids include, but are not limited to, prednisone, prednisolone, methylprednisolone, hydrocortisone, dexamethasone, cortisone acetate and the like.

### Administration Regimens

According to certain embodiments, multiple doses of an IL-4R antagonist may be administered to a subject over a defined time course. Such methods comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). Methods that comprise sequentially administering to the patient a single initial dose of an IL-4R antagonist, followed by one or more secondary doses of the IL-4R antagonist, and optionally followed by one or more tertiary doses of the IL-4R antagonist, are provided.

Methods comprising administering to a subject a pharmaceutical composition comprising an IL-4R antagonist at a dosing frequency of about four times a week, twice a week, once a week (qw or q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (q10w), once every eleven weeks (q11w), once every twelve weeks (q12w), or less frequently so long as a therapeutic response is achieved, are provided.

In certain embodiments involving the administration of a pharmaceutical composition comprising an anti-IL-4R antibody, once a week dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every two weeks dosing (every two weeks is used interchangeably with every other week, bi-weekly or q2w) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every three weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every four weeks dosing (monthly dosing) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every five weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every six weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every eight weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every twelve weeks dosing of an amount of about 100 mg, about 200 mg, about 300 mg, about 400 mg, about 500 mg or about 600 mg can be employed. In certain exemplary embodiments, the route of administration is subcutaneous.

The term “week” or “weeks” refers to a period of (n×7 days) ±3 days, e.g., (n×7 days) ±2 days, (n×7 days) ±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-4R antagonist, or may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. For example, one or more initial doses or loading doses of 600 mg or 400 mg of IL-4R antagonist may be administered followed by secondary doses or maintenance doses of about 75 mg to about 400 mg. In one embodiment, the secondary dose/maintenance dose may be equal to the initial dose/loading dose. For example, one or more initial doses/loading doses of 300 mg or 200 mg of IL-4R antagonist may be administered followed by secondary doses/maintenance doses of about 300 mg or about 200 mg, respectively. In one embodiment, a loading dose may be split, e.g., two or more doses administered at different time points, e.g., two loading doses wherein a second loading dose is administered two weeks after a first loading dose.

In certain embodiments, the initial dose is about 50 mg to about 600 mg of the IL-4R antagonist. In one embodiment, the initial dose is about 600 mg of the IL-4R antagonist. In another embodiment, the initial dose is about 400 mg of the IL-4R antagonist. In still other embodiments, the initial dose is about 300 mg of the IL-4R antagonist.

In certain embodiments, the secondary dose(s) are about 50 mg to about 600 mg of the IL-4R antagonist. In one embodiment, the maintenance dose is about 300 mg of the IL-4R antagonist. In one embodiment, the maintenance dose is about 200 mg of the IL-4R antagonist.

In certain embodiments, an initial dose is three times a maintenance dose. In certain embodiments, an initial dose is two times a maintenance dose. In certain embodiments, an initial dose is equal to a maintenance dose. In an exemplary embodiment, the initial dose is about 300 mg and the maintenance dose(s) are about 300 mg.

In some embodiments, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w). In other embodiments, the subject is an adult, the initial dose comprises about 300 mg of the antibody or antigen-binding fragment thereof, and the one or more secondary doses comprises about 300 mg of the antibody or antigen-binding fragment thereof administered every other week (every other week is used interchangeably with every two weeks, bi-weekly or q2w).

In some embodiments, a subject has moderate-to-severe COPD, and the initial dose comprises 300 mg of the antibody or antigen-binding fragment thereof, and the one or more subsequent doses comprises 300 mg of the antibody or antigen-binding fragment thereof administered every other week.

In some embodiments, a subject has COPD with Type 2 inflammation, and the initial dose comprises 300 mg of the antibody or antigen-binding fragment thereof, and the one or more subsequent doses comprises 300 mg of the antibody or antigen-binding fragment thereof administered every other week.

In some embodiments, a subject has moderate-to-severe COPD with Type 2 inflammation, and the initial dose comprises 300 mg of the antibody or antigen-binding fragment thereof, and the one or more subsequent doses comprises 300 mg of the antibody or antigen-binding fragment thereof administered every other week.

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-4R antagonist that is administered to a patient prior to the administration of the very next dose in the sequence with no intervening doses.

The methods may include administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of an IL-4R antagonist and a second therapeutic agent, to a patient to treat COPD or an associated condition are provided. In some embodiments, the methods comprise administering one or more doses of an IL-4R antagonist followed by one or more doses (e.g., 2, 3, 4, 5, 6, 7, 8, or more) of a second therapeutic agent. For example, one or more doses of about 75 mg to about 600 mg of the IL-4R antagonist may be administered after which one or more doses (e.g., 2, 3, 4, 5, 6, 7, 8, or more) of a second therapeutic agent (e.g., a LAMA, a LABA, an ICS, or a combination thereof) may be administered to treat, alleviate, reduce or ameliorate one or more symptoms of COPD. In some embodiments, the IL-4R antagonist is administered at one or more doses (e.g., 2, 3, 4, 5, 6, 7, 8, or more) resulting in an improvement in one or more COPD-associated parameters followed by the administration of a second therapeutic agent to prevent recurrence of at least one symptom of COPD. Alternative embodiments pertain to concomitant administration of an IL-4R antagonist and a second therapeutic agent. For example, one or more doses (e.g., 2, 3, 4, 5, 6, 7, 8, or more) of an IL-4R antagonist are administered and a second therapeutic agent is administered at a separate dosage at a similar or different frequency relative to the IL-4R antagonist. In some embodiments, the second therapeutic agent is administered before, after or concurrently with the IL-4R antagonist.

In some embodiments, a subject has moderate-to-severe COPD, and the initial dose comprises 300 mg of the antibody or antigen-binding fragment thereof, the one or more subsequent doses comprises 300 mg of the antibody or antigen-binding fragment thereof administered every other week, and the antibody or antigen-binding fragment thereof is administered in combination with a LAMA, a LABA, and an ICS.

In some embodiments, a subject has COPD with Type 2 inflammation, and the initial dose comprises 300 mg of the antibody or antigen-binding fragment thereof, and the one or more subsequent doses comprises 300 mg of the antibody or antigen-binding fragment thereof administered every other week, and the antibody or antigen-binding fragment thereof is administered in combination with a LAMA, a LABA, and an ICS.

In some embodiments, a subject has moderate-to-severe COPD with Type 2 inflammation, and the initial dose comprises 300 mg of the antibody or antigen-binding fragment thereof, and the one or more subsequent doses comprises 300 mg of the antibody or antigen-binding fragment thereof administered every other week, and the antibody or antigen-binding fragment thereof is administered in combination with a LAMA, a LABA, and an ICS.

In some embodiments, a subject has moderate-to-severe COPD, and the initial dose comprises 300 mg of the antibody or antigen-binding fragment thereof, the one or more subsequent doses comprises 300 mg of the antibody or antigen-binding fragment thereof administered every other week, and the antibody or antigen-binding fragment thereof is administered in combination with a LAMA and a LABA.

In some embodiments, a subject has COPD with Type 2 inflammation, and the initial dose comprises 300 mg of the antibody or antigen-binding fragment thereof, and the one or more subsequent doses comprises 300 mg of the antibody or antigen-binding fragment thereof administered every other week, and the antibody or antigen-binding fragment thereof is administered in combination with a LAMA and a LABA.

In some embodiments, a subject has moderate-to-severe COPD with Type 2 inflammation, and the initial dose comprises 300 mg of the antibody or antigen-binding fragment thereof, and the one or more subsequent doses comprises 300 mg of the antibody or antigen-binding fragment thereof administered every other week, and the antibody or antigen-binding fragment thereof is administered in combination with a LAMA and a LABA.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks, 52 weeks or more. In specific embodiments, the IL-4R antagonist is administered for at least 52 weeks.

In certain embodiments, a kit comprising a dosage form of an antibody, or an antigen-binding fragment thereof, that specifically binds interleukin-4 receptor (IL-4R), wherein the antibody or antigen-binding fragment thereof comprises three heavy chain CDR sequences comprising SEQ Id NOs: 3, 4, and 5, respectively, and three light chain CDR sequences comprising SEQ Id NOs: 6, 7, and 8, respectively, for the treatment of COPD is provided. In certain embodiments, the antibody or antigen-binding fragment thereof comprises a heavy chain variable region (HCVR) sequence of SEQ ID NO: 1 and a light chain variable region (LCVR) sequence of SEQ ID NO: 2. In certain embodiments, the antibody is dupilumab.

The kit can comprise a label or package insert, wherein the label or package insert comprises instructions for administering the dosage form for the treatment of COPD. The instructions can recite a dosing regimen described further herein for the treatment of COPD.

### Treatment Populations

The methods (or uses) featured in the disclosure include administering to a subject in need thereof a therapeutic composition comprising an IL-4R antagonist. The expression “a subject in need thereof” means a human or non-human animal that exhibits one or more symptoms or indicia of COPD (e.g., moderate-to-severe COPD and/or COPD with Type 2 inflammation), or who has been diagnosed with COPD. For example, “a subject in need thereof” may include, e.g., a subject who, prior to treatment, exhibits (or has exhibited) one or more COPD-associated parameters, such as, e.g., impaired FEV1 (e.g., less than 2.0 L), and/or has experienced one or more exacerbation of COPD events, e.g., acute exacerbation of COPD (AECOPD) events.

In some embodiments, a “subject in need thereof” has united airways disease (UAD). UAD is a concept based on the common pathophysiological and immunological mechanisms that underlie certain respiratory diseases. Under this concept, upper and lower airway diseases are frequently comorbid because they reflect manifestations of a single underlying disease at different sites of the respiratory tract. (See Kanda et al. “Regulation of Interaction Between the Upper and Lower Airways in United Airway Disease” Med Sci (Basel) 2019; 7(2) and Yii et al. “Precision Medicine in United Airways Disease: A Treatable Traits Approach” Allergy 2018; 73(10):1964-1978.) For example, UAD can be related to eosinophilic airway inflammation associated with Th2 cytokines (IL-4, IL-5, and IL-13) and/or IgE. (See Laidlaw, et al. “Chronic Rhinosinusitis with Nasal Polyps and Asthma” Journal of Allergy and Clinical Immunology: In Practice 2021; 9(3):1133-1141 and Fokkens et al. “EUFOREA Consensus on Biologics for CRSwNP with or without Asthma” Allergy 2018; 73(10):1964-1978.) The UAD concept is relevant to the comorbidity or multimorbidity of diseases related to Type 2 inflammation, these diseases can include, for example, asthma, chronic rhinosinusitis with nasal polyps (CRSwNP), chronic rhinosinusitis without nasal polyps, rhinitis, and COPD. (See Kanda et al. “Regulation of Interaction Between the Upper and Lower Airways in United Airway Disease” Med Sci (Basel) 2019; 7(2) and Yii et al. “Precision Medicine in United Airways Disease: A Treatable Traits Approach” Allergy 2018; 73(10):1964-1978.) In certain exemplary embodiments, the subject to be treated has a UAD (e.g., asthma, chronic rhinosinusitis with nasal polyps (CRSwNP), chronic rhinosinusitis without nasal polyps, bronchiectasis, or rhinitis, such as allergic rhinitis, allergic asthma rhinitis, local allergic rhinitis-non-allergic asthma, non-allergic rhinitis with eosinophilia syndrome-non-allergic eosinophilic asthma, asthma-chronic rhinosinusitis, COPD-chronic rhinosinusitis, etc.). In exemplary embodiments, the subject to be treated has UAD with COPD. In additional exemplary embodiments, the subject has UAD with COPD and one or more additional comorbid Type 2 inflammatory diseases (i.e., asthma, chronic rhinosinusitis with nasal polyps (CRSwNP), chronic rhinosinusitis without nasal polyps (CRSsNP), or rhinitis).

In a related embodiment, a “subject in need thereof” may be a subject who, prior to receiving an IL-4R antagonist, has been prescribed or is currently taking a LAMA, a LABA, and an ICS. In some embodiments, the subject has been prescribed or is currently taking a high dose ICS, a LAMA, and a LABA.

In other embodiments, a “subject in need thereof” may be a subject who, prior to receiving an IL-4R antagonist, has been prescribed or is currently taking a LAMA and a LABA. In some embodiments, an ICS is contraindicated for the subject.

In some embodiments, a “subject in need thereof” is a subject who has moderate-to-severe COPD. In some embodiments, a subject with moderate-to-severe COPD has a post-bronchodilator FEV1/FVC ratio <0.70 and post-bronchodilator FEV1% >30% and ≤70%. In some embodiments, a subject with moderate-to-severe COPD has a post-bronchodilator FEV1/FVC ratio of <0.70 and post-bronchodilator FEV1% of less than 30%, less than 25%, or less than 20%.

In some embodiments, a “subject in need thereof” is a subject who has COPD with Type 2 inflammation (i.e., COPD that is comorbid with at least one Type 2 inflammatory disease). In some embodiments, a subject with Type 2 inflammation has a blood eosinophil level ≥300 cells/microliter. Type 2 inflammatory diseases include, but are not limited to, atopic dermatitis (AD), asthma, chronic rhinosinusitis with nasal polyps (CRSwNP), chronic rhinosinusitis without nasal polyps (CRSsNP), eosinophilic esophagitis, chronic inducible urticaria, and prurigo nodularis.

In some embodiments, a “subject in need thereof” is a subject who has moderate-to-severe COPD with Type 2 inflammation.

In some embodiments, a “subject in need thereof” is a subject who has a medical research council (MRC) Dyspnea Scale grade ≥2. (See Bestall, et al. “Usefulness of the Medical Research Council (MRC) Dyspnea Scale as a Measure of Disability in Patients with Chronic Obstructive Pulmonary Disease” Thorax 1999; 54:581-586.) In certain exemplary embodiments, prior to the start of treatment the subject has a baseline Modified Medical Research Council (mMRC) Dyspnea Scale Grade score of ≥2. mMRC is described in ATS (1982) Am Rev Respir Dis. No; 126(5):952-6.

In some embodiments, a “subject in need thereof” is a subject who has a history of signs and symptoms of chronic bronchitis (chronic productive cough). In some embodiments, the subject has a history of signs and symptoms of chronic bronchitis for at least 3 months within the last year, or a productive cough of more than 3 months occurring within a span of 2 years.

In some embodiments, a “subject in need thereof” is a subject who has a history of high exacerbation risk. In some embodiments, high exacerbation risk is defined as exacerbation history of ≥2 moderate exacerbations or ≥1 severe exacerbations within the last year. In some embodiments, at least one moderate or severe exacerbation occurred while the subject was taking ICS/LAMA/LABA or LAMA/LABA.

In some embodiments, a “subject in need thereof” is a human. In some embodiments, the subject is an adult human. In some embodiments, the subject is between the ages of 40 and 80. In some embodiments, a “subject in need thereof” is a subject between the ages of 40 and 85. In some embodiments, the subject is at least 40 years old. In some embodiments, the subject is at least 65 years old. In some embodiments, the subject is 75 years of age or older. In some embodiments, the subject is younger than 40 years of age. In some embodiments, the subject is less than 80 years old. In some embodiments, the subject is less than 85 years old. In some embodiments, the subject is at least 18 years old.

In some embodiments, a “subject in need thereof” is a subject who is a current smoker. In some embodiments, the subject is a current smoker who smokes cigarettes. In some embodiments, the subject is a current smoker who has a smoking history of smoking greater than or equal to 10 packs of cigarettes per year. In some embodiments, the subject is a current smoker and has a smoking history of smoking fewer than 10 packs of cigarettes per year. In some embodiments, the subject is a current smoker and has a smoking history of smoking more than 1, 5, 10, 15, 20, 25, 30, 35, 40, 45, 50 or more packs of cigarettes per year. In some embodiments, the subject is a current smoker who has a smoking history of smoking for 6 months, 1 year, 2 years, 3 years, 5 years, 10 years or longer.

In some embodiments, a “subject in need thereof” is a subject who is a former smoker. In some embodiments, the subject is a former smoker who has a history of smoking cigarettes. In some embodiments, the subject is a former smoker who has a smoking history of smoking greater than or equal to 10 packs of cigarettes per year. In some embodiments, the subject is a former smoker who has a smoking history of smoking fewer than 10 packs per year. In some embodiments, the subject is a former smoker who has a smoking history of smoking more than 1, 5, 10, 15, 20, 25, 30, 35, 40, 45, 50 or more packs of cigarettes per year. In some embodiments, the subject is a former smoker who has a smoking history of smoking about 10, 15, 20, 25, 30, 35, 40, 45, 50 or more packs of cigarettes per year. In some embodiments, the subject is a former smoker who has a smoking history of smoking for 6 months, 1 year, 2 years, 3 years, 5 years, 10 years or longer. In some embodiments, the subject is a former smoker who has ceased smoking for at least 1, 2, 3, 4, 5, 6, 7, 8, 9, 10, 11, or 12 months or more. In some embodiments, the subject is a former smoker who has ceased smoking for at least 6 months. In some embodiments, the subject is a former smoker that intends to quit permanently.

In some embodiments, a “subject in need thereof” may be a subject that has a tested FEV1 value of less than 90%, 85%, 80%, 75%, 70%, 65%, 60%, 55%, 50%, 45%, 40%, 35%, 30%, 20%, 15%, or 10% or less than the predicted FEV1.

A normal IgE level in healthy subjects is less than about 100 kU/L (e.g., as measured using the IMMUNOCAP® assay [Phadia, Inc. Portage, MI]). In some embodiments, a “subject in need thereof” may be a subject who exhibits an elevated serum IgE level, which is a serum IgE level greater than about 100 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L.

In some embodiments, a “subject in need thereof” may be a subject with elevated levels of eotaxin-3, such as more than about 100 pg/ml, more than about 150 pg/ml, more than about 200 pg/ml, more than about 300 pg/ml, or more than about 350 pg/ml. Serum eotaxin-3 levels may be measured, for example, by ELISA.

In some embodiments, a “subject in need thereof” may be a subject with elevated levels of exhaled NO (FeNO), such as more than about 30 ppb, more than about 31 ppb, more than about 32 ppb, more than about 33 ppb, more than about 34 ppb, or more than about 35 ppb.

In some embodiments, the subjects are stratified into the following groups: a blood eosinophil count of ≥300 cells/μL (or cells/mm³) or ≥250 cells/μL (or cells/mm³) (high blood eosinophils); a blood eosinophil count of between 299 and 150 cells/μL (or cells/mm³) (moderate blood eosinophils); a blood eosinophil count of <150 cells/μL (or cells/mm³) (low blood eosinophils); or a blood eosinophil count of <300 cells/μL (or cells/mm³), and are administered an IL-4R antagonist at a dose or dosing regimen that is optionally based upon the eosinophil level.

In some embodiments, a “subject in need thereof” is a subject who has COPD with mucus plugging in the lung. In some embodiments, a “subject in need thereof” is a subject who has COPD with ventilation defects. In some embodiments, the subject treated with a method of the present disclosure has an improved mucus score. In some embodiments, the subject treated with a method of the present disclosure has improved ventilation. Mucus score can be assessed by CT (J Clin Invest 2018; 128: 997-1009; Chest 2019; 155: 1178-1189). Ventilation can be assessed by MRI, as measured by MRI ventilation defect percent (VDP), which is generated as the ventilation defect volume normalized to the thoracic cavity volume (Acad. Radiol. 2012; 19: 141-152).

### Methods for Assessing Pharmacodynamic COPD-Associated Parameters

Methods for assessing one or more pharmacodynamic COPD-associated parameters in a subject in need thereof, caused by administration of a pharmaceutical composition comprising an IL-4R antagonist, are provided. A reduction in the incidence of COPD symptoms or an improvement in a COPD-associated parameter may correlate with an improvement in one or more pharmacodynamic COPD-associated parameters; however, such a correlation is not necessarily observed in all cases.

Examples of “pharmacodynamic COPD-associated parameters” include, for example, the following: (a) biomarker expression levels and (b) serum protein and RNA analysis. An “improvement in a pharmacodynamic COPD-associated parameter” means, for example, a decrease from baseline of one or more biomarkers, such as pulmonary and activation-regulated chemokine (PARC), eotaxin-3, fibrinogen, IgE, blood or sputum eosinophils, blood or sputum neutrophils, or FeNO. As used herein, the term “baseline,” with regard to a pharmacodynamic COPD-associated parameter, means the numerical value of the pharmacodynamic COPD-associated parameter for a patient prior to or at the time of administration of a pharmaceutical composition described herein.

To assess a pharmacodynamic COPD-associated parameter, the parameter is quantified at baseline and at a time point after administration of the pharmaceutical composition. For example, a pharmacodynamic COPD-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic COPD-associated parameter (e.g., an increase or decrease, as the case may be, depending on the specific parameter being measured).

In certain embodiments, administration of an IL-4R antagonist to a patient causes a change, such as a decrease or increase, in expression of a particular biomarker. COPD-associated biomarkers include, but are not limited to pulmonary and activation-regulated chemokine (PARC), eotaxin-3, fibrinogen, IgE, blood or sputum eosinophils, blood or sputum neutrophils, or FeNO. For example, administration of an IL-4R antagonist to a COPD patient can cause a decrease in total serum IgE levels. The decrease can be detected at about week 1, about week 2, about week 3, about week 4, about week 5, or longer following administration of the IL-4R antagonist. Biomarker expression can be assayed by methods known in the art. For example, protein levels can be measured by ELISA (enzyme linked immunosorbent assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-4R antagonist or IL-4/IL-13 signaling (e.g., by measuring soluble IL-4Rα, IL-4, IL-13, etc.). In some embodiments, RNA samples are used to determine RNA levels (non-genetic analysis), e.g., RNA levels of biomarkers; and in other embodiments, RNA samples are used for transcriptome sequencing (e.g., genetic analysis).

### Formulations

In some embodiments, the antibody or antigen binding fragment thereof is formulated in a composition comprising: i) about 150 mg/mL of antibody or an antigen-binding fragment thereof that specifically binds to IL-4R, ii) about 20 mM histidine, iii) about 12.5 mM acetate, iv) about 5% (w/v) sucrose, v) about 25 mM arginine hydrochloride, vi) about 0.2% (w/v) polysorbate 80, wherein the pH of the formulation is about 5.9, and wherein the viscosity of the formulation is about 8.5 cPoise.

In alternative embodiments, the antibody or antigen binding fragment thereof is formulated in a composition comprising: i) about 175 mg/mL of antibody or an antigen-binding fragment thereof that specifically binds to IL-4R, ii) about 20 mM histidine, iii) about 12.5 mM acetate, iv) about 5% (w/v) sucrose, v) about 50 mM arginine hydrochloride, and vi) about 0.2% (w/v) polysorbate 80, wherein the pH of the formulation is about 5.9, and wherein the viscosity of the formulation is about 8.5 cPoise.

In specific embodiments, the antibody or antigen-binding fragment thereof comprises an HCVR comprising the amino acid sequence of SEQ ID NO: 1 and an LCVR comprising the amino acid sequence of SEQ ID NO: 2.

In specific embodiments, the antibody comprises dupilumab. Unless otherwise specified, the term “dupilumab” also includes any biosimilars thereof.

Suitable stabilized formulations are also set forth in U.S. Pat. No. 8,945,559, which is incorporated herein by reference in its entirety for all purposes.

The present disclosure is further illustrated by the following example which should not be construed as further limiting. The contents of the figures, tables and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### EXAMPLES

The following examples are put forth so as to provide those of ordinary skill in the art with a complete disclosure and description of how to make and use the methods and compositions featured in the disclosure, and is not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-4R antagonist used in the following Example is the human anti-IL-4R antibody named dupilumab (also referred to herein as “mAb1” or DUPIXENT®).

### Example 1. A Randomized, Double-Blind, Placebo-Controlled, Parallel-Group, 52-Week Pivotal Study to Assess the Efficacy, Safety, and Tolerability of Dupilumab in Patients with Moderate-to-Severe Chronic Obstructive Pulmonary Disease (COPD) with Type 2 Inflammation (2 Phase 3 Studies of Similar Design and Population—NCT03930732/EFC15804 (BOREAS) & NCT04456673/EFC15805 (NOTUS))

### Rationale

Chronic obstructive pulmonary disease (COPD) is a highly prevalent disease, associated with significant economic burden, and for which available standard-of-care therapies show insufficient treatment effect on symptoms, lung function, exacerbations and long term progression of the disease. This study was designed to investigate the efficacy and safety profile of dupilumab over one year in patients with COPD who were in need of an additional treatment added to their current management. The presence of a placebo arm was appropriate for the objectives of the study since it provided the most robust assessment of the efficacy and safety of dupilumab. All patients received standard of care background medication throughout the study. The study design provided the opportunity to investigate the efficacy of dupilumab on multiple COPD domains including lung function, prevention of moderate and severe exacerbations and symptom control. The effect on lung function and symptom control was evaluated over short term and long term.

### Objectives

### Primary Objective:

To evaluate the efficacy of dupilumab 300 mg q2w in patients with moderate or severe COPD as measured by annualized rate of acute moderate or severe COPD exacerbation (AECOPD).

### Secondary Objectives:

To evaluate the effect of dupilumab 300 mg q2w on pre-bronchodilator forced expiratory volume in 1 second (FEV1) over 12 weeks compared to placebo, health related quality of life, assessed by the change from baseline to week 52 in the St. George's Respiratory Questionnaire (SGRQ), and pre-bronchodilator FEV1 over 52 weeks compared to placebo.

Evaluate the effects of dupilumab 300 mg q2w on lung function assessments.

Evaluate the effect of dupilumab on moderate and severe COPD exacerbations.

To evaluate safety and tolerability.

To evaluate dupilumab systemic exposure and incidence of antidrug antibodies (ADA).

To evaluate the drug concentration of dupilumab in serum over time.

To explore the association of biomarkers with treatment response.

To evaluate the effects of dupilumab compared with placebo on FEV1 and FVC.

To evaluate the effects of dupilumab compared to placebo on annualized rate of moderate to severe COPD exacerbation utilizing the exacerbations of chronic pulmonary disease tool (EXACT).

To evaluate the effects of dupilumab compared to placebo on treatment failure requiring background medication change.

### Endpoints

### Primary Endpoint:

Annualized rate of moderate or severe COPD exacerbations over the 52-week treatment period compared to placebo. Moderate exacerbations were recorded by the investigator and defined as AECOPD that require either systemic corticosteroids (such as intramuscular, intravenous or oral) and/or antibiotics. Severe exacerbations were recorded by the investigator and defined as AECOPD requiring hospitalization, or observation for >24 hours in an emergency department/urgent care facility or resulting in death. For both moderate and severe events to be counted as separate events, they must have been separated by at least 14 days.

### Secondary Endpoints:

Exacerbation-associated systemic corticosteroids (SCS) intake duration.

Adjusted annualized total SCS courses.

Change in pre-bronchodilator FEV1 from baseline at week 12 and at week 52 compared to placebo, and change from baseline in prebronchodilator FEV1 at weeks 12 and 52 in patients with a baseline fractional exhaled nitric oxide (FeNO) level of ≥20 parts per billion (ppb).

Change from baseline to week 52 in SGRQ total score compared to placebo (score ranges from 0 to 100, with lower scores indicating a better quality of life. Minimum clinically important difference (MCID) 4 points).

Proportion of patients with SGRQ improvement ≥4 points at week 52.

Change in pre-bronchodilator FEV1 from baseline to week 52 compared to placebo.

Change in pre-bronchodilator FEV1 from baseline to weeks other than 12 and 52 (i.e., weeks 2, 4, 8, 24, 36, and 44) compared to placebo.

Change in post-bronchodilator FEV1 from baseline to week 2, 4, 8, 12, 24, 36 and 52 compared to placebo.

Change in forced expiratory flow (FEF) 25-75% from baseline to weeks 2, 4, 8, 12, 24, 36, 44, and 52.

Annualized rate of severe COPD exacerbations compared to placebo over the 52-week treatment period.

Time to first moderate or severe COPD exacerbation compared with placebo during the 52-week treatment period.

Adverse events (AEs)/treatment-emergent adverse events (TEAEs).

Potentially clinically significant laboratory abnormalities in hematology, biochemistry and urinalysis.

ADA against dupilumab.

Blood eosinophil levels over time.

### Tertiary/Exploratory Endpoints:

Serum functional dupilumab concentrations and PK profile.

Pharmacodynamic response of selected biomarkers: pulmonary and activation-regulated chemokine (PARC), eotaxin-3, fractional exhaled nitric oxide (FeNO postbronchodilator), and total IgE.

Fibrinogen.

Induced sputum for RNA expression.

Messenger ribonucleic acid (mRNA) sequencing or whole transcriptome analysis from blood and sputum.

Deoxyribonucleic acid (DNA) for assessment of pharmacogenomic effects.

Predictive effects of selected biomarkers on treatment response.

Annualized loss of lung function as assessed by a FEV1 slope analysis.

Change from baseline in FVC (% predicted and absolute values in mL) from baseline to week 12, week 24 and week 52.

Evaluation of clinical respiratory symptoms of COPD using the evaluating respiratory symptoms in COPD (E-RS: COPD) comprised in the EXACT tool.

Annualized rate of COPD exacerbations assessed by the EXACT over 52 weeks (score ranges from 0 to 40, with lower scores indicating less severe respiratory symptoms).

Increase in number of controller medication after exacerbation.

Increase in patient total daily dose of controller medication after exacerbation.

### Study Design

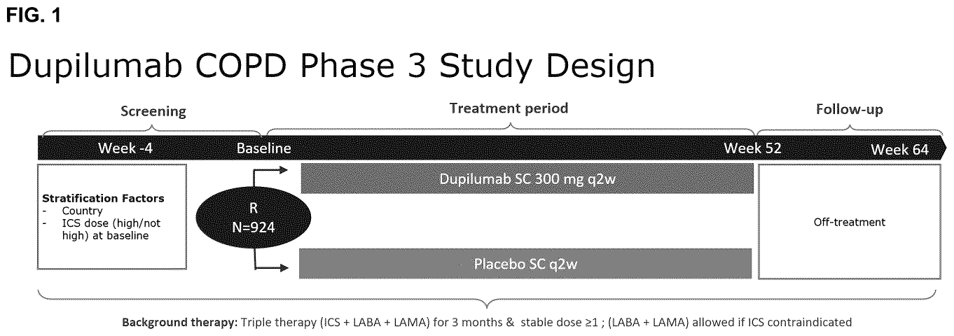

The BOREAS (NCT03930732/EFC15804) and NOTUS (NCT04456673/EFC15805) studies were multinational, randomized, double-blind, placebo-controlled, 52-week phase 3 studies to assess the efficacy, safety, and tolerability of dupilumab in patients with moderate-to-severe Type 2 inflammatory COPD, such as that driven by activation of IL-4, IL-5, and IL-13, on an established LABA, LAMA and/or ICS background therapy (triple therapy unless ICS was contraindicated). Study treatments were dupilumab 300 mg q2w or placebo q2w administered during the 52-week treatment period. A schema of the design of the studies is shown in FIG. 1.

The studies included 3 study periods. The screening period was 4 weeks ±1 week. The randomized investigational medicinal product (IMP) treatment period was 52 weeks ±3 days. The post IMP treatment period was 12 weeks ±5 days.

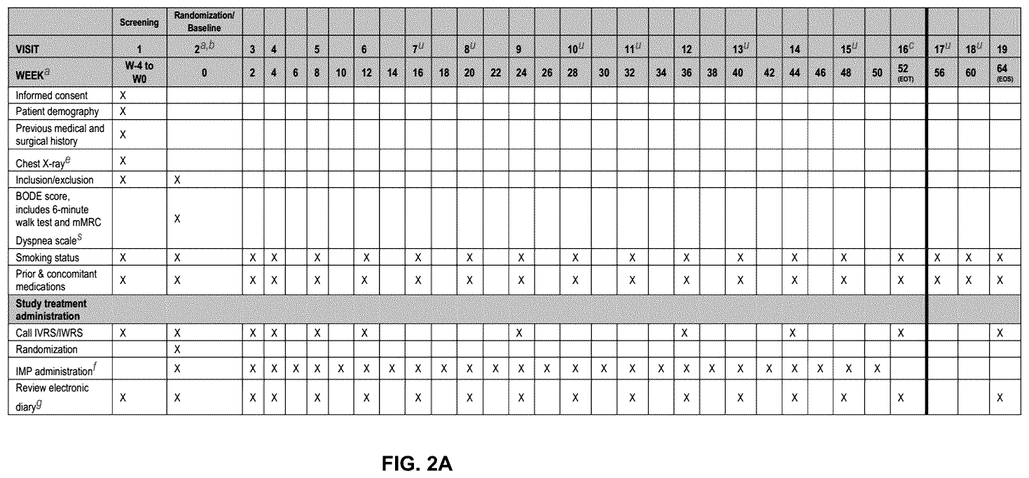

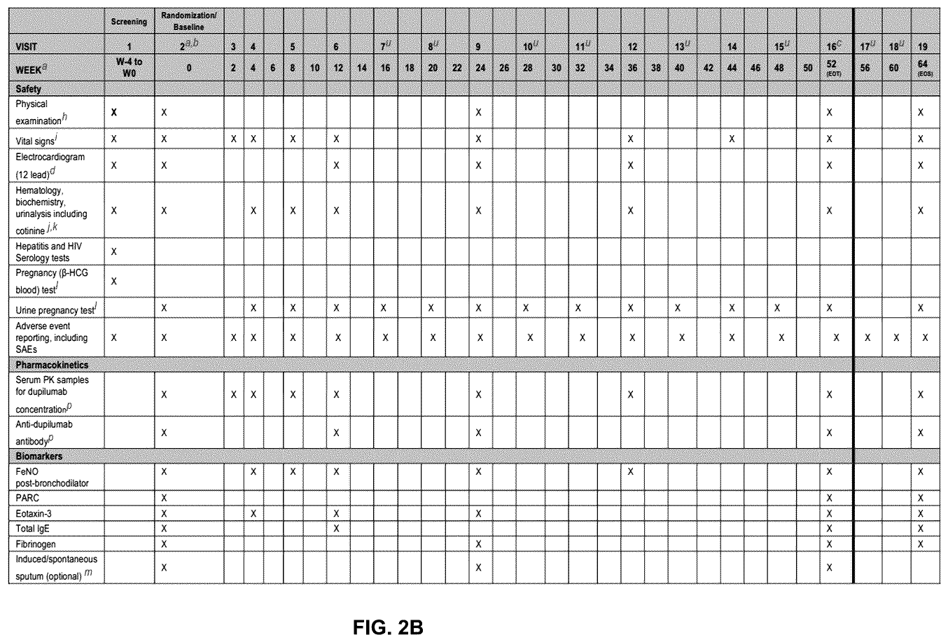

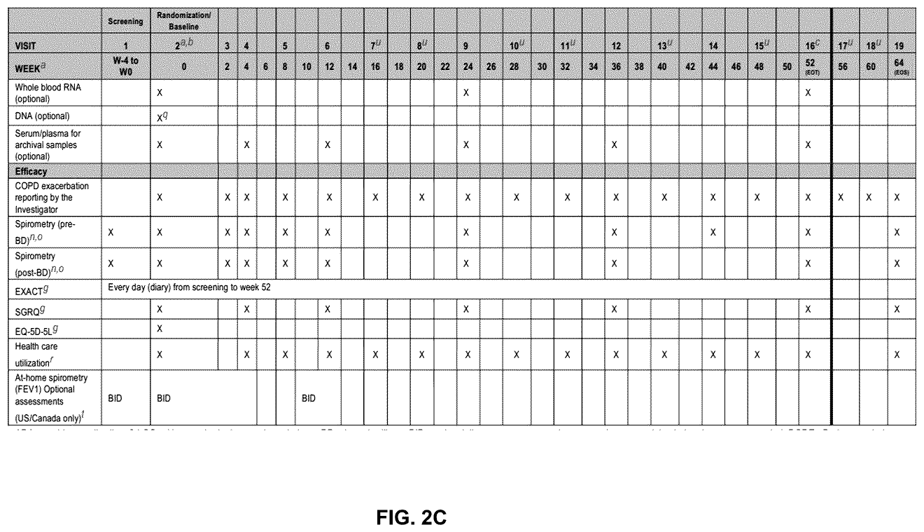

Patients who satisfied the inclusion and exclusion criteria were randomized (1:1) to one of the following IMP treatment period groups for administration for 52 weeks: dupilumab 300 mg, administered as 1 subcutaneous (SC) injection q2w or placebo administered as 1 SC injection. The schedule of activities for the studies is shown in FIG. 2A-C.

In each study approximately 924 patients were planned (actual 939 participants) to be randomly assigned to study intervention for an estimated total of 462 evaluable patients per intervention group.

Randomization was stratified by country and by ICS dose (high dose ICS) at baseline. As an example, the adult high dose of ICS for fluticasone propionate is >500 mcg (DPI or HFA) or 401-800 mcg (HFA) for Japanese population. Enrollment was to be capped at 30% current smokers (as defined by smoking status at screening visit).

Post IMP-treatment follow-up: Upon completing the 52-weeks randomized IMP treatment period, patients continued their triple background ICS/LABA/LAMA therapy (unless ICS is contraindicated) and entered 12-week safety follow-up period. Adjustment of background medication was allowed at the discretion of the investigator as clinically indicated during the post-treatment period. The use of e-cigarettes was not permitted during the study.

### Inclusion Criteria

Participants were eligible to be included in the study only if all of the following criteria applied:

### Age

For the BOREAS study, the participant must have been ≥40 to ≤80 years of age, at the time of signing the informed consent. For the NOTUS study, the participant must have been ≥40 to ≤85 years of age, at the time of signing the informed consent.

### Type of Participant and Disease Characteristics:

Participants with a Physician Diagnosis of COPD Who Meet the Following Criteria at Screening:

Current or former smokers with a smoking history of ≥10 pack-years. Current smokers are defined as those patients who are active smokers with ≥10 pack-years of smoking (active smoking includes cigarettes, e-cigarettes, cigars, pipes, etc.). Former smokers are defined as those patients who were active smokers with ≥10 pack-years of smoking (active smoking includes cigarettes, e-cigarettes, cigars, pipes, etc.) and who have stopped smoking for at least 6 months prior to visit 1.

Moderate to severe COPD (post-bronchodilator FEV1/FVC ratio <0.70 and post-bronchodilator FEV1% predicted >30% and ≤70%).

Medical Research Council (MRC) Dyspnea Scale Grade ≥2.

Patient-reported history of signs and symptoms of chronic bronchitis (chronic productive cough) for 3 months in the year up to screening in the absence of other known causes of chronic cough.

Documented history of high exacerbation risk defined as exacerbation history of ≥2 moderate or ≥1 severe within the year prior to inclusion. At least one exacerbation should have occurred while the patient was taking ICS/LAMA/LABA (or LAMA/LABA if ICS is contradicted). Moderate exacerbations were recorded by the investigator and defined as AECOPD that required either systemic corticosteroids (intramuscular (IM), intravenous, or oral) and/or antibiotics. One of the two required moderate exacerbations had to require the use of systemic corticosteroids. Severe exacerbations were recorded by the investigator and defined as AECOPD requiring hospitalization or observation >24 hours in emergency department/urgent care facility.

Background triple therapy (ICS+LABA+LAMA) for 3 months prior to randomization with a stable dose of medication for ≥1 month prior to Visit 1: (Double therapy: LABA+LAMA allowed if ICS is contraindicated).

Evidence of Type 2 inflammation: Patients with blood eosinophils ≥300 cells/microliter at Visit 1 (Screening).

Body mass index (BMI) ≥16 kg/m².

Male or Female. A female participant was eligible to participate if she was not pregnant and either not a woman of childbearing potential or who agreed to follow the contraceptive guidance during the intervention period and for at least 12 weeks after the last dose of study intervention.

Capable of giving signed informed consent.

### Exclusion Criteria

Participants were Excluded from the Study if any of the Following Criteria Applied:

COPD diagnosis for less than 12 months prior to randomization.

A patient with current diagnosis of asthma or history of asthma according to the Global Initiative for Asthma (GINA) guidelines, or other accepted guidelines.

Significant pulmonary disease other than COPD (e.g., lung fibrosis, sarcoidosis, interstitial lung disease, pulmonary hypertension, bronchiectasis, Churg-Strauss Syndrome, etc.) or another diagnosed pulmonary or systemic disease associated with elevated peripheral eosinophil counts.

Cor pulmonale, evidence of right cardiac failure.

Treatment with oxygen of more than 12 hours per day. Long-term treatment with oxygen >4.0 L/min OR if a participant requires more than 2.0 L/min in order to maintain oxygen saturation >88%.

Hypercapnia requiring bi-level positive airway pressure (BiPAP).

Acute exacerbation of COPD (AECOPD) within 4 weeks prior to or during the screening period.

Respiratory tract infection within 4 weeks prior to screening, or during the screening period.

History of, or planned pneumonectomy or lung volume reduction surgery. Patients who were participating in the acute phase of a pulmonary rehabilitation program, i.e., who started rehabilitation <4 weeks prior to screening (Note: patients in the maintenance phase of a rehabilitation program can be included).

Diagnosis of α-1 anti-trypsin deficiency.

Inability to follow the procedures of the study (e.g., due to language problems, psychological disorders) or unable to read, understand and fill out a questionnaire or use an e-Diary without any help.

Anti-immunoglobulin E (IgE) therapy (omalizumab) within 130 days prior to Visit 1 or any other biologic therapy (including anti-IL5 mAb) or immunosuppressant to treat inflammatory disease or autoimmune disease (e.g., rheumatoid arthritis, inflammatory bowel disease, primary biliary cirrhosis, systemic lupus erythematosus, multiple sclerosis, etc.) as well as other diseases within 2 months or 5 half-lives prior to Visit 1, whichever is longer.

Exposure to another investigative drug (small molecules as well as monoclonal antibodies) within a time period prior to Visit 1 that is less than 6 months. The minimum interval since exposure to any other (non-antibody) investigative study medication is 30 days prior to Visit 1.

History of systemic hypersensitivity or anaphylaxis to any biologic therapy, including any excipients.

Patients receiving medication or therapy that are prohibited as concomitant therapy.

Patient was the investigator, or any sub-investigator, research assistant, pharmacist, study coordinator, other staff or relative thereof directly involved in the conduct of the study.

Clinically significant abnormal electrocardiogram (ECG) at randomization that may affect the conduct of the study in the judgment of the investigator, e.g., prolonged QTc interval [male >450 msec, female >470 msec, Fredericia correction].

A patient with a history of clinically significant renal, hepatic, cardiovascular, metabolic, neurologic, hematologic, ophthalmologic, respiratory (other than COPD), gastrointestinal, cerebrovascular disease/condition, substance and/or alcohol abuse disorder, or history of or current other significant medical illness or disorder which, in the judgment of the investigator, could have interfered with the study or required treatment that might have interfered with the study. Specific examples include but are not limited to poorly controlled insulin-dependent diabetes, uncontrolled hypertension.

Prior history of malignancy or active malignancy, including lymphoproliferative diseases (except successfully treated carcinoma in-situ of the cervix, non-metastatic squamous cell or basal cell carcinoma of the skin) within 5 years prior to baseline.

Active tuberculosis or non-tuberculous mycobacterial infection, latent untreated tuberculosis or a history of incompletely treated tuberculosis were excluded from the study unless it was well documented by a specialist that the patient had been adequately treated and could start treatment with a biologic agent, in the medical judgment of the investigator and/or infectious disease specialist. Tuberculosis testing was performed on a country-by-country basis, according to local guidelines if required by regulatory authorities or ethics boards.

Acute myocardial infarction <6 months from screening visit.

TIA or stroke <6 months from screening visit.

Hospitalization for any CV or cerebrovascular event <6 months from screening visit.

Heart failure New York Heart Association Functional Classification (NYHA) Class III or IV.

Patients on cardiac medications not on a stable dose during the last 6 months (antiarrythmics, antihypertensives, and antidiuretics, etc). Dose modification of cholesterol-modifying agents and anticoagulants was allowed.

Cardiac arrhythmias including paroxysmal (e.g., intermittent) atrial fibrillation were excluded. Patients with persistent atrial fibrillation as defined by continuous atrial fibrillation for at least 6 months and controlled with a rate control strategy (i.e., selective beta blocker, calcium channel blocker, pacemaker placement, digoxin or ablation therapy) and stable appropriate level of anticoagulation for at least 6 months could have been considered for inclusion.

Unstable ischemic heart disease or other relevant cardiovascular disorder such as pulmonary embolism, deep vein thrombosis within ≤6 months from enrollment that in investigator's judgment may have put the patient at risk or negatively affect the study outcome.

Patients who are <80% compliant with controller therapy during screening.

Previous use of dupilumab.

Females who were lactating, breastfeeding or who were pregnant.

Women of childbearing potential (pre-menopausal female biologically capable of becoming pregnant) who: did not have a confirmed negative serum beta-hCG test at Visit 1 or negative urine pregnancy test at Visit 2 and/or were not willing to use one of the acceptable forms of effective contraception for the duration of the study. Postmenopausal women (defined as at least 12 consecutive months without menses) were not required to use additional contraception.

Diagnosed active parasitic infection (helminths), suspected or high risk of parasitic infection, unless clinical and (if necessary) laboratory assessments had ruled out active infection before randomization.

History of HIV infection or positive HIV 1/2 serology at Visit 1.

Known or suspected history of immunosuppression, including history of invasive opportunistic infections (e.g., histoplasmosis, listeriosis, coccidioidomycosis, pneumocystosis, aspergillosis), despite infection resolution; or unusually frequent, recurrent or prolonged infections, per investigator's judgment.

Evidence of acute or chronic infection requiring treatment with antibacterials, antivirals, antifungals, antiparasitics, or antiprotozoals within 4 weeks before Visit 1, significant viral infections within 4 weeks before Visit 1 that may not have received antiviral treatment (e.g., influenza receiving only symptomatic treatment).

Live, attenuated vaccinations within 4 weeks prior to Visit 1 or planned live, attenuated vaccinations during the study.

Patients with active autoimmune disease or patients who were using immunosuppressive therapy for autoimmune disease (e.g., inflammatory bowel disease, primary biliary cirrhosis, systemic lupus erythematosus, multiple sclerosis, etc.).

Patients with any of the following result at screening: positive (or indeterminate) HBsAg, positive IgM HBc Ab, positive total HBc Ab confirmed by positive HBV DNA, or positive HCV Ab confirmed by positive HCV RNA.

Any of the following values for clinically significant laboratory tests at screening: alanine transaminase (ALT) >3 times upper limit of normal range (ULN), hemoglobin <10 g/100 mL for male and <9 g/100 mL for female, platelets <100 000/mm³, or creatinine ≥150 μmol/L.

Patients on macrolide (e.g., azithromycin) therapy, unless on stable therapy for >12 months.

Patient who withdrew consent before enrollment/randomization.

Despite screening of the patient, enrollment/randomization was stopped at the study level.

### Study Intervention

Study intervention was defined as any investigational intervention(s), marketed product(s), placebo, or medical device(s) that was intended to be administered to a study participant according to the study protocol. An overview of the study interventions administered in presented in Table 8 below.

TABLE 8
Overview of Study Interventions Administered

| Study | | |
|---|---|---|
| intervention | | |
| name | Dupilumab 300 mg q2w | Placebo q2w |
| Dosage | Dupilumab 300 mg for SC | Placebo for dupilumab |
| formulation | administration is supplied | will be provided in |
| | as 150 mg/mL solution in | identically matched |
| | 2.25 mL prefilled glass | glass pre-filled syringe |
| | syringes to deliver 300 | to deliver 2 mL which |
| | mg in 2.0 mL | will match dupilumab |
| | | 300 mg. |
| Unit dose | 300 mg | Not applicable |
| strength(s)/ | | |
| Dosage level(s) | | |
| Route of | Subcutaneous injection | Subcutaneous injection |
| administration | sites should alternate | sites should alternate |
| | between the upper thighs, | between the upper thighs, |
| | 4 quadrants of the abdomen | 4 quadrants of the abdomen |
| | or the upper arms, so | or the upper arms, so |
| | that the same site is not | that the same site is not |
| | injected twice during | injected twice during |
| | consecutive injections | consecutive injections |
| Dosing | Every 14 ± 3 days | Every 14 ± 3 days |
| instructions | | |
| Packaging | Dupilumab will be supplied | Placebo will be supplied |
| and labeling | as one glass pre-filled | as one glass pre-filled |
| | syringe packed in a patient | syringe packed in a patient |
| | kit box. Both glass pre- | kit box. Both glass pre- |
| | filled syringe and box will | filled syringe and box will |
| | be labeled as required per | be labeled as required per |
| | country requirement. | country requirement. |

### Investigational Medicinal Products

The investigational medicinal product (IMP) was administered every 14±3 days (q2w) during the 52-week treatment period.

Investigational medicinal product (IMP) was administered by the investigator/health care professional or designee following clinic procedures and blood collection. Patients were monitored for at least 30 minutes after administration of all IMP injections. The monitoring period may have been extended as per country specific or local site-specific requirements.

Subcutaneous injection sites alternated between the upper thighs, 4 quadrants of the abdomen or the upper arms, so that the same site was not injected twice during consecutive administrations.

The first IMP administration was done by the investigator or delegate. The patients were allowed to self-inject IMP at home after at least 1 injection at investigational site, supervised by the investigator or delegate. To train patients how to prepare and inject IMP the investigator first trained the patient at the on-site visit during the treatment period. At this visit the patient performed the injection under the supervision of the investigator or delegate. This training was documented in the patient's study file. Patient injection was only performed in the abdomen or upper thighs. Patient were also be instructed to monitor for any reaction for at least 30 minutes (or longer per country specific or local site-specific requirements) following injection.

### Non-Investigational Medicinal Products

At Screening Visit 1, all patients must have been on triple background therapy, for 3 months prior to Visit 2/Randomization and at a stable dose of medication for at least 1 month prior to the Screening/Visit 1, including triple therapy: LABA+LAMA+ICS (Double therapy: LABA+LAMA allowed if ICS is contraindicated). The formulation was dry powder inhaler (DPI), metered dose inhaler (MDI) or pocket nebulizer. The route(s) of administration was oral inhalation. The dose regimen was as prescribed.

Throughout the study, patients continued their established background therapy for COPD, including dose and regimen.

Patients had to be willing to stay on their established background medication for COPD throughout the duration of the treatment period. After successful management of an acute exacerbation of COPD (e.g., with oral corticosteroids and/or antibiotics), all efforts were made to resume the initial background COPD treatment regimen if in the investigator's opinion this was medically acceptable. Background medications should not have been adjusted during Screening. After 1 severe or 2 moderate exacerbations of COPD, dose adjustments in background therapy were permitted for symptom control and as needed for the remainder of the trial period. Adjustment of background medication was allowed at the discretion of the investigator as clinically indicated during the post-treatment period.

### Reliever Medication

Reliever medications were supplied by sites and reimbursed as per country regulation or by sponsor's local affiliate as locally required. Patients were allowed to administer albuterol/salbutamol or levalbuterol/levosalbutamol or ipratropium or ipratropium/short-acting β agonists [SABA] combinations or terbutaline as needed during the study. Nebulizer solutions were allowed to be used as an alternative delivery method. The formulation was MDI, nebulizer solutions or DPI. The route of administration was oral inhalation. The dose regimen was as prescribed.

Study personnel converted salbutamol/albuterol nebulizer and levosalbutamol/levalbuterol nebulizer use as shown on the following tables:

TABLE 9
Reliever medication - Salbutamol/Albuterol Nebulizer Solution

| Salbutamol/Albuterol Nebulizer | Number |
|---|---|
| Solution -Total Daily Dose (mg) | of Puffs* |
| 2.5 | 4 |
| 5.0 | 8 |
| 7.5 | 12 |
| 10 | 16 |

*Conversion factor: salbutamol/albuterol nebulizer solution (2.5 mg) corresponds to 4 puffs.

An example of a salbutamol/albuterol nebulizer-to-puff conversion: patient received 3 salbutamol/albuterol nebulizer treatments (2.5 mg/treatment) between 7 and 11 AM. Total daily=7.5 mg or 12 puffs.

TABLE 10
Reliever medication - Levosalbutamol/Levalbuterol
Nebulizer Solution

| Levosalbutamol/Levalbuterol Nebulizer | Number |
|---|---|
| Solution -Total Dally Dose (mg) | of Puffs* |
| 0.63 | 2 |
| 1.25 | 4 |
| 2.5 | 8 |
| 3.75 | 12 |
| 5 | 16 |

*Conversion factor: levosalbutamol/levalbuterol nebulizer solution (1.25 mg) corresponds to 4 puffs.

An example of levosalbutamol/levalbuterol nebulizer-to-puff conversion: Patient received 3 levosalbutamol/levalbuterol nebulizer treatments (1.25 mg/treatment) between 7 and 11 AM. Total daily=3.75 mg or 12 puffs.

TABLE 11
Reliever medication - Ipratropium or Ipratropium/short-acting
β agonists [SABA] Nebulizer Solutions

| Total Daily Dose of | |
|---|---|
| Ipratropium (mg) in | |
| Ipratropium or Ipratropium/ | Number |
| SABA Nebulizer Solutions | of Puffs* |
| 0.5 | 4 |
| 1.0 | 8 |
| 1.5 | 12 |
| 2.0 | 16 |

*Conversion factor: ipratropium or ipratropium/short-acting β agonists (SABA) nebulizer solution (with 0.5 mg Ipratropium) corresponds to 4 puffs.

An example of ipratropium or ipratropium/SABA nebulizer-to-puff conversion: Patient received 3 ipratropium or ipratropium/SABA nebulizer treatments (0.5 mg Ipratropium/treatment) between 7 and 11 AM. Total daily=1.5 mg or 12 puffs.

### Storage and Handling

### Preparation of IMP

Dupilumab or matching placebo in glass pre-filled syringes was dispensed to the patients.

### Methods of Assigning Patients to Treatment Group

Patients were randomized in a 1:1 ratio to receive SC administrations of either: dupilumab 300 mg q2w or matching placebo for dupilumab q2w.

Randomization was stratified by country and by ICS dose (high dose ICS [yes/no]) at baseline. As an example, the adult high dose of ICS for fluticasone propionate is >500 mcg (DPI or HFA) or 401-800 mcg (HFA) for the Japanese population. Alerts were built into the IVRS/IWRS to limit enrolling patients who are current smokers (as defined by smoking status at screening) to less than or equal to 30% or 278 patients out of the total enrolled patients.

Investigational medicinal products were dispensed at the study visits summarized in the study of activities (FIG. 2A-C). Returned IMP from the patient's home was not re-dispensed to the participants.

### Methods of Blinding

Dupilumab and placebo were provided in identically matched 2 mL pre-filled syringes. To protect the blind, each treatment kit of 2 mL (dupilumab/placebo) glass pre-filled syringes was prepared such that the treatments (dupilumab and its matching placebo) were identical and indistinguishable and were labeled with a treatment kit number.

In accordance with the double-blind design, study patients, investigators, and study site personnel remained blinded to study treatment and did not have access to the randomization arm or to the IMP content (dupilumab or placebo).

### Concomitant Therapy

The following concomitant medications are not permitted during the study from screening onwards:

Use of any biologic agent within 5 half-lives of that compound before study entry (6 months if half-life is not known) and during the course of the study.

Use of PDE4 inhibitors (roflumilast) and of theophylline during the course of the study unless stable >6 months prior to screening visit.

New chronic use of macrolide antibiotics (e.g., azithromycin) with the exception of AECOPD, in which case macrolides may be used up to 28 days.

Systemic immunosuppressants (e.g., methotrexate, any anti-TNF mAbs, B and/or T-cell targeted immunosuppressive therapies) including chronic use of systemic corticosteroids.

Intravenous immunoglobulin (IVIG) therapy.

Live attenuated vaccines.

Beta-adrenergic receptor blockers (except for a selective beta-1 adrenergic receptor blocker used with dose stable 1 month prior to Visit 1).

Other investigational drugs.

The following is a list of permitted concomitant medications during the study:

Maintenance treatment of COPD with ICS, LABA, LAMA, at a stable dosage.

Systemic corticosteroids in case of acute exacerbation up to a maximum of 6 weeks.

Rescue medication with SABA or short acting antimuscarinics (e.g., atrovent).

Any medication or vaccine (including over-the-counter or prescription medicines, vitamins, and/or herbal supplements) that the participant was receiving at the time of enrollment or received during the study must have been recorded along with reason for use, dates of administration including start and end dates, and dosage information including dose and frequency.

Intervention after the End of the Study

Upon completion of the treatment period (52 weeks), or after early termination, study drug was no longer provided to participants.

### Discontinuation of Study Intervention

Withdrawal of consent for treatment (i.e., treatment discontinuation at patient request) was distinguished from (additional) withdrawal of consent for follow-up visits and from withdrawal of consent for non-patient contact follow-up (e.g., medical record checks). The site documented any case of withdrawal of consent.

The IMP was continued whenever possible. In case the IMP was stopped, it was determined whether the stop could be made temporarily and permanent IMP discontinuation was a last resort. Any IMP discontinuation was fully documented in the eCRF.

### Permanent Discontinuation

Permanent intervention discontinuation was any intervention discontinuation associated with the definitive decision from the investigator not to re-expose the patient to the IMP at any time during the study, or from the patient not to be re-exposed to the IMP whatever the reason.

All efforts were made to document the reason(s) for discontinuation of study intervention and were documented in the eCRF.

Patients Must be Withdrawn from the Study Intervention for the Following Reasons:

At their own request or at the request of their legally authorized representative (legally authorized representative means an individual or judicial or other body authorized under applicable law to consent on behalf of a prospective patient to the patient's participation in the procedure(s) involved in the research).

If, in the investigator's opinion, continuation on the study intervention would have been detrimental to the patient's well-being.

At the specific request of the Sponsor.

In the event of a protocol deviation, at the discretion of the investigator or the Sponsor.

Any code broken requested by the investigator led to permanent discontinuation of study intervention.

Pregnancy.

Anaphylactic reactions or systemic allergic reactions that are related to IMP and require treatment.

Diagnosis of a malignancy during study, excluding carcinoma in situ of the cervix, or squamous or basal cell carcinoma of the skin.

Any opportunistic infection, such as TB or other infections whose nature or course may suggest an immunocompromised status.

Serum ALT >3 ULN and Total Bilirubin >2ULN.

Serum ALT >5 ULN if baseline ALT ≤2 ULN or ALT >8 ULN if baseline ALT >2 ULN.

If the patient missed more than 3 consecutive doses, the patient was permanently discontinued from the study intervention.

Any abnormal laboratory value or ECG parameter was immediately rechecked for confirmation before making a decision of permanent discontinuation of the IMP for the concerned patient.

### Temporary Discontinuation of Study Intervention

Temporary discontinuation of study intervention may have been considered by the Investigator because of AEs. Re-initiation of treatment with the IMP was done under close and appropriate clinical/and or laboratory monitoring once the investigator had considered according to his/her best medical judgment that the AE was sufficiently resolved and unlikely to recur after resuming therapy with the IMP.

In addition, the following conditions(s) were causes for temporary discontinuation of study intervention: infections or infestations that did not respond to medical treatment and any laboratory abnormality that met temporary treatment discontinuation criteria.

For all temporary discontinuations of study intervention, the duration must have been recorded by the investigator in the eCRF. Following a temporary interruption or missed dose, the IMP treatment should have been reinitiated at the next scheduled dose, maintaining the original dose.

### Disease-Specific Efficacy Measures

### Severity of COPD Exacerbations as Defined by the Protocol

“Moderate exacerbations” were recorded by the investigator and defined as AECOPD that required either systemic corticosteroids (such as intramuscular, intravenous or oral) and/or antibiotics. “Severe exacerbations” were recorded by the investigator and defined as AECOPD requiring hospitalization, or observation for >24 hours in an emergency department/urgent care facility or resulting in death.

All other exacerbations were classified as “mild”.

For both moderate and severe events to have been counted as separate events, they must have been separated by at least 14 days.

### Clinical Symptoms of Exacerbations of COPD

In addition to the protocol-defined exacerbations of COPD listed above, clinical signs and symptoms of exacerbations of COPD were captured in the eCRF (including, but not limited to increase in dyspnea, increase in wheezing, increase in cough, increase in sputum volume and/or increase in sputum purulence).

### Management of Exacerbations of COPD

Exacerbations of COPD were treated as deemed necessary by the investigator. After successful management of an acute exacerbation of COPD (e.g., with oral corticosteroids and/or antibiotics), all efforts were made to resume the initial background COPD treatment regimen if in the investigator's opinion this was medically acceptable. After 1 severe or 2 moderate exacerbations of COPD, dose adjustments in background therapy were permitted for symptom control and as needed for the remainder of the trial period.

Protocol defined COPD exacerbation events were collected as efficacy endpoints via the exacerbation eCRF. These events should not have been reported as AEs unless they fulfilled a seriousness criterion.

### Spirometry

Spirometry at clinical site visits was to be performed in accordance with the European Respiratory Society (ERS)/American Thoracic Society (ATS) guidelines and prior to administration of investigational product. (See Miller, et al. “ATS/ERS TASK FORCE: Standardization of Lung Function Testing” Eur Resir J. 2005 August; 26(2):319-38).

For pre-bronchodilator measured parameters, including FEV1, FVC and forced expiratory flow (FEF) 25%-75%, spirometry was performed after a wash out period of bronchodilators according to their action duration, for example, withholding the last dose of salbutamol/albuterol or levosalbutamol/levalbuterol for at least 6 hours, withholding the last dose of LABA for at least 12 hours (ultra-long acting LABA like vilanterol should be withheld for at least 24 hours), withholding the last dose of ipratropium for at least 8 hours and withholding the last dose of LAMA for at least 24 hours. This was verified before performing the measurements.

When both pre- and post-bronchodilator spirometry was assessed, the post-bronchodilator spirometry was performed consistent with the mechanism of action of reliever (i.e., 30 minutes for albuterol or another SABA).

At all visits, spirometry was performed, usually in the morning; afternoon/evening was allowable in the exceptional circumstance when morning spirometry could not be performed; spirometry should have been done at approximately the same time at each visit throughout the study. Current smokers were reminded not to smoke for at least 1 hour before spirometry. The same spirometer and standard spirometric techniques, including calibration, were used to perform spirometry at all visits and, whenever possible, the same person performed the measurements.

Three measurements fulfilling the ATS acceptability and repeatability criteria were obtained at every visit, if possible.

### Patient Reported Outcome Questionnaires

At screening (Visit 1), patients were issued an electronic diary. Patients were instructed and trained by members of the clinical staff on the use of the device, and written instructions on the use of the electronic device were provided to the patients.

On a daily basis during screening and treatment, the patient used an electronic diary to: respond to the COPD exacerbation and symptom scale questions of the EXACT tool and record use of established controller inhalation therapy. The electronic diary was used for other patient related questionnaires such as SGRQ from the protocol. In the Post IMP Treatment Period, the patient's response in the electronic diary was not recorded daily. Patient-reported outcome questionnaires at a site visit were completed prior to any other assessments or procedures.

St. George's Respiratory Questionnaire (SGRQ)

The St. George's Respiratory Questionnaire (SGRQ) is a 50-item questionnaire designed to measure and quantify health status in adult patients with chronic airflow limitation. (See Jones et al. “The St George's Respiratory Questionnaire” Respir Med. 1991 September; 85 Suppl B:25-31; discussion 33-7). A global score ranges from 0 to 100. Scores by dimension are calculated for three domains: symptoms, activity, and impacts (Psycho-social) as well as a total score. Lower score indicates better quality of life (QoL).

The first part (“symptoms”) evaluates symptomatology, including frequency and severity of cough, sputum production, wheeze, breathlessness and the duration and frequency of attacks of breathlessness or wheeze. The second part has two components: “activity” and “impacts.” The “activity” section addresses disturbances to patients' daily physical activities. The “impacts” section covers a range of effects that chest troubles may have on patients' daily life and psycho-social functions (e.g., daily life activities and functioning, employment, physical functioning, emotional impact, stigmatization, and patients' perceptions when treated). The recall period of the questionnaire is over the past 4 weeks.

Psychometric testing has demonstrated its repeatability, reliability and validity. Sensitivity has been demonstrated in clinical trials. A minimum change in score of 4 units was established as clinically relevant after patient and clinician testing. The SGRQ has been used in a range of disease groups including asthma, COPD and bronchiectasis.

### Exacerbations of Chronic Pulmonary Disease Tool (EXACT)

The EXACT tool quantifies and measures exacerbations of COPD and assesses the symptomatic manifestations of these COPD exacerbations. The instrument is a daily diary composed of a total of 14 items representing the following domains: breathlessness (5 items), cough and sputum (2 items), chest symptoms (3 items), difficulty bringing up sputum (1 item), tired or weak (1 item), sleep disturbance (1 item), and scared or worried (1 item).

Development and validation history of the tool is consistent with guidelines proposed by the FDA, EMA and well-known measurement principles. The EXACT total score assesses COPD exacerbations. The higher the score, the more severe are the symptoms.

The Evaluating Respiratory Symptoms (E-RS) in COPD (E-RS: COPD) scale is a part of the EXACT tool. It is a derivative instrument used to measure the effect of treatment on the severity of respiratory symptoms in stable COPD. The E-RS utilizes the 11 respiratory symptom items contained in the 14-item EXACT. The RS-Total score represents respiratory symptom severity, overall. Three subscales can be used that assess: 1) breathlessness (RS-Breathlessness), 2) cough and sputum (RS-Cough and Sputum), and 3) chest-related symptoms (RS-Chest Symptoms). The higher the score the more severe are the symptoms.

### The 5-Level Euro Quality of Life-5 Dimension Questionnaire (EQ-5D-5L)

EQ-5D-5L is a standardized health-related QoL questionnaire developed by the EuroQol Group in order to provide a simple, generic measure of health for clinical and economic appraisal. EQ 5D is designed for self-completion by patients. The EQ-5D consists of a descriptive system and the EQ Visual Analog Scale (VAS). The descriptive system comprises five dimensions: mobility, self-care, usual activities, pain/discomfort, and anxiety/depression. Each dimension has 5 levels: no problems, slight problems, moderate problems, severe problems and extreme problems. The EQ VAS records the patient's self-rated health on a vertical visual analogue scale.

### Other Clinical Outcome Assessments

### Body-Mass Index, Airflow Obstruction, Dyspnea, and Exercise Capacity Index (BODE Index)

The BODE Index is a composite measure composed of a Performance Outcome Measure, a Patient-Reported Outcome Measure and a Biomarker. The BODE Index is a multidimensional grading system to assess the respiratory and systemic expressions of COPD. (Celli et al. “The Body-mass Index, Airflow obstruction, Dyspnea, and Exercise Capacity Index in Chronic Obstructive Pulmonary Disease” N Engl J Med. 2004 Mar. 4; 350(10):1005-12.) It comprises 4 domains: 1) degree of pulmonary impairment (FEV1); 2) patient's perception of symptoms (mMRC); and 2 independent domains: the 6 Minute Walking Distance (6MWD) and the Body-Mass Index (BMI). Each domain can be scored independently; the global score ranges from 0 to 10, with a higher score indicating a higher risk of death.

### Safety Assessments

The same safety assessments were applied across both arms. Adverse events, including SAEs and adverse events of special interest (AESI), were collected at every visit.

### Physical Examinations

A complete physical examination included skin, nasal cavities, eyes, ears, respiratory, cardiovascular, gastrointestinal, neurological, lymphatic, and musculoskeletal systems. All deviations from normal were recorded, including those attributable to the patient's disease.

Investigators paid special attention to clinical signs related to previous serious illnesses or signs of infection. Any new clinically significant finding or worsening of previous finding was reported as a new adverse event.

### Pharmacokinetics

Blood samples were collected for determination of functional dupilumab and anti-dupilumab antibodies in serum. The date and time of collection was recorded in the patient e-CRF.

### Pharmacodynamics

### Pharmacodynamic Variables/Biomarkers

Pharmacodynamic response of selected biomarkers was assayed including PARC, eotaxin-3, FeNO (postbronchodilator), total IgE, fibrinogen. Serum/plasma was collected for archival purposes. Induced sputum for RNA expression was also collected (this was optional for patients at a subset of sites).

### Whole Blood Biomarkers

Blood eosinophil and neutrophil counts were measured as part of the standard 5-part white blood cell (WBC) differential cell count on a hematology auto analyzer.

### Plasma/Serum Biomarkers

PARC was assayed using a validated enzyme immunoassay. Fibrinogen was also assayed. Eotaxin-3 was measured in heparinized plasma with a validated enzyme immunoassay. Total IgE was measured with a quantitative method (e.g., Phadia ImmunoCAP) approved for diagnostic testing.

### Exhaled Nitric Oxide

FeNO was analyzed using a NIOX instrument (Aerocrine AB, Solna, Sweden), or similar analyzer using a flow rate of 50 mL/s, and reported in parts per billion (ppb). This assessment was conducted prior to spirometry and following a fast of at least 1 hour.

### Pharmacogenomics

Pharmacogenetic/Pharmacogenomic testing was optional and voluntary. Written informed consent was obtained before sampling. For those patients who consented to the optional pharmacogenetic/pharmacogenomic sample collection section of the informed consent form (ICF), blood samples for exploratory genetic analysis of DNA or RNA were collected at the study visit as specified in the SoA, and these samples were stored for future analysis.

### Health Care Resource Utilization

A questionnaire of health care resource utilization (reliever medication, physician visit, hospitalization, emergency or urgent medical care facility visit, sick leaves including loss of usual activity days) was collected by the investigator for all participants throughout the study.

### Results

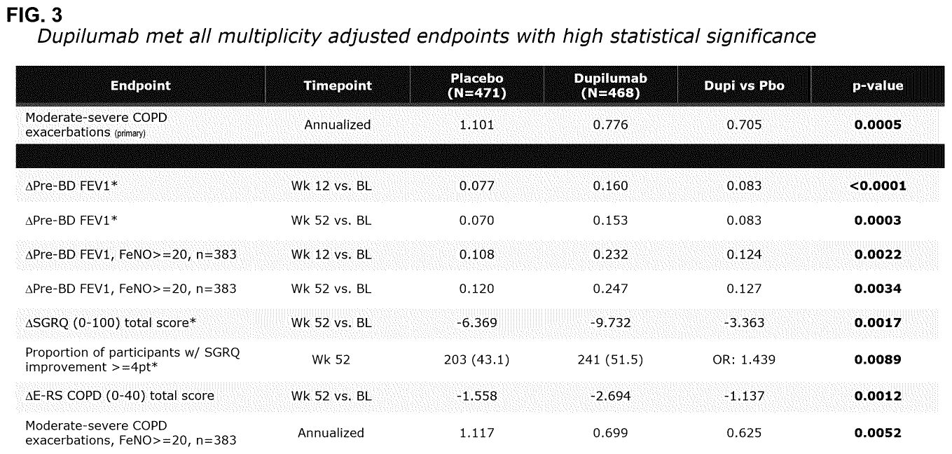

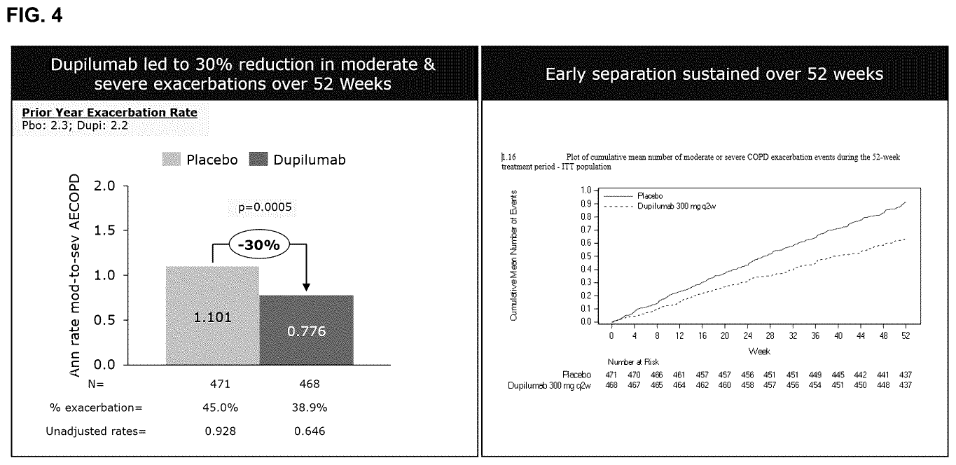

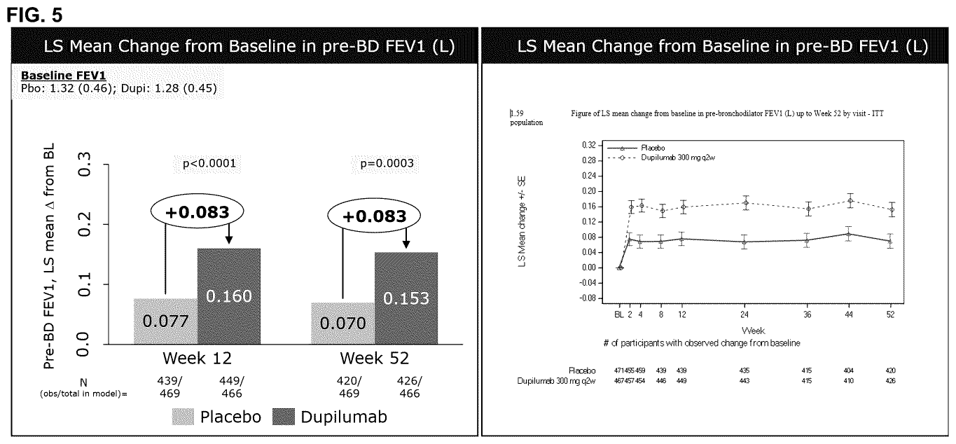

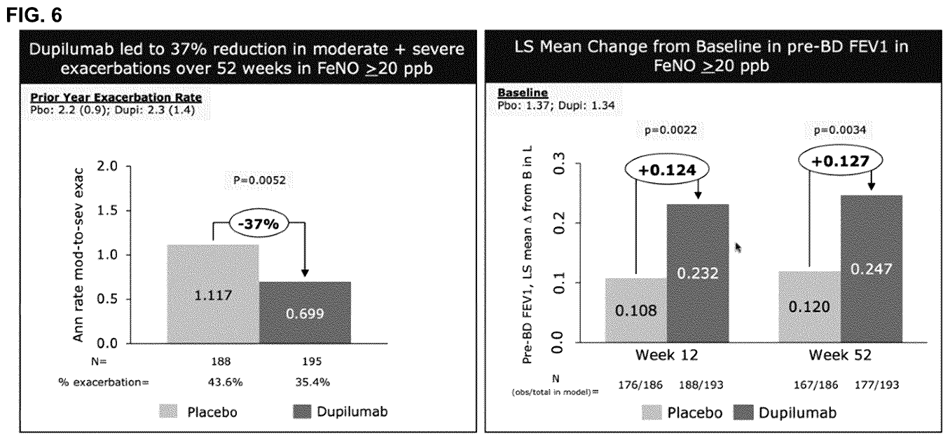

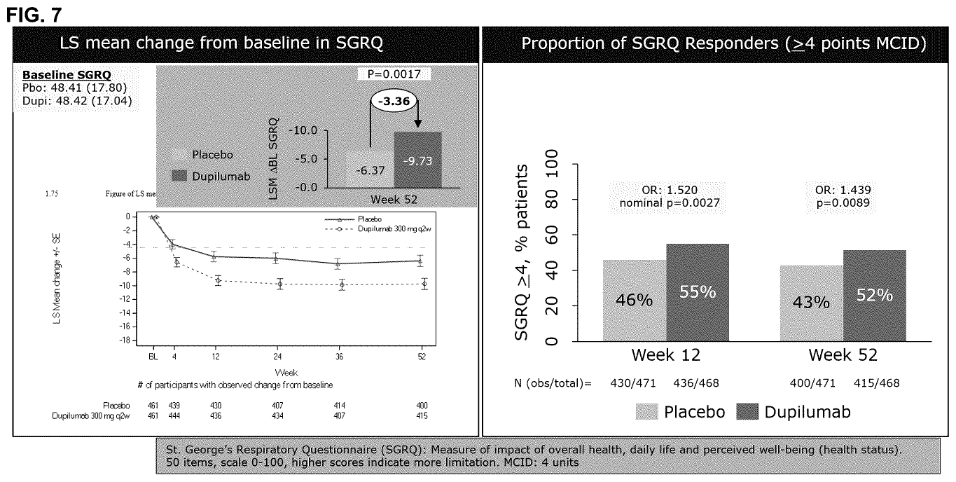

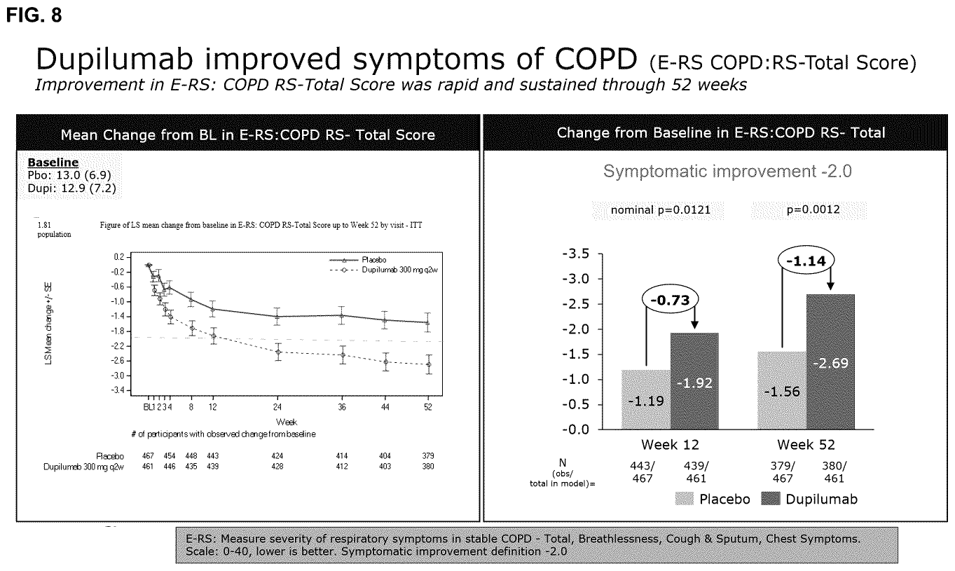

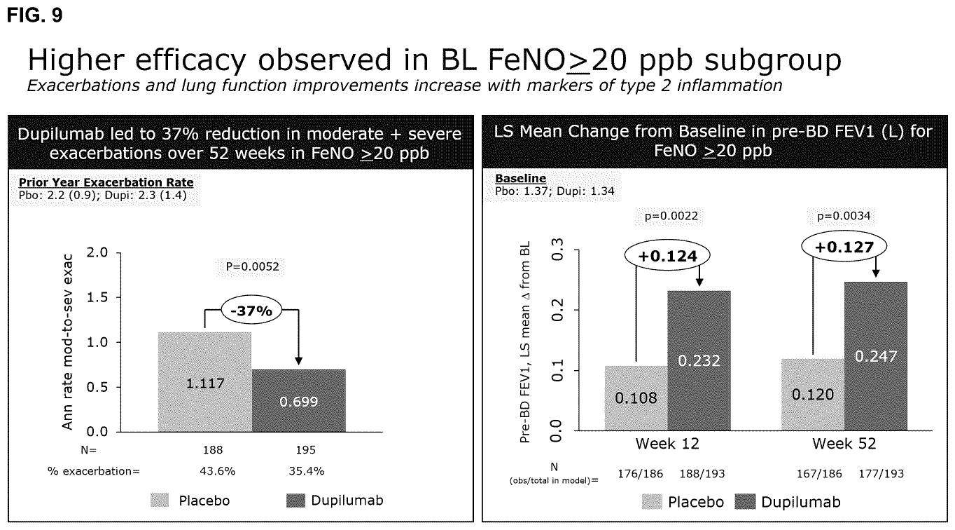

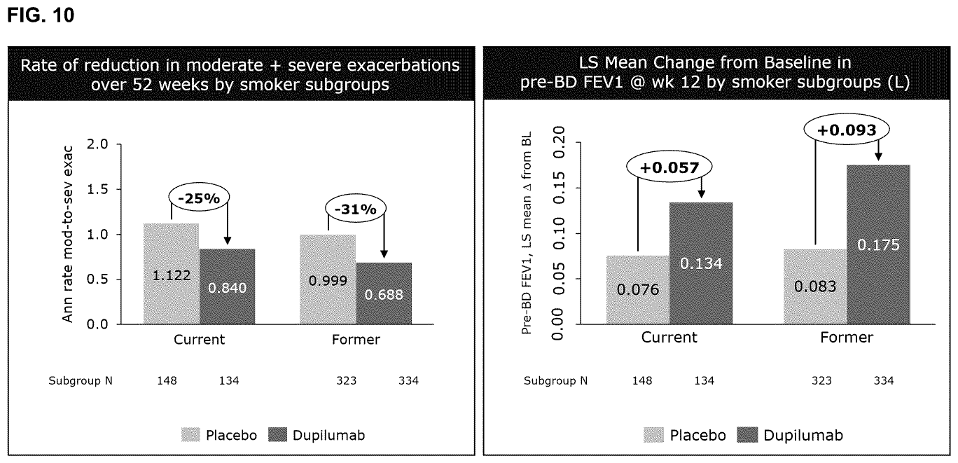

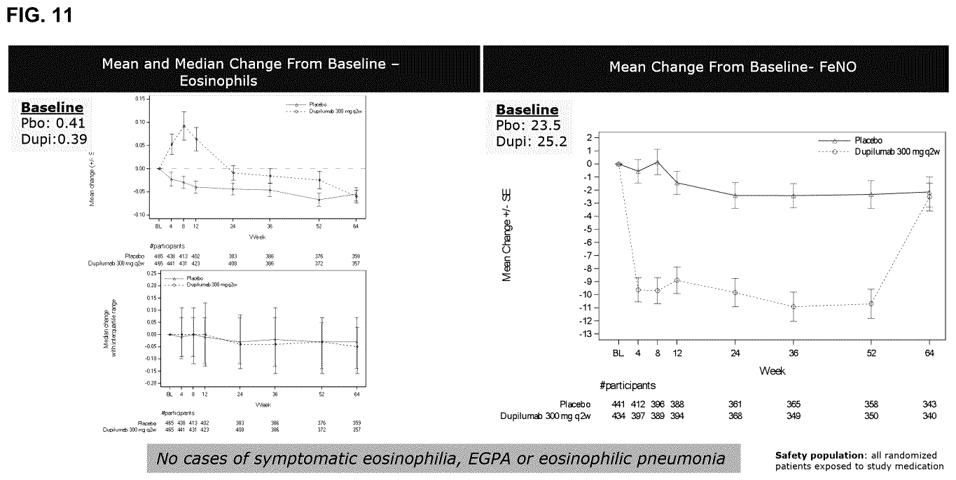

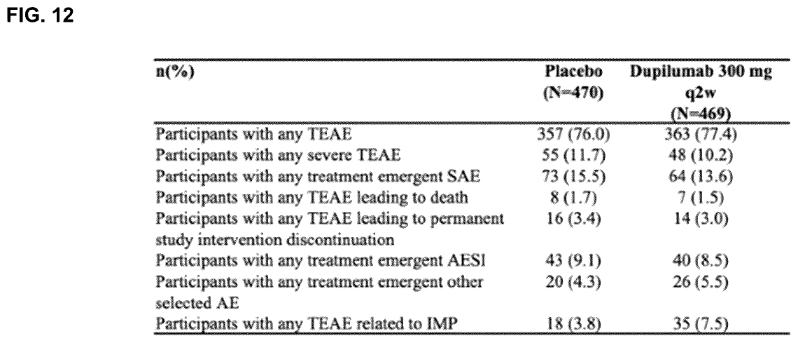

The results of the BOREAS trial are demonstrated in FIG. 3 to FIG. 10. In summary, Dupilumab was safe and effective in patients with uncontrolled COPD with type 2 inflammation. These patients have uncontrolled moderate to severe COPD despite standard of care (LABA+LAMA+ICS, unless ICS contraindicated) and type 2 inflammation (screening Eos ≥0.3 Giga/L)

The BOREAS trial met all multiplicity-controlled endpoints. Compared to baseline, dupilumab improved moderate-severe exacerbations, lung function (FEV1), HRQoL/health status as measured by SGRQ and symptoms as measured by ERS:COPD RS-Total Score in COPD patients with type 2 inflammation. The following results were observed:
statistically significant and clinically meaningful reduction in annualized rate of moderate to severe exacerbations (30% relative risk reduction (RRR), p=0.0005);
rapid & sustained improvement in lung function. At week 12, LSM diff FEV1 was +83 ml (p<0.0001), consistent at week 52;
consistent and higher benefit in the FeNO ≥20 ppb subgroup for exacerbations & FEV1 (multiplicity adjusted);
rapid and sustained improvement in HRQoL. (At week 52, LSM diff SGRQ −3.363 (p=0.0017) & SGRQ ≥4 (43.1% pbo vs. 51.5% dupi, p=0.0089));
rapid and sustained improvement in total symptoms (breathlessness, cough/sputum, chest). At week 52, LSM diff ERS-COPD tot-l−1.137 (p=0.0012); and
benefits were observed across multiple subgroups of demographic, clinical and biomarkers subgroups.

In addition, dupilumab therapy was well tolerated, with a safety profile generally consistent with the known safety profile of dupilumab:
TEAEs incidence was similar between dupilumab and placebo (76% pbo vs. 77% dupi). SAEs (16% pbo vs. 14% dupi), and TEAEs leading to treatment discontinuation were similar between treatment groups (3.4% pbo vs. 3.0% dupi);
TEAEs ≥2% with 1% difference—adverse effects more common in placebo (pbo) group include URTI, LRTI, pneumonia, COVID-19, hypertension, gastroenteritis, and adverse effects more common in in dupilumab (dupi) group include headache, back pain, UTI, diarrhea, gastritis, and toothache;
TEAE leading to death were balanced between treatment groups (1.7% pbo vs. 1.5% dupi), including OJ deaths (0.6% pbo vs. 0.2% dupi);
COPD relevant SOCs Respiratory, Cardiac, and Vascular, plus adj. MACE were more frequent in placebo (placebo vs. Dupixent 17% vs. 16%; 8.5% vs. 7.0%; 9.6% vs. 7.0%; 1.9% vs. 0.9%, respectively);
TEAEs COVID-19 was higher in placebo group (pbo 8.1% vs. dupi 6.2%);
Malignances were overall balanced between treatment groups (1.9% pbo vs. dupi 1.7%);
treatment emergent AESI or other selected AE were balanced between treatment groups. Few AESIs of opportunistic infections include one case of TB in dupi group and one post-treatment aspergillosis and two cases of herpes zoster in pbo group and one in dupi group;
no AEs associated with eosinophilia, and no reports of keratitis, severe conjunctivitis/blepharitis, or parasitic infection; and
no meaningful differences in the laboratory observations.

### Patients

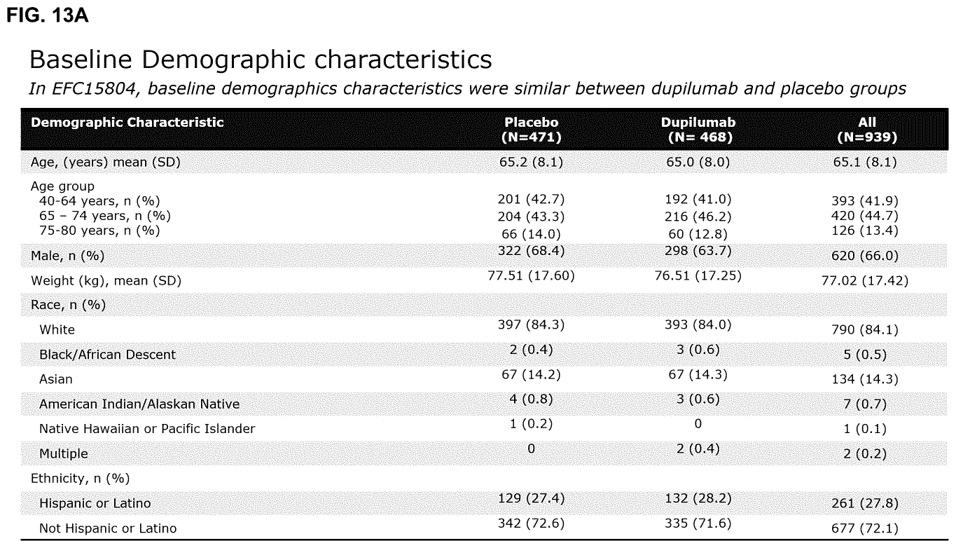

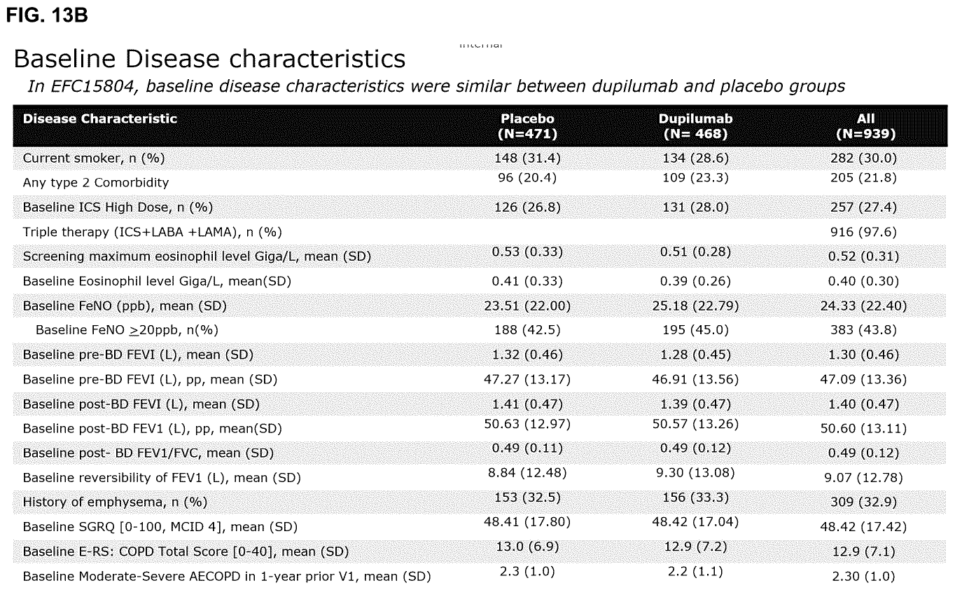

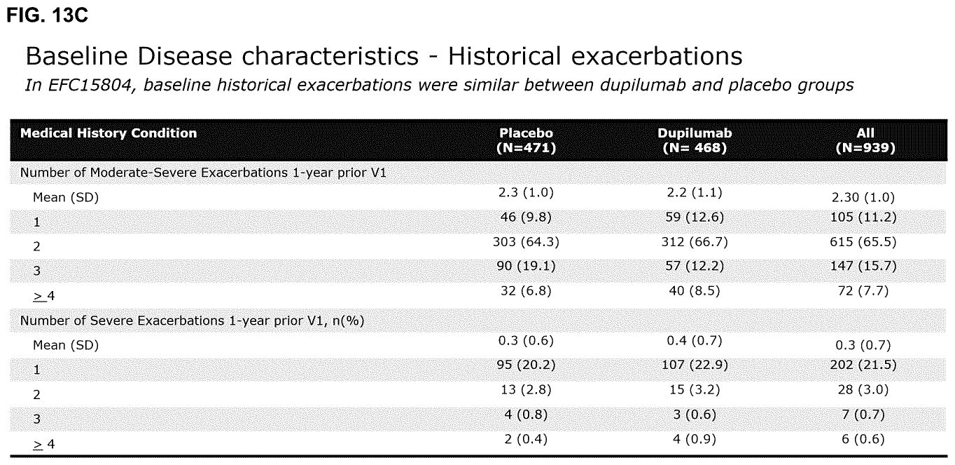

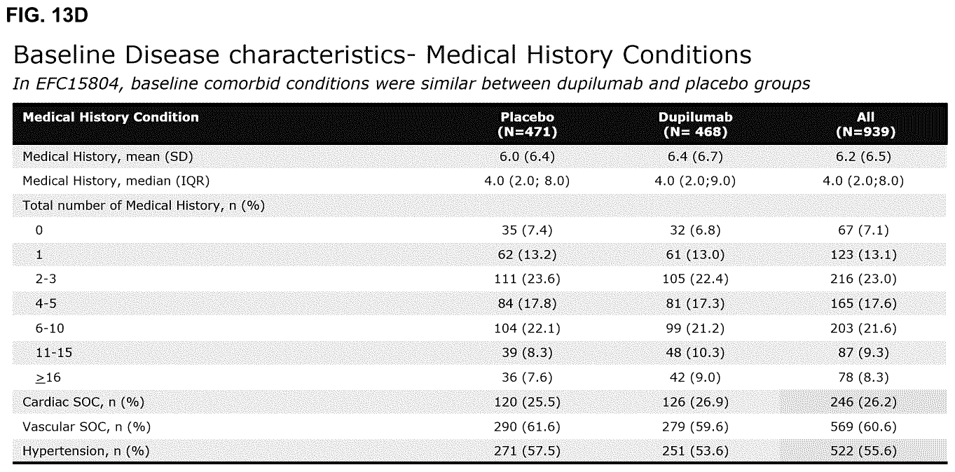

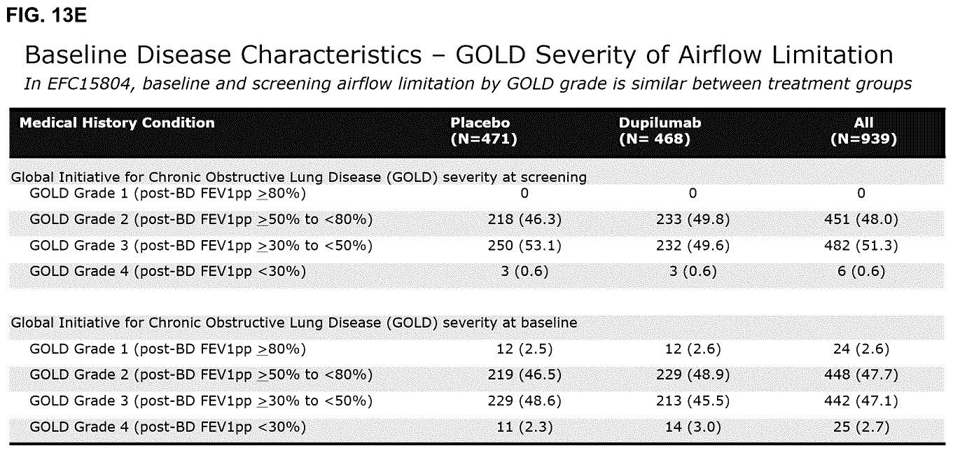

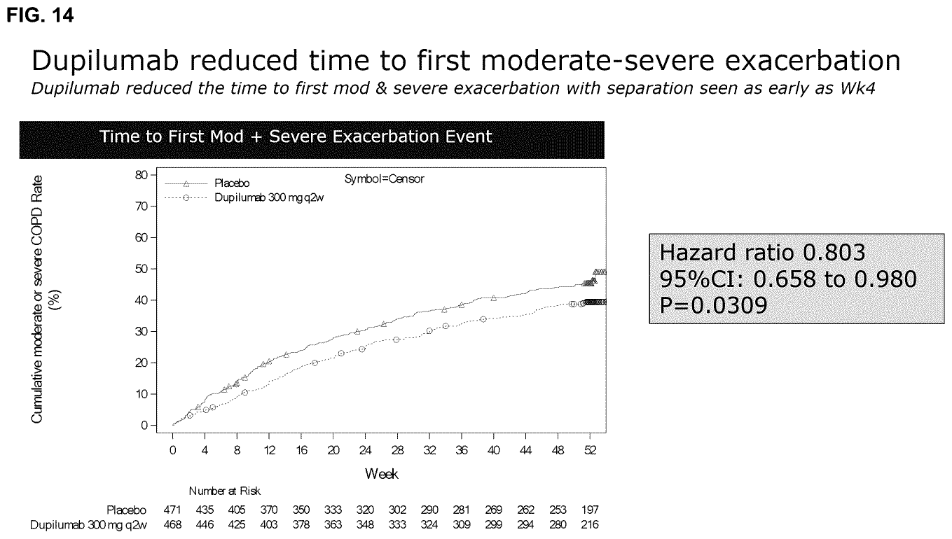

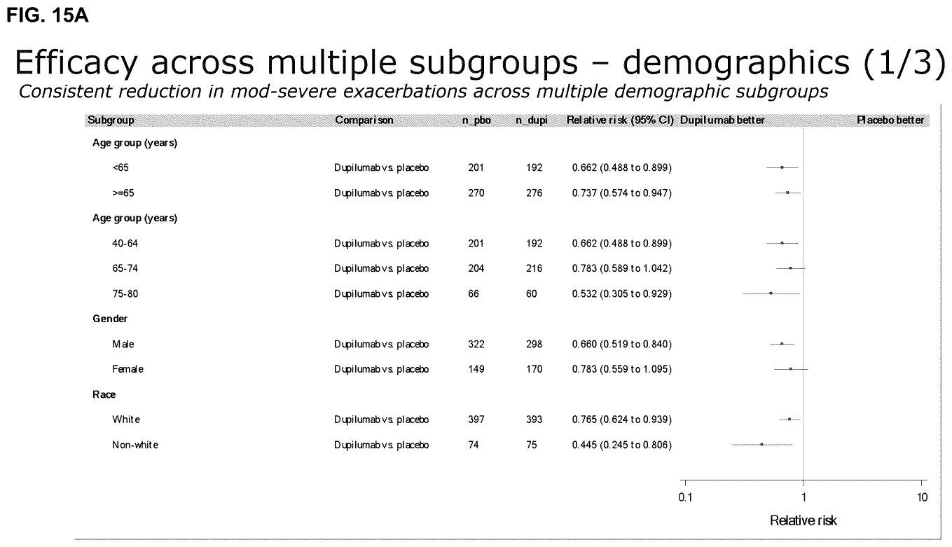

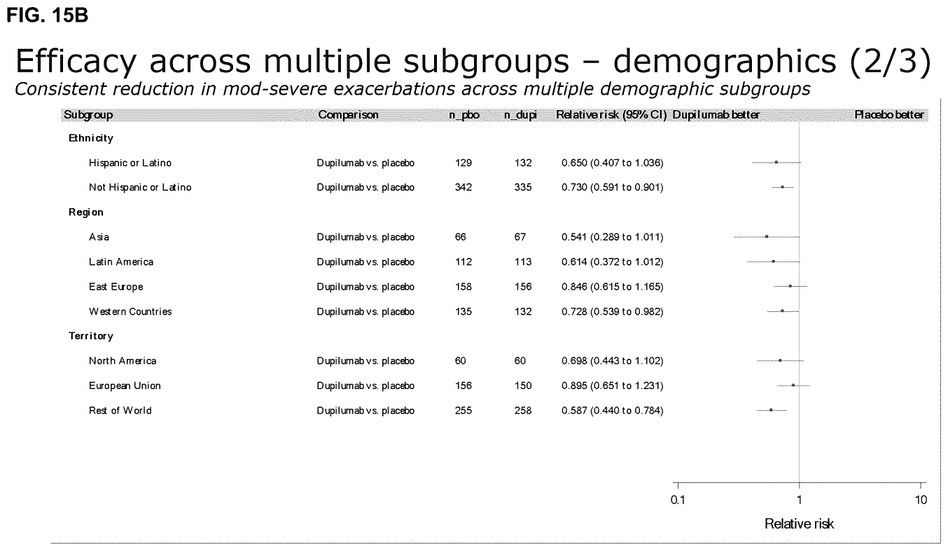

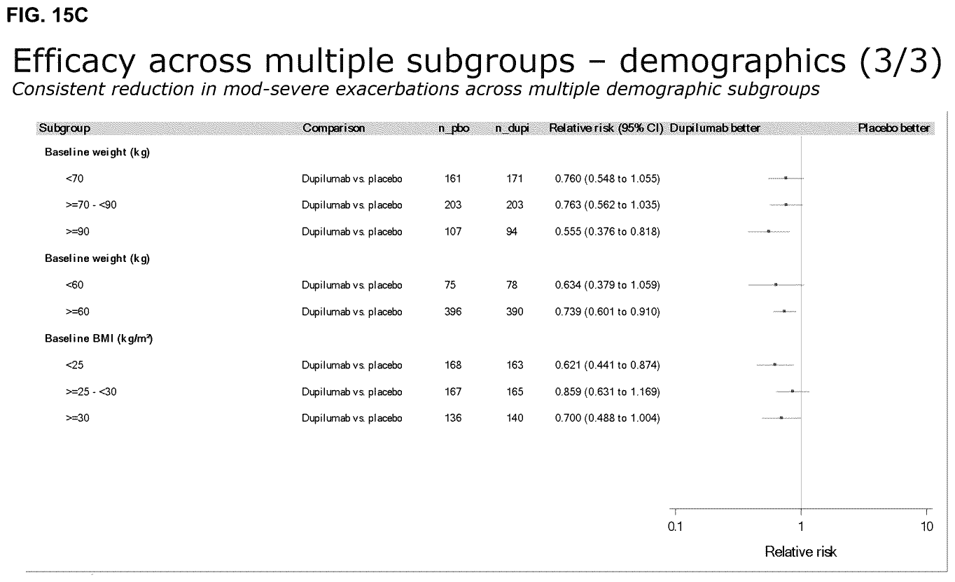

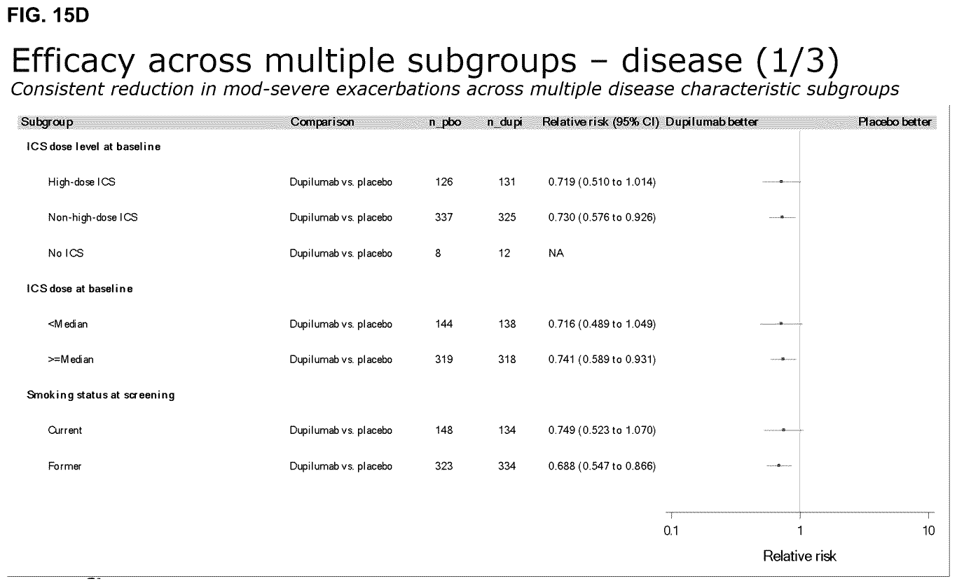

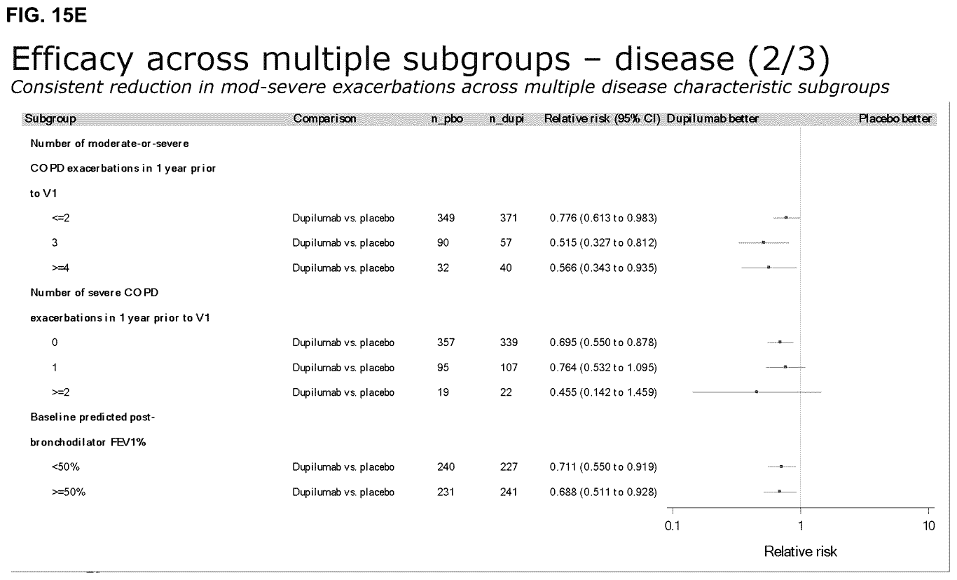

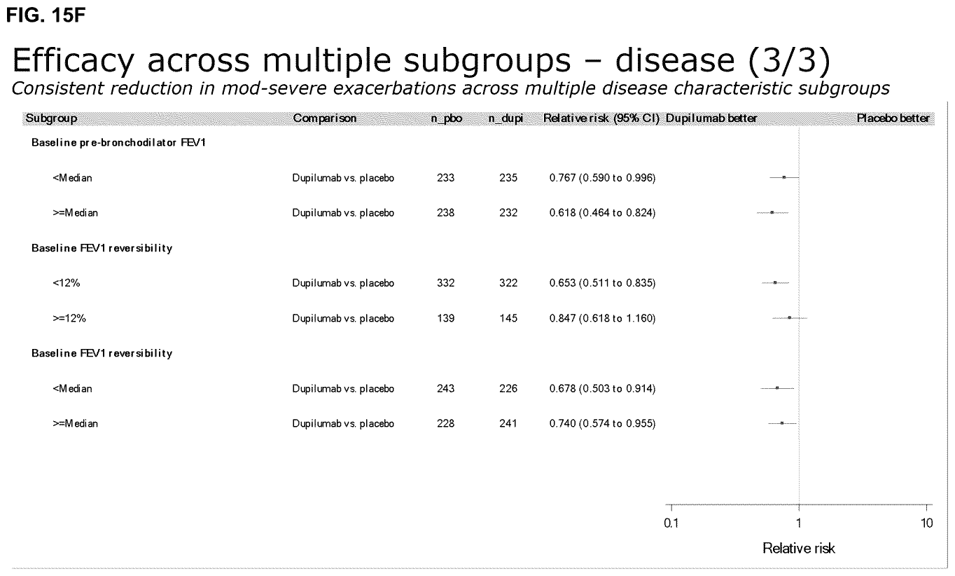

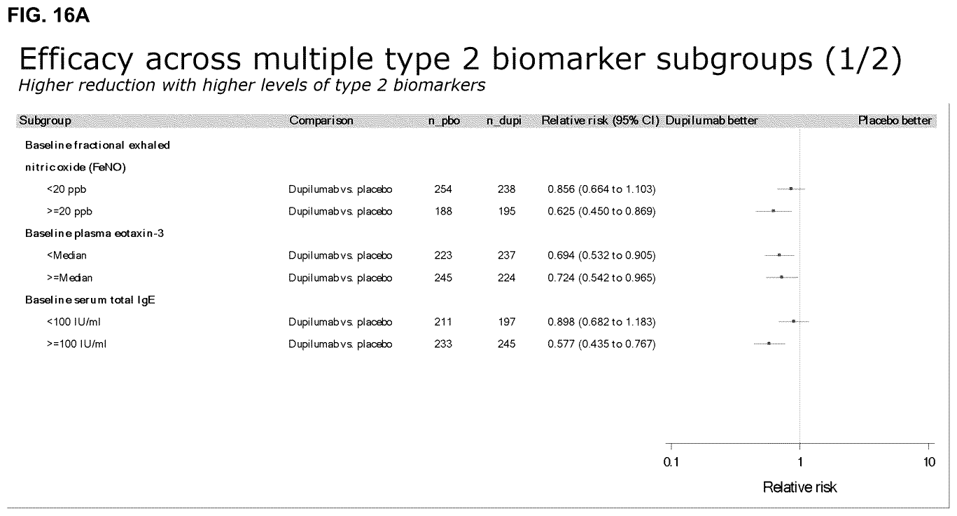

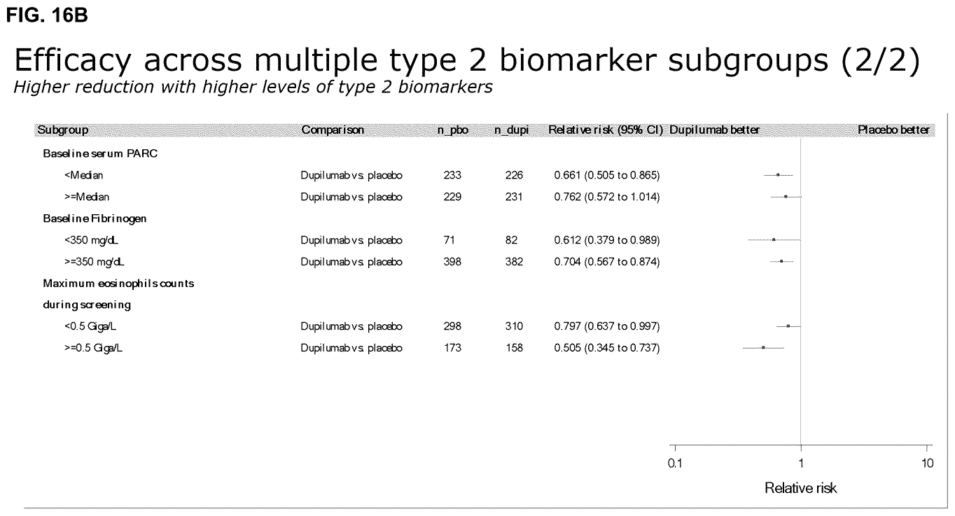

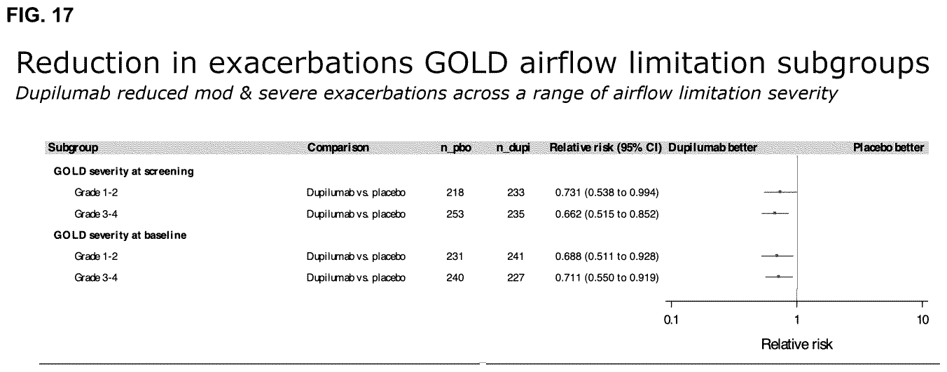

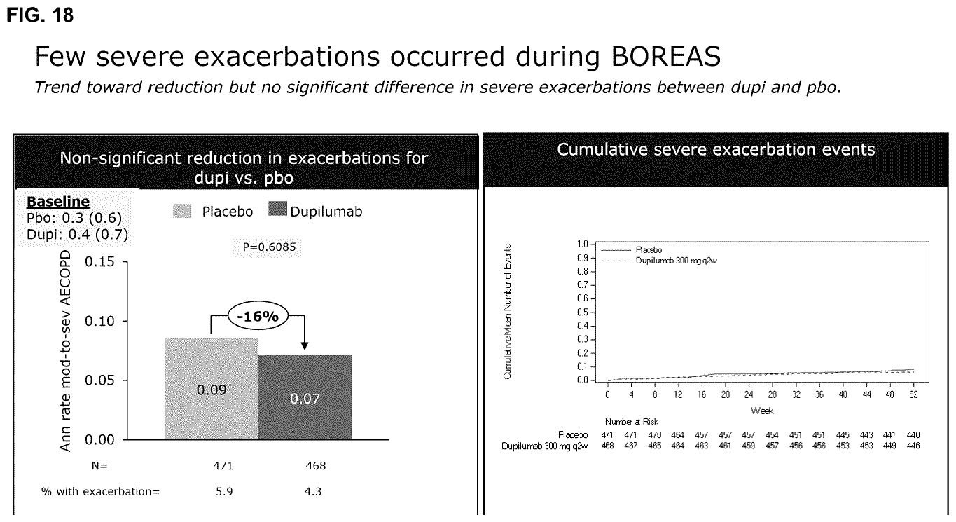

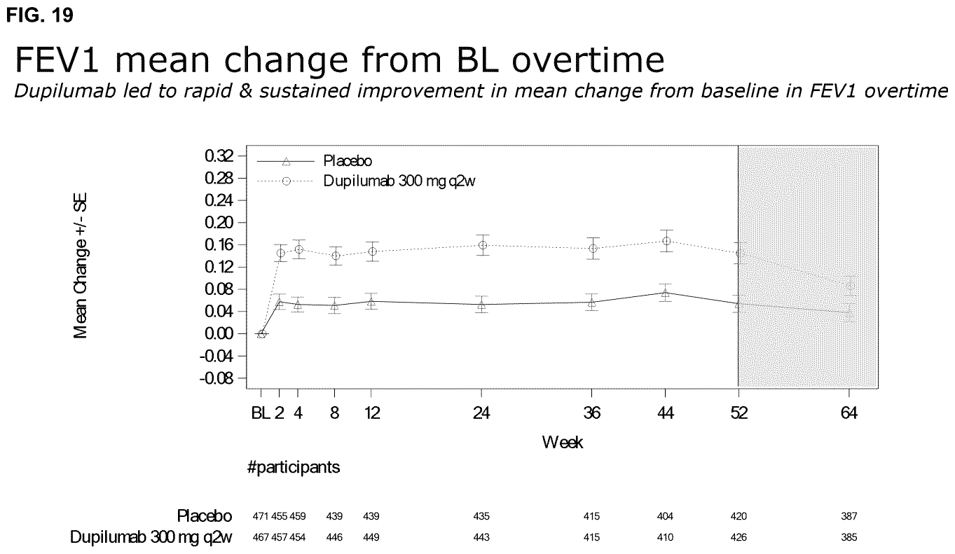

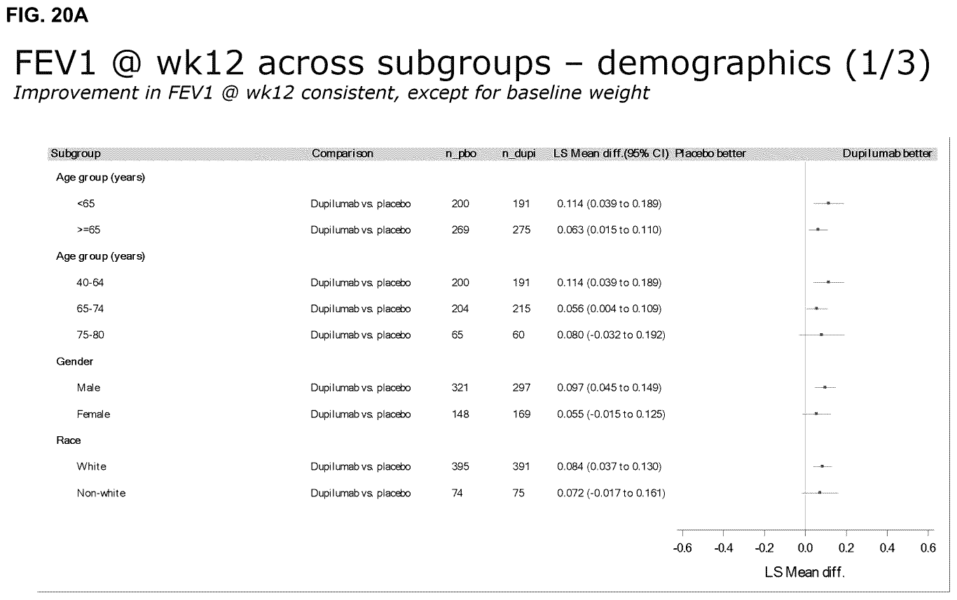

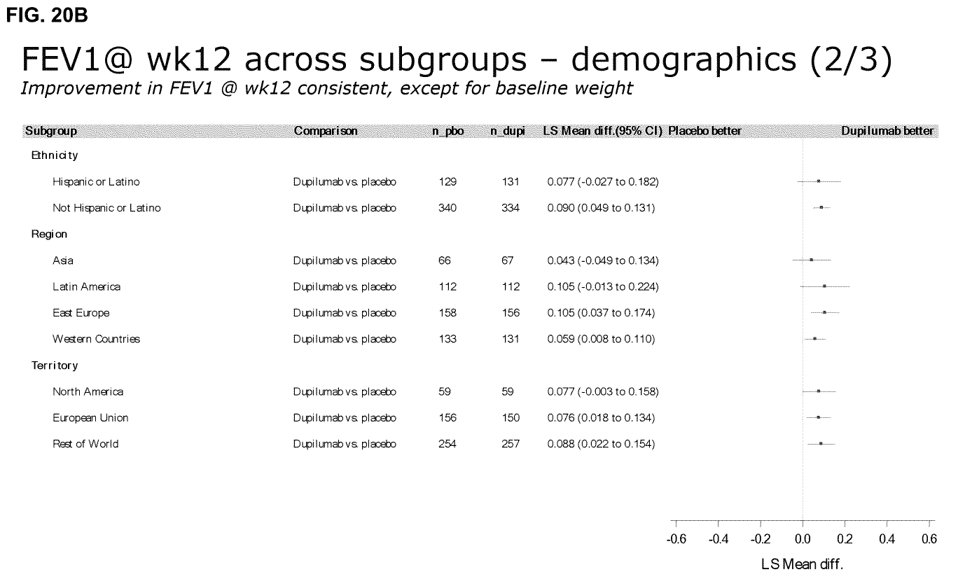

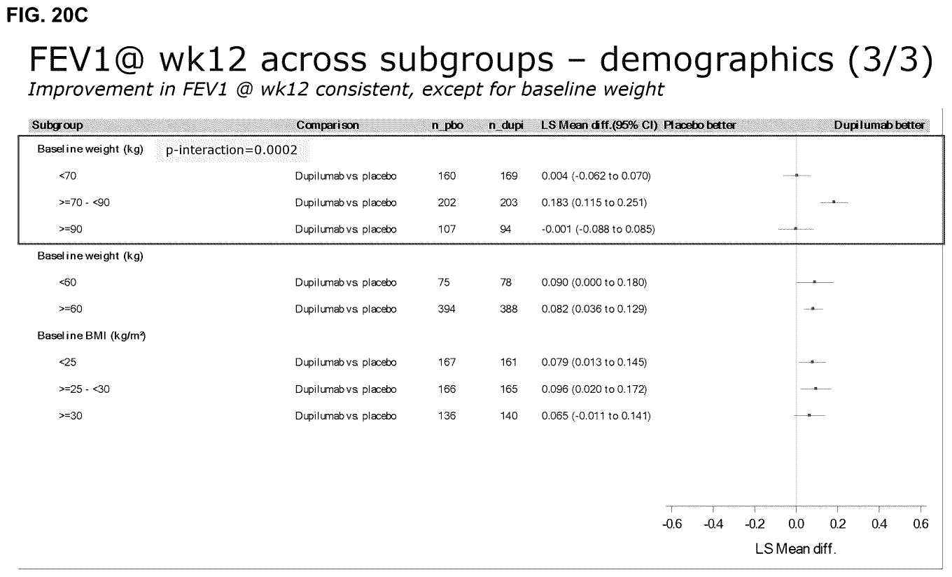

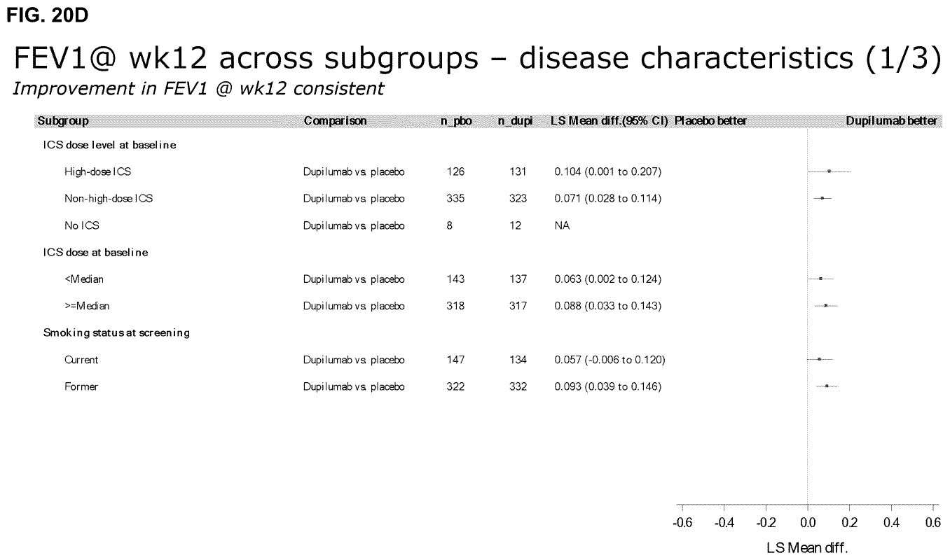

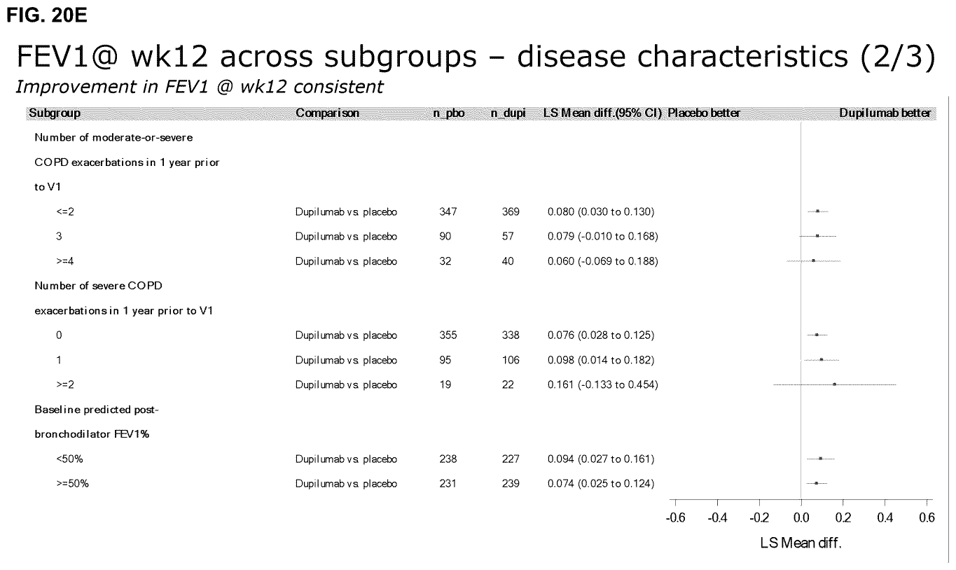

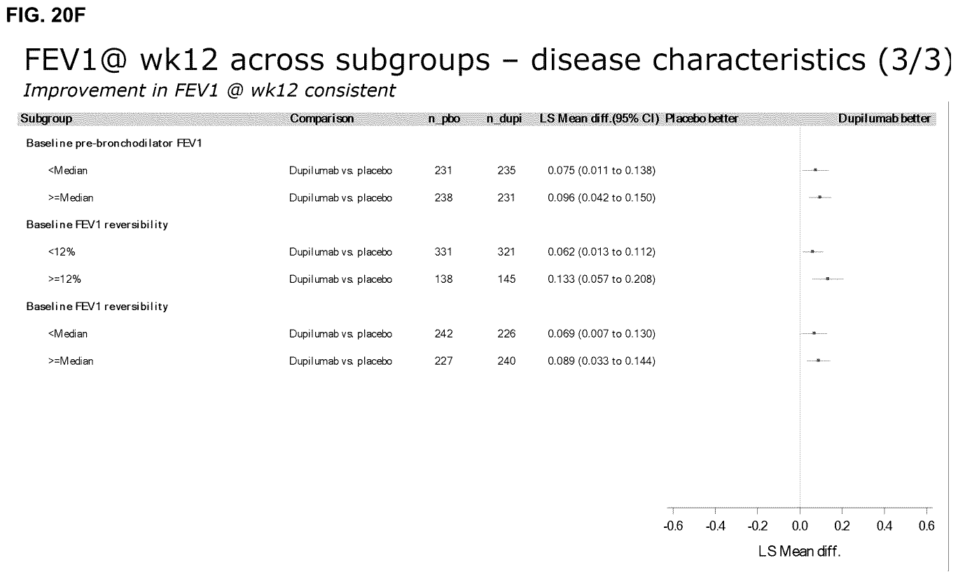

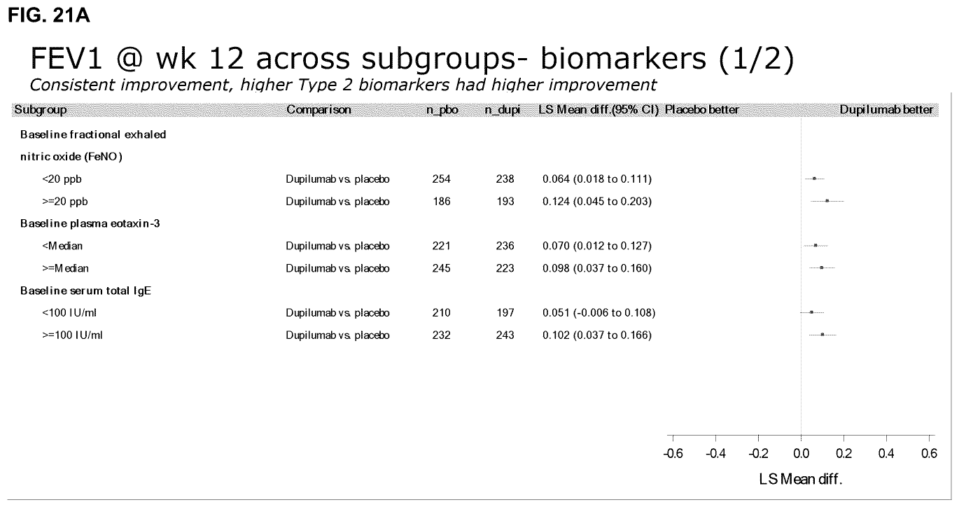

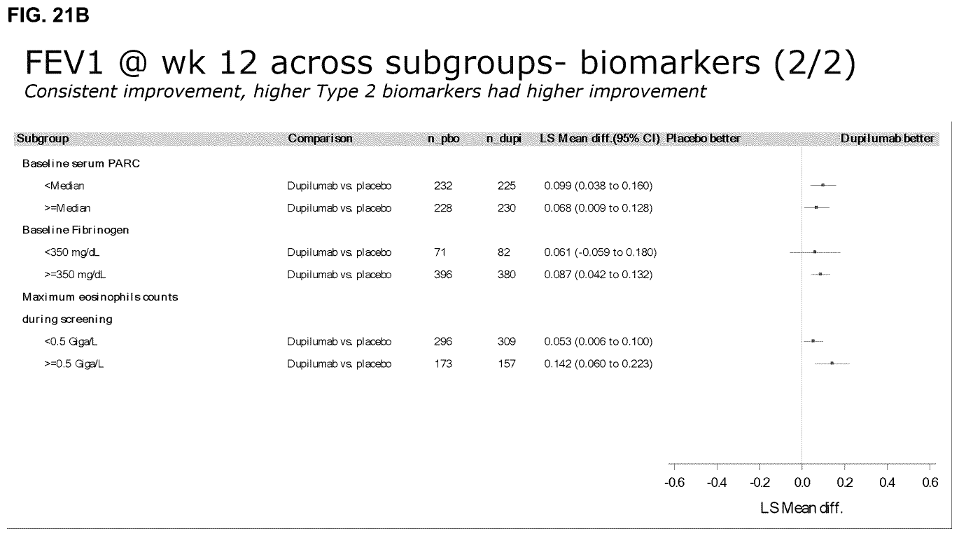

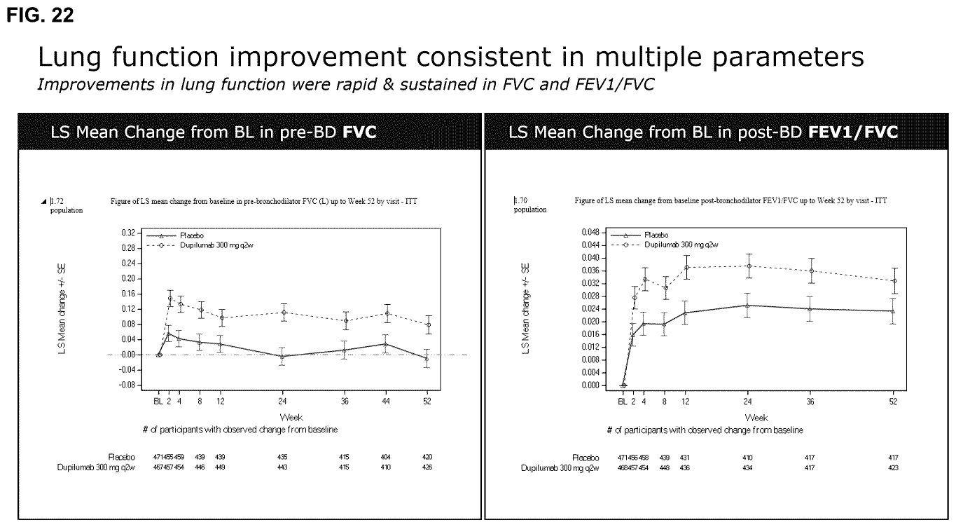

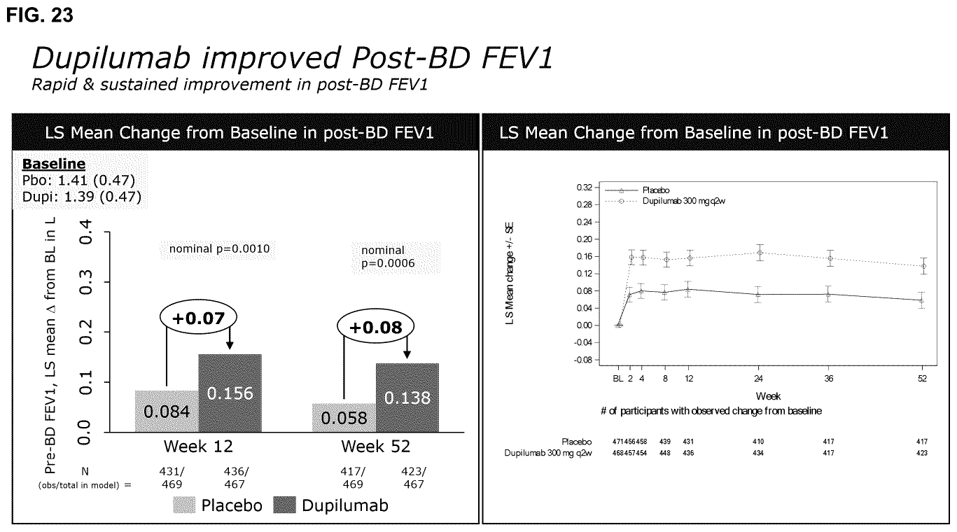

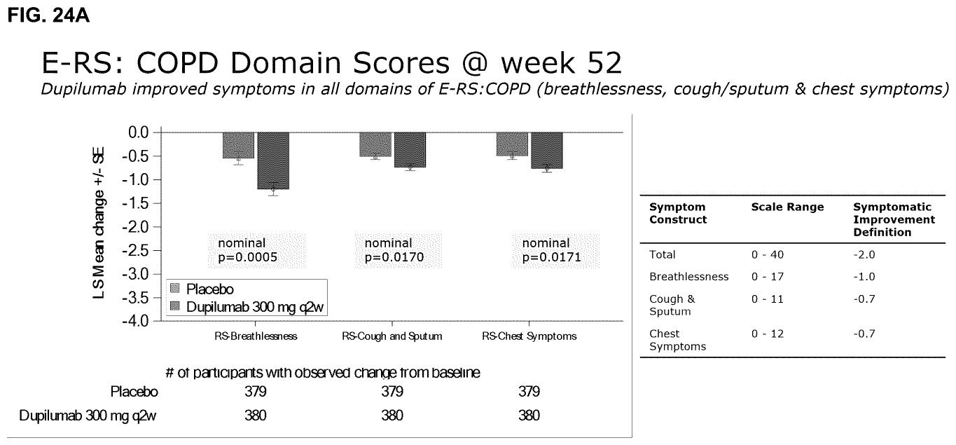

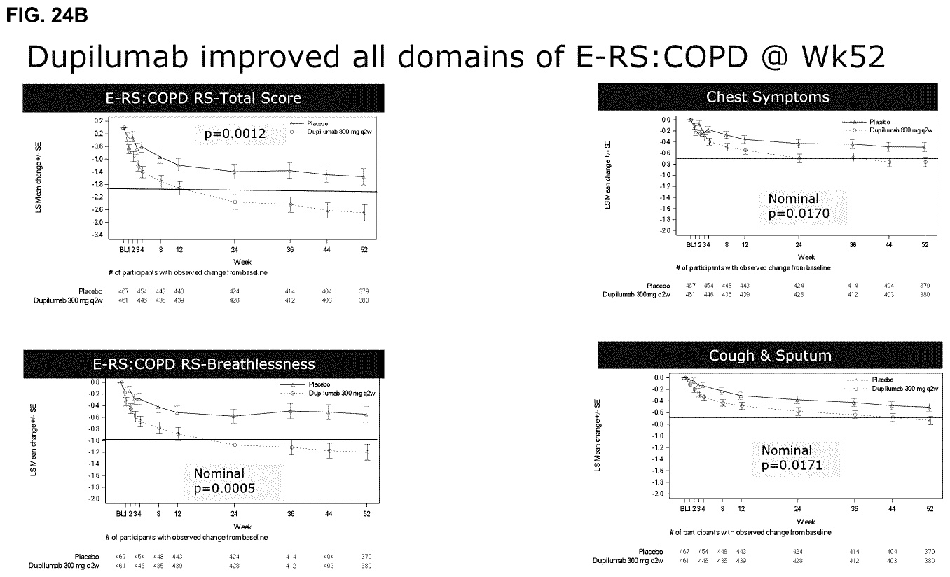

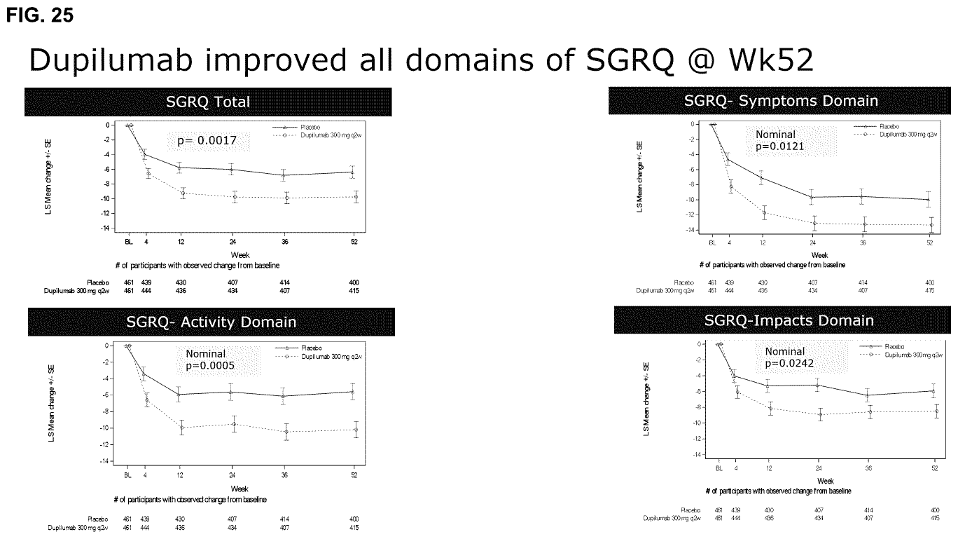

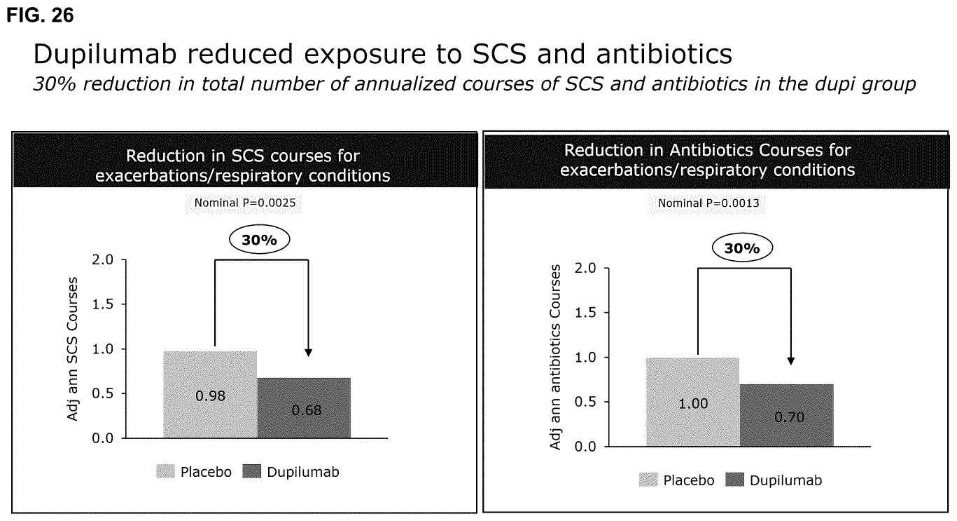

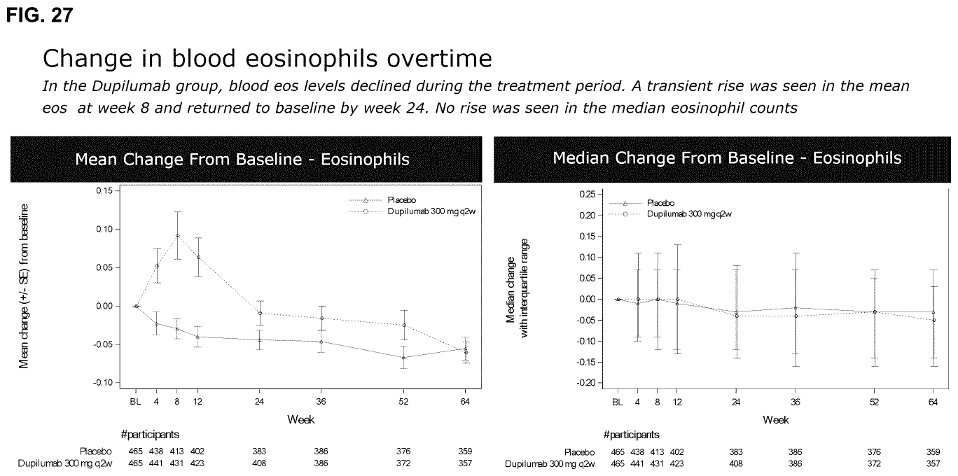

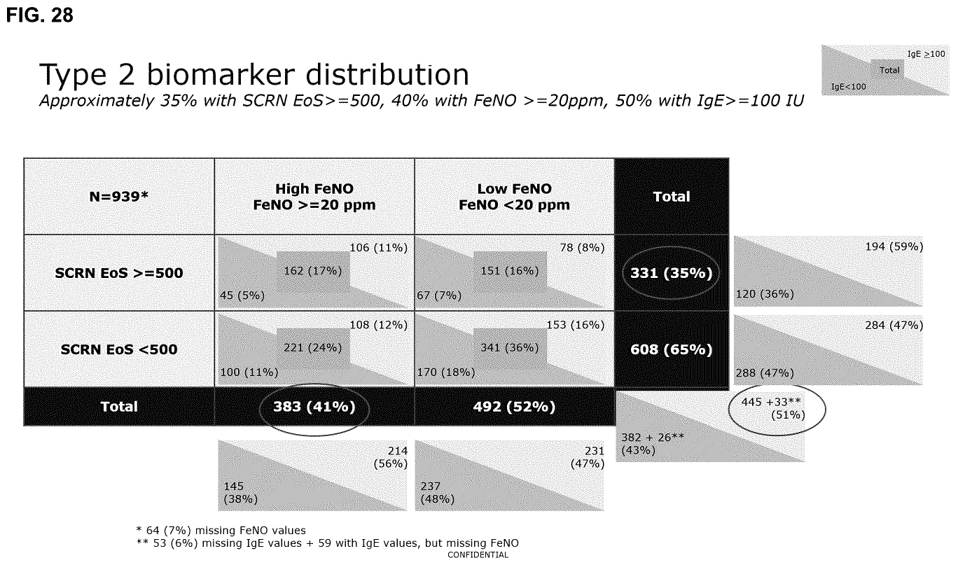

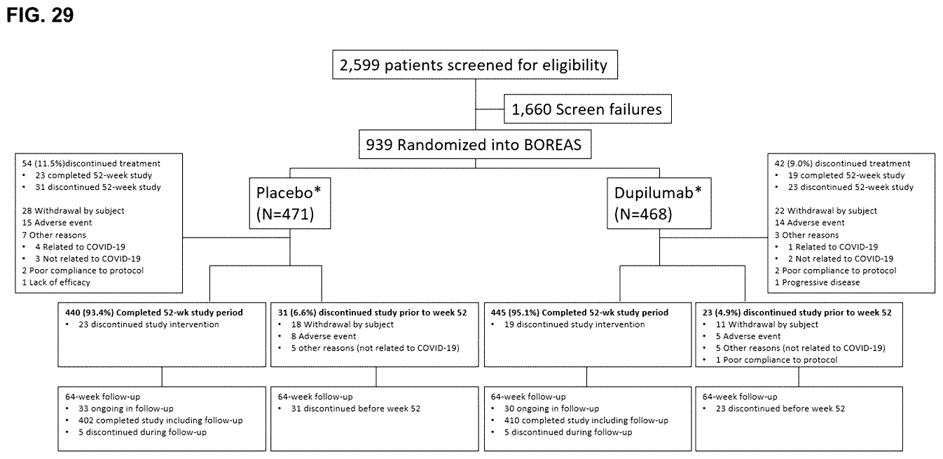

A total of 939 participants were randomized (468 dupilumab and 471 placebo). A high percentage of participants completed the 52-week study period (95.1% dupilumab vs. 93.4% placebo; FIG. 29). The demographics and disease characteristics of participants were balanced at baseline between groups (FIG. 13A to FIG. 13E). The mean age (SD) of the participants was 65.1 (8.1) years. Participants were predominantly male and non-Hispanic/Latino white. Overall, 30.0% of participants were current smokers, and 27.4% of participants were on high-dose ICS (≥1000 μg beclomethasone dipropionate equivalent (Table 12)).

TABLE 12
High-dose inhaled corticosteroid dosing regimen

Daily dose (mcg)

| | | |
|---|---|---|
| Inhaled corticosteroid | High dose | Lower doses |

Japan

| | | |
|---|---|---|
| Beclomethasone dipropionate (HFA) | 401-800 | ≤400 |
| Fluticasone propionate (HFA) | 401-800 | ≤400 |
| Ciclesonide (HFA) | 401-800 | ≤400 |
| Fluticasone propionate (DPI) | 401-800 | ≤400 |
| Budesonide (DPI) | 801-1600 | ≤800 |
| Budesonide inhalation suspension | 1 < x ≤ 2 | ≤1.0 |

Rest of world

| | | |
|---|---|---|
| Beclomethasone dipropionate (CFC) | >1000 | ≤1000 |
| Beclomethasone dipropionate (HFA) | >400 | ≤400 |
| Budesonide (DPI) | >800 | ≤800 |
| Ciclesonide (HFA) | >320 | ≤320 |
| Fluticasone propionate (DPI or HFA) | >500 | ≤500 |
| Mometasone furoate | >440 | ≤440 |
| Triamcinolone acetonide | >2000 | ≤2000 |

NOTE:
CFC chlorofluorocarbon, DPI dry powder inhaler, HFA hydrofluoroalkane.

Almost all participants (97.6%) were on triple therapy of ICS+LAMA+LABA. The mean number of moderate or severe exacerbations in the prior year, SGRQ score, and E-RS score were similar between groups. Mean (SD) baseline absolute blood eosinophil counts were 401 (298) cells/μL and FENO levels were 24.33 (22.40) ppb.

### Primary Endpoint

### Annualized Rate of COPD Exacerbations

The mean annualized rate of moderate or severe exacerbations in the dupilumab group was 0.776 per year, compared with 1.101 per year in the placebo group (rate ratio vs placebo 0.705; 95% confidence interval [CI] 0.581 to 0.857, p=0.0005), equivalent to a 30% relative risk reduction in the primary endpoint (Table 13).

TABLE 13
Summary of Multiplicity-Controlled Endpoints

| | | |
|---|---|---|
| | Placebo | Dupilumab |
| Hierarchy | (N = 471) | (N = 468) |

Primary endpoint, ITT Population

| | | | | | |
|---|---|---|---|---|---|
| Annualized rate of moderate- | 1 | | | | |
| to-severe exacerbations | | | | | |
| Adjusted annualized rate | | 1.101 | (0.931 to 1.301) | 0.776 | (0.645 to 0.934) |
| of moderate or severe | | | | | |
| exacerbations, events/yr | | | | | |

| | | | |
|---|---|---|---|
| Rate ratio vs placebo | — | 0.705 | (0.581 to 0.857) |
| (95% CI) | | | |

| | | |
|---|---|---|
| Adjusted P value vs placebo | — | <0.001 |

Key secondary and other hierarchy endpoints, ITT Population

| Change in pre-BD FEV₁ | 2 | | | | |
|---|---|---|---|---|---|
| from baseline to week 12 | | | | | |
| LS Mean (SE) - L | | 0.077 | (0.018) | 0.160 | (0.018) |
| LS Mean Difference (95% CI) | | — | | 0.083 | (0.042 to 0.125) |
| Adjusted P value vs placebo | | — | | <0.0001 | |
| Change in pre-BD FEV₁ | 3 | | | | |
| from baseline to week 52 | | | | | |
| LS Mean (SE) - L | | 0.070 | (0.019) | 0.153 | (0.019) |
| LS Mean Difference (95% CI) | | — | | 0.083 | (0.038 to 0.128) |
| Adjusted P value vs placebo | | — | | <0.001 | |
| F_{E}_{_{NO}} ≥20 ppb subgroup: | 4 | | | | |
| Change in pre-BD FEV₁ | | | | | |
| from baseline to Week 12 | | | | | |
| LS Mean (SE) - L | | 0.108 | (0.035) | 0.232 | (0.034) |
| LS Mean Difference (95% CI) | | — | | 0.124 | (0.045 to 0.203) |
| Adjusted P value vs placebo | | — | | 0.0022 | |
| F_{E}_{_{NO}} ≥20 ppb subgroup: | 5 | | | | |
| Change in pre-BD FEV₁ | | | | | |
| from baseline to Week 52 | | | | | |
| LS Mean (SE) | | 0.120 | (0.037) | 0.247 | (0.036) |
| LS Mean Diff, 95% CI | | — | | 0.127 | (0.042 to 0.212) |
| Adjusted P value vs placebo | | — | | 0.0034 | |
| Change from baseline to Week | 6 | | | | |
| 52 in SGRQ total score (0-100) | | | | | |
| LS Mean (SE) | | −6.369 | (0.816) | −9.732 | (0.810) |
| LS Mean Difference (95% CI) | | — | | −3.363 | (−5.459 to −1.266) |
| Adjusted P value vs placebo | | — | | 0.0017 | |
| Proportion of participants | 7 | | | | |
| with SGRQ improvement ≥4 | | | | | |
| points at week 52 | | | | | |
| Responders, no. (%) | | 203 | (43.1) | 241 | (51.5) |
| Odds ratio vs placebo (95% CI) | | — | | 1.439 | (1.096 to 1.890) |
| Adjusted P value vs placebo | | — | | 0.0089 | |
| Change in E-RS: COPD total | 8 | | | | |
| Score from baseline to week 52 | | | | | |
| LS Mean (SE) | | −1.558 | (0.256) | −2.694 | (0.257) |
| LS Mean Difference (95% CI) | | — | | −1.137 | (−1.823 to −0.450) |
| Adjusted P value vs placebo | | — | | 0.0012 | |
| F_{E}_{_{NO}} ≥20 ppb subgroup: | 9 | | | | |
| Annualized rate of moderate-to- | | | | | |
| severe exacerbations | | | | | |
| Adjusted annualized rate | | 1.117 | (0.831 to 1.502) | 0.699 | (0.510 to 0.958) |
| of moderate or severe | | | | | |
| exacerbations, events/yr | | | | | |

| | | | |
|---|---|---|---|
| Rate ratio vs placebo (95% CI) | — | 0.625 | (0.450 to 0.869) |

| | | |
|---|---|---|
| Adjusted P value vs placebo | — | 0.0052 |

BD bronchodilator, CI confidence interval, E-RS evaluating respiratory symptoms, F_{E}_{_{NO}} fractional exhaled nitric oxide, FEV₁ forced expiratory volume in one second, ITT intent-to-treat, L liter, LS least squares, ppb parts per billion, RS respiratory symptoms, SE standard error, SD standard deviation, SGRQ St. George's respiratory questionnaire.

The cumulative mean number of moderate or severe exacerbations during the 52-week treatment period was reduced in participants receiving dupilumab compared placebo. Dupilumab delayed the time to first moderate or severe exacerbation (hazard ratio 0.803 [95% CI 0.658 to 0.980], nominal p=0.0309).

A summary of the occurrence of moderate or severe exacerbations during the 52-week treatment period is presented in Table 14. All moderate or severe adjudicated exacerbation events that occurred during the 52-week treatment period are included, regardless of whether the participant was receiving treatment or not.

TABLE 14
Summary of Occurrence of Moderate or Severe COPD Exacerbations
During the 52-Week Treatment Period.

| | Placebo | Dupilumab |
|---|---|---|
| | (N = 471) | (N = 468) |
| Total number of moderate or severe | 422 | 296 |
| events | | |
| Number of severe events | 37 | 28 |
| Number of moderate events | 385 | 268 |
| Treated with systemic corticosteroid | 130 | 84 |
| medications only, n | | |
| Relative risk vs. placebo (95% CI) | | 0.631 (0.433-0.922) |
| Treated with systemic antibiotics | 255 | 184 |
| (with or without corticosteroids), n | | |
| Relative risk vs. placebo (95% CI) | | 0.722 (0.572-0.911) |

### Secondary and Other Endpoints

### Lung Function

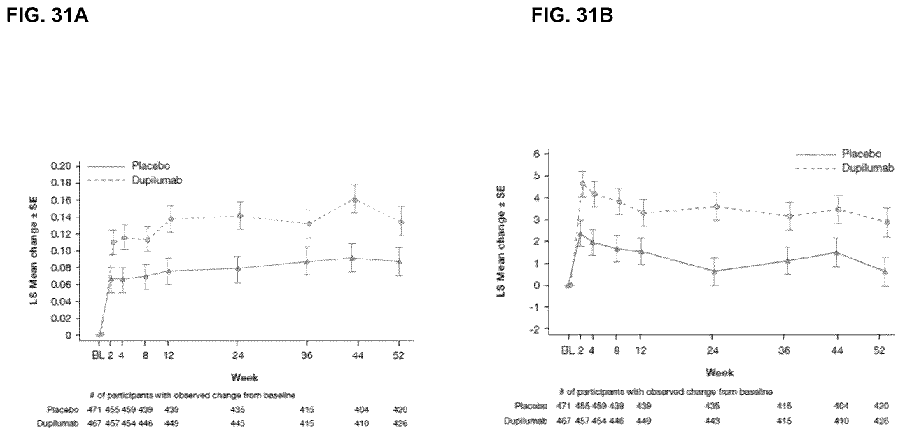

Treatment with dupilumab led to a statistically significant improvement in lung function (pre-BD FEV1) within the first two weeks of initial administration and was sustained throughout the study. At week 12, pre-BD FEV1 least squares mean (LSM) change from baseline was 0.160 L for dupilumab vs 0.077 L for placebo (LSM difference 0.083 L, 95% CI 0.042 to 0.125, p<0.0001), and this improvement was sustained through week 52 (LSM difference 0.083 L, 95% CI 0.038 to 0.128, p<0.001). Multiple lung function parameters improved with dupilumab compared to placebo, including the post-BD FEV1, post-BD FEV1/FVC ratio, pre-BD FVC and pre-BD forced expiratory flow from 25% to 75% (FEF25-75%) (FIG. 31A-FIG. 31B).

### Patient-Related Outcomes

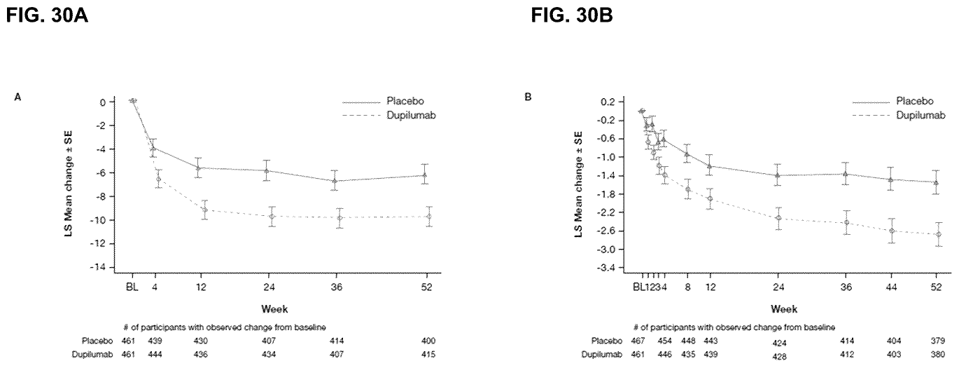

Dupilumab compared with placebo improved the SGRQ total score at week 52 (−9.732 vs −6.369, respectively; LSM difference −3.363, 95% CI −5.459 to −1.266, p=0.0017). This improvement was rapid and sustained (FIG. 30A). A higher proportion of participants in the dupilumab group than in the placebo group showed an improvement in SGRQ score of greater than the minimum clinically important difference of 4 units at week 52 (dupilumab 51.5% vs placebo 43.1%, odds ratio (OR) 1.439; 95% CI 1.096 to 1.890, p=0.0089).

Dupilumab treatment vs placebo was associated with a significantly greater reduction in E-RS: COPD total score from baseline to week 52 (LSM change −2.694 vs −1.558, LSM difference −1.137, 95% CI −1.823 to −0.450, p=0.0012) (FIG. 30B). Participants reported reduced severity of symptoms as early as week 2, and these reductions were sustained through week 52.

Subgroup with FeNO ≥20 ppb

In the subgroup of patients with FeNO ≥20 ppb, dupilumab reduced the annualized rate of moderate or severe exacerbations by 37% compared to placebo (rate ratio vs. placebo 0.625; 95% CI 0.450 to 0.869, p=0.0052) (Table 12). Dupilumab led to an improvement of 0.124 L (LSM-difference; 95% CI 0.045 to 0.203, p=0.0022) in pre-BD FEV1 at week 12 vs. placebo that was sustained through week 52 (LSM-difference 0.127 L, 95% CI 0.042 to 0.212, p=0.0034).

### Biomarkers of Type 2 Inflammation

Dupilumab had minimal effects on blood eosinophil counts while substantially reducing other markers of type 2 inflammation including FENO, serum IgE, pulmonary and activation-regulated chemokine (PARC) and eotaxin levels during the entire 52-week treatment period (Table 15).

TABLE 15
Change In Biomarkers from Baseline
to Week 52 - Safety Population

| | |
|---|---|
| Placebo | Dupilumab 300 |
| (N = 470) | (N = 469) |

Blood eosinophils, cells/μL
Baseline

| | | |
|---|---|---|
| N | 470 | 469 |
| Median | 335 | 340 |

Percent change from baseline

| | | |
|---|---|---|
| N | 376 | 372 |
| Median | −9.45 | −11.95 |

Eotaxin-3, pg/mL
Baseline

| | | |
|---|---|---|
| N | 467 | 462 |
| Median | 154.00 | 146.00 |

Percent change from baseline

| | | |
|---|---|---|
| N | 374 | 368 |
| Median | −1.85 | −35.53 |

PARC, pg/mL
Baseline

| | | |
|---|---|---|
| N | 461 | 458 |
| Median | 72100.0 | 72350.0 |

Percent change from baseline

| | | |
|---|---|---|
| N | 379 | 379 |
| Median | −0.8 | −14.4 |

Total IgE, IU/mL
Baseline

| | | |
|---|---|---|
| N | 443 | 443 |
| Median | 116.00 | 121.00 |

Percent change from baseline

| | | |
|---|---|---|
| N | 352 | 351 |
| Median | −3.44 | −65.84 |

Post-BD F_{E}_{_{NO}}, ppb
Baseline

| | | |
|---|---|---|
| N | 441 | 434 |
| Median | 17.0 | 17.0 |

Percent change from baseline

| | | |
|---|---|---|
| N | 358 | 350 |
| Median | −6.9 | −28.6 |

BD bronchodilator,
F_{E}_{_{NO}} fractional exhaled nitric oxide,
IgE immunoglobulin E,
IU international unit,
mL milliliter,
PARC pulmonary and activation-regulated chemokine,
pg picogram,
ppb parts per billion,
SD standard deviation

Subgroups with Blood Eosinophils < or ≥300 cells/μL at Baseline

Dupilumab demonstrated greater improvement from baseline in pre-BD FEV1 at week 52 in the subgroup of patients with baseline blood eosinophils ≥300 cells/μL (Table 16). Median change from baseline in blood eosinophils was −30.00 cells/μL in both groups at week 52.

No clinically symptomatic eosinophilia was observed.

TABLE 16
Change from baseline in pre-bronchodilator FEV1
(L) at week 52, in patients with blood eosinophils
< or ≥ 300 cells/μL at baseline

| | |
|---|---|
| | Dupilumab |
| Placebo | 300 mg q2w |
| (N = 471) | (N = 468) |

Baseline eosinophils
count <300 cells/μL
Baseline

| Number | 186 | | 179 | |
|---|---|---|---|---|
| Mean (SD) | 1.30 | (0.46) | 1.28 | (0.47) |

| | | |
|---|---|---|
| Median | 1.21 | 1.19 |
| Q1; Q3 | 0.97; 1.55 | 0.94; 1.58 |
| Min; Max | 0.5; 2.8 | 0.4; 3.4 |

Week 52

| Number | 166 | | 161 | |
|---|---|---|---|---|
| Mean (SD) | 1.36 | (0.52) | 1.43 | (0.61) |

| | | |
|---|---|---|
| Median | 1.27 | 1.32 |
| Q1; Q3 | 0.95; 1.61 | 1.02; 1.79 |
| Min; Max | 0.5; 3.2 | 0.4; 3.5 |

Change from baseline

| Number | 166 | | 161 | |
|---|---|---|---|---|
| Mean (SD) | 0.03 | (0.31) | 0.12 | (0.45) |
| Median | −0.02 | | 0.01 | |
| Q1; Q3 | −0.13; 0.16 | | −0.09; 0.20 | |
| Min; Max | −0.7; 1.8 | | −0.5; 2.7 | |
| Number of | 185 | | 179 | |
| participants | | | | |
| in the model | | | | |
| LS Mean | 0.043 | (0.033) | 0.108 | (0.033) |
| (SE)^{a} | | | | |
| LS Mean Diff, | | | 0.065 | (−0.012 to 0.143) |
| 95% Cl^{a} | | | | |

≥300 cells/μL
Baseline

| Number | 285 | 288 | |
|---|---|---|---|
| Mean (SD) | 1.33 (0.47) | 1.29 | (0.44) |

| | | |
|---|---|---|
| Median | 1.22 | 1.23 |
| Q1; Q3 | 1.00; 1.60 | 0.94; 1.57 |
| Min; Max | 0.5; 3.4 | 0.4; 2.7 |

Week 52

| Number | 254 | | 265 | |
|---|---|---|---|---|
| Mean (SD) | 1.41 | (0.53) | 1.45 | (0.55) |

| | | |
|---|---|---|
| Median | 1.34 | 1.37 |
| Q1; Q3 | 1.04; 1.69 | 1.02; 1.85 |
| Min; Max | 0.5; 3.6 | 0.5; 3.2 |

Change from baseline

| Number | 254 | | 265 | |
|---|---|---|---|---|
| Mean (SD) | 0.07 | (0.32) | 0.16 | (0.35) |
| Median | 0.02 | | 0.08 | |
| Q1; Q3 | −0.13; 0.19 | | −0.05; 0.27 | |
| Min; Max | −0.6; 1.3 | | −0.6; 2.0 | |
| Number of | 284 | | 287 | |
| participants | | | | |
| in the model | | | | |
| LS Mean | 0.085 | (0.023) | 0.181 | (0.022) |
| (SE)^{a} | | | | |
| LS Mean Diff, | | | 0.096 | (0.042 to 0.150) |
| 95% Cl^{a} | | | | |

| | |
|---|---|
| Overall p-value | 0.4662 |
| for interaction^{b} | |

MMRM: Mixed-effect Model with Repeated Measures;
ICS: Inhaled Corticosteroid;
FEV1: Forced Expiratory Volume in one second
^{a}Derived from MMRM model with the change from baseline in FEV1 values up to Week 52 as response variables, and factors for treatment group, age, sex, height, region (pooled country), ICS dose at baseline, smoking status at screening, visit, treatment-by-visit interaction, baseline pre-bronchodilator FEV1, and FEV1 baseline-by-visit interaction (except where subgroup of interest)
^{b}Derived from MMRM model with the change from baseline in pre-bronchodilator FEV1 up to Week 52 as response variables, and treatment group, age, sex, height, region (pooled country), ICS dose, smoking status at screening, visit, treatment-by-visit interaction, baseline pre-bronchodilator FEV1, and FEV1 baseline-by-visit interaction, subgroup (if different than the aforementioned covariates), subgroup-by-treatment interaction and subgroup-by-treatment-by-visit interaction as covariates.

### Safety

Dupilumab was generally well-tolerated with a safety profile consistent with the known safety profile in its other approved indications. Overall, more participants receiving dupilumab completed the study (dupilumab 95.1% vs placebo 93.4%) and the proportion of participants with TEAEs was similar between groups (dupilumab 77.4% vs placebo 76.0%). Adverse events (AEs) more commonly observed with dupilumab than with placebo included headache (8.1% vs 6.8%), diarrhea (5.3% vs 3.6%), and back pain (5.1% vs 3.4%). TEAEs leading to treatment discontinuation (3% dupilumab vs 3.4% placebo), SAEs, and TEAEs leading to death were balanced between groups. System organ class adverse events commonly reported with COPD such as respiratory, cardiac, and vascular events, including adjudicated major adverse cardiovascular events, were more frequent in the placebo group (placebo vs dupilumab 17.0% vs 16.0%; 8.5% vs 7.0%; 9.6% vs 7.0%; 1.9% vs 0.9%, respectively).

### Conclusions

Dupilumab significantly decreased moderate-to-severe exacerbations and improved lung function (FEV1), HRQoL/health status (SGRQ) and symptoms (E-RS:COPD RS-Total Score), shortened exacerbation-associated annualized total SCS treatment duration, and reduced total SCS courses due to exacerbations and moderate-to-severe exacerbations in COPD patients with type 2 inflammation in two phase 3 trials. Dupilumab efficacy was rapid, observed as early as week 2, and sustained thorough week 52 across all multiplicity adjusted endpoints. Dupilumab improved FEV1 (not pbo adjusted) by week 12 by +160 ml in ITT and +232 ml in bl FeNO approximately 20 ppb. Lung function improvements were observed in multiple parameters including post-BO FEV1, FVC and FEV1/FVC. Symptom improvements were observed in all the domains of E-RS:COPD (breathlessness, cough & sputum, chest symptoms). Consistent benefits were observed irrespective of demographics, ICS dose (high/low), smoking status, GOLD severity of airflow limitation (baseline), and history of exacerbations. Increasing improvement was observed with increased type 2 biomarker levels. The safety profile was generally consistent with other dupilumab indications.

Dupilumab reduced type 2 biomarkers over time (blood EOS, FeNO and IgE).

Dupilumab reduced the use of systemic steroids (courses and days) and antibiotics (courses and days).

Dupilumab was well-tolerated, with a safety profile consistent with the known safety profile of dupilumab. No new safety concerns were observed. Fewer cardiac and respiratory events were observed in patients treated with dupilumab. Malignancies and deaths were balanced overall.

Baseline post-BD FEV1 in BOREAS was mean (SD) 1.39 L (0.47) for Dupilumab and 1.41 L (0.47) for placebo. At week 12, the least squares (LS) mean change from baseline in post-BD FEV1 was 0.156 L (95% CI 0.121-0.192) for Dupilumab vs. 0.084 L (95% CI 0.048-¬0.120) for placebo (LS mean difference 0.072 L [95% CI 0.030-0.115]; nominal P=0.001); at week 52, LS mean change from baseline in post-BD FEV1 was 0.138 L (95% CI 0.101-0.174) for dupilumab vs 0.058 L (95% CI0.021-0.096) for placebo (LS mean difference 0.079 L [95% CI 0.034-0.124]; nominal P=0<001). The proportion of patients whose post-BD FEV1 increased ≥100 mL at week 52 was 38.3% for Dupilumab and 29.5% for placebo. The baseline post-BD FEV1/FVC ratio was mean [SD] 0.49 (0.12) for Dupilumab and 0.49 (0.11) for placebo. At week 12, the LS mean change from baseline in post-BD FEV1/FVC was 0.037 (95% CI 0.030-0.044) for Dupilumab vs. 0.023 (95% CI 0.015-0.030) for placebo (LS mean difference 0.014 [95% CI 0.005-0.023]; nominal P=0.002); at week 52, the LS mean change from baseline post-BD FEV1/FVC was 0.033 (95% CI 0.025-0.041) for Dupilumab vs. 0.023 (95% CI 0.016-0.031) for placebo (LS mean difference 0.010 [95% TBC −0.000-0.019]; nominal P=0.055). The results indicate that Dupilumab improved post-BD FEV₁ and FEV₁/FVC ratio in patients with COPD and T2 inflammation as early as week 2.

Baseline pre-BD FEV1 was mean (SD) 1.28 L (0.45) for Dupilumab and 1.32 L (0.46) for placebo. At week 12, the least squares (LS) mean change from baseline in pre-BD FEV1 was 0.160 L (95% CI 0.126-0.195) for Dupilumab vs. 0.077 L (95% CI 0.042-0.112) for placebo (mean difference 0.083 L [95% CI 0.042-0.125]; P<0.001); at week 52, the LS mean change from baseline in pre-BD FEV1 was 0.153 L (95% CI 0.116-0.189) for Dupilumab vs. 0.070 L (95% CI 0.033-0.107) for placebo (mean difference 0.083 L [95% CI 0.038-0.128]; P<0.001). The proportion of patients who had improved pre-BD FEV1 by ≥100 mL at week 52 was 41.1% for Dupilumab and 34.3% for placebo. The baseline pre-BD FVC was mean (SD) 2.71 L (0.74) for Dupilumab and 2.83 L (0.90) for placebo. At week 12, the LS mean change from baseline in pre-BD FVC was 0.098 L [95% CI 0.054-0.141] for dupilumab vs 0.029 L [95% CI −0.015-0.072] for placebo (LS mean difference 0.069 [95% CI 0.016-0.121]; nominal P=0.01); at week 52, the LS mean change from baseline pre-BD FVC was 0.079 L [95% CI 0.032-0.127] in the Dupilumab group vs. −0.009 L [95% CI −0.057-0.039] in the placebo group (LS mean difference 0.088 L [95% CI 0.029¬-0.148]; nominal P=0.004). These results indicate that Dupilumab improved pre-BD lung function measures in patients with COPD and T2 inflammation as early as week 2.

Individual annualized total exacerbation-associated systemic corticosteroids (SCS) intake was shorter for dupilumab (18.73 days) vs. placebo (20.99 days). At week 12 and week 52, dupilumab improved pre-BD FEV1; LS mean difference 82 mL, P=0.0001 and 62 mL, P=0.0182, respectively, vs. placebo. Dupilumab improved SGRQ score at week 52 (LS mean difference −3.37, nominal P=0.0068) vs. placebo. Adjusted annualized total SCS courses taken due to exacerbations was 0.639 (0.535-0.763) for dupilumab patients versus 0.966 (0.817-1.142) for placebo.

### Discussion

Dupilumab significantly reduced moderate or severe exacerbations in high exacerbation risk, symptomatic COPD patients who had evidence of type 2 inflammation and were receiving optimized inhaled therapies. Dupilumab also significantly improved lung function, health-related quality of life, and severity of respiratory symptoms, with benefits seen as early as two weeks after initiation of treatment and sustained through the 52-week treatment period. These results highlight a significant unmet need in this patient population, who display evidence of type 2 inflammation and remain symptomatic and develop exacerbations while on standard-of-care triple inhaler therapy.

Studies investigating biologics that target IL-5 or its receptor, a pathway that specifically promotes eosinophil maturation and development with limited effects on other aspects of type 2 inflammation, have so far produced mixed results with respect to exacerbation reduction and no evidence of improvement in lung function, quality of life, or symptoms despite the depletion of peripheral blood eosinophils seen with these agents (Pavord 2017; Criner 2019).

In the BOREAS study, a blood eosinophil count of ≥300 cells/μL was used as a biomarker to identify COPD patients with type 2 inflammation who constituted a COPD subpopulation at higher risk of exacerbations. IL-4 and IL-13 are broadly involved in multiple key type 2 inflammatory pathological processes in COPD, including inflammatory infiltrates, airway fibrosis and remodeling, epithelial barrier dysfunction, goblet cell hyperplasia, mucociliary dysfunction, and mucus hypersecretion (GOLD 2023, Bade 2014).

The clinical benefits of dupilumab, a fully human monoclonal antibody that blocks the shared receptor component for IL-4 and IL-13 in the BOREAS study confirms the role of IL-4 and/or IL-13 in the pathophysiology of this COPD subpopulation with type 2 inflammation.

Dupilumab treatment resulted in a sustained reduction in FeNO, a biomarker that reflects IL-13 activity (Suresh 2007), with dupilumab having greater exacerbation reduction and lung function improvement in patients with FeNO ≥20 ppb at baseline. The greater magnitude of response in participants with FeNO ≥20 ppb is consistent with the known key and central role of IL-4/IL-13 in type 2 inflammation (Higham 2021, Kolsum 2017, Garudadri 2018, Barnes 2019). Through its inhibition of the IL-4/IL-13 pathway, dupilumab may play a unique role in reducing goblet cell hyperplasia, mucus hypersecretion and airway remodeling. Without intending to be bound by scientific theory, in the present study, mucus changes, decreased airway inflammation, reduced edema, and/or lowered airway resistance could have resulted in less airway obstruction and hence improved FEV1, and the observed FVC improvement may reflect an improvement in air trapping. Additionally, dupilumab could have reduced upregulation of nitric oxide synthase, Th2 cell differentiation, and recruitment of type 2 inflammation-related effector cells such as eosinophils, mast cells, and basophils. These mechanisms of action could all theoretically contribute to improvements in exacerbations, lung function, and bronchitis symptoms (Brightling 2010; Suresh 2007).

The strengths of the BOREAS trial include that it was a large multi-national trial, had a strict exclusion of patients with current and prior history of asthma, and exhibited consistency in the clinically relevant results across multiple aspects of important COPD outcomes whilst maintaining a similar safety profile between treatment groups. The study was conducted during the Covid-19 pandemic, which impacted clinical research conduct and patient exposures/behaviors worldwide and led to BOREAS trial limitations, including challenges in recruitment and decreased rates of exacerbation. Notwithstanding these limitations, BOREAS had low drop-out rates and compared to placebo dupilumab demonstrated potent efficacy. Treatment was robust and consistent across multiple subgroups and geographies. Despite extensive recruitment efforts to ensure a representative population, there was an underrepresentation of self-identified Black/African American participants. Finally, although treatment randomization was not stratified specifically by smoking status, the final treatment groups were balanced in this domain, indicating that this potential limitation is unlikely to have impacted the outcomes.

By the year 2060, the World Health Organization estimates that there will be over 5.4 million deaths annually attributable to COPD and related comorbidities (World Health Organization, 2023). Exacerbations of COPD, irrespective of severity, lead to increased hospitalizations, higher mortality and poorer quality of life (Bollmeier 2020, Sato 2016, Seemungal 1998, Bélanger 2018, Prudente 2021, David 2021, Ortega 2018).

BOREAS is the first study of a biologic to demonstrate significant reduction in exacerbations and improvement in lung function, quality of life, and symptoms in COPD patients with type 2 inflammation.

### REFERENCES

Kerkhof M, Voorham J, Dorinsky P, et al. Association between COPD exacerbations and lung function decline during maintenance therapy. Thorax. 2020; 75(9):744-753.
The Global Initiative for Chronic Obstructive Lung Diseases (GOLD). Global Strategy for Diagnosis, Management and Prevention of COPD 2023 Report. goldcopd.org/wp-content/uploads/2022/11/GOLD-2023-ver-1.0-14Nov2022_WMV.pdf. Accessed Apr. 3, 2023.
Celli B R, Fabbri L M, Aaron S D, et al. Differential Diagnosis of Suspected COPD Exacerbations in the Acute Care Setting: Best Practice. Am J Respir Crit Care Med. Jan. 26, 2023 [Online ahead of print] atsjournals.org/doi/abs/10.1164/rccm.202209-1795CI.
MacLeod M, Papi A, Contoli M, et al. Chronic obstructive pulmonary disease exacerbation fundamentals: Diagnosis, treatment, prevention and disease impact. Respirology 2021; 26(6):532-551.
Garudadri S, Woodruff P G. Targeting chronic obstructive pulmonary disease phenotypes, endotypes, and biomarkers. Ann Am Thorac Soc. 2018; 15(Suppl 4):5234-5238.
Higham A, Beech A, Wolosianka S, Jackson N, Long G, Kolsum U, Southworth T, Pham T H, Sridhar S, McCrae C, Newbold P, Singh D. Type 2 inflammation in eosinophilic chronic obstructive pulmonary disease. Allergy. 2021; 76(6):1861-1864.
Kolsum U, Damera G, Pham T H, et al. Pulmonary inflammation in patients with chronic obstructive pulmonary disease with higher blood eosinophil counts. J Allergy Clin Immunol. 2017; 140(4):1181-1184.
Barnes P J. Inflammatory endotypes in COPD. Allergy 2019; 74(7):1249-1256.
Bade G, Khan M A, Srivastava A K, et al. Serum cytokine profiling and enrichment analysis reveal the involvement of immunological and inflammatory pathways in stable patients with chronic obstructive pulmonary disease. Int J Chron Obstruct Pulmon Dis 2014; 9:759-73.
Le Floc'h A, Allinne J, Nagashima K, et al. Dual blockade of IL-4 and IL-13 with dupilumab, an IL-4Rα antibody, is required to broadly inhibit type 2 inflammation. Allergy 2020; 75(5):1188-1204.
Ghandi N A, Pirozzi G, Graham N M H. Commonality of the IL-4/IL-13 pathway in atopic diseases. Expert Review of Clinical Immunology. 2017; 13(5):425-437.
Simpson E L, Akinlade B, Ardeleanu M. Two Phase 3 Trials of Dupilumab versus Placebo in Atopic Dermatitis. N Engl J Med 2016; 375:2335-2348 Dellon S et al. Dupilumab in Adults and Adolescents with Eosinophilic Esophagitis. N Engl J Med 2022; 376(11):1090-1
Dellon E S, Rothenberg M E, Collins M H, et al. Dupilumab in Adults and Adolescents with Eosinophilic Esophagitis. N Engl J Med 2022; 387:2317-2330.
Bachert C, Han J K, Desrosiers M, et al. Efficacy and safety of dupilumab in patients with severe chronic rhinosinusitis with nasal polyps (LIBERTY NP SINUS-24 and LIBERTY NP SINUS-52): results from two multicentre, randomised, double-blind, placebo-controlled, parallel-group phase 3 trials. The Lancet 2019; 394(10209):1638-1650.
Castro M et al. Dupilumab Efficacy and Safety in Moderate-to-Severe Uncontrolled Asthma. N Engl J Med 2018; 378:2486-2496.
Pavord I D, Chanez P, Criner G J, et al. Mepolizumab for eosinophilic chronic obstructive pulmonary disease. N Engl J Med. 2017; 377(17):1613-1629.
Criner G J, Celli B R, Brightling C E, et al. Benralizumab for the prevention of COPD exacerbations. N Engl J Med. 2019; 381(11):1023-1034.
Suresh V, Mih J D, George S C. Measurement of IL-13-induced iNOS-derived gas phase nitric oxide in human bronchial epithelial cells. Am J Respir Cell Mol Biol. 2007; 37(1):97-104.
Brightling C E, Saha S, Hollins F. Interleukin-13: prospects for new treatments. Clin Exp Allergy. 2010; 40(1):42-49.
World Health Organization. Chronic obstructive pulmonary disease. https://www.who.int/news-room/fact-sheets/detail/chronic-obstructive-pulmonary-disease-(COPD). Accessed Apr. 3, 2023.
Bollmeier S G, Hartmann A P. Management of chronic obstructive pulmonary disease: a review focusing on exacerbations. Am J Health Syst Pharm. 2020; 77(4):259-268.
Sato M, Chubachi S, Sasaki M, et al. Impact of mild exacerbation on COPD symptoms in a Japanese cohort. Int J Chron Obstruct Pulmon Dis. 2016; 11:1269-1278.
Seemungal T A, Donaldson G C, Paul E A, Bestall J C, Jeffries D J, Wedzicha J A. Effect of exacerbation on quality of life in patients with chronic obstructive pulmonary disease. Am J Respir Crit Care Med 1998; 157(5 Pt 1):1418-1422.
Bélanger M, Couillard S, Courteau J, et al. Eosinophil counts in first COPD hospitalizations: a comparison of health service utilization. Int J Chron Obstruct Pulmon Dis 2018; 13:3045-3054.
Prudente R, Ferrari R, Mesquita C B, et al. Peripheral blood eosinophils and nine years mortality in COPD patients. Int J Chron Obstruct Pulmon Dis 2021; 16:979-985.
David B, Bafadhel M, Koenderman L, De Soyza A. Eosinophilic inflammation in COPD: from an inflammatory marker to a treatable trait. Thorax 2021; 76:188-195.
Ortega H, Llanos J P, Lafeuille M H, et al. Burden of disease associated with a COPD eosinophilic phenotype. Int J Chron Obstruct Pulmon Dis 2018; 13:2425-2433.
Jones P W, Quirk F H, Baveystock C M, Littlejohns P. A self-complete measure of health status for chronic airflow limitation. The St. George's Respiratory Questionnaire. Am Rev Respir Dis 1992; 145(6):1321-7.
Bafadhel M, Peterson S, De Blas M A, Calverley P M, Rennard S I, Richter K, Fagerås M. Predictors of exacerbation risk and response to budesonide in patients with chronic obstructive pulmonary disease: a post-hoc analysis of three randomised trials. Lancet Respir Med. 2018 February; 6(2):117-126.
Pascoe S, Locantore N, Dransfield M T, Barnes N C, Pavord I D. Blood eosinophil counts, exacerbations, and response to the addition of inhaled fluticasone furoate to vilanterol in patients with chronic obstructive pulmonary disease: a secondary analysis of data from two parallel randomised controlled trials. Lancet Respir Med. 2015 June; 3(6):435-42. doi: 10.1016/S2213-2600(15)00106-X. Epub 2015 Apr. 12. Erratum in: Lancet Respir Med. 2015 June; 3(6):e19.
Siddiqui S H, Guasconi A, Vestbo J, Jones P, Agusti A, Paggiaro P, Wedzicha J A, Singh D. Blood Eosinophils: A Biomarker of Response to Extrafine Beclomethasone/Formoterol in Chronic Obstructive Pulmonary Disease. Am J Respir Crit Care Med. 2015 Aug. 15; 192(4):523-5.

### Example 2. Dupixent Significantly Reduced COPD Exacerbations in Second Positive Phase 3 Trial, Accelerating FDA Submission and Confirming Potential to Become First Approved Biologic for this Serious Disease

In an interim analysis, the NOTUS trial met its primary endpoint with overwhelming efficacy, showing Dupixent significantly reduced exacerbations by 34% compared to placebo in patients with moderate-to-severe COPD and evidence of type 2 inflammation (i.e., blood eosinophils ≥300 cells per μL), confirming results from the landmark BOREAS pivotal trial. Dupixent also rapidly and significantly improved lung function (139 mL in FEV1) compared to placebo (57 mL in FEV1).

An interim analysis of the second investigational Phase 3 chronic obstructive pulmonary disease (COPD) trial (NOTUS) confirmed positive published results from the landmark Phase 3 BOREAS trial showing that Dupixent significantly reduced (34%) exacerbations. The trial also confirmed that treatment with Dupixent led to rapid and significant improvements in lung function by 12 weeks, and was sustained up to 52 weeks. The NOTUS trial evaluated the investigational use of Dupixent compared to placebo in adults currently on maximal standard-of-care inhaled therapy (triple therapy) with uncontrolled COPD and evidence of type 2 inflammation (i.e., blood eosinophils ≥300 cells/μL).

This is the first and only time an investigational biologic in COPD has shown a significant and clinically meaningful reduction in exacerbations in two Phase 3 trials. The NOTUS trial included 935 adults who were current or former smokers aged 40 to 85 years and randomized to receive Dupixent (n=470) or placebo (n=465), which was added to maximal standard-of-care inhaled therapy. Patients receiving Dupixent compared to placebo experienced: 34% reduction in moderate or severe acute COPD exacerbations over 52 weeks (p=0.0002), the primary endpoint. Improved lung function from baseline by 139 mL at 12 weeks compared to 57 mL for placebo (p=0.0001), with the benefit versus placebo sustained through week 52 (115 mL for Dupixent v 54 mL for placebo, p=0.0182), both of which were key secondary endpoints.

The safety results were generally consistent with the known safety profile of Dupixent in its approved indications. Overall rates of adverse events (AE) were 67% for Dupixent and 66% for placebo. AEs more commonly observed with Dupixent (≥5% and ≥1% imbalance) compared to placebo included COVID-19 (9.4% Dupixent, 8.2% placebo), nasopharyngitis (6.2% Dupixent, 5.2% placebo), and headache (7.5% Dupixent, 6.5% placebo). AEs more commonly observed with placebo compared to Dupixent included chronic obstructive pulmonary disease (7.8% placebo, 4.9% Dupixent). AEs leading to deaths were 2.6% for Dupixent and 1.5% for placebo.

The efficacy results in NOTUS were consistent with the previously announced results in BOREAS: 30% reduction in moderate or severe acute COPD exacerbations over 52 weeks (p=0.0005), the primary endpoint. Improved lung function from baseline by 160 mL at 12 weeks compared to 77 mL for placebo (p<0.0001), with the benefit versus placebo sustained through week 52 (p=0.0003).

The safety in NOTUS was also consistent with those previously announced in BOREAS: Overall rates of adverse events (AEs) were 77% for Dupixent and 76% for placebo. AEs more commonly observed with Dupixent compared to placebo included headache (8.1% Dupixent, 6.8% placebo), diarrhea (5.3% Dupixent, 3.6% placebo) and back pain (5.1% Dupixent, 3.4% placebo). AEs more commonly observed with placebo compared to Dupixent included upper respiratory tract infection (9.8% placebo, 7.9% Dupixent), hypertension (6.0% placebo, 3.6% Dupixent) and COVID-19 (5.7% placebo, 4.1% Dupixent). AEs leading to deaths were 1.5% for Dupixent and 1.7% for placebo.

NOTUS and BOREAS are replicate, randomized, phase 3, double-blind, placebo-controlled trials that evaluated the efficacy and safety of Dupixent in adults who were current or former smokers with moderate-to-severe COPD aged 40 to 85 years in NOTUS and 40 to 80 years in BOREAS. Enrolling a total of 1,874 patients, all patients in NOTUS and BOREAS had evidence of type 2 inflammation, as measured by blood eosinophils ≥300 cells/μL. Patients with a diagnosis or history of asthma were excluded from the trials.

During the 52-week treatment period, patients in NOTUS and BOREAS received Dupixent or placebo every two weeks added to a maximal standard-of-care inhaled triple therapy of inhaled corticosteroids (ICS), long-acting beta agonists (LABA), and long-acting muscarinic antagonists (LAMA). Double maintenance therapy, which included LABA and LAMA, was allowed if ICS was contraindicated.

The primary endpoint for NOTUS and BOREAS evaluated the annualized rate of acute moderate or severe COPD exacerbations. Moderate exacerbations were defined as those requiring systemic steroids and/or antibiotics. Severe exacerbations were defined as those: requiring hospitalization; requiring more than a day of observation in an emergency department or urgent care facility; or resulting in death.

The Dupilumab COPD program consisted of two Phase III (BOREAS & NOTUS), 52-week DBPC trials of dupilumab 300 mg Q2W in patients with uncontrolled moderate-to-severe COPD despite standard of care (SOC) (LABA+LAMA+ICS, unless ICS contraindicated) and type 2 inflammation (screening eosinophils ≥0.3 Giga/L).

BOREAS (EFC15804) n=939, met all multiplicity-controlled endpoints, NOTUS (EFC15805) n=935 IA, 92% information fraction (this KRM) NOTUS replicated efficacy observed in the in BOREAS trial. Dupilumab improved moderate-severe exacerbations, lung function (FEV1), and HRQoL/health status (SGRQ, nominal) in COPD patients with type 2 inflammation.

Higher efficacy was observed in FeNO ≥20 ppb for exacerbations (nominal p<0.0001) and lung function (week 12 p=0.0010). (Table 17.)

TABLE 17

| | | BOREAS | | NOTUS | |
|---|---|---|---|---|---|
| | | Dupixent | BOREAS | Dupixent | NOTUS |
| Endpoint | Timepoint | vs. placebo | p-value | vs. placebo | p-value |
| Moderate-severe COPD | annualized | 0.705 | 0.0005 | 0.664 | 0.0002 |
| exacerbations (primary) | | (RRR 30%) | | (RRR 34%) | |
| Δpre-BD FEV1 | Week 12 vs. BL | 0.083 | 0.0003 | 0.082 | 0.0001 |
| Δpre-BD FEV1 | Week 52 vs. BL | 0.124 | 0.0022 | 0.062 | 0.0182 |
| ΔSGRQ (0-100) | Week 52 vs. BL | 3.363 | 0.0017 | −3.371 | 0.0068 |
| total score | | | | | (NS) |

Dupilumab was well-tolerated with an acceptable safety profile in the COPD population. No new safety concerns were identified in the NOTUS study. TEAEs (66% placebo vs. 67% Dupixent), SAEs (16% placebo vs. 13% Dupixent), and TEAEs leading to treatment discontinuation (2.6% placebo vs. 3.8% Dupixent) were generally similar between treatment groups. TEAEs (≥500 with ≥100 imbalance) more common to dupilumab were COVID19 (8.2% pbo vs. 9.4% Dupixent), nasopharyngitis (5.2% placebo vs. 6.2% Dupixent), headache (6.5% placebo vs. 7.5% Dupixent); TEAEs more common to placebo were COPD (7.8% placebo vs. 4.9% Dupixent)

TEAE leading to death were higher in Dupixent (1.5% placebo vs. 2.6% Dupixent), with no imbalance in CV deaths (0.2% placebo vs. 0.2% Dupixent). MACE events were more frequent in placebo (1.5% placebo vs. 0.6% Dupixent).

COPD relevant SOCs included respiratory, thoracic, and mediastinal disorders, cardiac disorders, and vascular disorders (placebo vs. Dupixent: 11.4% vs. 10.2%; 4.7% vs. 4.9%; 6.0% vs. 4.5%; respectively).

AESIs were higher in Dupilumab-treated patients (7.1% placebo vs. 8.3% Dupixent). More common in Dupixent: AESI infections (6.7% placebo vs. 8.1% Dupixent), one case of severe conjunctivitis/keratitis in Dupixent; few AESIs of opportunistic infections (two participants with herpes zoster in Dupixent and none in placebo). No clinically symptomatic eosinophilia was observed. No anaphylaxis was observed with in Dupixent, one event was observed with placebo (venom). Other selected AEs that were higher in Dupixent were (2.4% placebo vs. 4.9% Dupixent): conjunctivitis (broad, 0.9% placebo vs. 2.1% Dupixent), injection site reaction (0.4% placebo vs 1.9% Dupixent).

Malignances overall were balanced between treatment groups (1.1% placebo vs. Dupixent 1.1%).

### Key Eligibility Criteria

Age 40-85 (Age 40-80 in BOREAS).

Moderate-to-severe COPD (post-BD FEV1/FVC <0.70 and post-BD FEV1% predicted >30% and ≤70%).

History of high exacerbation risk: >2 moderate or ≥1 severe exacerbations within the year prior to inclusion.

Symptoms (MRC Dyspnea Scale >2).

Signs and symptoms of chronic bronchitis for 3 months in the year prior to screening

EOS ≥300 cells/μL at screening.

Current or former smokers with >10 pack years smoking history (Current smokers capped at 30%).

### Key Exclusion Criteria

COPD diagnosis for <12 months.

Current diagnosis or history of asthma.

Significant pulmonary disease other than COPD.

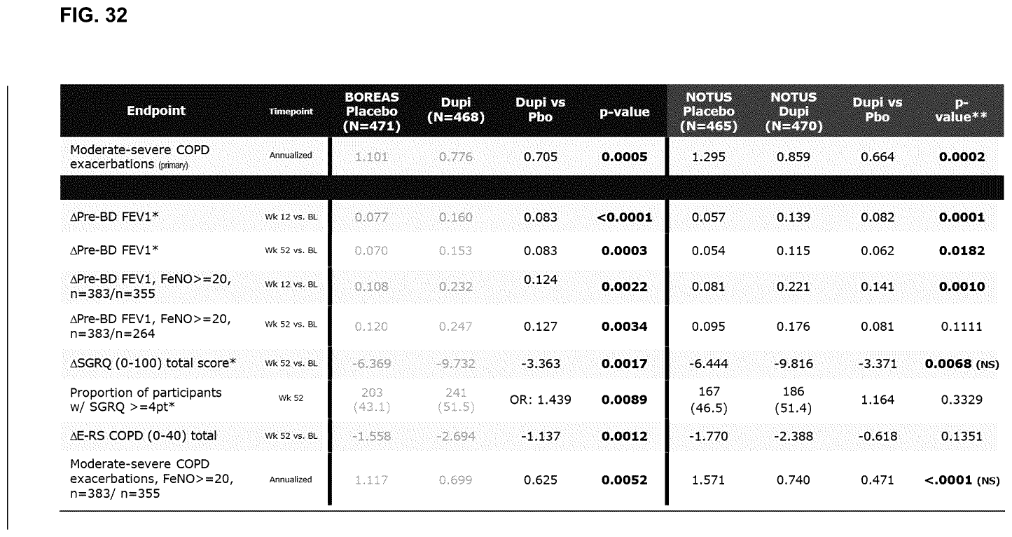

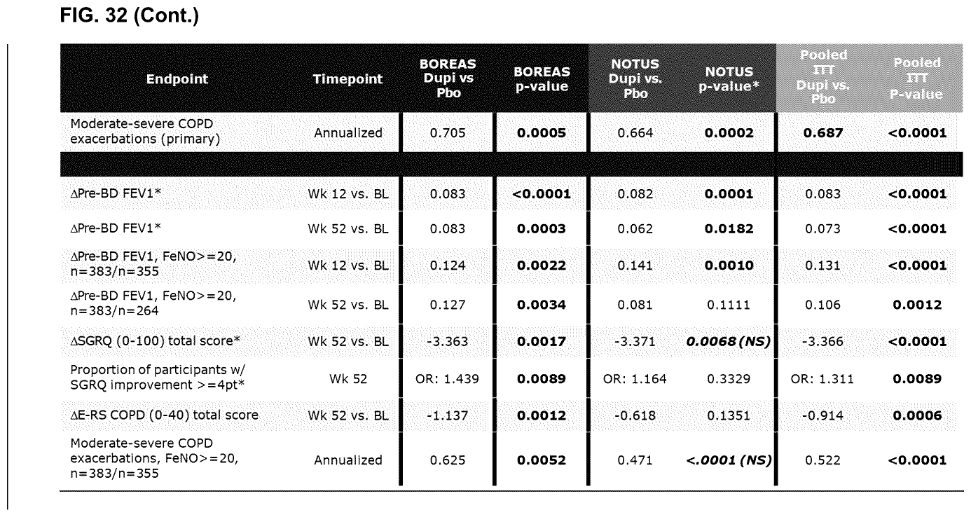

Oxygen therapy was allowed if >4 L min. or if it required >2 L/min. to maintain SpO₂ >88%. See FIG. 32.

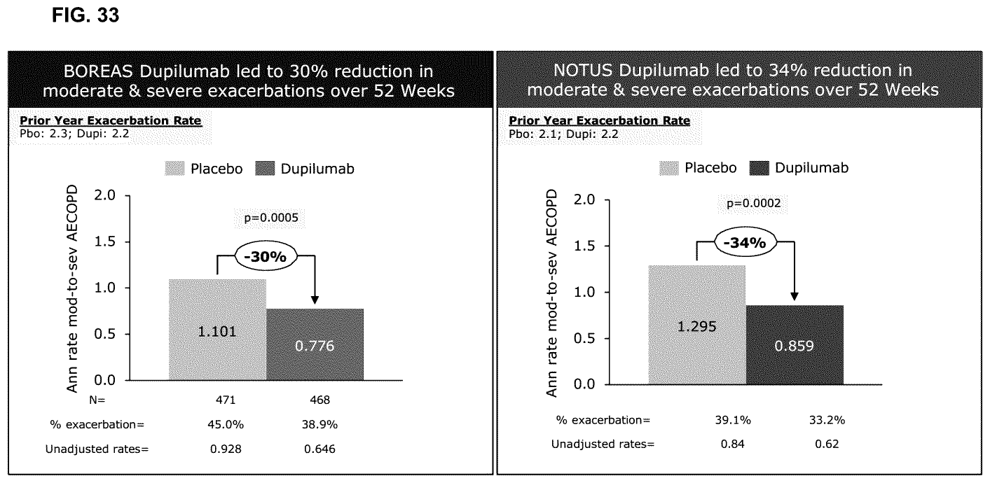

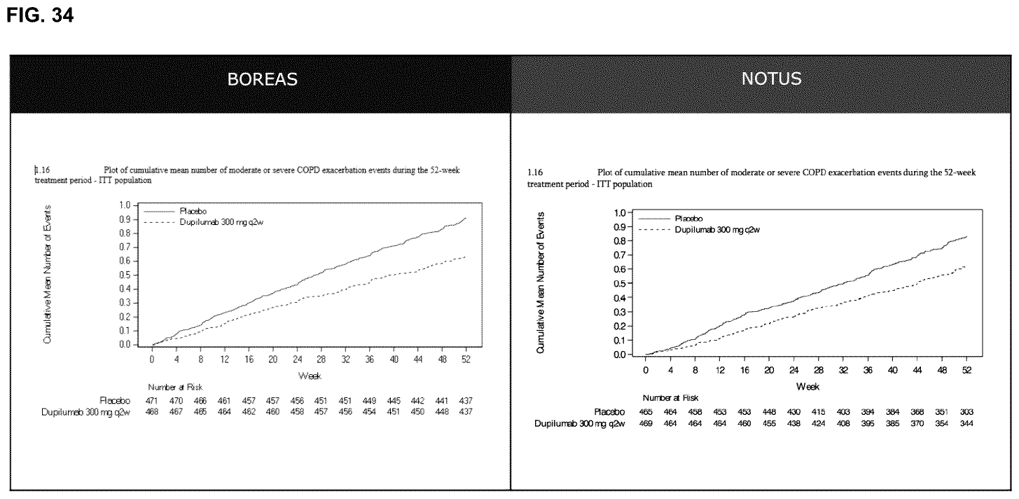

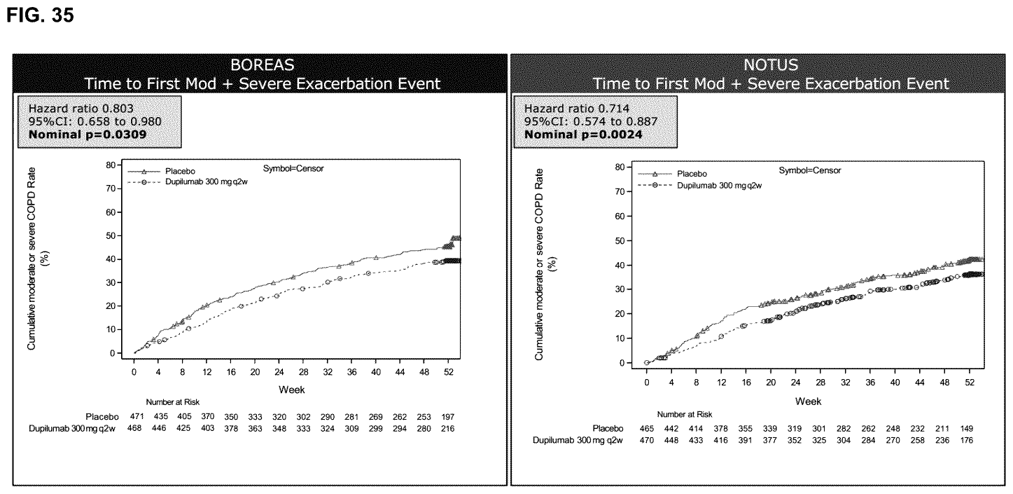

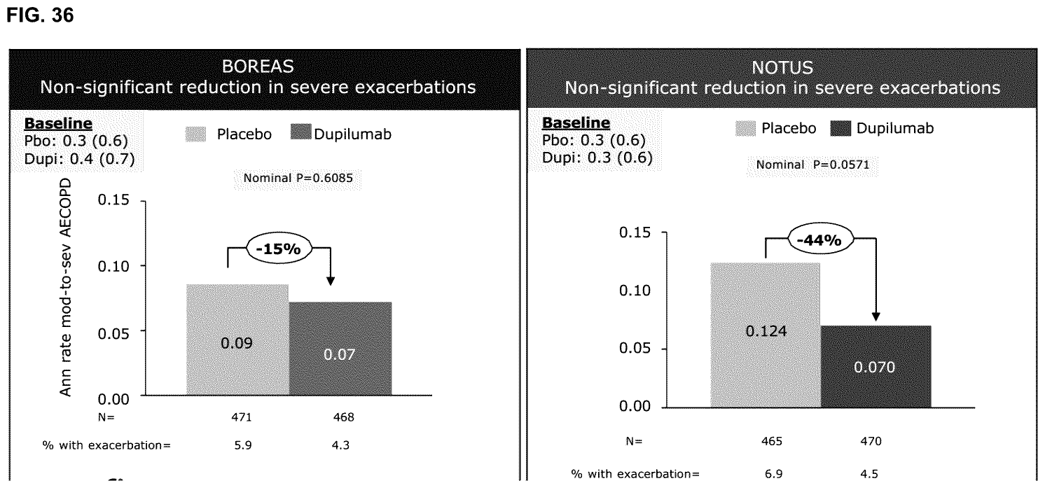

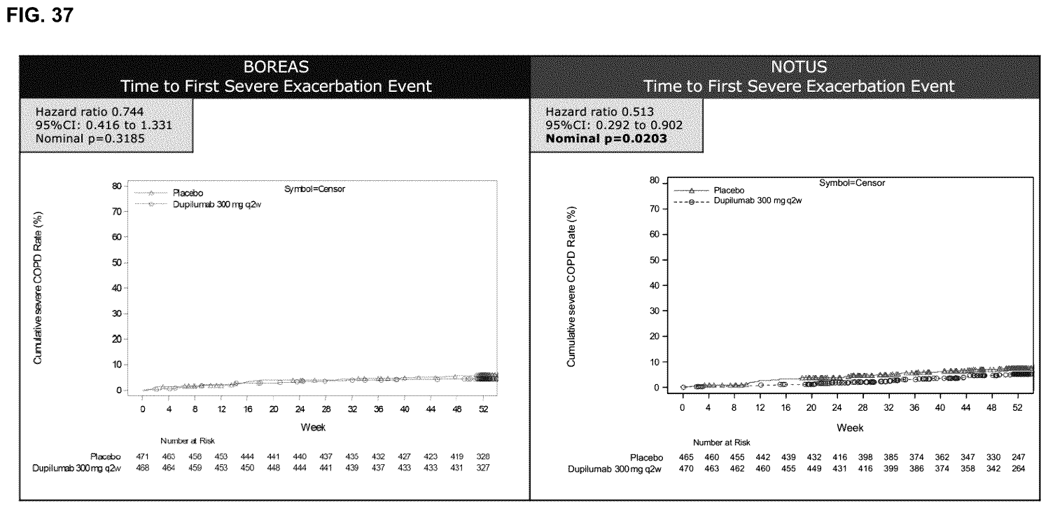

Dupilumab reduced the rate of moderate and severe exacerbations. The exacerbation reduction of 34% in NOTUS was statistically significant and clinically meaningful (0.86; 95% confidence interval [CI], 0.70 to 1.06 vs 1.30; 95% CI, 1.05 to 1.60; relative risk vs placebo 0.66 [95% CI, 0.54 to 0.82]; P<0.001). (FIG. 33). Early and sustained separation was observed in both the BOREAS and NOTUS trials. (FIG. 34). Dupilumab reduced the time to moderate and severe exacerbations after week four (FIG. 35). A greater reduction in sever exacerbations was observed in the NOTUS trial. (FIG. 36). Dupilumab reduced the time to the first severe exacerbation (FIG. 37).

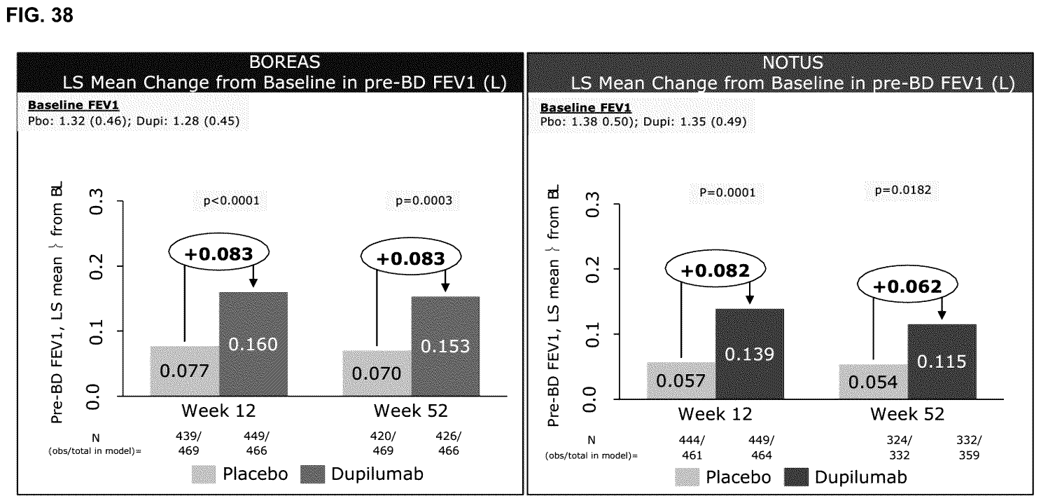

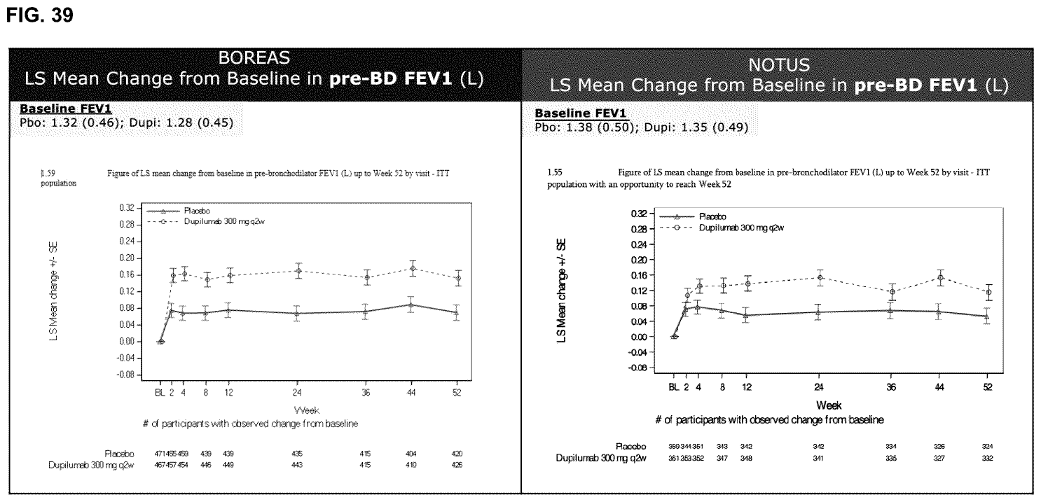

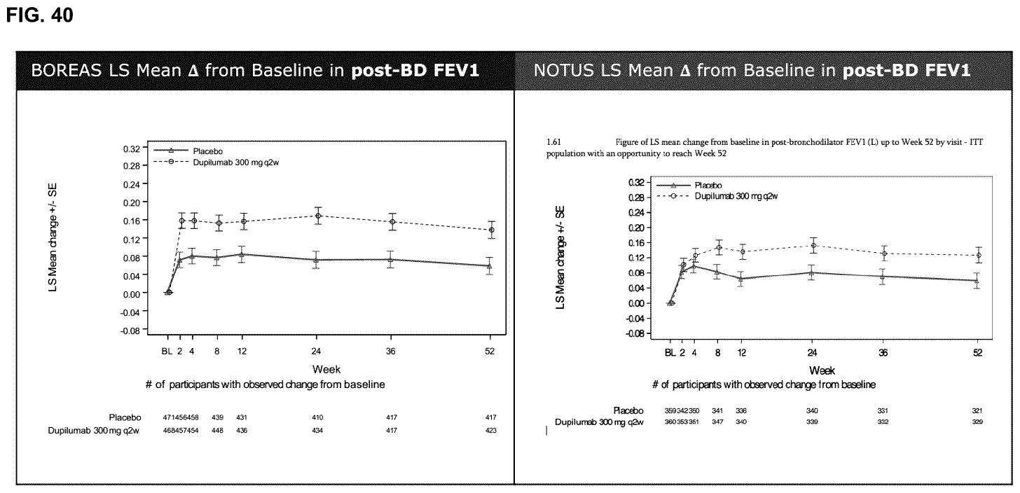

Dupilumab significantly and meaningfully improved ling function as measured by pre-bronchodilator (pre-BD) FEV1 at weeks 12 and 52. (FIGS. 38 and 39). A rapid and sustained improvement was observed in post-BD FEV1. (FIG. 40).

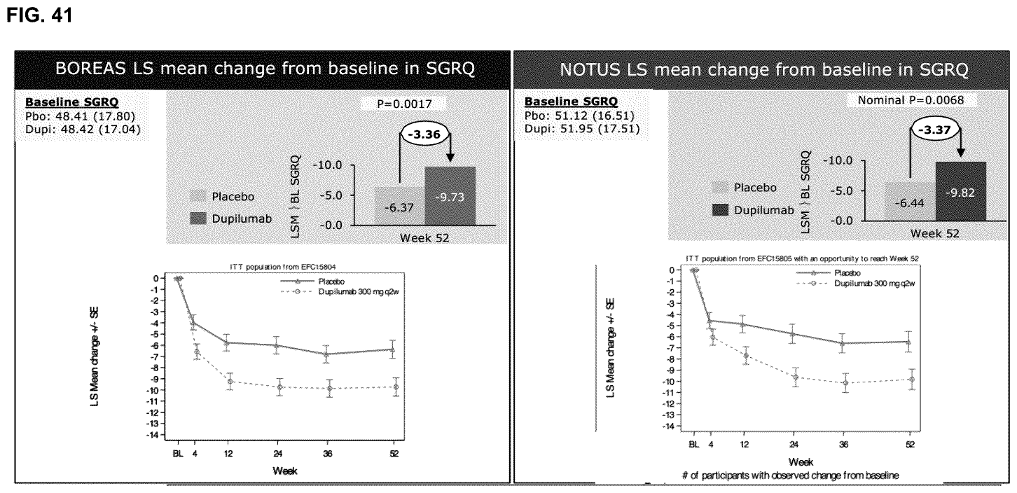

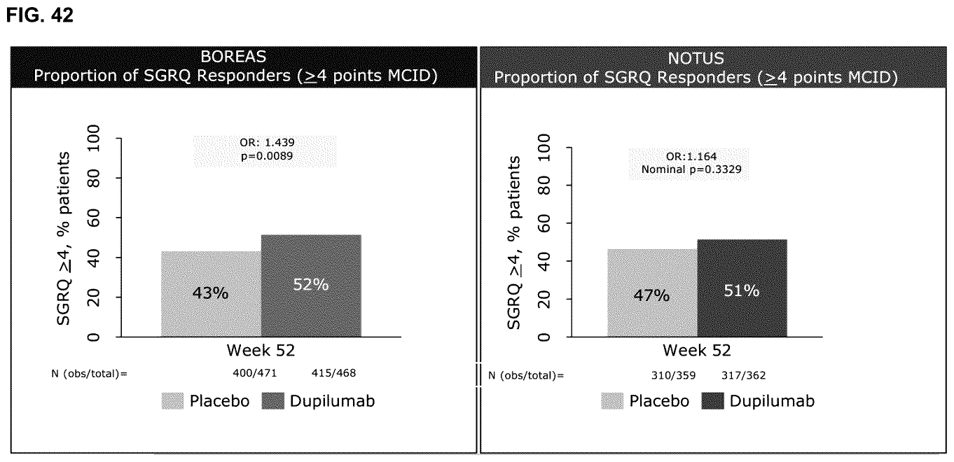

In the NOTUS study, an improvement of St. George's Respiratory Questionnaire (SGRQ) was observed by week 12 and sustained through week 52. (FIG. 41). SGRQ is a measure of overall health, daily life and perceived well-being (health status). SGRQ is a scale of 50 items, rated 0-100, with higher scores indicative of more limitation. MCID: 4 units. The BOREAS study showed a significant improvement in health status as measured by SGRQ. The Dupixent response was consistent in both trials and a differential result was driven by a higher placebo response in the NOTUS study. Dupilumab improved health status, as measured by SGRQ (FIG. 42).

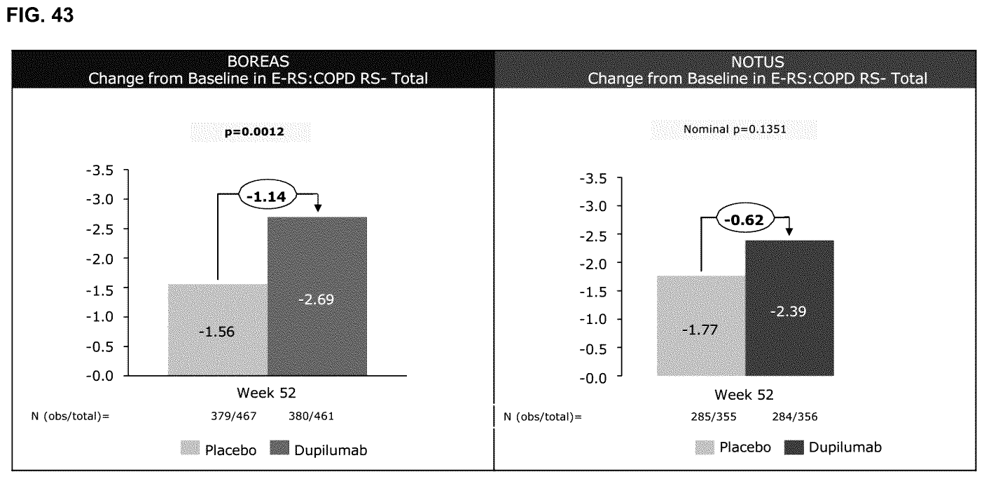

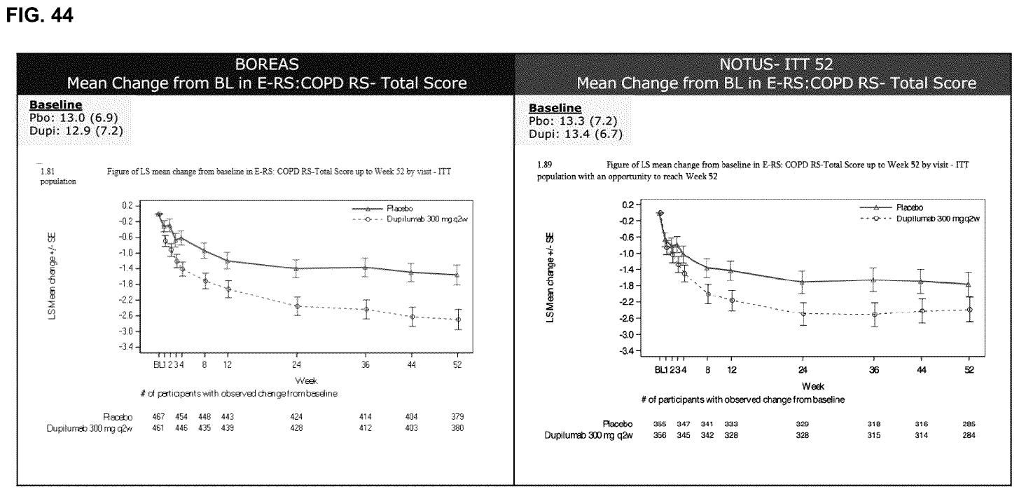

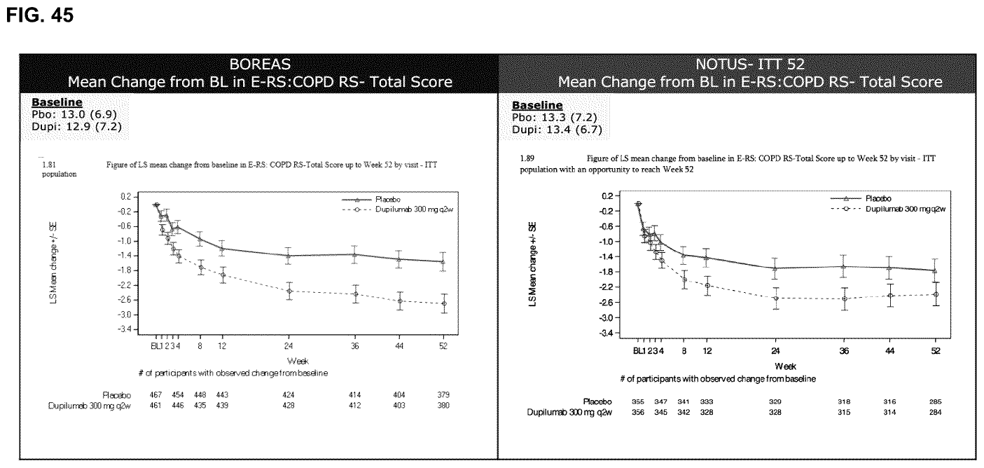

Dupixent improved symptoms of COPD as measured by E-RS COPD: RS-Total Score (E-RS measures the severity of respiratory symptoms in stable COPD: total breathlessness, cough and sputum, and chest symptoms. It has a scale of 0-40, with a lower number being better. Symptomatic improvement definition is −2.0). (FIG. 43). In the BOREAS study, a significant improvement in E-RS: COPD RS-Total Score was rapid and sustained through week 52. In NOTUS, a trend to improvement in symptoms was observed that was not significant at week 52. (FIG. 44).

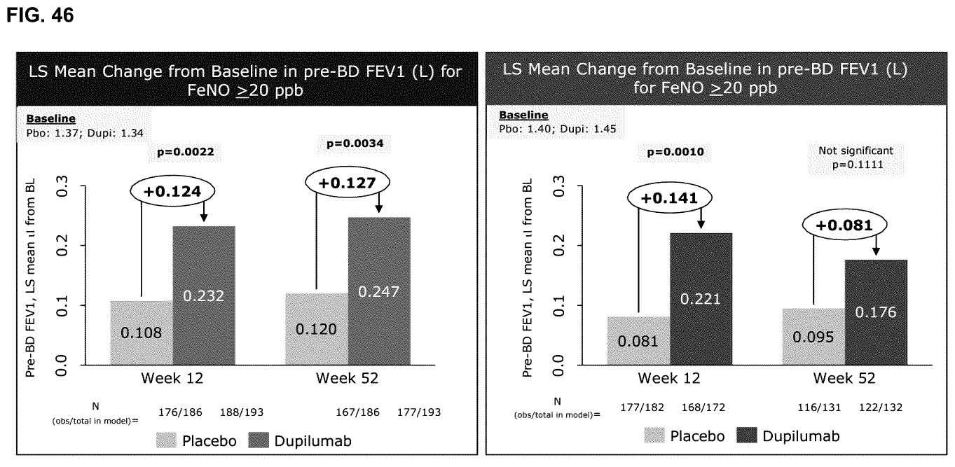

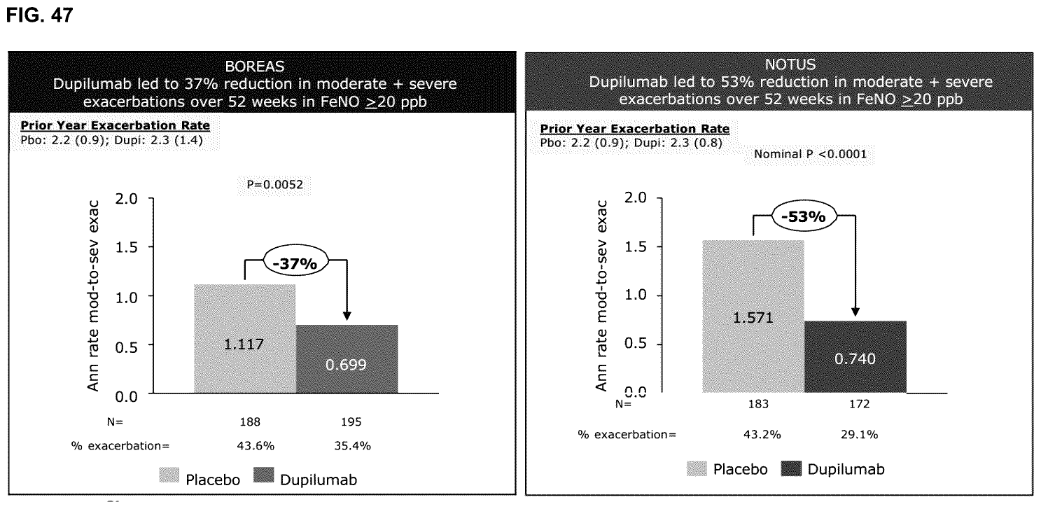

A higher efficacy was observed in the baseline FeNO ≥20 ppb subgroup. (FIG. 46). Lung function improvements increased with high FeNO. In NOTUS, although not significant was observed at week 52, there was a clear trend of improvement. Exacerbation improvements were observed with an increase with markers of type 2 inflammation, with a greater increase observed in the NOTUS study. Dupilumab led to a 37% reduction over 52 weeks in BOREAS and a 53% reduction in NOTUS. (FIG. 47).

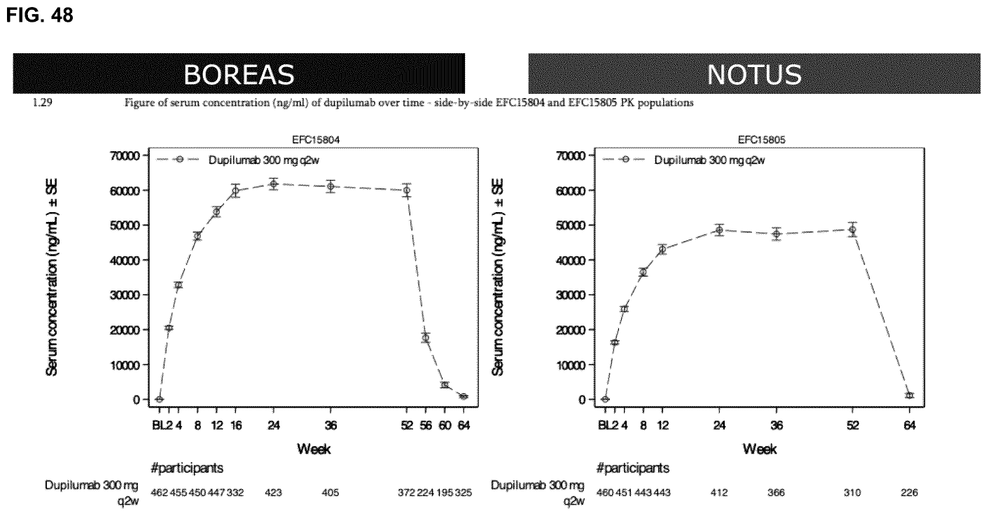

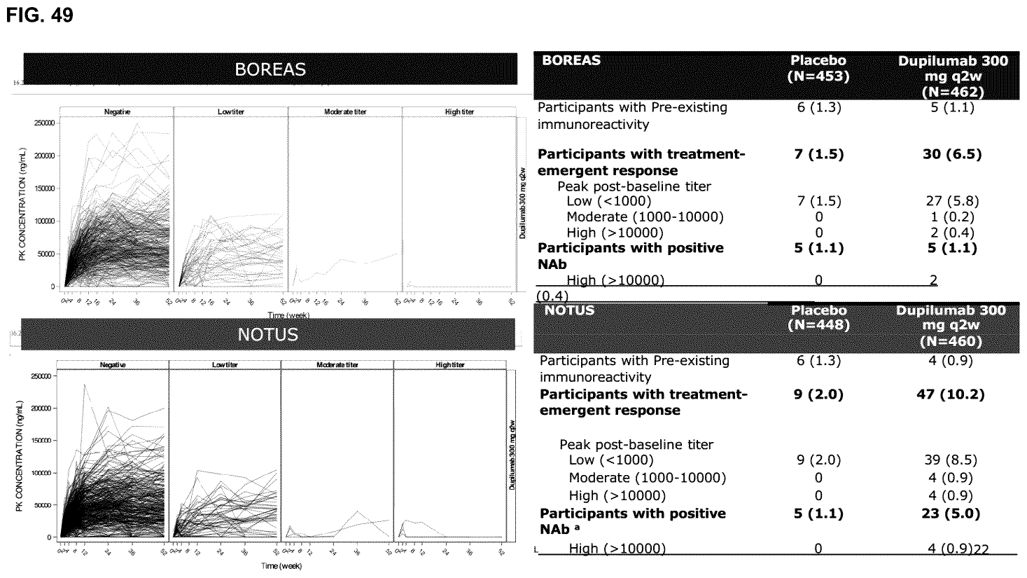

Pharmacokinetics (PK) were higher in BOREAS than NOTUS (week 24 mean was 61.8 mg/L in BOREAS vs. 48.6 mg/L in NOTUS). (FIG. 48). Serum PK over time is illustrated in FIG. 49.

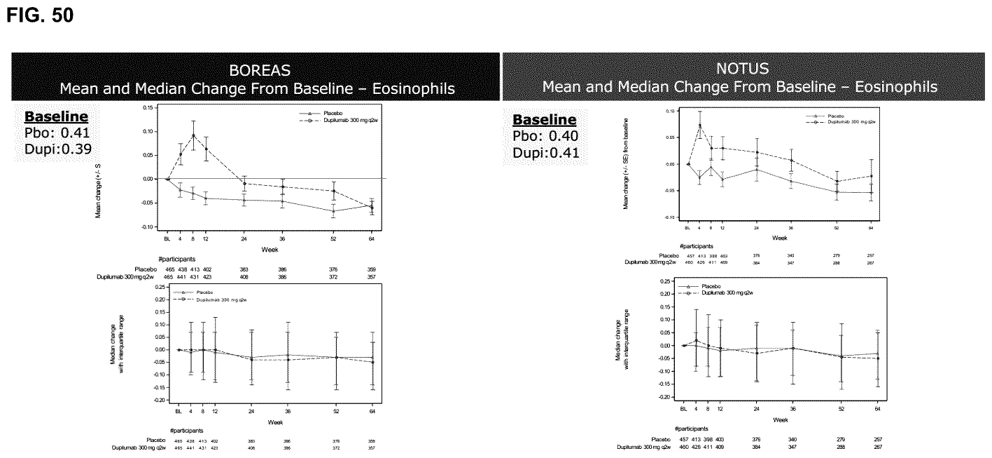

Dupilumab decreased eosinophil levels (after a transient increase) over time. (FIG. 50). No cases of symptomatic eosinophilia, EGPA or eosinophilic pneumonia were observed.

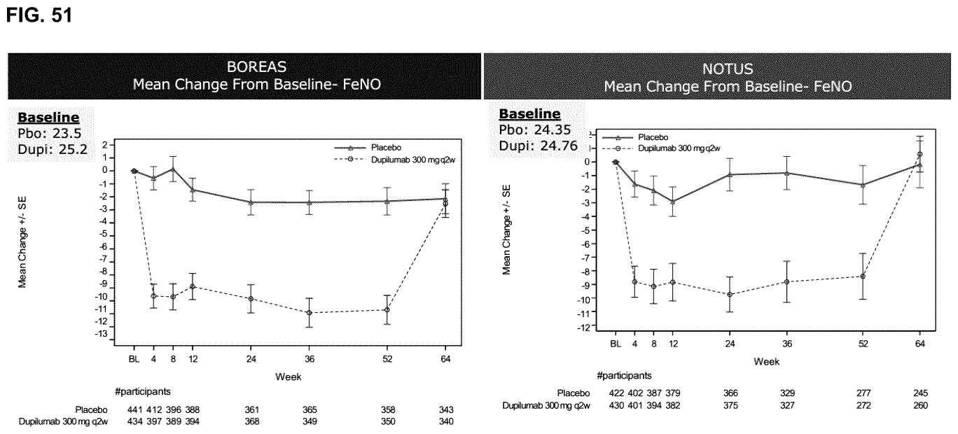

Dupilumab decreased FeNO levels over time. (FIG. 51).

### Conclusions

Dupilumab was well-tolerated and had an acceptable safety profile in the COPD population. No new safety concerns were identified.

Dupilumab significantly decreased moderate-severe exacerbations and improved lung function. Dupilumab's reduction in moderate-to-severe exacerbations of 34% is consistent with the efficacy observed in BOREAS. A delayed time to first moderate-to-severe exacerbations (nominal p=0.0024) and first severe exacerbations (nominal p=0.0203) was observed. A trend toward reduction in severe exacerbations (44% reduction, nominal p=0.0571) was observed.

In the NOTUS study, Dupilumab improved FEV1 by week 12 (+82 mL vs. PBO) and week 52 (+62 mL vs. placebo), similar to the BOREAS study.

Lung function improvements were observed using multiple parameters, including post-BD FEV1.

Consistent benefits were observed irrespective of demographics and disease characteristics for exacerbations and lung function.

Increasing efficacy was observed with higher (FeNO), with moderate-to-severe exacerbation reduction of 53% (nominal p<0.0001).

Improvements were observed for HRQoL and SGRQ (nominal p=0.0068), consistent with the BOREAS study.

Improvements were observed for SGRQ domains (symptoms, activity, impacts) and E-RS:COPD breathlessness.

Dupilumab reduced type 2 biomarkers over time (e.g., blood EOS and FeNO levels), consistent with the BOREAS study.

Dupilumab was well tolerated with an acceptable safety profile in the COPD population.

No new safety concerns were identified in the NOTUS study or in the pooled safety data.

TEAE deaths were comparable with numerically higher deaths in Dupilumab (1.5% pbo vs. 2.6% dupi) in the NOTUS study, with no increased frequency of CV deaths or MACE events in patients receiving Dupilumab. Deaths were balanced in the pooled safety population (1.6% placebo vs. 2.0% Dupilumab).

Injection site reactions were higher with Dupixent vs. placebo in the NOTUS study and the pooled dataset.

Few opportunistic infections, balanced malignancy, one severe conjunctivitis and keratitis were observed in the NOTUS study and the pooled dataset.

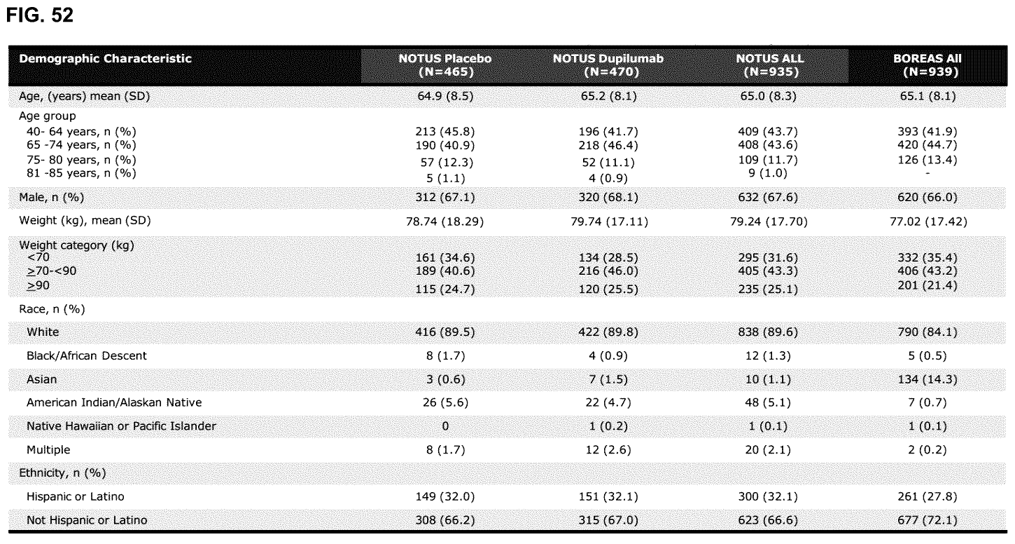

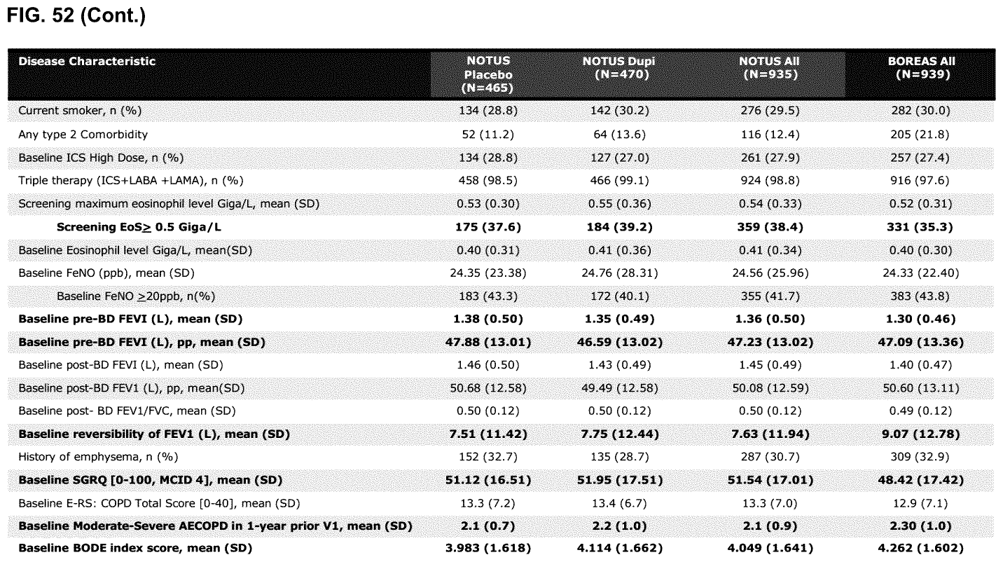

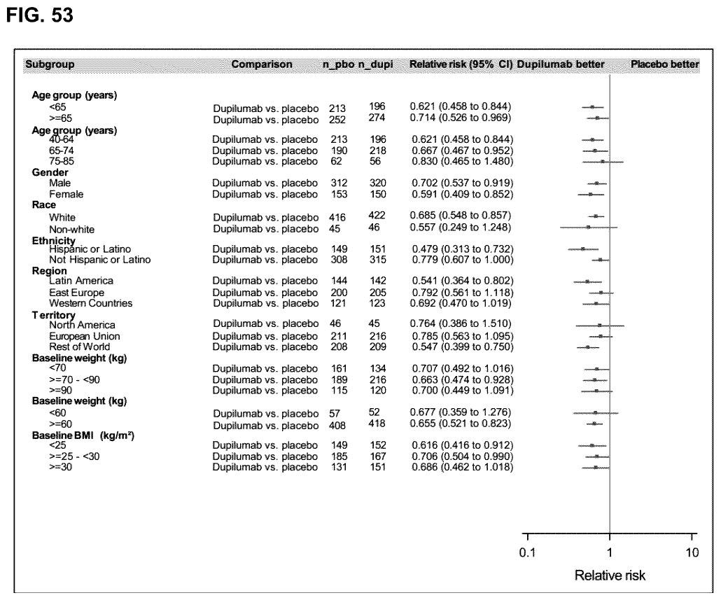

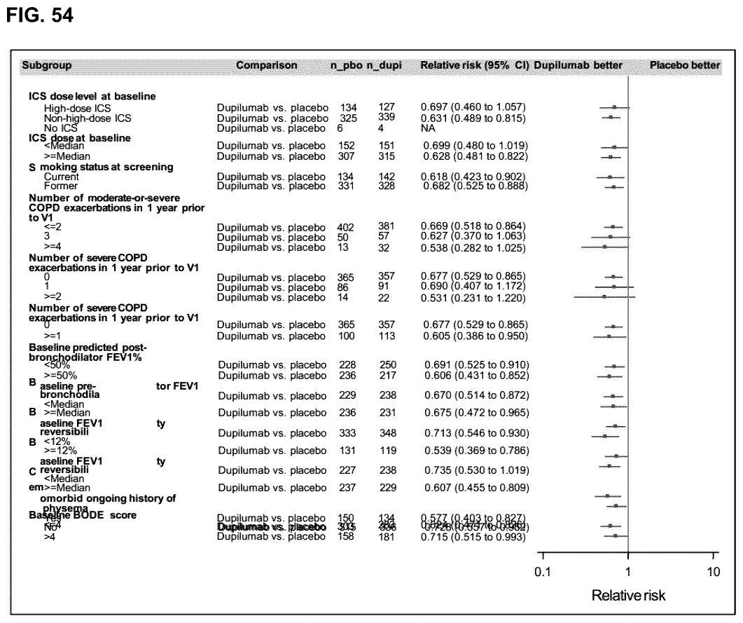

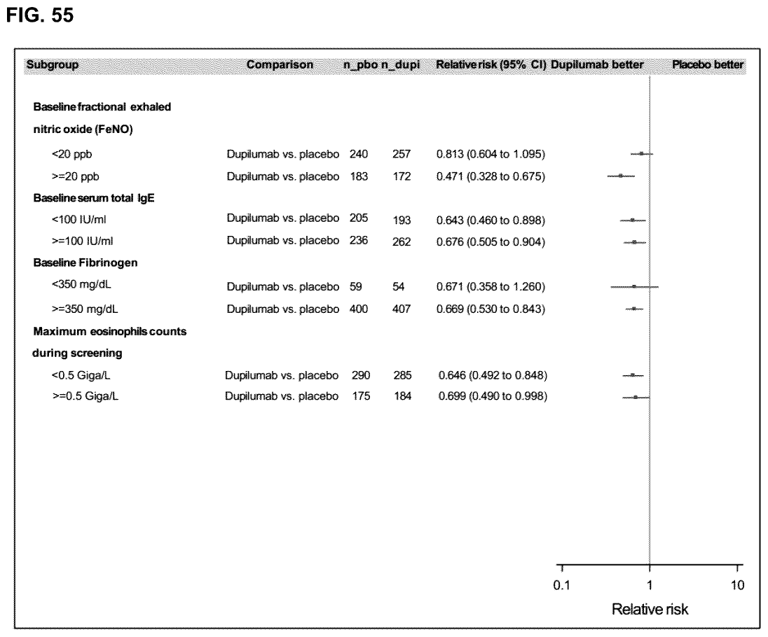

Baseline demographics are set forth at FIG. 52. NOTUS baseline demographic characteristics were similar to those in BOREAS. Efficacy across multiple subgroups based on demographics is depicted at FIG. 53. A consistent reduction was observed in moderate-to-severe exacerbations across all demographic subgroups. Efficacy across multiple subgroups by disease is depicted at FIG. 54. A consistent reduction was observed in moderate-to-severe exacerbations across all disease characteristic subgroups. Efficacy across multiple type 2 biomarker subgroups is depicted at FIG. 55. (P−interaction (FeNO)=0.0265). A higher reduction was observed with higher levels of type 2 biomarkers.

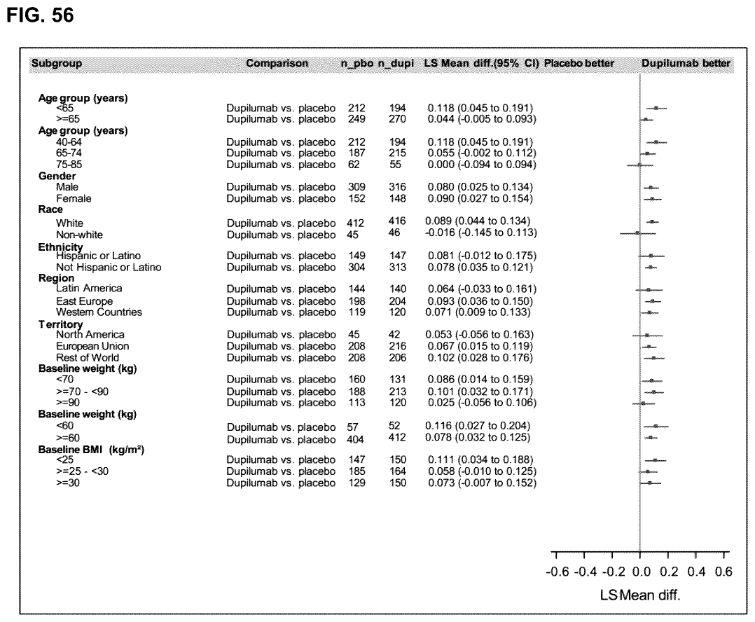

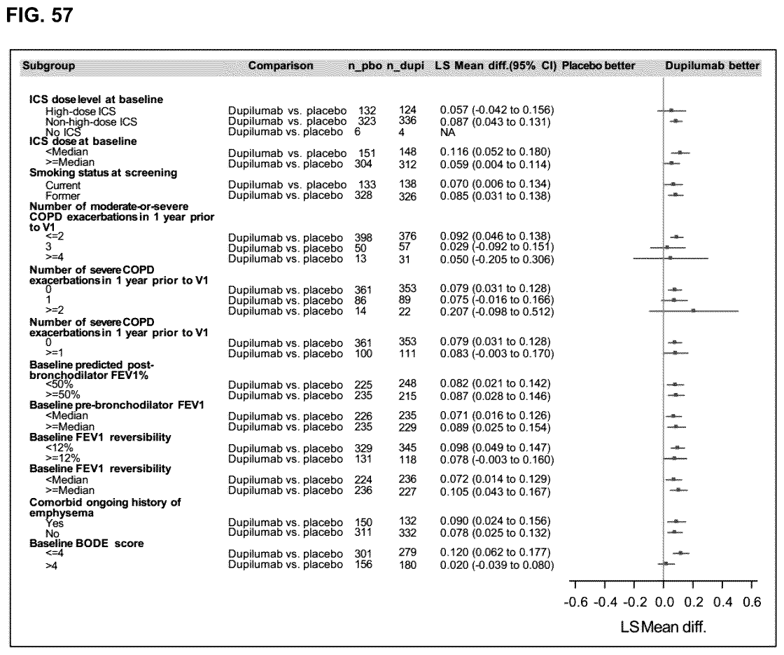

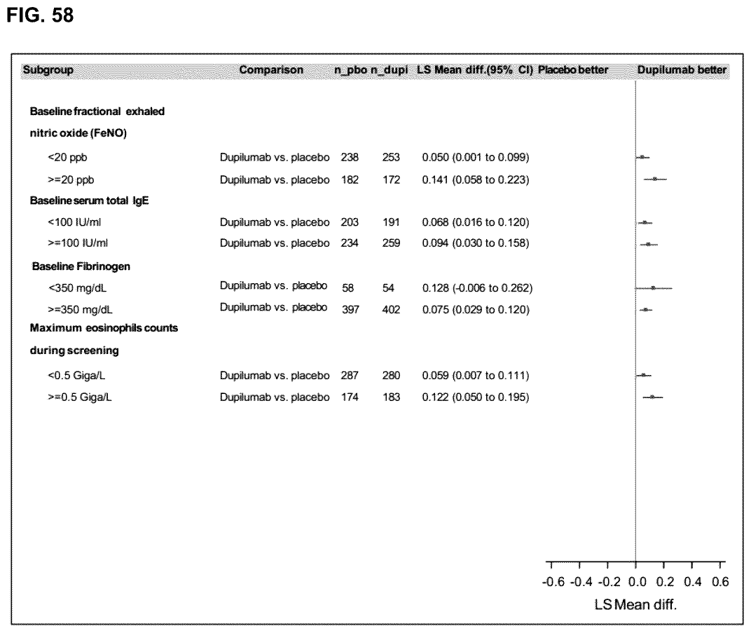

FEV1 levels across subgroups based on demographics is depicted at FIG. 56. Improvement in FEV1 at week 12 was consistent across multiple demographic subgroups. FEV1 levels across subgroups based on disease characteristics is depicted at FIG. 57. Improvement in FEV1 at week 12 was consistent across multiple disease characteristics subgroups. (P−interaction (BODE)=0.0268). FEV1 levels across subgroups based on biomarkers is depicted at FIG. 58. A higher improvement was observed with higher levels of type 2 biomarkers.

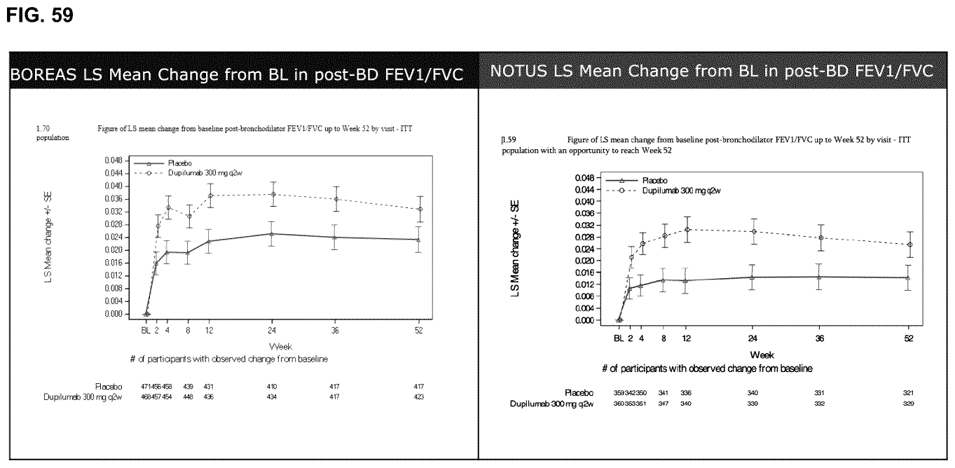

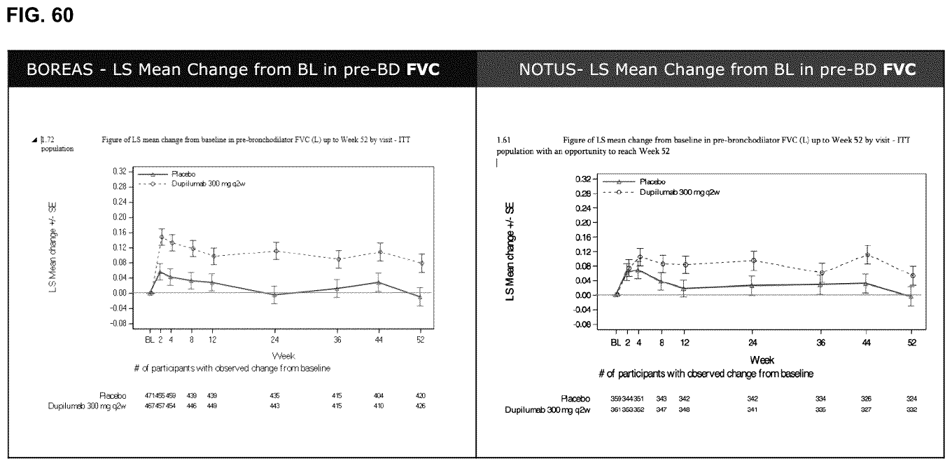

Improvements in lung function were rapid and sustained for post-BD FEV1/FVC and pre-BD FVC. Improvements in lung function were observed for the Dupilumab groups in both studies, although the magnitude was smaller in the NOTUS study (FIG. 59 and FIG. 60).

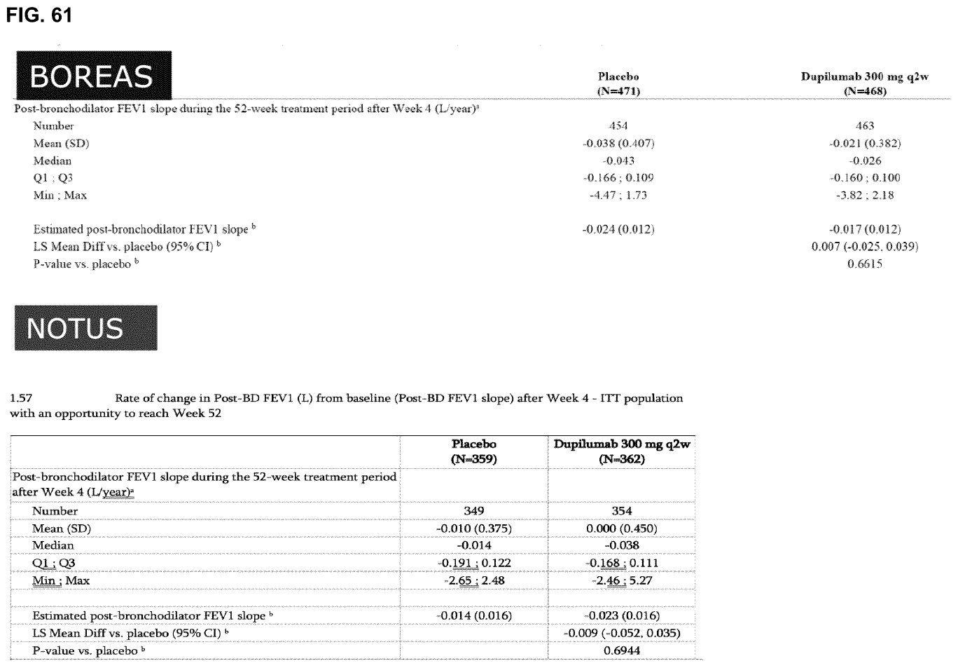

No significant post-BD FEV1 slope was observed for Dupixent vs. placebo (FIG. 61).

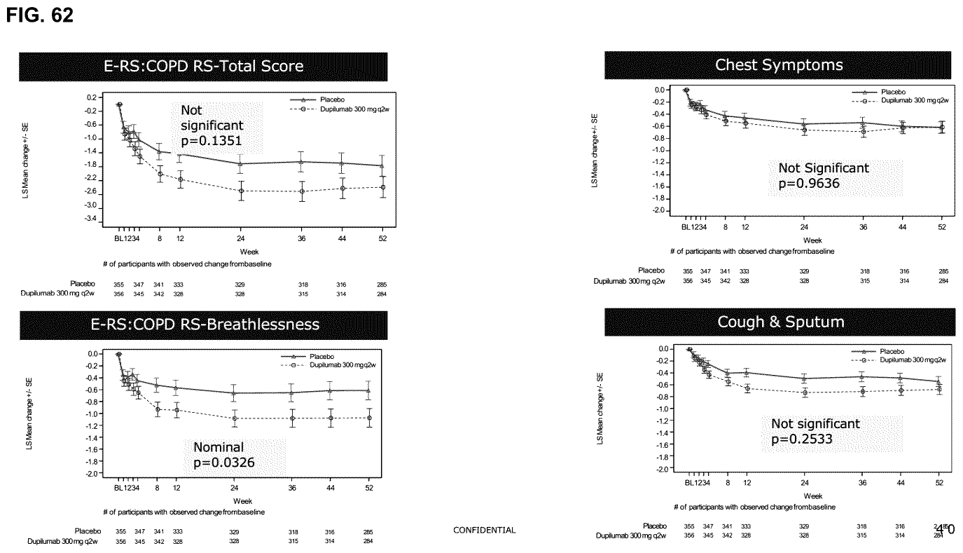

Dupilumab improved breathlessness on E-RS:COPD at week 52 (FIG. 62).

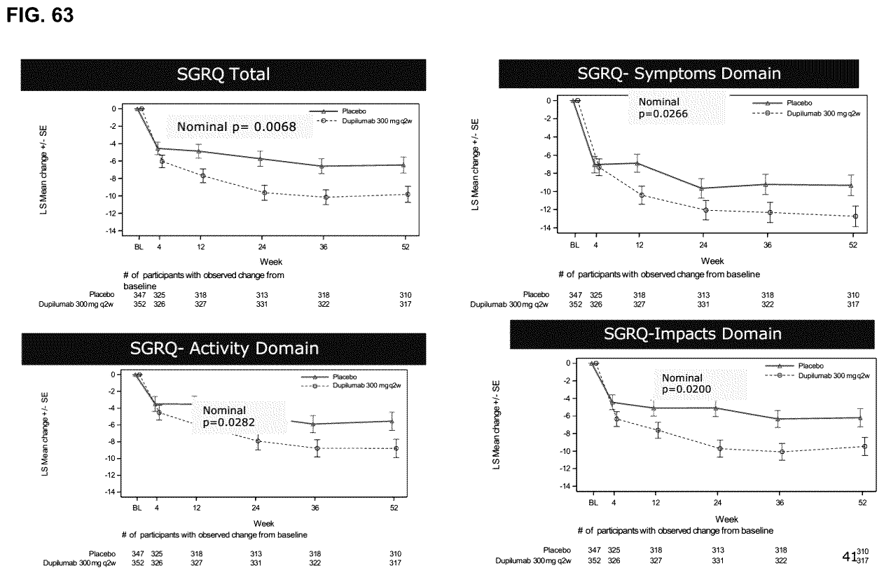

Dupilumab improved all domains of SGRQ at week 52 (FIG. 63).

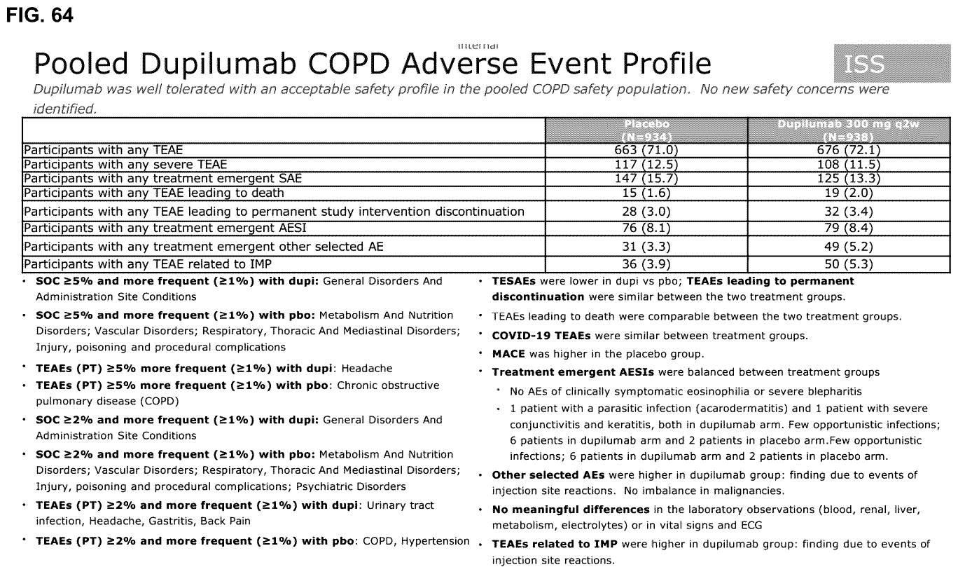

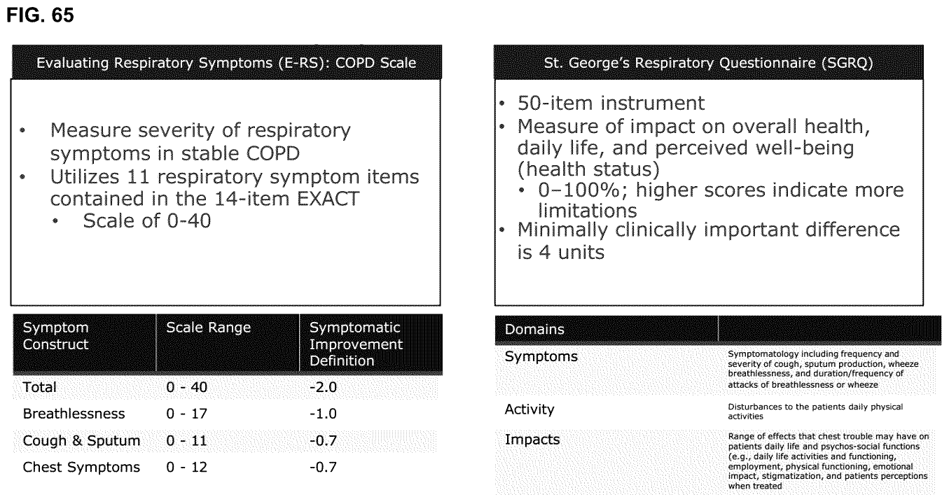

A pooled Dupilumab COPD adverse event profile is set forth at FIG. 64.

### Summary of Endpoint Definitions

Annualized Rate of Moderate-to-severe Exacerbations: Moderate exacerbations are defined as AECOPD that requires systemic steroids and/or antibiotics. Severe exacerbations are defined as AECOPD requiring hospitalization, or observation for >24 hrs. in ED/urgent care or resulting in death.

Pre-BD FEV1 (L) is a change in pre-BD FEV1 (L) from baseline to week 12 or a change in pred-BD FEV1 (L) from baseline to week 52. 100 mL is considered clinically important. Lung function decline in COPD is progressive.

St. George's Respiratory Questionnaire (SGRQ) is a change measured from baseline to week 52. The proportion of patients with SGRQ improvement >4 points at week 52 is measured. SGRQ is a measure of impact of overall health, daily life and perceived well-being (health status) that includes 50 items, on a scale of scale 0-100, with higher scores indicating more limitation. MCID: 4 units.

Evaluation Respiratory Symptoms (E-RS): COPD Scale is a change in E-RS COPD total symptom score from baseline to week 52. E-RS is a measure severity of respiratory symptoms in stable COPD including total, breathlessness, cough & sputum, chest symptoms, on a scale of 0-40.

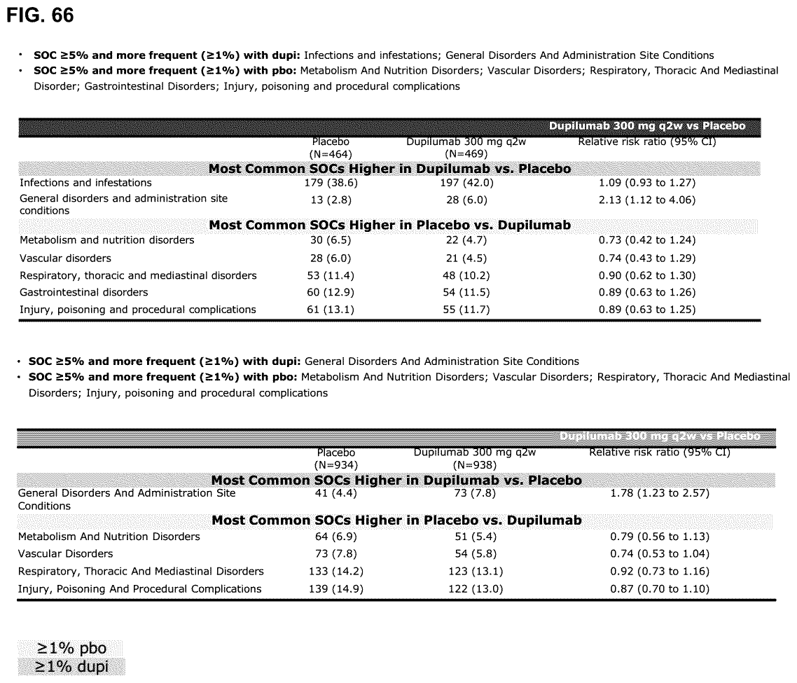

The most common SOCs: ≥5% incidence and ≥1% difference between treatment arms are shown at FIG. 66.

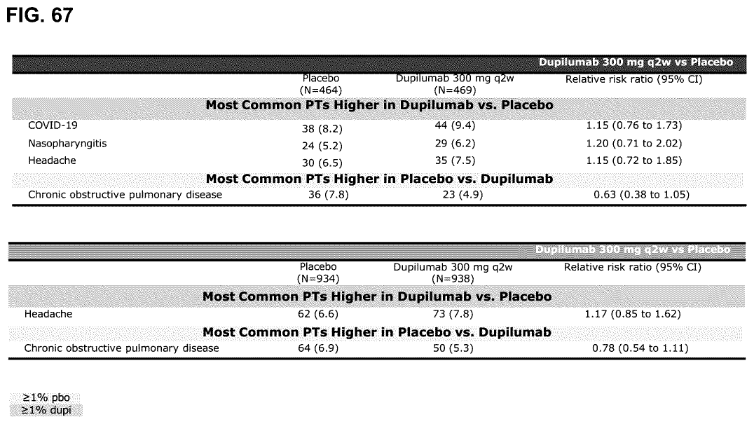

The most common TEAEs ≥5% incidence and ≥1% difference between treatment arms are shown at FIG. 67.

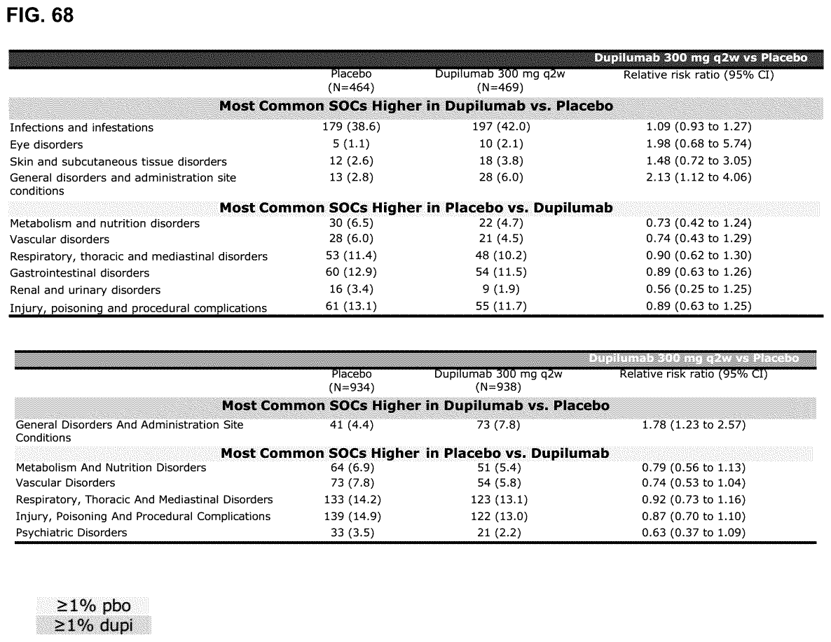

The most common SOCs: ≥2% incidence and ≥1% difference between treatment arms are shown at FIG. 68.

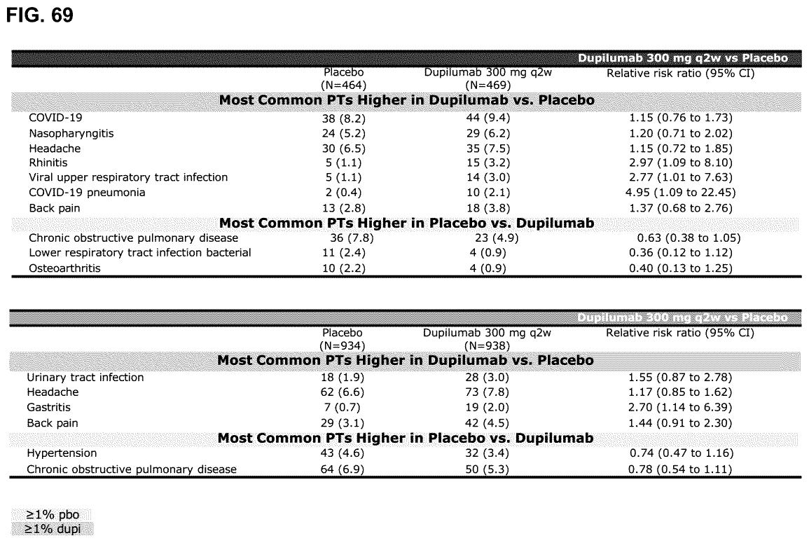

The most common TEAEs ≥2% incidence and ≥1% difference between treatment arms are shown at FIG. 69.

Treatment-emergent serious adverse events (TESAEs) were more frequent in placebo vs. Dupixent. Common SAEs (SOC ≥2% and ≥1% difference) that were higher in placebo vs. Dupilumab were the respiratory, thoracic and mediastinal disorders SOC, while infections and infestations were higher in Dupilumab vs. placebo. The only SOC with ≥1% difference between placebo (7.5%) and Dupixent (5.7%) was the respiratory, thoracic, and mediastinal disorders category. Comparable AESI was observed between placebo vs. Dupixent. Other selected AEs were higher in the Dupixent group due to injection site reactions and conjunctivitis. The percentage of participants with TEAEs classified as cardiovascular was higher in placebo vs. Dupixent. The percentage of participants with TEAEs classified as cardiovascular was higher in placebo vs. Dupixent. No trends were noted across different MACE categories for both placebo and Dupixent. The percentage of participants with TEAEs associated with COVID-19 was higher in the Dupilumab group compared to the placebo group. In the pooled safety population, TEAEs associated with COVID-19 were balanced between the placebo and the Dupixent groups.

With respect to TEAEs leading to death, there were seven in the Placebo (1.5%) group and 12 in the Dupilumab (2.6%) group. In the pooled safety population, TEAEs leading to death were similar between the placebo and the Dupixent groups. TEAEs leading to death were generally consistent with what is anticipated in a population of patients with COPD.

### NOTUS Inclusion Criteria

Adults aged 40-85 years;
Physician diagnosis of COPD for ≥12 months prior to randomization, meeting the following criteria: Current or former smokers with a smoking history of ≥10 pack-years;
Current: active smoking (active smoking includes cigarettes, e-cigarettes, cigars, pipes, etc.);
Former: stopped at least 6 months prior to V1;
Moderate-to-severe COPD (post-BD FEV1/FVC ≤70% and post-BD FEV1% predicted >30% and ≤70%);
MRC Dyspnea Scale grade ≥2;
Patient-reported history of signs and symptoms of chronic bronchitis (chronic productive cough) for three months in the year up to screening;
Documented history of high exacerbation risk (defined as an exacerbation history of ≥2 moderate (requiring SCS and/or antibiotics; one of which must involve use of SCS) or ≥1 severe (requiring hospitalization or >24 hours of observation in an ED/urgent care facility) exacerbations within the year prior to inclusion);
Evidence of type 2 inflammation (blood EOS ≥300 cells/μL at Visit 1).
(AECOPD, acute exacerbation of COPD; BD, bronchodilator; ED, emergency department; EOS, eosinophils; FEV1, forced expiratory volume in 1 second; FVC, forced vital capacity; MRC, Medical Research Council; SCS, systemic corticosteroids.)

### NOTUS Exclusion Criteria

Current diagnosis or history of asthma;
Significant pulmonary disease other than COPD;
Diagnosis of α-1 antitrypsin deficiency;
Other diagnosed pulmonary or systemic disease associated with elevated peripheral EOS;
Cor pulmonale, evidence of right cardiac failure;
Treatment with oxygen of >12 hours/day;
Hypercapnia requiring bi-level ventilation;
AECOPD or respiratory tract infection within 4 weeks prior to screening, or during the screening period;
History of, or planned, pneumonectomy or lung volume reduction surgery Long-term treatment with oxygen >4.0 L/min or if a participant requires more than 2.0 L/min in order to maintain oxygen saturation >88%.

In summary, the interim analysis of the second Dupixent (dupilumab) investigational Phase 3 chronic obstructive pulmonary disease (COPD) trial (NOTUS) confirmed positive published results from the landmark Phase 3 BOREAS trial showing that Dupixent significantly reduced (34%) exacerbations. The trial also confirmed that treatment with Dupixent led to rapid and significant improvements in lung function by 12 weeks that were sustained up to 52 weeks. The NOTUS trial evaluated the investigational use of Dupixent compared to placebo in adults currently on maximal standard-of-care inhaled therapy (triple therapy) with uncontrolled COPD and evidence of type 2 inflammation (i.e., blood eosinophils ≥300 cells/μL). The NOTUS trial included 935 adults who were current or former smokers aged 40 to 85 years and randomized to receive Dupixent (n=470) or placebo (n=465), which was added to maximal standard-of-care inhaled therapy. Patients receiving Dupixent compared to placebo experienced (1) 34% reduction in moderate or severe acute COPD exacerbations over 52 weeks (p=0.0002), the primary endpoint, and (2) improved lung function from baseline by 139 mL at 12 weeks compared to 57 mL for placebo (p=0.0001), with the benefit versus placebo sustained through week 52 (115 mL for Dupixent vs. 54 mL for placebo, p=0.0182), both of which were key secondary endpoints.

The safety results were generally consistent with BOREAS and the known safety profile of Dupixent in its approved indications. Overall rates of adverse events (AE) were 67% for Dupixent and 66% for placebo. AEs more commonly observed with Dupixent (≥5% and ≥1% imbalance) compared to placebo included COVID-19 (9.4% Dupixent, 8.2% placebo), nasopharyngitis (6.2% Dupixent, 5.2% placebo), and headache (7.5% Dupixent, 6.5% placebo). AEs more commonly observed with placebo compared to Dupixent included chronic obstructive pulmonary disease (7.8% placebo, 4.9% Dupixent). AEs leading to deaths were 2.6% for Dupixent and 1.5% for placebo.

### Example 3. Dupilumab for COPD with Type 2 Inflammation Indicated by Eosinophil Counts: NOTUS

### Background

Dupilumab demonstrated efficacy and safety in the phase 3 BOREAS trial (NCT03930732) in patients with chronic obstructive pulmonary disease (COPD) and type 2 inflammation. NOTUS was the second pivotal, phase 3, randomized, double-blind, placebo-controlled trial evaluating the efficacy and safety of dupilumab in patients with COPD with blood eosinophil count ≥300 cells/μl and an elevated exacerbation risk, despite being on inhaled triple therapy.

### Methods

Participants (N=935) were randomized 1:1 to receive subcutaneous dupilumab 300 mg (N=470) or placebo (N=465) every 2 weeks. The primary endpoint was annualized rate of moderate or severe exacerbations. Secondary endpoints included change from baseline in prebronchodilator forced expiratory volume in 1 second (FEV1) at weeks 12 and 52 and St. George's Respiratory Questionnaire (SGRQ) total score at week 52, and safety.

### Results

Dupilumab reduced the annualized rate of moderate or severe exacerbations vs. placebo by 34% (0.86; 95% confidence interval [CI], 0.70 to 1.06 vs 1.30; 95% CI, 1.05 to 1.60; relative risk vs. placebo 0.66 [95% CI, 0.54 to 0.82]; P<0.001). Dupilumab significantly increased prebronchodilator FEV1 vs. placebo at week 12 (least-squares mean difference 82 ml; P<0.001) and week 52 (62 ml, P=0.02). The number of adverse events was similar between groups.

### Conclusions

In patients with COPD and type 2 inflammation, dupilumab reduced exacerbations, improved lung function, and safety was generally consistent with the known safety profile. Dupilumab was effective in patients with COPD and type 2 inflammation, with a reduction in moderate or severe exacerbations, improvements in lung function, HRQoL, and symptoms, and was well-tolerated with safety consistent with the known safety profile.

### Trial Design and Oversight

The NOTUS trial was a phase 3, multicenter, international, double-blind, randomized, placebo-controlled, 52-week trial conducted at 329 sites across 29 countries. Eligible patients were randomized 1:1 to receive add-on subcutaneous dupilumab 300 mg or matched placebo every 2 weeks. Randomization was stratified according to country, baseline ICS dose, and smoking status at screening.

The trial was approved by the appropriate regulatory authorities and ethics committees. All patients provided written informed consent. Oversight of the trial was provided by an external, blinded independent data and safety monitoring committee. The trial protocol and statistical analysis plan are available at NEJM.org.

### Patients

Eligible patients were aged 40 to 85 years, had physician-diagnosed COPD for at least 12 months, and were on background triple inhaler therapy (ICS+LAMA+LABA or LAMA+LABA alone if ICS was contraindicated) for ≥3 months with a stable dose for at least 1 month. In the year before screening, patients must have experienced at least two moderate or one severe exacerbation, at least one moderate exacerbation must have required use of systemic steroids, and at least one exacerbation had to occur while the patient was on background triple therapy.

Eligible patients were either current (capped at 30% of total enrollment) or former (those who had stopped smoking ≥6 months prior to screening) smokers, with a smoking history of at least 10 pack-years. At screening, patients were required to have a postbronchodilator ratio of forced expiratory volume in 1 s (FEV₁) to forced vital capacity (FVC) of less than 0.70 and postbronchodilator FEV₁ of >30-70% of the predicted normal value. Patients had symptomatic COPD, as indicated by a dyspnea score of ≥2 on the Medical Research Council dyspnea scale, and reported symptoms of chronic bronchitis (chronic productive cough) for 3 months in the year before screening, in the absence of other known causes of chronic cough. Patients with symptomatic COPD are at an increased risk of exacerbations and encompass both the chronic bronchitis and emphysema phenotypes.

Patients had an absolute blood eosinophil count ≥300 cells/μl during screening. Importantly, a current diagnosis or a history of asthma was an exclusion criterion.

### Endpoints

The primary endpoint was the annualized rate of moderate or severe COPD exacerbations during the 52-week trial period. A moderate exacerbation was defined as an exacerbation requiring systemic glucocorticoid and/or antibiotic treatment. A severe exacerbation was defined as an exacerbation leading to hospitalization or emergency/urgent medical care visit with observation >24 hours or resulting in death.

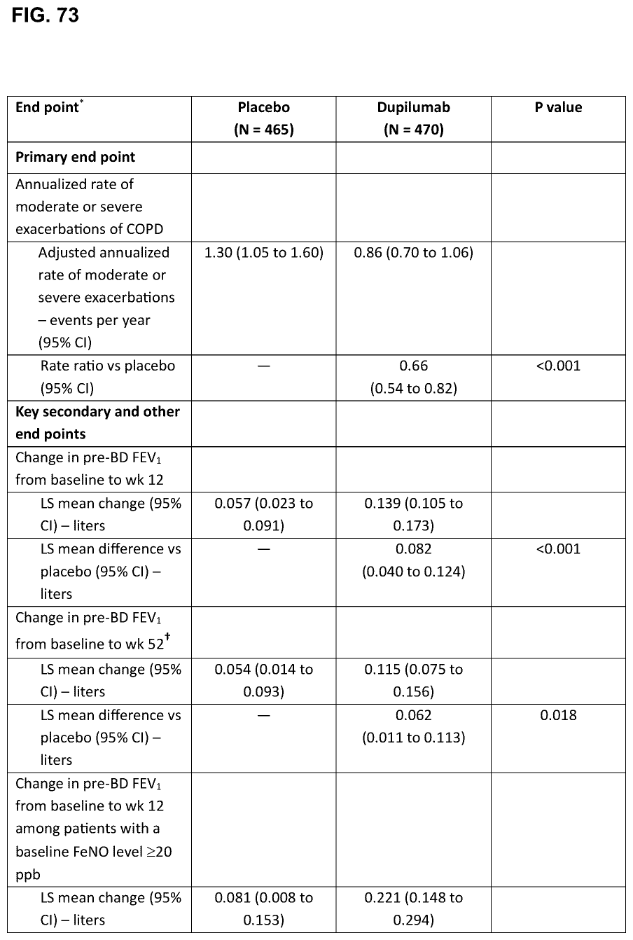

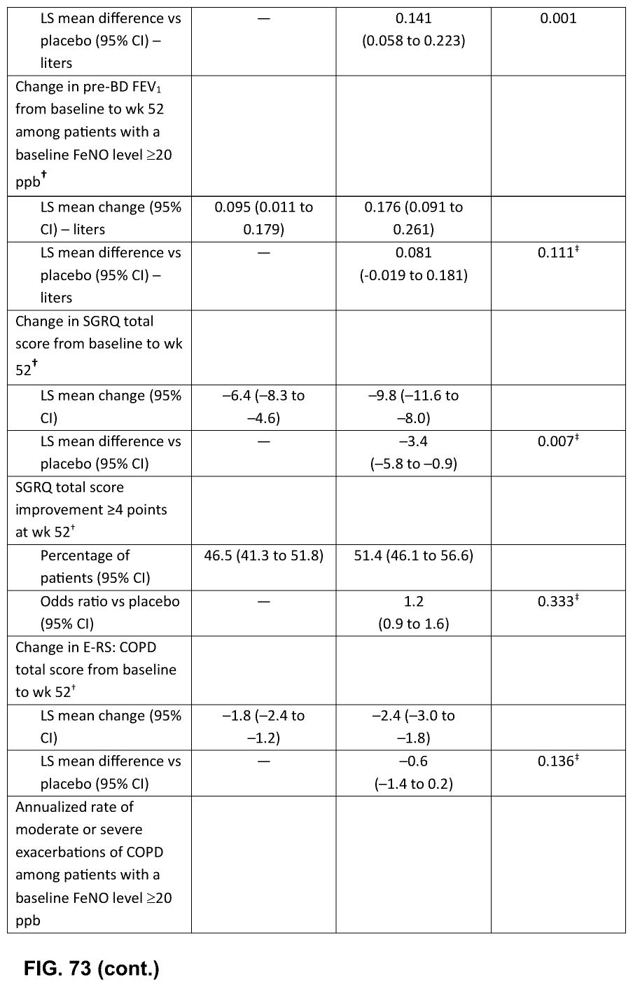

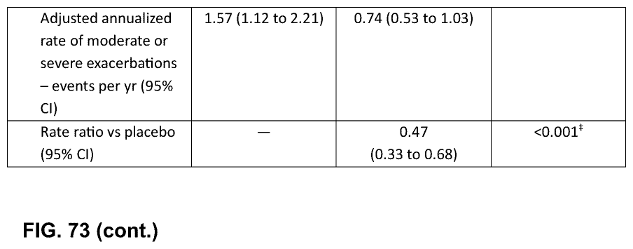

Secondary and other endpoints that were adjusted for multiplicity in order of hierarchical testing included change from baseline in prebronchodilator FEV₁ at weeks 12 and 52, change from baseline in prebronchodilator FEV₁ at weeks 12 and 52 in patients with a baseline fractional exhaled nitric oxide (FeNO) level of ≥20 parts per billion (ppb), change from baseline in St. George's Respiratory Questionnaire (SGRQ) total score at week 52 (score ranges from 0 to 100, with lower scores indicating a better quality of life; minimum clinically important difference [MCID] 4 points), proportion of patients with a change of ≥4 points in SGRQ total score at week 52, change from baseline in Evaluating Respiratory Symptoms in COPD (E-RS: COPD) total score at week 52 (score ranges from 0 to 40, with lower scores indicating less severe respiratory symptoms), and annualized rate of moderate or severe COPD exacerbations in patients with baseline FeNO levels of ≥20 ppb. The hierarchical testing procedure broke atprebronchodilator FEV₁ at week 52 in the subgroup of patients with baseline FeNO levels of ≥20 ppb; therefore, P values for end points below this are considered nominal (FIG. 73).

Key safety end points were adverse and serious adverse events that occurred after initiation of dupilumab or placebo.

### Statistical Analysis

At the time of primary analysis, all patients were enrolled and a 0.92 information fraction was available for the primary endpoint. Efficacy for primary and week 12 end points was evaluated in the intention-to-treat (ITT) population. The week 52 efficacy end points (continuous and proportion type) were analyzed in the ITT population in those patients who had an opportunity to reach week 52 (first 721 randomized patients) at the time of the primary analysis. Safety was evaluated in the safety population, i.e., all patients who received at least one full or partial dose of dupilumab or placebo.

A sample size of 924 (462 per group) provided 90% power to detect a between group difference in the annualized rate of moderate or severe exacerbations of 25% at week 52 at a two-sided alpha of 0.05.

A negative binomial regression model was used to analyze the primary endpoint, with total number of events occurring during the 52-week trial period as the response variable, and trial group, region, baseline ICS dose, smoking status, baseline disease severity, and number of moderate or severe COPD exacerbations in the previous year as covariates. The natural log of treatment duration was an offset variable. Key secondary end points were evaluated using a mixed-effect model with repeated measures, with trial group, region, baseline ICS dose, smoking status, visit, trial group-by-visit interaction, baseline value, baseline value-by-visit interaction, and other model-specific factors as covariates. When the primary end point met statistical significance, end points were evaluated using a hierarchical testing procedure.

### Results

### Patients

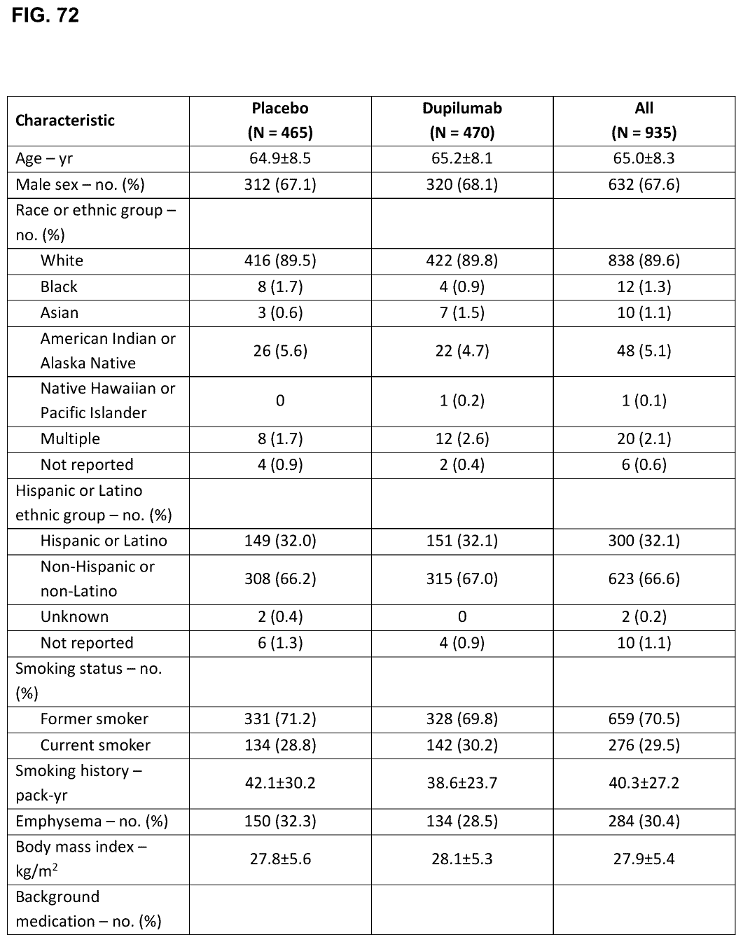

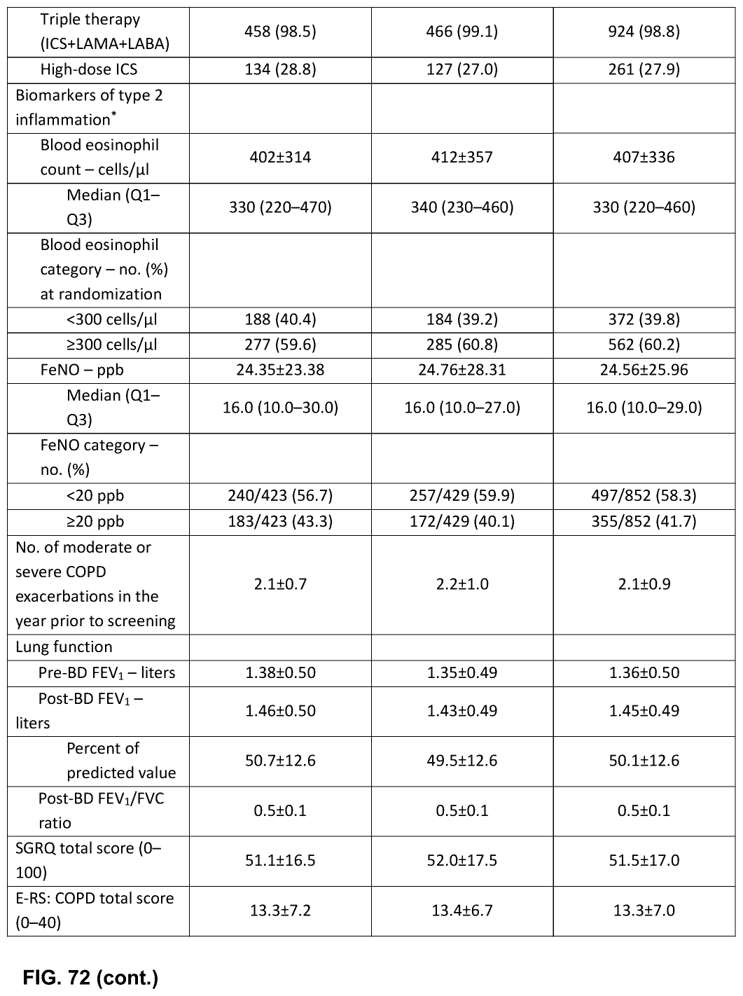

935 patients were randomized: dupilumab (N=470), placebo (N=465). Baseline demographics and clinical characteristics were similar between groups (FIG. 72). The mean (±SD) age of participants was 65.0±8.3 years and 29.5% were current smokers. Nearly all (98.8%) patients were on ICS+LAMA+LABA. The mean number of moderate or severe exacerbations in the year prior to screening was 2.1±0.9. The mean baseline prebronchodilator and postbronchodilator FEV₁ percent predicted values were 47.2±13.0 and 50.1±12.6, respectively. The mean baseline blood eosinophil count was 407±336 cells/μl and 39.8% of patients had a blood eosinophil count <300 cells/μl at randomization. The mean baseline FeNO level was 24.56±25.96 ppb and 41.7% of patients had a baseline FeNO level ≥20 ppb (FIG. 72).

### Primary Endpoint

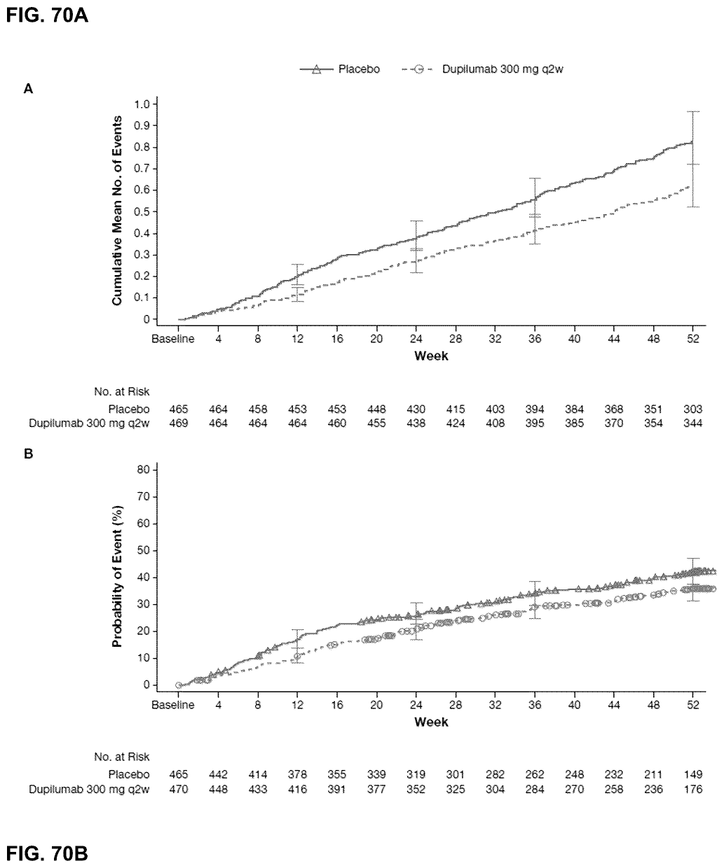

During the 52-week trial period, the annualized rate of moderate or severe COPD exacerbations was lower with dupilumab (0.86; 95% confidence interval (CI), 0.70 to 1.06) compared with placebo (1.30; 95% CI, 1.05 to 1.60), resulting in a 34% relative risk reduction (0.66; 95% CI, 0.54 to 0.82; P<0.001) (FIG. 70, FIG. 73). Similar results were observed in pre-specified subgroups, and in pattern mixture model-multiple imputation (PMM-MI) and tipping point sensitivity analyses. In the pre-specified subgroup with baseline FeNO levels ≥20 ppb, the annualized rate of moderate or severe exacerbations was lower among patients in the dupilumab group than in the placebo group, resulting in a 53% relative risk reduction (0.47; 95% CI, 0.33 to 0.68; P>0.001).

### Secondary and Other Endpoints

### Lung Function

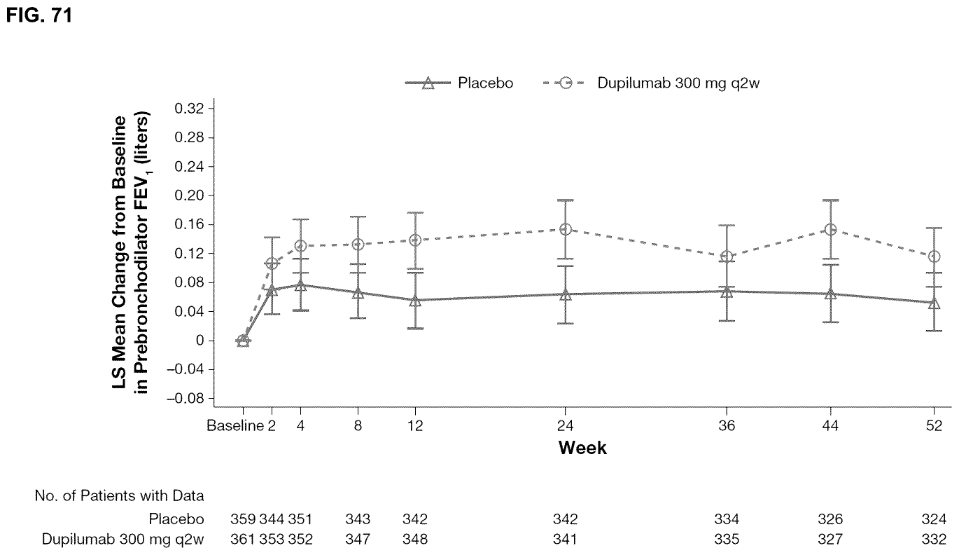

Changes from baseline in prebronchodilator FEV₁ during the 52-week trial period are shown in FIG. 71. At week 12, the least-squares (LS) mean change in prebronchodilator FEV₁ was 139 mL (95% CI, 105 to 173) in the dupilumab group vs. 57 mL (95% CI, 23 to 91) in the placebo group (LS mean difference, 82 ml; 95% CI, 40 to 124; P<0.001). This improvement was maintained at week 52 (LS mean difference, 62 ml 95% CI, 11 to 113; P=0.02) (FIG. 71, FIG. 73). Results were similar in pre-specified subgroups and sensitivity analyses.

In pre-specified analyses of patients with baseline FeNO levels ≥20 ppb, the LS mean change from baseline in prebronchodilator FEV₁ at week 12 was 221 mL (95% CI, 148 to 294) and 81 mL (95% CI, 8 to 153) for dupilumab and placebo, respectively (LS mean difference, 141 ml; 95% CI, 58 to 223; P=0.001), and at week 52 was 176 mL (95% CI, 91 to 261) and 95 mL (95% CI, 11 to 179) for dupilumab and placebo, respectively (LS mean difference, 81 ml; 95% CI, −19 to 181). The hierarchical testing procedure broke at this endpoint (FIG. 73).

### Patient-Reported Outcomes

At week 52, the change from baseline in SGRQ total score was −9.8 (95% CI, −11.6 to −8.0) for dupilumab and −6.4 (95% CI, −8.3 to −4.6) for placebo (LS mean difference, −3.4; 95% CI, −5.8 to −0.9) (FIG. 73). At week 52, improvement in the SGRQ total score ≥4 points (the MCID) occurred in 51.4% of patients treated with dupilumab and 46.5% of patients in the placebo group (odds ratio, 1.16; 95% CI, 0.86 to 1.58) (FIG. 73).

Change from baseline in the E-RS: COPD total score at week 52 was −2.4 (95% CI, −3.0 to −1.8) and −1.8 (95% CI, −2.4 to −1.2) in the dupilumab and placebo groups, respectively (LS mean difference, −0.6; 95% CI, −1.4 to 0.2 (FIG. 73).

### Additional Endpoints

The time to the first moderate or severe exacerbation for dupilumab vs placebo is shown in FIG. 70B (hazard ratio, 0.71; 95% CI, 0.57 to 0.89) and time to first severe exacerbation in (hazard ratio, 0.51; 95% CI, 0.29 to 0.90). The severe exacerbation rate was 0.07 (95% CI, 0.04 to 0.12) and 0.12 (95% CI, 0.07 to 0.22) in the dupilumab and placebo groups, respectively (relative risk reduction vs placebo, 0.56; 95% CI, 0.31 to 1.02).

### Safety

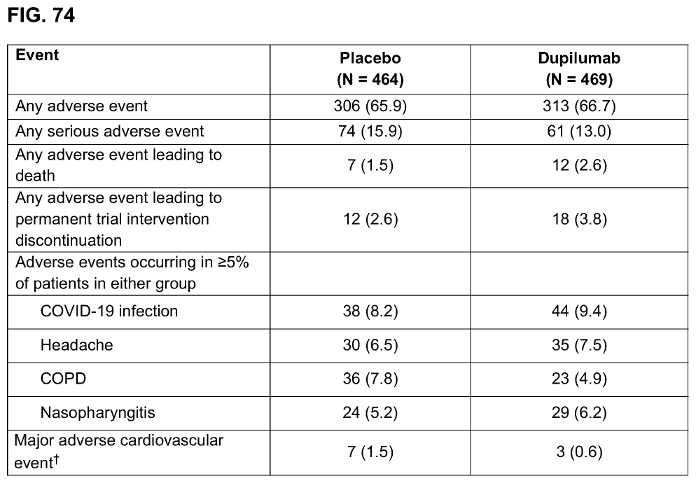

The proportion of patients experiencing adverse events during the trial was similar between dupilumab (66.7%) and placebo (65.9%) (FIG. 74). The most common adverse events were COVID-19 infection, nasopharyngitis, headache, and COPD. Serious adverse events were reported in 13.0% and 15.9% of patients in the dupilumab and placebo groups, respectively. Adverse events that resulted in death occurred in 2.6% and 1.5% of patients in the dupilumab and placebo groups, respectively. Deaths adjudicated as cardiovascular deaths occurred in one patient in each group. Adverse events that were classified as major adverse cardiovascular events occurred in 0.6% and 1.5% of patients in the dupilumab and placebo groups, respectively. Persistent anti-drug antibody responses were observed in 14 (3.0%) patients in the dupilumab group and 3 (0.7%) patients in the placebo group.

### Discussion

NOTUS demonstrated that dupilumab, when added to inhaled triple therapy, reduced the rate of moderate or severe exacerbations and improved lung function in patients with COPD and type 2 inflammation. These results further confirm the role of type 2 inflammation in the pathobiology of a subset of patients with COPD and the role of dupilumab in treating this distinct COPD endotype.

This pivotal trial, combined with BOREAS, confirms the importance of IL-4 and IL-13 in driving the inflammatory process in a subset of patients with COPD. While blood eosinophils serve as practical and accessible markers of type 2 inflammation, they do not necessarily reflect pathobiology solely driven by blood eosinophils. Previous trials of biologics that specifically target blood eosinophils (e.g., anti-IL-5) demonstrated inconsistent reduction of exacerbations and lacked evidence of improvement in lung function or quality of life. In BOREAS and NOTUS, robust and consistent clinical efficacy of IL-4 and IL-13 blockade was observed in patients with COPD with evidence of type 2 inflammation (guided by blood eosinophil count ≥300 cells/μl during screening). A greater magnitude of response was observed among patients with additional elevated markers of type 2 inflammation (e.g., elevated FeNO level). Collectively, these findings highlight the importance of targeting IL-4 and IL-13 as drivers of type 2 inflammation in COPD.

In NOTUS, dupilumab was efficacious across multiple subgroups of demographic and disease characteristics. Notably, similar reductions in the frequency of moderate or severe exacerbations were observed in all pre-specified subgroups, including age, sex, smoking status, baseline lung function, history of exacerbations, and presence of emphysema.

The strengths of the NOTUS trial include that it was an adequately powered, large, multi-national trial in a patient population without other significant pulmonary disease (notably asthma). The 34% relative reduction in moderate or severe exacerbations vs. placebo observed in this trial was clinically significant and numerically greater than that observed for inhaled treatment components.

This second pivotal trial confirmed that add-on dupilumab treatment significantly reduced exacerbations and increased lung function in patients with COPD with type 2 inflammation, demonstrating that this form of targeted therapy benefited patients with this specific endotype.

### REFERENCES

1. Adeloye D, Song P, Zhu Y, et al. Global, regional, and national prevalence of, and risk factors for, chronic obstructive pulmonary disease (COPD) in 2019: a systematic review and modelling analysis. Lancet Respir Med 2022; 10(5):447-458.
2. Kerkhof M, Voorham J, Dorinsky P, et al. Association between COPD exacerbations and lung function decline during maintenance therapy. Thorax 2020; 75(9):744-753.
3. Dransfield M T, Kunisaki K M, Strand M J, et al. Acute exacerbations and lung function loss in smokers with and without chronic obstructive pulmonary disease. Am J Respir Crit Care Med 2017; 195(3):324-330.
4. Whittaker H, Rubino A, Müllerová H, et al. Frequency and severity of exacerbations of COPD associated with future risk of exacerbations and mortality: a UK routine health care data study. Int J Chron Obstruct Pulmon Dis 2022; 17:427-437.
5. Halpin D M, Decramer M, Celli B, Kesten S, Liu D, Tashkin D P. Exacerbation frequency and course of COPD. Int J Chron Obstruct Pulmon Dis 2012; 7:653-661.
6. Halpin D M, Dransfield M T, Han M K, et al. The effect of exacerbation history on outcomes in the IMPACT trial. Eur Respir J 2020; 55(5):1901921.
7. Bhatt S P, Rabe K F, Hanania N A, et al. Dupilumab for COPD with type 2 inflammation indicated by eosinophil counts. N Engl J Med 2023; 389(3):205-214.
8. Bafadhel M, McKenna S, Terry S, et al. Acute exacerbations of chronic obstructive pulmonary disease: identification of biologic clusters and their biomarkers. Am J Respir Crit Care Med 2011; 184(6):662-671.
9. Singh D, Agusti A, Martinez F J, et al. Blood eosinophils and chronic obstructive pulmonary disease: a Global Initiative for Chronic Obstructive Lung Disease Science Committee 2022 review. Am J Respir Crit Care Med 2022; 206:17-24.
10. Brightling C E. Biomarkers that predict and guide therapy for exacerbations of chronic obstructive pulmonary disease. Ann Am Thorac Soc 2013; 10 Suppl:S214-9.
11. Yun J H, Lamb A, Chase R, et al. Blood eosinophil count thresholds and exacerbations in patients with chronic obstructive pulmonary disease. J Allergy Clin Immunol 2018; 141(6):2037-47.e10.
12. Rabe K F, Rennard S, Martinez F J, et al. Targeting type 2 inflammation and epithelial alarmins in chronic obstructive pulmonary disease: a biologics outlook. Am J Respir Crit Care Med 2023; 208(4):395-405.
13. Higham A, Beech A, Wolosianka S, et al. Type 2 inflammation in eosinophilic chronic obstructive pulmonary disease. Allergy 2021; 76(6):1861-1864.
14. Fieldes M, Bourguignon C, Assou S, et al. Targeted therapy in eosinophilic chronic obstructive pulmonary disease. ERJ Open Res 2021; 7(2):00437-2020.
15. Macdonald L E, Karow M, Stevens S, et al. Precise and in situ genetic humanization of 6 Mb of mouse immunoglobulin genes. Proc Natl Acad Sci USA 2014; 111(14):5147-5152.
16. Murphy A J, Macdonald L E, Stevens S, et al. Mice with megabase humanization of their immunoglobulin genes generate antibodies as efficiently as normal mice. Proc Natl Acad Sci USA 2014; 111(14):5153-5158.
17. US Food and Drug Administration. DUPIXENT® (dupilumab). Highlights of Prescribing Information. Feb. 4, 2024. (https://www.regeneron.com/sites/default/files/Dupixent_FPI.pdf)
18. Regeneron. Japan First in the World to Approve Dupixent (Dupilumab) for Chronic Spontaneous Urticaria (CSU). Feb. 16, 2024. (https://investor.regeneron.com/news-releases/news-release-details/japan-first-world-approve-dupixentr-dupilumab-chronic)
19. Kim V, Han K M, Vance G B, et al. The chronic bronchitic phenotype of COPD: an analysis of the COPD Gene Study. Chest 2011; 140(3):626-633.
20. Kim V, Zhao H, Boriek A M, et al. Persistent and newly developed chronic bronchitis are associated with worse outcomes in chronic obstructive pulmonary disease.20. Jones P W. St. George's Respiratory Questionnaire: MCID. COPD 2005; 2:75-79.
21. Pavord I D, Chanez P, Criner G J, et al. Mepolizumab for eosinophilic chronic obstructive pulmonary disease. N Engl J Med 2017; 377(17):1613-1629.
22. Criner G J, Celli B R, Brightling C E, et al. Benralizumab for the prevention of COPD exacerbations. N Engl J Med 2019; 381(11):1023-1034.
23. Maspero J, Adir Y, Al-Ahmad M, et al. Type 2 inflammation in asthma and other airway diseases. ERJ Open Res 2022; 8(3):00576-2021.
24. Lipson D A, Barnhart F, Brealey N, et al. Once-daily single-inhaler triple versus dual therapy in patients with COPD. N Engl J Med 2018; 378(18):1671-1680.
25. Rabe K F, Martinez F J, Ferguson G T, et al. Triple inhaled therapy at two glucocorticoid doses in moderate-to-very-severe COPD. N Engl J Med 2020; 383:35-48.

### Example 4. Dupilumab Efficacy in Patients with COPD and Type 2 Inflammation, and High Mortality Risk—BOREAS BODE Study

In phase 3 BOREAS (NCT03930732), dupilumab reduced exacerbations and improved lung function and quality of life in patients with moderate-to-severe COPD and type 2 inflammation (blood eosinophils ≥300 cells/μL).

### Rationale

The BOREAS study investigated the efficacy and safety profile of dupilumab over one year in patients with moderate-to-severe COPD with type 2 inflammation, as evidenced by blood eosinophils ≥300 cells/μL, who are in need of an additional treatment added to their current management of triple therapy with inhaled corticosteroids (ICS), long-acting β2-agonists (LABA), and long-acting muscarinic antagonists (LAMA) (or LABA/LAMA if ICS is contraindicated). As described in the example, dupilumab efficacy, stratified by baseline body-mass index, airflow obstruction, dyspnea, and exercise capacity (BODE) index ≤/>4, a predictor of 5-year mortality in COPD, was investigated.

### Methods

Study: BOREAS.

Population: ITT population.

Endpoints/Visit: Proportion of patients with baseline BODE 0-2, 3-4, 5-6, 7-10; Moderate or severe AECOPD by baseline BODE category (≤4, >4); LS mean difference in pre-BD FEV1 by baseline BODE category (≤4, >4).

Treatment arms: Dupilumab 300 mg q2w, placebo.

Patients received add-on dupilumab 300 mg or placebo every 2 weeks for 52 weeks alongside standard-of-care treatment. Endpoints were adjusted annualized rates of moderate or severe acute exacerbation of COPD and least squares (LS) mean differences of change from baseline at week 12 in pre-bronchodilator (BD) forced expiratory volume in 1 second (FEV1).

### Results

Of 469 patients receiving dupilumab, 278 had a BODE index of ≤4 and 186 had >4. Of 471 patients receiving placebo, 299 had BODE≤4, and 171 had BODE>4. Dupilumab vs. placebo reduced exacerbation rates by 28.2% (BODE≤4) and 34.4% (BODE>4). At week 12, dupilumab vs. placebo improved pre-BD FEV1 from baseline by 0.10 L (95% CI: 0.05, 0.15; BODE≤4) and 0.06 L (95% CI: −0.01, 0.14; BODE>4) (Table 18).

TABLE 18
Efficacy by Baseline BODE Scores (Placebo vs. Dupilumab)

| | | |
|---|---|---|
| | | Dupilumab |
| | Placebo | 300 mg q2w |
| Characteristic | (N = 471) | (N = 468) |

Adjusted Annualized rate of
moderate or severe COPD exacerbations

| BODE index ≤4 | n = 299 | | n = 278 | |
|---|---|---|---|---|
| Estimate (95% CI) | 0.879 | (0.684 to 1.129) | 0.631 | (0.482, 0.827) |
| Relative risk vs | | | 0.718 | (0.547, 0.944) |
| placebo (95% CI) | | | | |
| Risk reduction, % | | | −28.2% | |
| BODE index >4 | n = 171 | | n = 186 | |
| Estimate (95% CI) | 1.464 | (1.172 to 1.828) | 0.960 | (0.746, 1.234) |
| Relative risk vs | | | 0.656 | (0.496, 0.868) |
| placebo (95% CI) | | | | |

| | |
|---|---|
| Risk reduction, % | −34.4% |
| Overall P-value | P = 0.52 |
| for interaction | |

Change from baseline to Week 12
in pre-bronchodilator FEV₁ (L)

| BODE index ≤4 | n = 299 | | n = 277 | |
|---|---|---|---|---|
| LS mean (SE) | 0.05 | (0.02) | 0.15 | (0.02) |
| LS mean difference | | | 0.10 | (0.05, 0.15) |
| vs placebo (95% CI) | | | | |
| BODE index >4 | n = 171 | | n = 186 | |
| LS mean (SE) | 0.12 | (0.03) | 0.19 | (0.03) |
| LS mean difference | | | 0.06 | (−0.01, 0.14) |
| vs placebo (95% CI) | | | | |

| | |
|---|---|
| Overall P-value | P = 0.47 |
| for interaction | |

BODE, Body-Mass Index, Airflow Obstruction, Dyspnea, and Exercise Capacity; CI, Confidence Interval; COPD, chronic obstructive pulmonary disease; FEV₁, forced expiratory volume in 1 second; LS, least squares; SE, standard error. Adjusted annualized exacerbation rates and change from baseline to week 12 in pre-BD FEV1 in patients with moderate-to-severe COPD and type 2 inflammation, stratified by BODE index score.

### Conclusions

In patients with COPD and type 2 inflammation, dupilumab reduced exacerbation rates and improved lung function in patients, irrespective of baseline BODE scores, including those with high scores.

### Example 5. Dupilumab Efficacy and Safety in Patients with Moderate-To-Severe COPD with Type 2 Inflammation: Pooled Analysis of BOREAS and NOTUS Trials

BOREAS (NCT03930732) and NOTUS (NCT04456673) were 52-week, phase 3, randomized, double-blind, placebo-controlled trials that demonstrated dupilumab efficacy and safety data in patients with COPD and type 2 inflammation and high exacerbation risk.

### Rationale

The objective of this example was to evaluate the safety and efficacy of dupilumab in a pooled analysis combining both BOREAS and NOTUS.

### Methods

Study: Ph3 BOREAS and Ph3 NOTUS

Population: ITT.

Endpoints/Visit: annualized rate of moderate or severe exacerbations over 52-week treatment period; change in pre-BD FEV1 from baseline to week 12 vs placebo; change in pre-BD FEV1 from baseline to week 52 vs placebo; safety—TEAEs, SAEs, severe TEAEs, TEAEs leading to death, TEAEs leading to treatment discontinuation.

Treatment arms: dupilumab 300 mg q2w, placebo.

In BOREAS and NOTUS, patients with moderate-to-severe COPD and type 2 inflammation (blood eosinophils ≥300 cells/μL) on triple therapy (ICS+LABA+LAMA), or LABA/LAMA if ICS was contraindicated, received add-on dupilumab 300 mg q2w vs placebo for 52 weeks. The intention to treat (ITT) populations were pooled for the primary endpoint of annualized rate of moderate or severe exacerbations, key secondary endpoint of pre-BD FEV1 and safety.

### Results

In total, 1874 participants were randomized (936 to placebo and 938 to dupilumab). In the pooled population, there was a 31% reduction in the annualized rate of moderate-to-severe exacerbations (nominal P<0.0001, Table 19). At week 12, change from baseline in pre-BD FEV1 was greater with dupilumab (LS mean difference 83 mL, nominal P<0.0001) compared to placebo. This improvement was maintained at week 52 (LS mean difference 73 mL, nominal P<0.0001). Dupilumab was well tolerated. Treatment-emergent adverse events (TEAEs) were balanced between arms across both groups (Table 18).

TABLE 19
Efficacy and safety outcomes in the ITT population.

| | | | |
|---|---|---|---|
| | | Difference | |
| | | (95% CI)* for | |
| | | dupilumab | |
| | | (n = 939) | |
| Placebo | Dupilumab | vs placebo | |
| (N = 936) | (N = 938) | (n = 936) | P-value^{a} |

Efficacy: primary endpoint

| | | | | |
|---|---|---|---|---|
| Annualized rate | 1.156 | 0.794 | 0.687 | <0.0001 |
| of moderate or | | | (0.595 to 0.793) | |
| severe COPD | | | | |
| exacerbation | | | | |
| over 52-Week | | | | |
| treatment | | | | |
| period | | | | |

Efficacy: key secondary endpoints

| | | | | |
|---|---|---|---|---|
| Change in pre- | 0.064 | 0.147 | 0.083 | <0.0001 |
| BD FEV₁ | (0.013) | (0.013) | (0.053 to 0.112) | |
| from baseline | | | | |
| to Week 12 | | | | |
| compared to | | | | |
| placebo | | | | |
| Change in pre- | 0.059 | 0.133 | 0.073 | <0.0001 |
| bronchodilator | (0.015) | (0.015) | (0.040 to 0.107) | |
| FEV₁ from | | | | |
| baseline to | | | | |
| Week 52 | | | | |
| compared to | | | | |
| placebo^{b}, L | | | | |

| | |
|---|---|
| | Dupilumab |
| Placebo | 300 mg q2w |
| (n = 934) | (n = 938)^{c} |

Safety: treatment-emergent events, n (%)

| | | | | |
|---|---|---|---|---|
| Participants with | 663 | (71.0) | 676 | (72.1) |
| any TEAE | | | | |
| Participants with | 117 | (12.5) | 108 | (11.5) |
| any severe TEAE | | | | |
| Participants with any | 147 | (15.7) | 125 | (13.3) |
| treatment-emergent SAE | | | | |
| Participants with any | 15 | (1.6) | 19 | (2.0) |
| TEAE leading to death | | | | |
| Participants with any | 28 | (3.0) | 32 | (3.4) |
| TEAE leading to permanent | | | | |
| study intervention discontinuation | | | | |

*Difference was relative risk for the primary endpoint and LS mean difference for key secondary endpoints.
^{a}P-values shown were nominal.

### CONCLUSIONS

Dupilumab reduced moderate-to-severe exacerbations, improved lung function, and was well-tolerated, with safety consistent with the known safety profile in COPD patients with type 2 inflammation.

1. A method for treating a subject having chronic obstructive pulmonary disease (COPD) comprising administering to the subject an antibody or an antigen-binding fragment thereof that specifically binds interleukin-4 receptor (IL-4R), wherein the antibody or antigen-binding fragment thereof comprises three heavy chain complementarity determining region (CDR) sequences comprising SEQ ID NOs: 3, 4, and 5, respectively, and three light chain CDR sequences comprising SEQ ID NOs: 6, 7, and 8, respectively.

2-8. (canceled)

9. The method of claim 1, wherein the subject has:
a baseline blood eosinophil count of ≥300 cells/μL, ≥350 cells/μL, ≥400 cells/μL, ≥450 cells/μL, or ≥500 cells/μL;
a baseline blood eosinophil count of below 300 cells;
a baseline fractional exhaled nitric oxide (FeNO) level of ≥20 ppb, ≥25 ppb, ≥30 ppb, ≥35 ppb, or ≥40 ppb;
a baseline FeNO level of below 20 ppb;
a baseline immunoglobulin E level (IgE) of ≥100 kU/L;
a baseline IgE level below 100 kU/L;
oxygen-dependent COPD;
chronic bronchitis;
emphysema;
status as a current smoker; or
status as a former smoker, or any combinations thereof.

10-17. (canceled)

18. The method of claim 1, wherein the method comprises further a background therapy in addition to the antibody or an antigen-binding fragment thereof, optionally wherein:
the background therapy comprises an inhaled corticosteroid (ICS), a long-acting beta-agonist (LABA), a leukotriene receptor antagonist (LTRA), a long-acting muscarinic antagonist (LAMA), a methylxanthine, an inhibitor of phosphodiesterase that is optionally roflumilast or theophylline, or a mixture thereof; or
the background therapy comprises a LABA, a LAMA, and an ICS, and optionally the COPD is uncontrolled at baseline despite the background therapy alone;
the background therapy comprises a high dose of ICS;
the background therapy comprises a non-high dose of ICS;
the background therapy comprises a LABA and a LAMA, and optionally the COPD is uncontrolled at baseline despite the background therapy alone;
an inhaled corticosteroid (ICS) is contraindicated in the subject;
the background therapy comprises roflumilast or theophylline; or
one or more COPD-associated parameter(s) are improved in the subject.

19-28. (canceled)

29. The method of claim 1, wherein one or more COPD-associated parameter(s) are improved in the subject and are selected from the group consisting of:
(1) annualized rate of acute moderate or severe exacerbations of COPD (AECOPD);
(2) annualized rate of severe AECOPD;
(3) time to first moderate or severe AECOPD;
(4) forced expiratory volume in 1 second (FEV1) (pre-bronchodilator or post-bronchodilator);
(5) forced vital capacity (FVC);
(6) forced expiratory flow (FEF) 25%-75%;
(7) fractional exhaled nitric oxide (FeNO);
(8) Exacerbations of Chronic Obstructive Pulmonary Disease Tool (EXACT);
(9) St. George's Respiratory Questionnaire (SGRQ);
(10) Evaluating Respiratory Symptoms in COPD (E-RS:COPD);
(11) Body mass index, airflow Obstruction, Dyspnea, Exercise performance (BODE) index;
(12) Euro Quality of Life-5 Dimension Questionnaire (EQ-5D);
(13) Modified British Medical Research Council Questionnaire (mMRC);
(14) Health-Related Quality of Life Questionnaire (HRQoL);
(15) Courses in days of steroids, optionally systemic corticosteroids;
(16) Courses in days of an antibiotic;
(17) resting respiratory rate;
(18) FEV₁/FVC ratio;
(19) mucus plugging;
(20) blood eosinophil (Eos) count;
(21) serum immunoglobulin E (IgE) level;
(22) eotaxin level that is optionally eotaxin-3; and
(23) pulmonary and activation-regulated chemokine (PARC) level,
or any combination thereof.

30-31. (canceled)

32. The method of claim 1, wherein the antibody or antigen-binding fragment thereof is administered to the subject as an initial dose followed by one or more secondary doses, optionally wherein:
the initial dose is about 300 mg and the one or more secondary doses are each about 300 mg; or
the secondary doses are administered every other week (q2w).

33-34. (canceled)

35. The method of claim 1, wherein:
the subject is at least 40 years old;
prior to the start of treatment, the subject has a baseline Medical Research Council (MRC) Dyspnea Scale Grade score of ≥2 or a baseline Modified Medical Research Council (mMRC) Dyspnea Scale Grade score of ≥2;
the subject has a history of high exacerbation risk;
the subject has two or more severe exacerbations annually leading to hospital admission;
the treatment reduces the level of one or more biomarkers in the subject selected from the group consisting of blood eosinophil (Eos) count, fractional exhaled nitric oxide (FeNO) level, serum immunoglobulin E level (IgE), eotaxin (e.g., eotaxin-3) level, and pulmonary and activation-regulated chemokine (PARC) level; or
the method further comprises the step of determining the baseline level of a biomarker selected from the group consisting of PARC, eotaxin-3, FeNO (postbronchodilator), total IgE, fibrinogen, and any mixture thereof in the subject.

36-38. (canceled)

39. The method of claim 1, wherein the antibody or antigen-binding fragment thereof comprises a heavy chain variable region (HCVR) sequence of SEQ ID NO: 1 and a light chain variable region (LCVR) sequence of SEQ ID NO: 2.

40. The method of claim 39, wherein the antibody is dupilumab.

41. The method of claim 1, wherein:
the antibody or antigen-binding fragment thereof is administered using an autoinjector, a needle and syringe, a pen, or a prefilled device; or
the antibody or antigen-binding fragment thereof is administered subcutaneously.

42-44. (canceled)

45. A method of: improving one or more symptoms in a subject; reducing or preventing moderate to severe exacerbations in a subject; slowing the progression of lung function decline in a subject; improving health-related quality of life in a subject; or delaying time to first moderate or severe exacerbation in a subject, wherein the subject is selected from the group consisting of:
(1) a subject having chronic obstructive pulmonary disease (COPD);
(2) a subject having moderate-to-severe COPD;
(3) a subject having COPD with Type 2 inflammation;
(4) a subject having moderate-to-severe COPD with Type 2 inflammation;
(5) a subject having COPD not adequately controlled on background therapy;
(6) a subject having COPD with united airways disease (UAD); and
(7) a subject having COPD, wherein the COPD is comorbid with at least one Type 2 inflammatory disease, and
wherein the method comprises administering to the subject an antibody or an antigen-binding fragment thereof that specifically binds interleukin-4 receptor (IL-4R).

46-48. (canceled)

50. A method for treating a subject having uncontrolled chronic obstructive pulmonary disease (COPD) comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

51. The method of claim 50, wherein the subject is an adult.

52. The method of claim 50, wherein the subject receives double therapy or triple therapy in addition to the antibody or antigen-binding fragment thereof, optionally wherein:
the double therapy comprises treatment with a long-acting beta-agonist (LABA) and a long-acting muscarinic antagonist (LAMA); or
the triple therapy comprises treatment with an inhaled corticosteroid (ICS), a LABA, and a LAMA.

53. (canceled)

54. The method of claim 50, wherein ICS use is contraindicated, and wherein the subject receives double therapy in addition to the antibody or antigen-binding fragment thereof.

55. (canceled)

56. The method of claim 50, wherein the antibody or an antigen-binding fragment thereof is administered as:
an add-on treatment;
a maintenance treatment; or
an add-on maintenance treatment.

57-58. (canceled)

59. The method of claim 50, wherein the subject has a history of COPD exacerbations, or an elevated level of a biomarker of Type 2 inflammation relative to a control, wherein the biomarker is optionally blood eosinophil count.

60-61. (canceled)

62. The method of claim 50, wherein:
the subject has a baseline blood eosinophil count of ≥300 cells/μL, ≥350 cells/μL, ≥400 cells/μL, ≥450 cells/μL, or ≥500 cells/μL; or
the subject has a baseline blood eosinophil count of below 300 cells/μL.

63. (canceled)

64. The method of claim 50, wherein the antibody or antigen-binding fragment thereof comprises a heavy chain variable region (HCVR) sequence of SEQ ID NO: 1 and a light chain variable region (LCVR) sequence of SEQ ID NO: 2, or wherein the antibody is dupilumab.

65-85. (canceled)

86. The method of claim 50, wherein the subject has:
a baseline blood eosinophil count of ≥300 cells/μL, ≥350 cells/μL, ≥400 cells/μL, ≥450 cells/μL, or ≥500 cells/μL;
a baseline blood eosinophil count of below 300 cells;
a baseline fractional exhaled nitric oxide (FeNO) level of ≥20 ppb, ≥25 ppb, ≥30 ppb, ≥35 ppb, or ≥40 ppb;
a baseline FeNO level of below 20 ppb;
a baseline immunoglobulin E level (IgE) of ≥100 kU/L;
a baseline IgE level below 100 kU/L;
oxygen-dependent COPD;
chronic bronchitis;
emphysema;
status as a current smoker; or
status as a former smoker, or any combinations thereof.

87. (1) A method for treating a subject having moderate-to-severe chronic obstructive pulmonary disease (COPD) comprising administering to the subject an antibody or an antigen-binding fragment thereof that specifically binds interleukin-4 receptor (IL-4R);
(2) a method for treating a subject having chronic obstructive pulmonary disease (COPD) with Type 2 inflammation comprising administering to the subject an antibody or an antigen-binding fragment thereof that specifically binds interleukin-4 receptor (IL-4R);
(3) a method for treating a subject having moderate-to-severe chronic obstructive pulmonary disease (COPD) with Type 2 inflammation comprising administering to the subject an antibody or an antigen-binding fragment thereof that specifically binds interleukin-4 receptor (IL-4R);
(4) a method for treating a subject having chronic obstructive pulmonary disease (COPD) not adequately controlled on background therapy, comprising administering to the subject an antibody or an antigen-binding fragment thereof that specifically binds interleukin-4 receptor (IL-4R);
(5) a method for treating a subject having chronic obstructive pulmonary disease (COPD) with united airways disease (UAD) comprising administering to the subject an antibody or an antigen-binding fragment thereof that specifically binds interleukin-4 receptor (IL-4R); or
(6) a method for treating a subject having chronic obstructive pulmonary disease (COPD), wherein the COPD is comorbid with at least one Type 2 inflammatory disease, comprising administering to the subject an antibody or an antigen-binding fragment thereof that specifically binds interleukin-4 receptor (IL-4R).
